(19) United States
(12) Patent Application Publication
(10) Pub. No.: US 2025/0051393 A1
(43) Pub. Date: Feb. 13, 2025

(54) COMPOSITIONS AND METHODS FOR MODULATING INTERFERON REGULATORY FACTOR-5 (IRF-5) ACTIVITY

(71) Applicant: ENTRADA THERAPEUTICS, INC., Boston, MA (US)

(72) Inventors: Ziqing QIAN, Wellesley, MA (US); Natarajan SETHURAMAN, Hanover, NH (US); Mahboubeh KHEIRABADI, Natick, MA (US); Haoming LIU, Canton, MA (US); Xiulong SHEN, Waltham, MA (US); Xiang LI, Belmont, MA (US)

(21) Appl. No.: 18/289,943

(22) PCT Filed: May 9, 2022
(86) PCT No.: PCT/US2022/028277

§ 371 Date: Nov. 8, 2023

Related U.S. Application Data
(60) Provisional application No. 63/186,664, filed on May 10, 2021.
(60) Provisional application No. 63/210,866, filed on Jun. 15, 2021.
(60) Provisional application No. 63/239,671, filed on Sep. 1, 2021.
(60) Provisional application No. 63/298,587, filed on Jan. 11, 2022.
(60) Provisional application No. 63/318,201, filed on Mar. 9, 2022.
(60) Provisional application No. 63/362,295, filed on Mar. 31, 2022.

(51) Int. Cl. C07K 7/64; A61K 47/64; C12N 15/113

(52) U.S. Cl. CPC C07K 7/64; A61K 47/6455; C12N 15/113; C12N 2310/11; C12N 2310/3233; C12N 2310/3513; C12N 2320/33

(57) ABSTRACT

Compounds that include a cyclic cell penetrating peptide and a therapeutic moiety modulates Interferon Regulatory Factor-5 (IRF-5) activity in an immune cell. The therapeutic moiety is a therapeutic protein, a small molecule, or an antisense compound. Methods include administering the compound to cells or subjects to modulate IRF-5 activity. Methods include treating diseases associated with IRF-5 using the compounds.

### CROSS REFERENCE TO RELATED APPLICATIONS

This application claims the benefit of U.S. Provisional Application Ser. No. 63/186,664, filed on May 10,2021; 63/210,866, filed on Jun. 15, 2021; 63/298,587, filed on Jan. 11, 2022; 63/318,201, filed on Mar. 9, 2022; 63/362,295, filed on Mar. 31, 2022; and 63/239,671, filed on Sep. 1, 2021, which are incorporated by reference herein in their respective entireties.

### FIELD OF THE INVENTION

Provided herein are compositions and methods for modulating Interferon Regulatory Factor-5 (IRF-5) activity, in particular, compositions and methods are provided for downregulating IRF-5 activity.

### BACKGROUND

Type I interferons (IFNs) play a key role in autoimmune diseases. Increased expression of IFN-induced genes is associated with autoimmune diseases such as systemic lupus erytheniatosus (SLE), rheumatoid arthritis (RA), Sjögren's syndrome and multiple sclerosis (MS) (Kristjansdottir et al. J. Med. Genet. (2008), 45:362-369.

Interferon regulatory factors (IRFs) are a family of transcription factors that regulate genes activated by Type I interferons and are involved in both innate and adaptive immunity. There are currently 9 known mammalian IRFs-IRF1, UF2, IRF3, IRF4/PIP/ICSAT, IRE-5, IRF6, IRF7, IRF8/ICSBP, and IRF9/p48/ISGF3γ (Thompson et al., Front. Immunol. (2018) doi.org/10.3389/fimmu.2018.02622). The RFs have a conserved N-terminal DNA-binding domain (DBD), which recognizes a core DNA sequence within interferon-stimulated response elements and a C-terminal IRF-associated domain (IAD), which mediates protein-protein interactions between FIRs and other proteins to form transcriptional complexes (Almuttaqi and Udalova, FEBS J. (2019), 286:1624-1637). Dysregulation of IRFs can lead to either suppression or hyperactivation, either of which may contribute to disease development (Ibid).

IRF-5 mediates induction of proinflammatory cytokines such as interleukin-6 (11-6), IL-12, and tumor necrosis factor-alpha (TNF-α). In humans, IRF-5 exists as multiple distinct isoforms that are generated by alternatively spliced transcripts that show cell-type specific expression, subcellular localization, and function (Almuttaqi and Udalova, FEBS J, (2019), 286:1624-1637).

IRF-5 upregulation and polymorphisms have been implicated in the development of numerous inflammatory and autoimmune diseases, including, but not limited to, Rheumatoid Arthritis (RA), systemic sclerosis, multiple sclerosis (IS), inflammatory bowel disease (IBD), systemic lupus erythematosus (SLE) and Sjörgens syndrome (Thompson et al., Front. Immunol. (2018), doi.org/10.3389/fimmu.2018.02622; Almuttaqi and Udalova, FEBS J. (2019), 286:1624-1637.

There remains an unmet need for effective compositions and methods for treating autoimmune and inflammatory diseases associated with IRE-5 activity.

### SUMMARY

Compounds, compositions, and methods for modulating Interferon Regulatory Factor-5 (IRF-5) level and/or activity are described herein. In embodiments, this disclosure relates to compounds that include a therapeutic moiety (TM) and a cell penetrating peptide (CPP), such as a cyclic CPP (cCPP). The TM modulates IRF-5 level and/or activity. In embodiments, the TM is an IRF-5 inhibitor. The cCPP facilitates intracellular localization of the TM. The compounds may comprise an endosomal escape vehicle (EEV). The EEV may be conjugated to or chemically linked to the TM. The EEV may comprise the cCPP.

In embodiments, the TM decreases IRE-5 activity or reduces the level of IRF-5. In embodiments, the TM modulates IRF-5 expression. In embodiments, the TM decreases IRF-5 expression. In embodiments, the TM inhibits activation of IRF-5 In embodiments, the IRE-5 inhibits phosphorylation of IRE-5. In embodiments, the TNT inhibits ubiquitination of IRE-5. In embodiments, the TM inhibits nuclear localization of IRF-5. In embodiments, the TM inhibits DNA-binding by IRE-5. In embodiments, the TM inhibits IRE-5 dimer formation.

In embodiments, the TM comprises an antisense compound (AC). The AC may modulate expression of IRF-5. In embodiments, the AC decreases IRF-5 expression. In embodiments, the AC modulates polyadenylation of an IRF-5 transcript. In embodiments, the AC modulates splicing of an IRF-5 transcript.

In embodiments, the cCPP is of Formula (A):

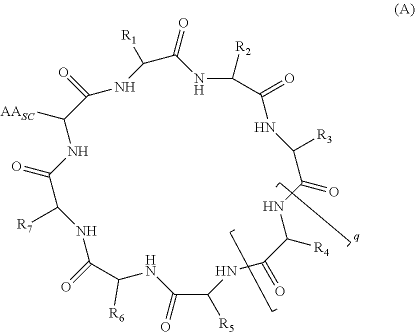

or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the cCPP is of Formula (A) is of Formula (I):

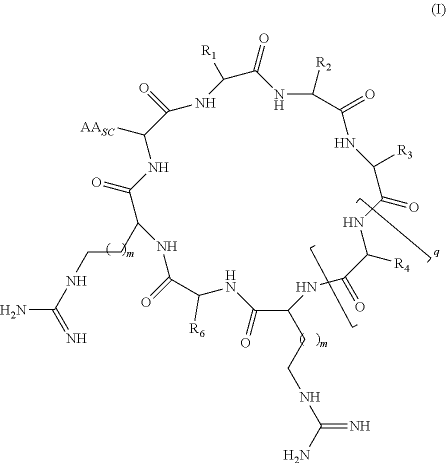

or a protonated form or salt thereof,
wherein each m is independently an integer from 0-3.

In embodiments, the cCPP is of Formula (A) is of Formula (I-1):

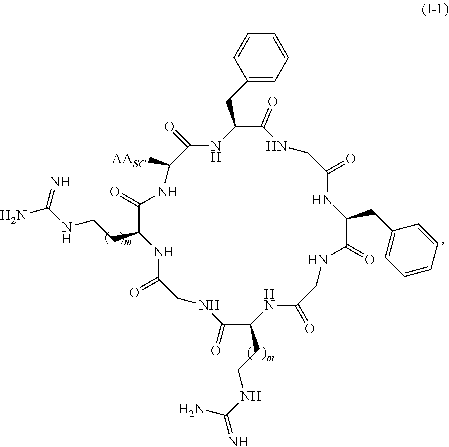

or a protonated form or salt thereof.

In embodiments, the cCPP is of Formula (A) is of Formula (I-2):

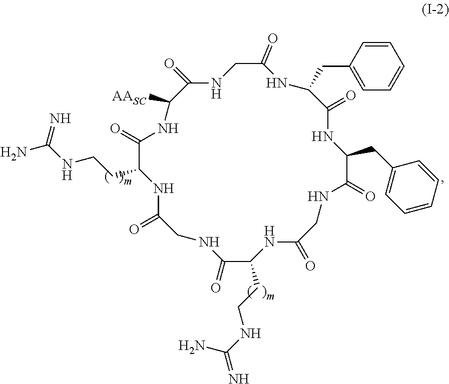

or a protonated form or salt thereof.

In embodiments, the cCPP is of Formula (A) is of Formula (I-3):

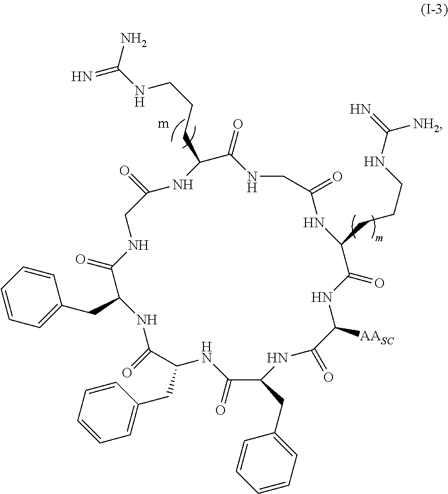

or a protonated form or salt thereof.

In embodiments, the cCPP is of Formula (A) is of Formula (I-4):

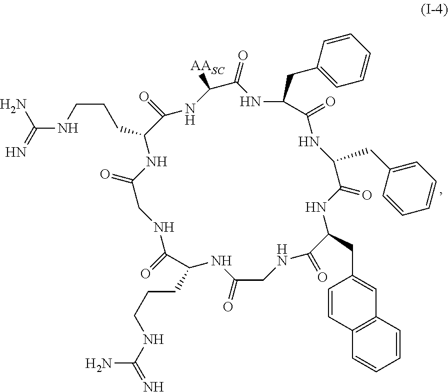

or a protonated form or salt thereof.

In embodiments, the cCPP is of Formula (A) is of Formula (I-5):

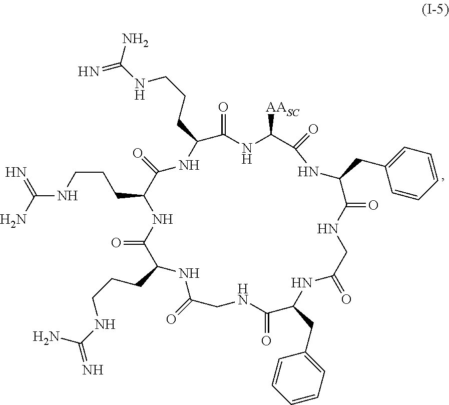

or a protonated form or salt thereof.

In embodiments the cCPP is of Formula (A) is of Formula (I-6):

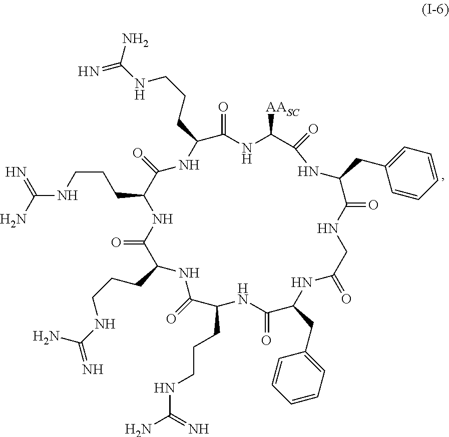

or a protonated form or salt thereof.

In embodiments, the cCPP is of Formula (II):

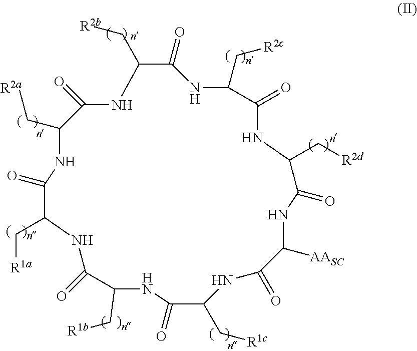

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

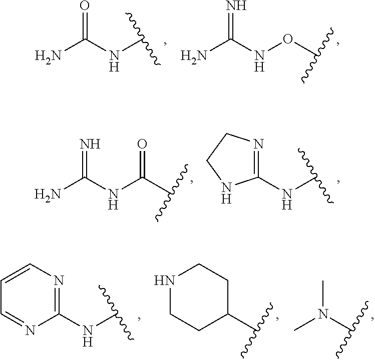

or a protonated form or salt thereof;
at least one of R^{2a}, R^{2b}, R^{2c} and R^{2d} is guanidine or a protonated form or salt thereof;
each n″ is independently an integer from 0 to 5;
each n′ is independently an integer from 0 to 3; and
if n′ is 0 then R^{2a}, R^{2b}, R^{2b} or R^{2d} is absent.

In embodiments, the cCPP of Formula (II) is of Formula (II-1):

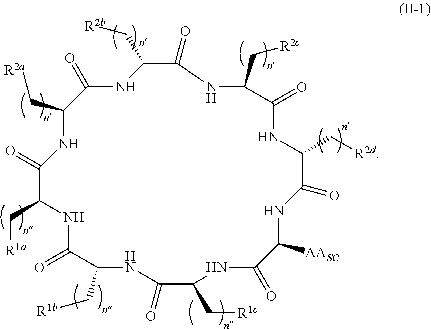

In embodiments, the cCPP of Formula (II) is of Formula (IIa):

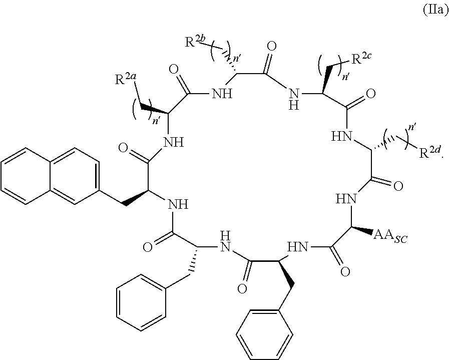

In embodiments, the cCPP of Formula (II) is of Formula (IIb):

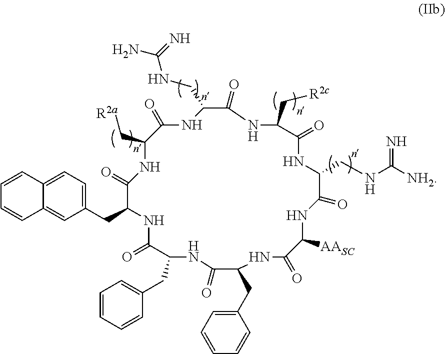

In embodiments, the cCPP of Formula (II) is of Formula (IIc):

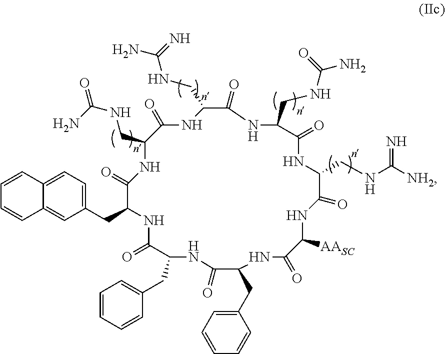

or a protonated form or salt thereof.

In embodiments, the cCPP has the structure:

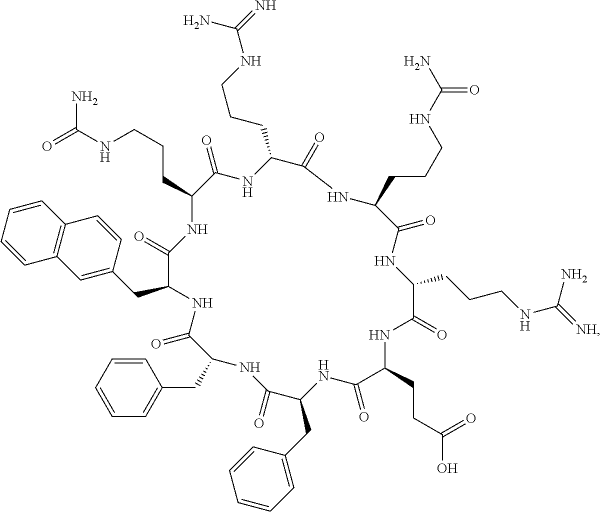 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

or a protonated form or salt thereof, wherein at least one atom of an amino acid side chain is replaced by the therapeutic moiety or a linker or at least one lone pair forms a bond to the therapeutic moiety or the linker.

In embodiments, the cCPP has the structure:

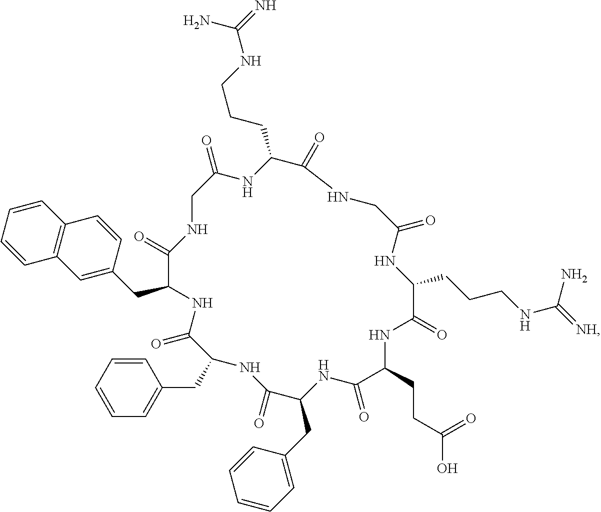 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

or a protonated form or salt thereof, wherein at least one atom of an amino acid side chain is replaced by the therapeutic moiety or a linker or at least one lone pair forms a bond to the therapeutic moiety or the linker.

In embodiments, the compound comprises an exocyclic peptide (EP). In embodiments, the EP comprises one of the following sequences: KK, KR, RR, HH, HK, HR, RH, KKK, KGK, KBK, KBR, KRK, KRR, RKK, RRR, KKH, KHK, HKK, HRR, HRH, HR, HBH, HHH, HHHH KHKK, KKHK, KKKH, KHKH, HKHK, KKKK, KKRK, KRKK, KRRK, RKKR, RRRR, KGKK, KKGK, HBHBH, JBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine.

In embodiments, the compound is of Formula (C):

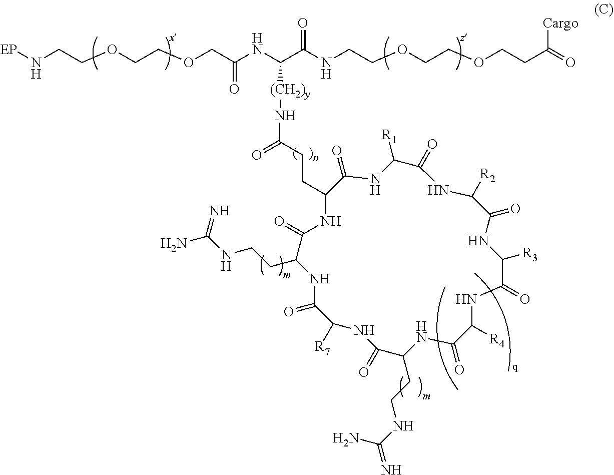

or a protonated form or salt thereof, wherein:
R₁, R₂, and R₃ are each independently H or a side chain comprising an aryl or heteroaryl group, wherein at least one of R₁, R₂, and R₃ is a side chain comprising, an aryl or heteroaryl group;
R₄ and R₇ are independently H or an amino acid side chain;
EP is an exocyclic peptide;
each m is independently an integer from 0-3;
n is an integer from 0-2;
x′ is an integer from 1-23;
y is an integer from 1-5;
q is an integer from 1-4;
z′ is an integer from 1-23, and
Cargo is the therapeutic moiety.

In embodiments, the compound comprises the structure of Formula (C-1), (C-3), or (C-4):

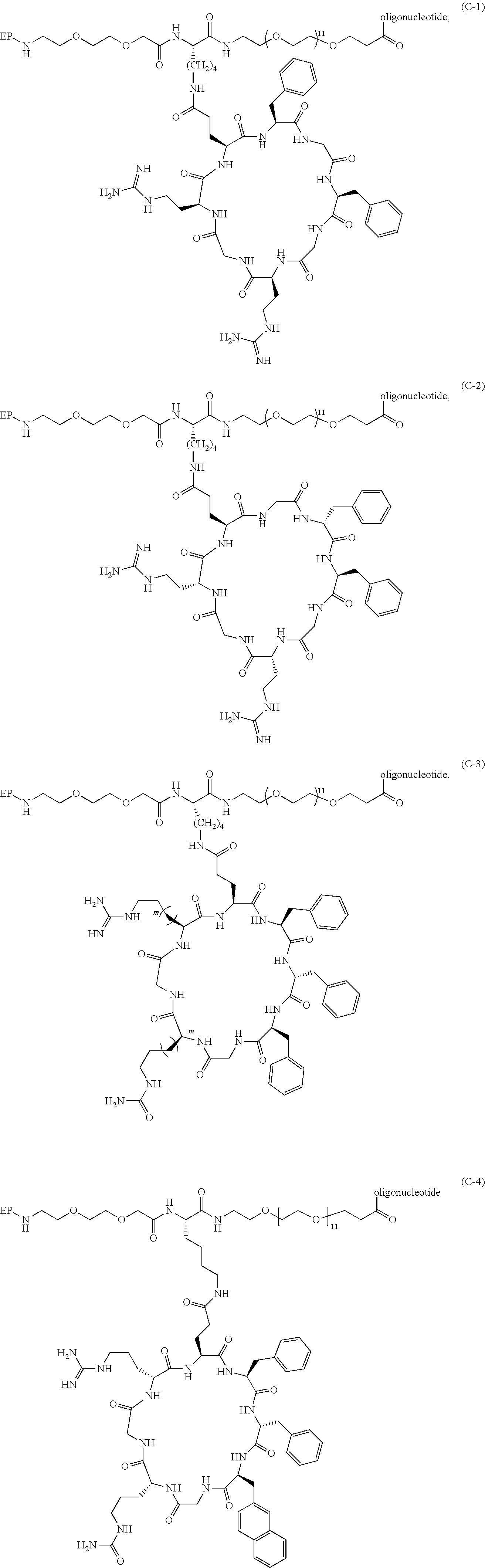

or a protonated form or salt thereof, wherein EP is an exocyclic peptide, and oligonucleotide is the therapeutic moiety.

### BRIEF DESCRIPTION OF THE DRAWINGS

FIG. 1 shows modified nucleotides used in antisense oligonucleotides described herein. Structures 1-3 (1=Phosphorothioate; 2=(S_{C5}—Rₚ)-α,β-CAN; 3=PMO) are phosphate backbone modifications; 4 (2-thio-dT) is a base modification; 5-8 (5=2′-OMe-RNA; 6=2′O-MOE-RNA; 7=2′F-RNA; 8=2′F-ANA) are 2′ sugar modifications; 9-11 are constrained nucleotides; 12-14 (9=LNA; 10=(S)-cET; 1=tcDNA; 12=FHNA; 13=(S)5′-C-methyl; 14=UNA) are additional sugar modification; and 15-18 (15=E-VP; 16=Methyl phosphonate; 17=5′ phosphorothioate; 18=(S)-5′-C-methyl with phosphate) are 5′ phosphate stabilization modifications; 19 is a morpholino sugar. Reformatted from Khvorova, A., et al., Nat. Biotechnol. 2017 March; 35(3): 238-248.

FIGS. 2A-D provide structures of the adenine (A), cytosine (B), guanine (C), and thymine (D) morpholino subunit monomers used in synthesizing phosphorodiamidate-linked morpholino oligomers (PMOs).

FIGS. 3A-D illustrate conjugation chemistries for connecting AC to a cyclic cell penetrating peptide. FIG. 3A shows the amide bond formation between peptides with a carboxylic acid group or with TFP activated ester and primary amine residues at the 5′ end of AC. FIG. 3B shows the conjugation of secondary amine or primary amine modified AC at 3′ and peptide-TFP ester through amide bond formation. FIG. 3C shows the conjugation of peptide-azide to the 5′ cyclooctyne modified AC via copper-free azide-alkyne cycloaddition. FIG. 3D demonstrates another exemplary conjugation between 3′ modified cyclooctyne ACs or 3′ modified azide ACs and CPP containing linker-azide or linker-alkyne/cyclooctyne moiety, via a copper-free azide-alkyne cycloaddition or cupper catalyzed azide-alkyne cycloaddition, respectively (click reaction).

FIG. 4 shows the conjugation chemistry for connecting AC and CPP with an additional linker modality containing a polyethylene glycol (PEG) moiety.

FIGS. 5A-B are a schematic drawing showing splicing elements and splicing regulatory elements (A), and the general splicing reactions (two transesterification reactions) (B).

FIG. 6 is a schematic drawing showing antisense compound mediated exon skipping to create a premature termination codon and lead to nonsense mediated decay of the target transcript.

FIG. 7 is a schematic representation of RNA before and after cleavage and addition of the poly(A) tail showing the location of the polyadenylation signal (PAS), the cleavage site ((S), and the downstream element (DSE) and the intervening sequence (IS) between the PAS and the CS.

FIG. 8A-8B are schematics showing the components of an IgG antibody, F(ab), Fv fragment, scFv, sdAb, and F(ab)2 antigen binding fragments. FIG. 8B is a schematic of a camelid antibody and a sdAb thereof. FIG. 8C is a schematic representation of a degradation construct and the mechanisms of action of a degradation construct.

FIGS. 9A and 9B are graphs showing the in vivo distribution in the liver (A) and kidney (B) of the vehicle (G1), compound 1 (G2), compound 2 (G4), and compound 3 (G3) of mice 24 hours post-dosing.

FIG. 10 shows the IRF-5 expression levels in THP1 cells transfected with various PMOs targeting the polyadenylation sequence (PAS) of IFR-5. NT is no nucleofection and no treatment/no nucleic acid, and nucleofection is nucleofected but not treated.

FIG. 11A show the IRF-5 expression levels RAW 264.7 Monocyte/Macrophage cells after treatment with various concentrations of PMO-EEVs 277-1120 and 278-1120. P≥0,05=NS; P≤0.05=*; P≤0.01=**; P≤0.001=***.

FIG. 11B is a bar graph of exon skipping percentage at various time points after RAW 264.7 Monocyte/Macrophage cells were treated with EEV-PMO 278-1120. NT=No treatment.

FIGS. 12A-B are a bar graphs showing the levels of IRF-5 expression (A) and exon 4 skipping percentage (B) in RAW 264.7 Monocyte/Macrophage cells after treatment with various EEV-PMOs at various concentrations followed by R848 stimulation.

FIGS. 13A-B are plots show the IRF-5 exon 4 and exon 5 skipping levels in human THP1 cells after treatment with the various EEV-PMOs at various concentrations.

FIGS. 14A-C show the expression levels of IRF-5 in mouse TiA tissue (A), liver tissue (B), and small intestine tissue (C), after mice were treated with two doses of a PMO or EEV-PMO.

FIG. 15A-C show the IRF-5 expression levels in mouse liver (A), kidney (B), and tibialis anterior (C) tissue after mice were treated with one dose of PMO 278 or PMO-EEV 278-1120. P>0.05=NS; P≤0.05=*; P≤0.01=**; P≤0.001=***. MPK=mg per kg.

FIG. 16A-C are plots showing the level of IRF-5 expression the liver (A), small intestine (B), and tibialis anterior (C) of mice treated with various concentrations of an EEV-PMO. (P>0.05=NS; P≤0.05=*; P≤0.01=**; P≤0.001=***).

FIG. 17A-B are plots showing the level of IRF-5 expression in an in vitro experiment where mouse macrophage cells were treated with various concentrations of EE #1-PMO and either not stimulated with R848 (A) or stimulated with R848 (B). (P>0.05=NS; P≤0.05=*; P≤0.01=*; P≤0,001=***).

### DETAILED DESCRIPTION

### Interferon Regulatory Factor-5 (IRF-5)

In embodiments, a compound is provided for modulating the level and/or activity of Interferon Regulatory Factor-5 (IRF-5). In embodiments, a compound is provided for inhibiting the activity of IRF-5 or reducing the level of IRF-5. The compounds described herein include a cyclic cell penetrating peptide (cCPP) and a therapeutic moiety (TM). The cCPP can enhance intracellular delivery of the TM. The TM may modulate in any suitable manner. In embodiments, the TM comprises a small molecule, a polypeptide, or antisense compound that modulates IRF-5 level and/or activity. In embodiments, the TM is an IRF-5 inhibitor.

IRF-5 is a member of the IRF family of transcription factors and is involved in innate and adaptive immunity, macrophage polarization, cell growth regulation and differentiation, antiviral defense, the production of proinflammatory cytokines, and apoptosis.

IRF-5 exists in multiple isoforms that are generated by three alternative non-coding 5′ exons and at least nine alternatively spliced mRNAs. The sequences for the IR-5 isoforms are publicly available, for example, through the online UniProt database. The isoforms show cell-type specific expression, subcellular localization and function. Some isoforms are associated with risk of autoimmune disease. For example, Isoform 2 is linked to overexpression of IRF-5 and susceptibility to autoimmune disease such as systemic lupus erythematosus.

The gene encoding IRF-5 includes 9 exons (exon 1, exon 2, exon 3, exon 4, exon 5, exon 6, exon 7, exon 8, and exon 9. Exon 1 is in the 5′-untranslated region (5′-UTR) and has three variants, exon IA, exon 1B, exon 1C, and exon 1D. The predominant isoform includes Exon 1A. Exon 1B is associated with IRF-5 hyperactivation and disease progression. Single-nucleotide polymorphisms (SNP) (e.g., rs2004640) that introduce a donor splice site can lead to increased expression of Exon 1B transcripts and reduced expression of Exon 1C-derived transcripts. Other SNPs (e.g., rs2280714) are also associated with elevated IRF-5 expression (Kozyrev et al., Arthritis and Rheumatology. (2007), 56(4):1234-1241).

In embodiments, a TM modulates level and/or activity of one or more isoform of IRF-5. In embodiments, the TM modulates level and/or activity of IRF-5 having an amino acid sequence of IRF-5 Isoform 1. In embodiments, the TM modulates level and/or activity of IRF-5 having an amino acid sequence of IRF-5 Isoform 2. In embodiments, the TM modulates level and/or activity of IRF-5 having an amino acid sequence of IRF-5 Isoform 3. In embodiments, the TM modulates level and/or activity of IRF-5 having an amino acid sequence of IRF-5 Isoform 4. In embodiments, the TM modulates level and/or activity of IRF-5 having an amino acid sequence of IRF-5 Isoform 5. In embodiments, the TM modulates level and/or activity of IRF-5 having an amino acid sequence of IRF-5 Isoform 6. In embodiments, the TM modulates level and/or activity of IRF-5 having an amino acid that differs by one or more amino acids of IRF-5 Isoform 1, IRF-5 Isoform 2, IRF-5 Isoform 3, IRF-5 Isoform 4, IRF-5 Isoform 5, or IRF-5 Isoform 6. In embodiments, the TM modulates level and/or activity of IRF-5 having an amino acid sequence that shares 100% identity to an amino acid sequence of IRF-5 Isoform 1, IRF-5 Isoform 2, IRF-5 Isoform 3, IRF-5 Isoform 4, IRF-5 Isoform 5, or IRF-5 Isoform 6. In embodiments, the TM modulates level and/or activity of IRF-5 having an amino acid sequence that shares less than 100% identity to an amino acid sequence of IRF-5 Isoform 1, IRF-5 Isoform 2, IRF-5 Isoform 3, IRF-5 Isoform 4, IRF-5 Isoform 5, or IRF-5 Isoform 6. In embodiments, the TM modulates level and/or activity of IRF-5 having an amino acid sequence that is at least about 80%, about 85%, about 90%, about 91%, about 92%, about 93%, about 94%, about 95%, about 96%, about 97%, about 98%, or about 99% identical to an amino acid sequence of IRF-5 Isoform 1, IRF-5 Isoform 2, IRF-5 Isoform 3, IRF-5 Isoform 4, IRF-5 Isoform 5, or IRF-5 Isoform 6.

The sequences of IRF-5 Isoform 1, IRF-5 Isoform 2, IRF-5 Isoform 3, IRF-5 Isoform 4, IRF-5 Isoform 5, or IRF-5 Isoform 6 are provided below:

HUMAN Interferon regulatory factor-
5 (IRF-S) (Isoform 1)
(SEQ ID NO: 151)
MNQSIPVAPTPPRRVRLKPWLVAQVNSCQYPGLQWVNGEKKLFCI
PWRHATRHGPSQDGDNTIFKAWAKETGKYTEGVDEADPAKWKANL
RCALNKSRDFRLIYDGPRDMPPQPYKIYEVCSNGPAPTDSQPPED
YSFGAGEEEEEEEELQRMLPSLSLTEDVKWPPTLQPPTLRPPTLQ
PPTLQPPVVLGPPAPDPSPLAPPPGNPAGFRELLSEVLEPGPLPA
SLPPAGEQLLPDLLISPHMLPLTDLEIKFQYRGRPPRALTISNPH
GCRLFYSQLEATQEQVELFGPISLEQVRFPSPEDIPSDKQRFYTN
QLLDVLDRGLILQLQGQDLYAIRLCQCKVFWSGPCASAHDSCPNP
IQREVKTKLFSLEHFLNELILFQKGQTNTPPPFEIFFCFGEEWPD
RKPREKKLITVQVVPVAARLLLEMESGELSWSADSIRLQISNPDL
KDRMVEQFKELHHIWQSQQRLQPVAQAPPGAGLGVGQGPWPMHPA
GM,
HUMAN Interferon regulatory factor-
5 (IRF-5) (Isoform 2)
(SEQ ID NO: 152)
MNQSIPVAPTPPRRVRLKPWLVAQVNSCQYPGLQWVNGEKKLFCI
PWRHATRHGPSQDGDNTIFKAWAKETGKYTEGVDEADPAKWKANL
RCALNKSRDFRLIYDGPRDMPPQPYKIYEVCSNGPAPTDSQPPED
YSFGAGEEEEEEEELQRMLPSLSLTDAVQSGPHMTPYSLLKEDVK
WPPTLQPPTLRPPTLQPPTLQPPVVLGPPAPDPSPLAPPPGNPAG
FRELLSEVLEPGPLPASLPPAGEQLLPDLLISPHMLPLTDLEIKF
QYRGRPPRALTISNPHGCRLFYSQLEATQEQVELFGPISLEQVRF
PSPEDIPSDKQRFYTNQLLDVLDRGLILQLQGQDLYAIRLCQCKV
FWSGPCASAHDSCPNPIQREVKTKLFSLEHFLNELILFQKGQTNT
PPPFEIFFCFGEEWPDRKPREKKLITVQVVPVAARLLLEMFSGEL
SWSADSIRLQISNPDLKDRMVEQFKELHHIWQSQQRLQPVAQAPP
GAGLGVGQGPWPMHPAGMQ,
HUMAN Interferon regulatory factor-
5 (IRF-5) (Isoform 3)
(SEQ ID NO: 153)
MNQSIPVAPTPPRRVRLKPWLVAQVNSCQYPGLQWVNGEKKLFCI
PWRHATRHGPSQDGDNTIFKAWAKETGKYTEGVDEADPAKWKAN
LRCALNKSRDFRLIYDGPRDMPPQPYKIYEVCSNGPAPTDSQPPE
DYSFGAGEEEEEEEELQRMLPSLSLIDAVQSGPHMTPYSLLKEDV
KWPPTLQPPTLQPPVVLGPPAPDPSPLAPPPGNPAGFRELLSEVL
EPGPLPASLPPAGEQLLPDLLISPHMLPLTDLEIKFQYRGRPPRA
LTISNPHGCRLFYSQLEATQEQVELFGPISLEQVRFPSPEDIPSD
KQRFYTNQLLDVLDRGLILQLQGQDLYAIRLCQCKVFWSGPCASA
HDSCPNPIQREVKTKLFSLEHELNELILFQKGQTNTPPPFEIFFC
FGEEWPDRKPREKKLITVQVVPVAARLLLEMESGELSWSADSIRL
QISNPDLKDRMVEQFKELHHIWQSQQRLQPVAQAPPGAGLGVGQG
PWPMHPAGMQ,
HUMAN Interferon regulatory factor-
5 (IRF-5) (Isoform 4)
(SEQ ID NO: 154)
MNQSIPVAPTPPRRVRLKPWLVAQVNSCQYPGLQWVNGEKKLFCI
PWRHATRHGPSQDGDNTIFKAWAKETGKYTEGVDEADPAKWKANL
RCALNKSRDFRLIYDGPRDMPPQPYKIYEVCSNGPAPTDSQPPED
YSFGAGEEEEEEEELQRMLPSLSLTEDVKWPPTLQPPTLQPPVVL
GPPAPDPSPLAPPPGNPAGFRELLSEVLEPGPLPASLPPAGEQLL
PDLLISPHMLPLTDLEIKFQYRGRPPRALTISNPHGCRLFYSQLE
ATQEQVELFGPISLEQVREPSPEDIPSDKQRFYTNQLLDVLDRGL
ILQLQGQDLYAIRLCQCKVFWSGPCASAHDSCPNPIQREVKIKLF
SLEHFLNELILFQKGQTNTPPPFEIFFCFGEEWPDRKPREKKLIT
VQVVPVAARLLLEMESGELSWSADSIRLQISNPDLKDRMVEQFKE
LHHIWQSQQRLQPVAQAPPGAGLGVGQGPWPMHPAGMQ,
HUMAN Interferon regulatory factor-
5 (IRF-S) (Isoform 5)
(SEQ ID NO: 155)
MNQSIPVAPTPPRRVRLKPWLVAQVNSCQYPGLQWVNGEKKLFCI
PWRHATRHGPSQDGDNTIFKAWAKETGKYTEGVDEADPAKWKANL
RCALNKSRDFRLIYDGPRDMPPQPYKIYEVCSNGPAPTDSQPPED
YSFGAGEEEEEEEELQRMLPSLSLTVIDLEIKFQYRGRPPRALTI
SNPHGCRLFYSQLEATQEQVELEGPISLEQVREPSPEDIPSDKQR
FYTNQLLDVLDRGLILQLQGQDLYAIRLCQCKVFWSGPCASAHDS
CPNPIQREVKTKLFSLEHFLNELILFQKGQTNTPPPFEIFFCFGE
EWPDRKPREKKLITVQVVPVAARLLLEMFSGELSWSADSIRLQIS
NPDLKDRMVEQFKELHHIWQSQQRLQPVAQAPPGAGLGVGQGPWP
MHPAGMQ,
and
HUMAN Interferon regulatory factor-
5 (IRF-5) (Isoform 6)
(SEQ ID NO: 156)
MNQSIPVAPTPPRRVRLKPWLVAQVNSCQYPGLQWVNGEKKLFCI
PWRHATRHGPSQDGDNTIFKAWAKETGKYTEGVDEADPAKWKANL
RCALNKSRDFRLIYDGPRDMPPQPYKIYETPSPIRITLLVQERRR
KKRKSCRGCCQA.

In embodiments, the TM modulates expression of IRF-5 of a gene or transcript encoding one or more IRF-5 isoform. In embodiments, the TM modulates expression of IRF-5 of a gene or transcript encoding IRF-5 that differs by one or more nucleic acids from a nucleotide sequence encoding IRF-5 Isoform 1, IRF-5 Isoform 2, IRF-5 Isoform 3, IRF-5 Isoform 4, IRF-5 Isoform 5, or IRF-5 Isoform 6. the TM modulates expression of IRF-5 of a gene or transcript encoding IRF-5 that differs by one or more polymorphisms (e.g., Single Nucleotide Polymorphisms or SNPs). the TM modulates expression of IRF-5 of a gene or transcript encoding IRF-5 that shares less than 100% sequence identity with a nucleotide sequence encoding IRF-5 Isoform 1, IRF-5 Isoform 2, IRF-5 Isoform 3, IRF-5 Isoform 4, IRF-5 Isoform 5, or IRF-5 Isoform 6. In embodiments, IRF-5 is encoded by nucleotide sequence that is at least about 80%, about 85%, about 90%, about 91%, about 92%, about 93%, about 94%, about 95%, about 96%, about 97%, about 98%, or about 99% identical to a nucleic acid sequence encoding IRF-5 Isoform 1, IRF-5 Isoform 2, IRF-5 Isoform 3, IRF-5 Isoform 4, IRF-5 Isoform 5, or IRF-5 Isoform 6. the TM modulates expression of IRF-5 of a gene or transcript encoding IRF-5 that is encoded by nucleotide sequence that is 80% to 100%, 90% to 100%, 95% to 100%, or 99% to 100% identical to a nucleic acid sequence encoding IRF-5 Isoform 1, IRF-5 Isoform 2, IRF-5 Isoform 3, IRF-5 Isoform 4, IRF-5 Isoform 5, or IRF-5 Isoform 6

IRF-5 includes a N-terminal DNA-binding domain (DBD), found in all IRF family members, a C-terminal IRF association domain (IAD), and a serine-rich domain located at the C terminus. In embodiments, the TM affects the DBD, IAD, and/or the serine-rich domain located at the C terminus of IRF-5.

IRF-5 also includes an N-terminal nuclear localization signal (NLS) and a C-terminal NLS, which are responsible for nuclear localization of IRF-5, which may be important for transcriptional modulation of target gene expression by IRF-5 (Barnes et al., Mol. Cell Biol. (2022), 22(16):5721-5740. In embodiments, the TM affects the N-terminal NLS and/or the C-terminal NLS of IRF-5.

IRF-5 activation, mechanisms of action, signaling pathways, and regulatory elements have been reviewed (Song et al., J. Clin. Invest. (2020), 130(12):6700-6717; Almutaqqi and Udalova FEBS J. (2018), 286:1624-1637; Banga et al., Sci. Adv. (2020), 6:eaay1057; Thompson et al., Front. Immunol. (2018), 9:2622). In embodiments, the TMs described herein affect IRF-5 activation. In embodiments, a TM affects an IRF-5 signaling pathway. In embodiments, a TM affects an IRF-5 regulatory element.

IRF-5 is localized in the cytoplasm as an inactive monomer. In the inactive conformation, the C-terminal autoinhibitory domain (AID) of IRF-5 masks the N-terminal DNA-binding domain (DBD) and/or the C-terminal protein interaction domain (IAD), which are needed for homo- or heterodimerization. Upon activation by posttranslational modification, IRF-5 undergoes a conformational change that exposes the IAD for dimerization and nuclear localization signals (NLSs) for translocation. Song et al. (2020) J. Clin. Invest. 130(12):6700-6717. In embodiments, a TM modulates dimerization and/or nuclear localization of IRF-5.

It is believed that ubiquitination of IRF-5 plays an important role in nuclear translation target gene regulation. Carboxyl terminal phosphorylation of IRF-5 is believed to be important for IRF-5 transcriptional activity. Phosphorylation sites towards the serine-rich region of IRF-5 include S425, S427, S430 and S436 (human isoforms 3 and 4). Phosphorylation of S436 contributes to the stabilization of the activated dimer. Phosphorylation of S425, S427 and S430 are important for release of the autoinhibitory conformation. In embodiments, a TM modulates ubiquitination and/or phosphorylation of IRF-5.

Interactions between Helix 2 and Helix 5 of IRF-5 monomers is important for dimerization. (Banga et al. (2020) “Sci. Adv. 6:eaay1057). In embodiments, a TM modulates interaction between Helix 2 and Helix 5 of IRF-5 monomers.

IRF-5 regulates the toll-like receptor (TLR) dependent activation of inflammatory cytokines and functions downstream of the TLR-myD88 pathway where it is activated by MyD88 and TNF receptor associated factor 6 (TRAF6). Briefly, upon ligand binding to TLR7/8, MyD88 is recruited along with IRAK 1/4 and TRAF6, which leads to the autophosphorylation of IRAK4 and ubiquitination of IRF-5 by TRAF6. IRAK4 then activates TAK1, which then phosphorylates IKKβ. The ubiquitinated IRF-5 is then phosphorylated by IKKβ (or other kinases), resulting in homodimerization and translocation of the IRF-5 homodimer to the nucleus and the production of downstream cytokines. In addition to its role in homo- and hetero-dimerization, phosphorylation is important for the interaction between IRF-5 with histone acetyltransferases (HATs) (Thompson et al., Immunol. (2018), 9:2622.

Aberrant IRF-5 expression is associated with a variety of diseases. For example, upregulation of IRF-5 can lead to increased production of IFNs, which is linked to the development of numerous inflammatory diseases, including autoimmune disease, infectious disease, cancer, obesity, neuropathic pain, cardiovascular disease (e.g., artherosclerosis) and metabolic dysfunction (Banga et al. (2020) Sci. Adv. 6:eaay1057). Additionally, IRF-5 gene polymorphisms related to higher IRF-5 expression are associated with susceptibility to inflammatory and autoimmune diseases including rheumatoid arthritis (RA), inflammatory bowel disease (IBD), multiple sclerosis (MS) inflammatory bowel disease (IBD), systemic lupus erythematosus (SLE) and Sjögrens syndrome (Almuttaqi and Udalova, FEBS J. (2018), 286:1624-1637; Thompson et al., Front. Immunol. (2018), 9:2622). IRF-5 in particular operates as a master switch in macrophages and is implicated in proinflammatory cytokine release and fibrosis formation across a range of diseases. IRF-5 polymorphisms related to higher expression have been associated with susceptibility to inflammatory and autoimmune diseases including rheumatoid arthritis (RA), inflammatory bowel disease (IBD), multiple sclerosis (MS) (Krausgruber et al., Nat. Immunol. (2010), 12(3):231-238).

In embodiments, a compound described herein inhibits aberrant expression of IRF-5. In embodiments, such a compound or composition comprising such a compound may be administered to a subject in need thereof to treat one or more diseases associated with aberrant expression of IRF-5.

Increased IRF-5 mRNA levels are strongly correlated with disease pathology. Particularly, IRF-5 activation is a master switch implicated in the inflammatory and fibrotic processes associated with non-alcoholic steatohepatitis, systemic lupus erythematosus, inflammatory bowel disease, rheumatoid arthritis, type 2 diabetes, asthma and neuropathic pain, among many others. IRF-5 knockout mice have been shown to have reduced inflammatory phenotype and relevant fibrosis in many disease models including, but not limited to, cancer, systemic sclerosis, non-alcoholic steatohepatitis (NASH), systemic lupus erythematosus (SLE), primary biliary cirrhosis, inflammatory bowel disease (IBD), rheumatoid arthritis (RA), asthma and neuropathic pain, among many others.

In embodiments, a compound described herein decreases IRF-5 mRNA levels. In embodiments, such a compound or composition comprising such a compound may be administered to a subject in need thereof to treat one or more diseases associated with increased IRF-5 mRNA levels.

IRF-5 involvement in various diseases has been document (see for example, Graham et al., Nat Genet. (2006), 38(5):550-5; Rueda et al., Arthritis Rheum. (2006), 54(12):3815-9; Henrique da Mota, Clin Rheumatol. (2015), 34(9):1495-501; Sigurdsson et al., Hum Mol Genet. (2008), 17(6):872-81: Peng, et al., Nephrology (Carlton) (2010), 15(7):710-3; Ishimura et al., J Clin Immunol. (2011), 31(6): 946-51; Summers et al., J Rheumatol. (2008), 35(11):2106-18; Ni et al., Inflammation (2019), 2(5):1821-1829; Dideberg et al., Hum Mol Genet. (2007), 16(24):3008-16; Lim et al., J. Dig. Dis. (2015), 16(4):205-16; Nordal et al., Ann. Rheum. Dis. (2012), 71(7):1197-202; Rebora, Int. J. Dermatol. (2016), 55(4):408-16; Zhao et al., Rheumatol. Int. (2017), 37(8):1303-131; Carmona et al., PLoS One (2013), 8(1):e54419; Flesch et al., Tissue Antigens (2011), 78(1):65-8; Heijde et al., Arthritis Rheum. (2007), 56(12):3989-94; Hafler et al., Genes Immun. (2009), 10(1):68-76; Balasa et al., Eur. Cytokine Netw. (2012), 23(4):166-72; Byre et al., Mucosal Immunol. (2017), 10(3):716-726; Wang et al., Gene (2012), 10, 504(2):220-5; Pimenta et al., Mol. Cancer (2015), 14(1):32; Rambod et al., Clin Rheumatol. (2018), 37(10):2661-2665; Davi et al., J Rheumatol. (2011), 38(4):769-74; Zimmerman et al., Kidney 360 (2020), 1(3): 179-190; Pandey et al., Mucosal. Immunol. (2019), 12(4):874-887; Masuda et al., Nat. Commun. (2014), 5: 3771; Alzaid et al., JCI Insight (2016), 1(20): e88689; Senevirante et al., Circulation (2017), 136(12): 1140-1154; Cevik et al., J. Biol. Chem. (2017), 292(52):21676-21689; Sharif et al., Ann. Rheum. Dis. (2012), 71(7):1197-1202; and Yang et al., J Pediatr. Surg. (2017), 52(12):1984-1988). In embodiments, a compound or composition described herein is administered to a subject in need thereof to treat one or more of the diseases associated with IRF-5 that are known in the art.

IRF-5 is involved in innate and adaptive immunity, macrophage polarization, cell growth regulation, and apoptosis. Upregulation of IRF-5 can lead to increased production of IFNs, which is linked to the development of numerous inflammatory diseases, including autoimmune disease, infectious disease, cancer, obesity, neuropathic pain, cardiovascular disease (e.g., artherosclerosis), and metabolic dysfunction (Banga et al., (2020) Sci. Adv. (2020), 6:eaay1057). Polymorphisms in IRF-5 genes are also associated with autoimmune disease susceptibility, including, but not limited to, rheumatoid arthritis (RA), systemic sclerosis (SSc), multiple sclerosis (MS), inflammatory bowel disease (IBD) and systemic lupus erythematosus (SLE) (Thompson et al. (2018) Front. Immunol. 9:2622).

In embodiments, a compound described herein inhibits upregulation of IRF-5. In embodiments, such a compound or a composition comprising such a compound is administered to a subject in need thereof to treat a disease associated with upregulation of IRF-5. In embodiments, a compound described herein modulates level and/or activity of one or more polymorph of IRF-5. In embodiments, such a compound or a composition comprising such a compound is administered to a subject in need thereof to treat a disease associated with an IRF-5 polymorph.

In addition to autoimmune and viral disease pathogenesis, IRF-5 is implicated in diseases such as including cancer, obesity, neuropathic pain, cardiovascular disease, and metabolic dysfunction. In embodiments, a compound or a composition described herein is administered to a subject in need thereof to treat cancer, obesity, neuropathic pain, cardiovascular disease, and metabolic dysfunction.

IRF-5 has been shown to influence inflammatory macrophage phenotype (Almuttaqi and Udalova FEBS J. (2018), 286:1624-1637). Macrophages can be classified as M1 (classically activated macrophages) or M2 (alternatively activated macrophages) and can be converted to each other depending on the tissue microenvironment. There are three classes of alternately activated macrophages (M2a, M2b and M2c). In normal tissue, the ratio of M1 to M2 macrophages is highly regulated. An imbalance between M1 and M2 macrophages can result in pathologies such as asthma, chronic pulmonary disease, artherosclerosis, or osteoclastogenesis in rheumatoid arthritis. Interferon Regulatory Factor-5 (IRF-5) is a major regulator of proinflammatory M1 macrophage polarization (Weiss et al., Mediators of Inflammation (2013), Dx.doi.org/10.1155/2013/245804). IRF-5 expression in macrophages is reversibly induced by inflammatory stimuli and contributes to macrophage polarization. IRF-5 upregulates expression of M1 macrophages and downregulates expression of M2 macrophages (Krausgruber et al. (2010) Nat. Immunol. (2010), 12(3):231-238).

In embodiments, a TM modulates IRF-5 level and/or activity, which results in modulating the ratio of M1 to M2 macrophages. In embodiments, a compound comprising such a TM or a composition comprising such a compound is administered to a subject in need thereof to treat one or more diseases associated with imbalance of the ratio of M1 to M2 macrophages. In embodiments, a compound or composition described herein is administered to a subject in need thereof to treat one or more of asthma, chronic pulmonary disease, artherosclerosis, and osteoclastogenesis.

### Compounds

In embodiments, compounds are provided that modulate the level and/or activity of IRF-5. In embodiments, the compounds of the present disclosure include cyclic cell penetrating peptide (cCPP) and a therapeutic moiety (TM). The cCPP facilitates entry of the TM into the cell. The cCPP may form an ensomal escape vehicle (EEV). In embodiments, the compounds comprise an exocyclic peptide (EP) that facilitates escape from endosomes. The EEV may permit the TM to enter the cytosol or a cellular compartment to interact with IRF-5.

### Therapeutic Moieties

Generally, the TM is the effector moiety that elicits a response. In embodiments, the TM induces the downregulation of the IRF5-5 target gene, target transcript, and/or a target protein. In embodiments, the TM induces the downregulation of a downstream gene, downstream transcript, and/or downstream protein that is regulated by IRF-5. In embodiments, the TM induces the upregulation of the IRF-5 target gene, target transcript, and/or a target protein. In embodiments, the TM induces the upregulation of a downstream gene, downstream transcript, and/or downstream protein that is regulated by IRF-5.

In embodiments, the TM comprises an oligonucleotide, a peptide, an antibody, and/or a small molecule. The class and identity of the TM depends on the mechanism being used to modulate the level and/or activity of IRF-5.

In embodiments, the TM mechanism functions by targeting RNA processing. In embodiments, the TM targets the splicing process of IRF-5 to alter the level and/or activity of an IRF-5 target transcript. In embodiments, the TM induces exon skipping. In embodiments, the TM induces exon skipping to produce a frameshift. In embodiments, the frameshift produced a premature termination codon in an IRF-5 target transcript. In embodiments, the frameshift results in the nonsense mediated decay of an IRF-5 target transcript. In embodiments, the TM targets the polyadenylation process of IRF-5 to modulate the level and/or activity of IRF-5. In embodiments, the TM functions by binding to, or proximate to, one or more polyadenylation sequence elements to inhibit polyadenylation and translation of the IRF-5 target transcript. In embodiments where the TM mechanism functions by targeting RNA processing, the TM may be an oligonucleotide.

In embodiments, the TM mechanism functions by targeting the translated protein. In embodiments, the TM induces proteasomal degradation of an IRF-5 target transcript. In embodiments where the TM mechanism functions by targeting the translated protein, the TM may be a peptide, antibody, and/or a small molecule.

TM Mechanisms that Function by Targeting RNA Processing

A gene is a deoxyribonucleic acid (DNA) sequence that encodes a functional gene product, such as a protein. The process of converting the code of the gene into the functional gene product includes the steps of transcribing RNA (transcript) from genetic DNA and translating the RNA into a protein. RNA is first transcribed from DNA as immature “pre-mRNA” that undergoes processing to become a mature messenger RNA (mRNA) that can be translated into a protein. In eukaryotes, the processing steps include addition of a single-nucleotide modified guanine (G) nucleotide cap to the 5′ end of the RNA; addition of a poly-adenosine sequence to the 3′ end of the RNA (poly-A tail); and RNA splicing.

In embodiments, compounds, compositions, and methods are provided to modulate the polyadenylation process. In embodiments, compounds and methods are provided to modulate the splicing of an IRF-5 target transcript.

### Splicing

Splicing refers to a process in which introns (intervening sequences) are removed from the pre-mRNA and exons (coding sequences) are ligated together to form a mature mRNA. Introns are regions of a primary transcript (or the DNA encoding it) that are not included in the coding sequence of the mature mRNA. Exons are regions of a primary transcript that remain in the mature mRNA when it reaches the cytoplasm. The exons are spliced together to form the mature mRNA sequence.

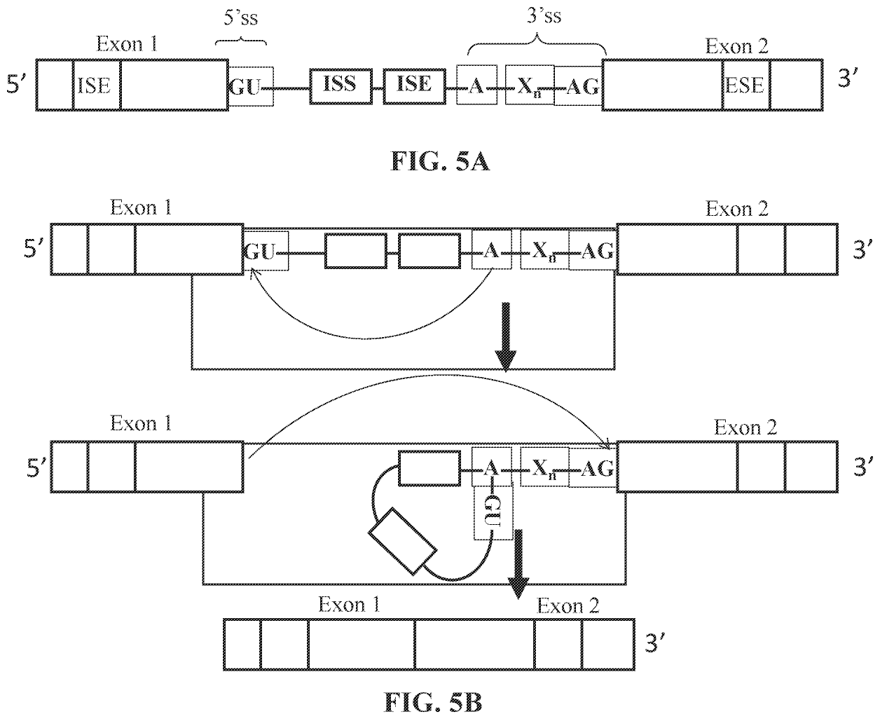

Splicing is governed in part by splice elements (SE). As used herein, “splice elements” are sequence elements found in pre-mRNA that are necessary for canonical splicing to occur (FIG. 5A). SEs include a 5′ splice site (5′ ss) and a 3′ splice site (3′ ss). The 5′ ss, also referred to as a donor splice site, includes a nearly invariant “GU” dinucleotide sequence along with less conserved downstream residues. The 5′ splice site also includes an exon/intron junction. As used herein, the exon/intron junction is the nucleotide sequence 10 nucleotides upstream and 10 nucleotides (+10 and −10) from the G of the GU sequence of the 5′ ss. The 3′ ss, or acceptor splice site, includes three conserved elements: a branch splice point (BSP; sometimes called the branch point of branch nucleotide), a polypyrimidine or Py tract, and a terminal “AG.” The BSP is typically an adenosine that is located about 18 to about 40 nucleotides from the 3′ ss. The Py tract typically includes about 15 to about 20 pyrimidine residues, particularly uracil (U). Atypical branch points exist, however; they are more distant from the 3′ splice site and/or utilize a non-adenosine base (Montes et al. (2019) “RNA splicing and disease: animal models to therapies,” Trends Genet. 35(1):68-87). The 3′ ss also includes an intron/exon junction. As used herein, the intron/exon junction is the nucleotide sequence 10 nucleotides upstream and 10 nucleotides (+10 and −10) from the G of the AG sequence of the 3′ ss.

Exons are recognized in most splicing reactions by specific base-pairing interactions with small nuclear RNA (snRNA) components of five small ribonucleoproteins (snRNPs); U1, U2, U4, U5, and U6 (Havens, et al. (2014) Wiley Interdiscip. RNA. 2013, 4(3), 247-266. doi.10.1002/wrna.1158; Wahl M. C. et al., Cell (2009), 136: 701-718). Each snRNP includes a small nuclear RNA that is configured to recognize specific nucleotide sequences and one or more proteins.

Exon splicing includes two sequential spliceosome catalyzed transesterification reactions (FIG. 5B). In general, the splicing reaction is initiated by U1 binding to the 5′ ss, followed by U2 binding the branch splice point (BPS), and finally U4, U5, and U6 bind near the 5′ and 3′ splice sites. U1 and U4 are then displaced followed by the first transesterification reaction where 2′-OH of a branch-point nucleotide within an intron performs a nucleophilic attack on the first nucleotide of the intron at the 5′ splice site forming a lariat intermediate. In a second reaction, the 3′-OH of the released 5′ exon performs a nucleophilic attack at the last nucleotide of the intron at the 3′ splice site thus joining the exons and releasing the intron lariat. U4, U5, and U6 are released as well.

In addition to SEs, splicing is regulated in part by splicing regulatory elements (SREs). SREs include cis-regulatory elements and trans-acting splicing factors. The cis-regulatory elements and trans-acting splicing factors may promote canonical splicing, alternative splicing, or cryptic splicing.

Cis-regulatory elements are nucleotide sequences within the transcript that suppress or enhance splicing. Trans-acting splicing factors are proteins and/or oligonucleotides that are not located within the transcript and work to enhance or suppress splicing. Cis-regulatory elements generally function to recruit trans-acting splicing factors which activate or suppress splicing. Trans-acting splice factors regulate splicing by associating with cis-regulatory elements. Trans-acting splice factors include serine/arginine rich (SR-rich) proteins and heterogenous nuclear ribonucleoproteins (hnRNPs).

Splicing cis-regulatory elements include exonic splicing enhancer (ESE) sequences, exonic splicing silencers (ESS) sequences, intronic splicing enhancer (ISE) sequences, and intronic splicing silencer (ISS) sequences (FIG. 5A). ESE sequences promote the inclusion of the exon they reside in into the mRNA. ESS sequences inhibit the inclusion of the exon they residue in into the mRNA. ISE sequences enhance the use of alternate splice sites from their location within an intron. ISS sequences inhibit the use of alternate splice sites from their location within an intron. Typically, ISSs are between 8 and 16 nucleotides in length and are less conserved than the splice sites at exon-intron junctions.

Pre-mRNA splicing may also be regulated by the formation of secondary structures such as terminal stem loops (TSL) within the transcript that may affect the binding of spliceosome or other regulatory proteins. Terminal stem loop sequences may be an SRE and are typically from about 12 to about 24 nucleotides and form a secondary loop structure due to the complementarity, and hence binding, within the 12 to 24 nucleotide sequence.

Each SE and/or SRE is separated from an adjacent SRE and/or SE by an intervening sequence (IS).

In embodiments, a compound described herein includes a TM that modulates splicing. In embodiments, the TM binds to at least a portion of a SE or a cis-splicing regulatory element (SRE) to modulate splicing. In embodiments, the TM binds in sufficiently close proximity to a SE or a cis-SRE to modulate splicing. In embodiments, a TM that does not bind to a SE or cis-SRE bind but binds in sufficiently close proximity to the SE or the cis-SRE to modulate splicing sterically block interaction of a splicing factor to a target transcript (e.g., an IRF-5 transcript). In embodiments, the TM binds within 10, 9, 8, 7, 6, 5, 4, 3, 2, or 1 nucleotide of an SE or cis-SRE.

### Exon Skipping

In embodiments, compounds and methods are provide that induce exon skipping.

Most eukaryotic pre-mRNA can be spliced differently, often by skipping an exon, to produce distinct mature mRNA isoforms in a process called alternative splicing. The term “alternative splicing” refers to the joining of exons in different combinations (e.g., different 5′ and 3′ splice sites are joined). Alternative splicing can insert or remove amino acids, shift the reading frame, and/or introduce a termination codon, which contributes to the complexity, flexibility, and abundance of genes and proteins expressed from a gene. Alternative splicing can also affect gene expression by removing or inserting regulatory elements, controlling translation, mRNA stability, and/or localization. Mutations that disrupt splicing are estimated to account for up to a third of all disease-causing mutations (Havens, et al. (2014) Wiley Interdiscip RNA. 2013, 4(3), 247-266. doi:10.1002/wrna.1158; Lim K. H., et al., Proc. NatI. Acad. Sci. USA (2011), 108: 11093-11098; Faustino and Cooper, Genes & Dev. (2003), 17:419-437; and Sterne-Weiler T., et al., Genome Res. (2011), 21: 1563-1571).

Mutations that impact the splicing process can occur in many different ways (Havens, et al., (2014) Wiley Interdiscip. RNA. 2013, 4(3), 247-266. doi:10.1002/wrna.1158). For example, intronic mutations may disrupt the core splice sites (sequences within the 5′ ss or 3′ ss, the Py tract or BPS), resulting in the skipping of an exon(s) upstream or downstream from the mutated splice site (5′ ss and/or 3 ss) or the retention of an intron. Often, when a splice site is mutated, a pseudo splice site is activated within a flanking exon or intron, which after splicing results in an alternative transcript. Mutations within an intron can also disrupt or create de novo splicing silencers and/or enhancers and/or create de novo cryptic splice sites. Intronic splice site mutations may account for approximately 10-15% of disease mutations (Havens, et al. (2014) Wiley Interdiscip RNA. 2013, 4(3), 247-266. doi:10.1002/wrna.1158; Stenson P. D., et al., The Human Gene Mutation Database: 2008 update. Genome Med 2009, 1:13). Mutations that occur within coding exons (exonic mutations), can result in the creation of a de novo cryptic splice site, disruption of an RNA secondary structure that has a regulatory function, and/or disruption of a splicing silencer or enhancer rendering a splice site unrecognizable by a sequence-specific RNA-binding protein that is required for splicing. Analysis of exonic mutations predict that as many as 25% of mutations within exons can alter splicing (Ibid; Proc. Natl. Acad. Sci. USA (2011), 108: 11093-11098). Cryptic splicing is caused by sequences in the pre-mRNA that are not normally used as splice sites, but which are activated by mutations that either inactivate the canonical splice site or create splice sites where one did not exist before (Arechavala-Gomeza, et al. The Application of Clinical Genetics (2014), 4(7), 245-252; Roca X., et al. “Genes Dev. (2013); 27(2):129-144). Additionally, alternative splicing, which contributes to the different proteins generated from pre-mRNA, can cause disease by shifting expression from one isoform to a different isoform associated with a disease (Ibid).

Targeting the splicing reaction or sequence elements involved in splicing (e.g., SEs and/or SREs) to induce aberrant splicing can be used to disrupt gene expression of proteins involved in disease pathogenesis. For example, splicing can be targeted to cause the skipping of exons, thereby introducing a frameshift or a stop codon that results in a non-functional or truncated protein or degradation of the RNA transcript (Stenson P. D., et al., Genome Med. 2008; 1 (13)). Splicing-induced reading frame correction, reframing, and/or nonsense mediated decay of target transcripts provides an opportunity for treating many diseases and disorders.

Exon Skipping to Modulate IRF-5 Levels and/or Activity

TMs can be designed to cause exons to be skipped resulting in increased or decreased expression or activity of IRF-5 and/or a downstream protein that is regulated by IFRF-5. In embodiments, the TM is an antisense compound (AC).

ACs are described elsewhere herein. Briefly, an AC is an oligonucleotide that includes DNA bases, modified DNA bases, RNA bases, modified RNA bases, or combinations thereof. Generally, the AC includes a nucleotide sequence that is complementary, or at least partially complementary to a target nucleotide sequence found within an IRF-5 target transcript. The AC hybridizes to (e.g., binds to) the target nucleotide to elicit a response. The ACs may have any properties described elsewhere herein. For example, in embodiments, the AC may be an antisense oligonucleotide (ASO). The term antisense oligonucleotide refers to oligonucleotides that are complementary to a targeted polynucleotide sequence. In embodiments, the ASO is a phosphorodiamidate morpholinos PMO (described elsewhere herein).

In embodiments, a compound is provided that includes an AC that binds an IRF-5 target transcript to result in generation of an mRNA that encodes a truncated IRF-5 protein and/or a nonfunctional IRF-5 protein. In embodiments, a compound is provided that includes an AC that binds an IRF-5 target transcript to result in generation of an mRNA that encodes a truncated IRF-5 protein and/or a nonfunctional IRF-5 protein through alternative splicing. In embodiments, a compound is provided that includes an AC that binds an IRF-5 target transcript to result in degradation of an IRF-5 target transcript, for example, through nonsense mediated decay. In embodiments, a compound is provided that includes an AC that binds an IRF-5 target transcript to result in generation of an alternate mRNA isoform of IRF-5 that has beneficial properties.

A compound that includes an antisense compound (AC) can be used to modulate splicing in any suitable manner. In embodiments, the AC can be designed to sterically block access to a splice site, or at least a portion of a splicing element (SE) and/or a splicing regulatory element (SRE), thereby redirecting splicing to a cryptic or de novo splice site. In embodiments, the AC can be targeted to a splicing enhancer sequence (e.g., ESE an/or ISE) or splicing silencer sequence (e.g., ESS and/or ISS) to prevent binding of trans-acting regulatory splicing factors at the target site and effectively block or promote splicing. In embodiments, the AC can be designed to base-pair across the base of a splicing regulatory stem loop to strengthen the stem-loop structure.

In embodiments, the AC hybridizes with target nucleotide sequence that includes at least a portion of a SRE of an IRF-5 target transcript. In embodiments, the AC hybridizes with a target nucleotide sequence that includes an entire SRE of an IRF-5 target transcript. In embodiments, the AC hybridizes with a target nucleotide sequence that includes multiple SREs of an IRF-5 target transcript. In embodiments, the AC hybridizes with a target nucleotide sequence that includes multiple SREs of an IRF-5 target transcript and the intervening sequences between the SREs.

In embodiments, the target nucleotide sequence includes the entire SE and/or SRE and one or more flanking sequences that are upstream and/or downstream of the SE and/or SRE of an IRF-5 target transcript. In embodiments, the target nucleotide sequence includes a portion, but not the entirety, of a SE and/or a SRE and one or more flanking sequences that are upstream and/or downstream of the SE and/or the SRE of an IRF-5 target transcript.

In embodiments, the flanking sequence includes 1 or more, 2 or more, 3 or more, 4 or more, 5 or more, 10 or more, 15 or more, or 20 or more bases on one or both sides of an SE and/or SRE. In embodiments, the flanking sequence includes 25 or less, 20 or less, 15 or less, 10 or less, 5 or less, 4 or less, 3 or less, or 2 or less bases on one or both sides of an SE and/or SRE. In embodiments, the flanking sequence includes 1 to 25, 1 to 20, 1 to 15, 1 to 10, 1 to 5, 1 to 4, 1 to 3 or 1 to 2 bases on one or both sides of an SE and/or SRE. In embodiments, the flanking sequence includes 2 to 25, 2 to 20, 2 to 15, 2 to 10, 2 to 5, 2 to 4, or 2 to 3 bases on one or both sides of an SE and/or SRE. In embodiments, the flanking sequence includes 3 to 25, 3 to 20, 3 to 15, 3 to 10, 3 to 5, or 3 to 4 bases on one or both sides of an SE and/or SRE. In embodiments, the flanking sequence includes 4 to 25, 4 to 20, 4 to 15, 4 to 10, or 4 to 5 bases on one or both sides of an SE and/or SRE. In embodiments, the flanking sequence includes 5 to 25, 5 to 20, 5 to 15, or 5 to 10 bases on one or both sides of an SE and/or SRE. In embodiments, the flanking sequence includes 10 to 25, 10 to 20, or 10 to 15 bases on one or both sides of an SE and/or SRE. In embodiments, the flanking sequence includes 15 to 25 or 15 to 20 bases on one or both sides of an SE and/or SRE. In embodiments, the flanking sequence includes 20 to 25 bases on one or both sides of an SE and/or SRE. In embodiments, the flanking sequence includes an intervening sequence or a portion thereof.

In embodiments, the AC hybridizes with a target nucleotide sequence that includes at least a portion of a 5′ ss of an IRF-5 target transcript. In embodiments, the AC hybridizes with a target nucleotide sequence that includes at least a portion of the exon/intron junction of an IRF-5 target transcript. In embodiments, the AC hybridizes with a target nucleotide sequence that includes at least a portion of a 3′ ss of an IRF-5 target transcript. In embodiments, the AC hybridizes with a target nucleotide sequence that includes at least a portion of a Py tract, BPS, terminal “AG,” and/or the intron/exon junction of an IRF-5 target transcript.

In embodiments, the AC hybridizes with target nucleotide sequence that includes at least a portion of a splice regulatory element (SRE) of an IRF-5 target transcript. In embodiments, the AC hybridizes with a target nucleotide sequence that includes an entire SRE of an IRF-5 target transcript. In embodiments, the AC hybridizes with a target nucleotide sequence that includes multiple SREs of an IRF-5 target transcript. In embodiments, the AC hybridizes with a target nucleotide sequence that includes multiple SRE of an IRF-5 target transcript and the intervening sequences between the SREs of a target transcript. In embodiments, the AC hybridizes with a target nucleotide sequence that includes at least a portion of an ESE of an IRF-5 target transcript. In embodiments, the AC hybridizes with a target nucleotide sequence that includes at least a portion of an ISE of an IRF-5 target transcript. In embodiments, the AC hybridizes with a target nucleotide sequence that includes at least a portion of an ESS of an IRF-5 target transcript. In embodiments, the AC hybridizes with a target nucleotide sequence that includes at least a portion of an ISS of an IRF-5 target transcript.

In embodiments, the AC hybridizes with a target nucleotide sequence that includes at least a portion of a terminal stem loop (TLS) of an IRF-5 target transcript.

In embodiments, the AC hybridizes with at least a portion of an aberrant SE and/or SRE of an IRF-5 target transcript where the aberrant SE and/or SRE resulted from a mutation in the target gene.

In embodiments, the AC hybridizes with a target nucleotide sequence that includes at least a portion of a SE and/or SRE, an exon/intron junction, or an intron/exon junction of an IRF-5 target transcript. In embodiments, the AC hybridizes with a target nucleotide sequence that includes an aberrant fusion junction due to a rearrangement or a deletion of an IRF-5 target transcript. In embodiments, the AC hybridizes with a particular exon in alternatively spliced mRNAs of an IRF-5 target transcript.

In embodiments, the AC binds to a target nucleotide sequence that does not include at least a portion of an SE or at least a portion of an SRE of an IRF-5 target transcript. In embodiments, the AC binds to a target nucleotide sequence that is in sufficiently close proximity to an SE and/or an SRE to modulate splicing of the IRF-5 target transcript.

In embodiments, the AC binds to a target nucleotide sequence with a 3′ end and/or 5′ end that is 1 or more, 2 or more, 3 or more, 4 or more, 5 or more, 10 or more, 15 or more, or 20 or more nucleotides from the 5′ end and/or 3′ end of a SE and/or and SRE of an IRF-5 target transcript. In embodiments, the AC binds to a target nucleotide sequence with a 3′ end and/or 5′ end that is 25 or less, 20 or less, 15 or less, 10 or less, 5 or less, 4 or less, 3 or less, or 2 or less nucleotides from the 5′ end and/or 3′ end of a SE and/or and SRE of an IRF-5 target transcript. In embodiments, the AC binds to a target nucleotide sequence with a 3′ end and/or 5′ end that is 1 to 25, 1 to 20, 1 to 15, 1 to 10, 1 to 5, 1 to 4, 1 to 3, or 1 to 2 nucleotides form the 5′ end and/or 3′ end of a SE and/or and SRE of an IRF-5 target transcript. In embodiments, the AC binds to a target nucleotide sequence with a 3′ end and/or 5′ end that is 2 to 25, 2 to 20, 2 to 15, 2 to 10, 2 to 5, 2 to 4, or 2 to 3 nucleotides form the 5′ end and/or 3′ end of a SE and/or and SRE of an IRF-5 target transcript. In embodiments, the AC binds to a target nucleotide sequence with a 3′ end and/or 5′ end that is 3 to 25, 3 to 20, 3 to 15, 3 to 10, 3 to 5, or 3 to 4 nucleotides form the 5′ end and/or 3′ end of a SE and/or and SRE of an IRF-5 target transcript. In embodiments, the AC binds to a target nucleotide sequence with a 3′ end and/or 5′ end that is 4 to 25, 4 to 20, 4 to 15, 4 to 10, or 4 to 5 nucleotides form the 5′ end and/or 3′ end of a SE and/or and SRE of an IRF-5 target transcript. In embodiments, the AC binds to a target nucleotide sequence with a 3′ end and/or 5′ end that is 5 to 25, 5 to 20, 5 to 15, or 5 to 10 nucleotides form the 5′ end and/or 3′ end of a SE and/or and SRE of an IRF-5 target transcript. In embodiments, the AC binds to a target nucleotide sequence with a 3′ end and/or 5′ end that is 10 to 25 or 10 to 20 nucleotides form the 5′ end and/or 3′ end of a SE and/or and SRE of an IRF-5 target transcript. In embodiments, the AC binds to a target nucleotide sequence with a 3′ end and/or 5′ end that is 20 to 25 nucleotides form the 5′ end and/or 3′ end of a SE and/or and SRE of an IRF-5 target transcript.

In embodiments, the AC induces the addition or deletion of one or more nucleotides in a resulting processed transcript, such as a mRNA. If the number of nucleotides added or removed from the open reading is divisible by three to produce a whole number, the resultant transcript may be translated into a functioning or non-functioning protein having more or less amino acids than a counterpart protein expressed from a transcript but otherwise has the same amino acid sequence, other than the added or deleted amino acids, as a protein expressed from a transcript that did not have the nucleotides added or removed. If the number of nucleotides added or removed from the open reading frame is not divisible by three to produce a whole number, the open reading frame of the resulting processed transcript, such as an mRNA, is shifted. For example, the number of nucleotides added or deleted to induce a such a “frameshift” alteration may be 1, 2, 4, 5, 7, 8, 10, 11, 13, 14, 16, 17, 19, 20, 22, 23, etc. Due to the triplet nature of the genetic code, the addition or deletion of a number of nucleotides that is not divisible by three, shifts the reading frame of the resulting processed transcript, such as an mRNA, downstream of frameshift. The shifted reading frame may result in nonsense mediated decay, may result in a premature stop codon within the nonsense downstream of the frameshift, and/or may result in expression of a protein having a completely different sequence of amino acids downstream of the frameshift.

In embodiments, the AC induces introduction of a premature termination codon (PTC) into the open reading frame. As used herein, “premature termination codon” is a stop codon in phase with the translational start codon and located upstream of the physiological stop codon that is in phase with the translation start codon. A target transcript having a PTC may be destabilized and degraded through various mechanisms including nonsense mediated decay.

Nonsense mediated decay is a surveillance mechanism that recognizes initiates exo- and endonucleolytic degradation pathways to remove mRNA transcripts having a PCT in order to prevent the expression of a truncated protein that may have deleterious effects on the cell. Several nonsense mediated decay pathways have been contemplated and reviewed (Lejeune et al., Biomedicines (2020), 10(1):141; Brogna et al., Nature Structural and Molecular Biology (2009), 16, 108-113, Karousis et al., Wiley Interdiscip. Rev. RNA (2016), 7(5). 661-682). In embodiments where the target gene is overexpressed in disease, inducing nonsense mediated decay may be used to reduce the concentration of a target protein, and therefore, treat the disease.

In embodiments, the AC induces exon skipping to result in nonsense mediated decay of the IRF-5 target transcript. This is in contrast to conventional exon skipping which aims to skip an exon to induce expression of a particular protein isoform to correct for missplicing, alternative splicing, and/or to avoid deleterious mutations in specific exons.

In embodiments, the AC induces exon skipping of an exon within the IRF-5 target transcript where the exon has a has a number of nucleotides not divisible by three. In embodiments, the AC induces exon skipping of an exon that has a number of nucleotides not divisible by three resulting in a PCT within the IRF-5 target transcript. In embodiments, the AC induces exon skipping of an exon that has a number of nucleotides not divisible by three resulting in a PCT within the IRF-5 target transcript which leads to nonsense mediated decay of the IRF-5 target transcript. In embodiments, inducing nonsense mediated decay of a target transcript results in a decreased concentration of the IRF-5 target transcript. In embodiments, inducing nonsense mediated decay of a target transcript results in a decreased concentration of the IRF-5 target protein. In embodiments, inducing nonsense mediated decay of a target transcript results in increased and/or decreased levels of proteins of downstream genes regulated by the target gene.

FIG. 6 shows an example of AC induced exon skipping resulting in nonsense mediated decay of a target transcript or premature termination of translation of a protein. The AC binds to pre-mRNA. In the illustrative embodiment, the AC binds the at the intron/exon junction of exon three. In other embodiments, the AC can bind to the target transcript in various other places to induce exon skipping resulting in nonsense mediated decay of the target transcript (discussed elsewhere). The number of nucleotides in exon three is not divisible by three, for example, 52, 106, 232, 365, and the like. Binding of the AC to the intron/exon junction induces exon skipping of exon three through a variety of possible mechanism. For example, the binding of the AC to the intron/exon junction prevents the splicing machinery from accessing the splicing elements. Additionally, or alternatively, the binding of the AC to the intron/exon junction prevents the completion of one or both of the transesterification reactions needed to complete the splicing process. As a result of the AC binding to the target transcript, exon three is skipped and the resultant transcript includes exon two connected with exon four. As a result of the AC binding to the target transcript and skipping exon three, the reading frame in exon four of the resultant transcript is shifted. The shift in reading frame in the illustrated embodiment introduces a PTC in the resulting transcript. As a result of the AC binding to the target transcript, skipping exon three, and exon four having a PTC, the resultant transcript is targeted for and undergoes nonsense mediated decay.

In embodiments, a compound includes an antisense compound (AC) that induces skipping of one or more of exons 2, 3, 4, 5, 6, 7, and/or 8 of human and/or mouse IRF-5. In embodiments, a compound includes an AC that induces skipping of one or more exons to produce an out of frame frameshift leading to the IRF-5 target transcript being degraded (e.g., nonsense mediate decay) or being translated into an IRF-5 protein with reduce or no activity. In embodiments, a compound includes an AC that induces skipping of one or more of exons 3, 4, 5, and/or 8 produce an out of frame frameshift.

In embodiments, the AC includes any one of SEQ ID NOs:157-161 in Table 1. In embodiments the AC includes 10 to 25, 10 to 20, or 10 to 15 consecutive bases of anyone of SEQ ID NOs:157-161. In embodiments, SEQ ID NOs:157 and 158, or a fragment thereof, induces skipping of exon 4 to produce a premature termination codon in exon 5. In embodiments, SEQ ID NOs:157 and 158, or a fragment thereof induce exon skipping of exon 4 leading to nonsense mediated decay of the IRF-5 target transcript. In embodiments, SEQ ID NOs:157 and 158, or a fragment thereof, induces skipping of exon 4 to produce a premature stop codon. In embodiments, SEQ ID NOs:159-161, or a fragment thereof induce exon skipping of exon 5 resulting in a premature termination codon in exon 6. In embodiments, SEQ ID NOs:159-161, or a fragment thereof induce exon skipping of exon 5 leading to nonsense mediated decay of the IRF-5 target transcript.

TABLE 1
AC sequences for inducing exon skipping

| SEQ | | Exon to |
|---|---|---|
| ID | | be |
| NO: | Sequence 5′-3′ | skipped |
| 157 | AGA ACG TAA TCA TCA GTG GGT TGG C | 4 |
| 158 | ACG TAA TCA TCA GTG GGT TGG CTC T | 4 |
| 159 | TTGGCAACATCCTCTGCAGCTGAAG | 5 |
| 160 | GCAACATCCTCTGCAGCTG | 5 |
| 161 | TCAGGCTTGGCAACATCCTCTGCAG | 5 |

In embodiments, a compound includes an AC that induces skipping of one or more exons to produce an in-frame deletion in a IRF-5 target transcript. In embodiments, a compound includes an AC that induces skipping of one or more of exons 6 and/or 7 to produce an in-frame deletion in an IRF-5 target transcript.

### Polyadenylation

Pre-mRNA processing includes the addition of polyadenosine tail on the 3′ untranslated region. The poly(A) tails of processed mRNA in eukaryotes influence the stability of the mRNA, the translation or translation efficiency, and/or the transport of the mRNA from the nucleus to the cytoplasm and thereby ultimately governs the production of a protein. More specifically, the poly(A) tail allows for the transport of the RNA molecule from the nucleus to the cytoplasm, enhances translation efficiency, and controls RNA degradation (Nourse et al., Biomolecules (2020), 10(915) doi:10.3390/biom10060915). Formation of the poly(A) tail is also connected to other transcriptional and post-transcriptional processes, including for example splicing and transcriptional termination.

The process of adding the poly(A) tail is termed polyadenylation. The polyadenylation process is generally a two-step process involving a cleavage reaction followed by the addition of the poly(A) tail. The cleavage reaction involves endonucleolytic cleavage. Following cleavage, in almost all cases, multiple adenosines (e.g., 50 to 300) are enzymatically added to the resulting 3′ cleaved end to generate the poly(A) tail (Tian et al., Nuc. Acid. Res. (2005), 33(1):201-212 and Neve et al., RNA Biology (2017), 14(7):865-890).

The polyadenylation process is governed by more than 80 RNA-binding proteins; however, fewer than 20 factors make up the core of the polyadenylation protein complex needed to mediate cleavage and polyadenylation in vitro (Marsollier et al., Int. J. Mol. Sci. (2018), 19, 1347, doi:10.3390/ijms19051347). These 20 factors are distributed in eight complexes: cleavage and polyadenylation specific factor (CPSF); cleavage stimulation factor (CstF); Symplekin; Mammalian cleavage factor I (CFIm); Mammalian cleavage factor II (CFIIm); Poly(A) polymerase (PAP); RNA polymerase II (PollI); and PolII C-Terminal Domain (CTDX ibid).

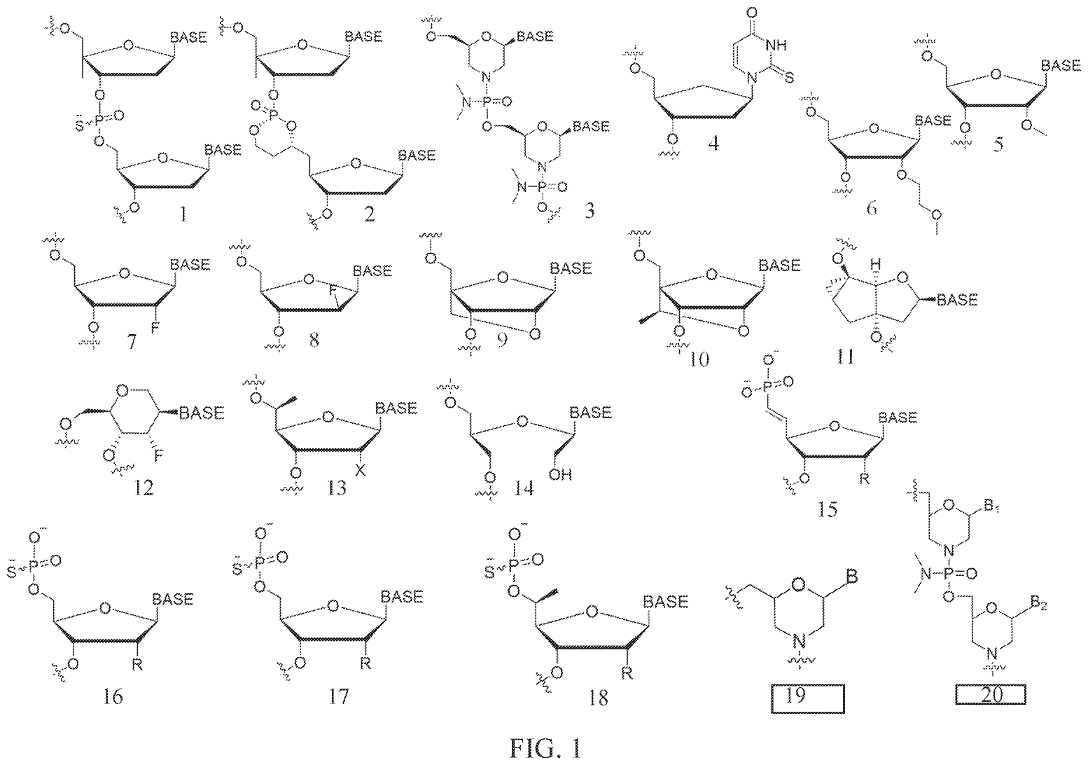

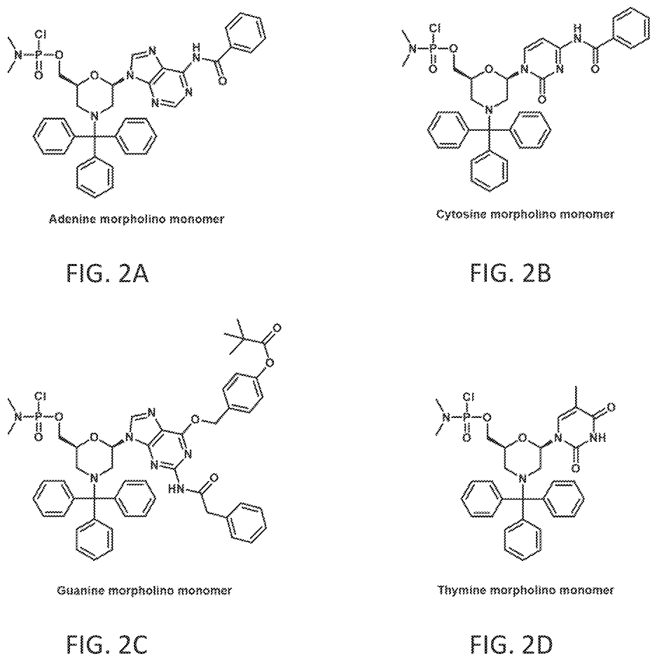

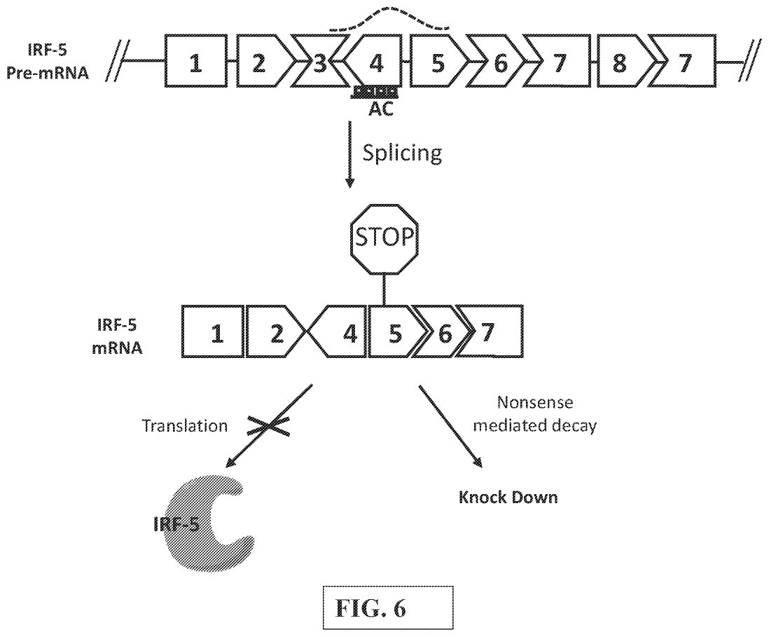

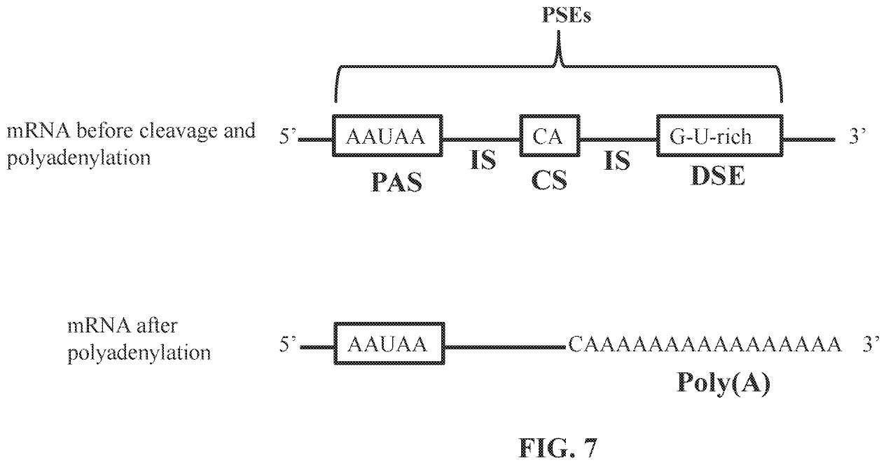

The factors interact with (e.g., bind to) a polyadenylation sequence element (PSE) during the polyadenylation process. Generally, the polyadenylation sequence elements include a polyadenylation signal (PAS), a cleavage stie (CS), and a GU-rich downstream element (DSE) (FIG. 7). In some cases, the polyadenylation sequence elements may also include one or more of an auxiliary upstream element (USE), a G-rich sequence (GRS) auxiliary downstream element (AUX DSE), and/or a sequence downstream of a core U-rich element (URE) (not depicted in FIG. 1; Chen and Wilusz, Nuc. Acid. Rec. (1998) 26(12):2891-2898). Each PSE may be separated by intervening nucleotide sequence (IS) (FIG. 7). Each intervening IS may be a PSE in and of itself (Venkataraman et al., Genes and Dev. (2005), 19:1315-1327).

The PAS is an adenosine-rich hexamer sequence that includes a canonical AATAAA hexamer or a variant differing by a single nucleotide (e.g., AAUAAA, AUUAAA, UAUAAA, AGUAAA, AAGAAA, AAUAUA, AAUACA, CAUAAA, GAUAAA, CAUAAA, GAUAAA, AAUGAA, UUUAAA, ACUAAA, AAUAGA, AAAAAG, AAAACA, GGGGCU; Marsollier et al. Int. J. Mol. Sci. (2018) 19, 1347, doi:10.3390/ijms19051347; Beaudoing, et al., Genome Res. (2000), 10, 1001-1010; and Tian, B. et al., Nucleic Acids Res. (2005), 33, 201-212). The PAS is typically found upstream of the CS. The hexamer sequence of the PAS serves as the binding site for a cleavage and polyadenylation specific factor (CPSF). The PAS can also be determined by the presence of other auxiliary elements, such as upstream U-rich elements (USE) (See, Tian et al., Nuc. Acid. Res. (2005), 33(1):201-212 and Neve et al., RNA Biology (2017), 14(7):865-890).

The DSE is a U-rich or U/G-rich element that serves as the binding site for a cleavage stimulatory factor (CstF). The DSE is typically found downstream of the CS. The DSE may be followed by a stretch of three or more uracil bases present downstream of the CS, often within 20 to 40 nucleotides of the CS. In mammals, CA and UA are the most frequent dinucleotides that precede the cleavage site (CS), although the actual cleavage site is known to be heterogeneous.

CPSF and CstF, two multi-subunit complexes, cooperate with each other and two additional factors (cleavage factors I and II) to cleave the mRNA sequence. Poly(A) polymerase (PAP), a single-subunit enzyme is also involved in cleavage of most pre-mRNAs, as is RNA polymerase II. CPSF and PAP together with a poly(A) binding protein II and CstF are involved in the addition of the poly(A) tail (Takagaki and ManleyMol Cell Biol. (2000), 20(5):1515-1525).

Methods for identifying polyadenylation sequence elements are known and can include but are not limited to, for example, the methodologies described by: Tian et al., Nuc. Acid. Res. (2005) 33(1):201-212; Beaudoing, et al., Genome Res. (2000), 10, 1001-1010; Marsollier et al., Int. J. Mol. Sci. (2018), 19, 1347, doi:10.3390fijms19051347; Chen, Molec. Therapy (2016), 24(8) 1405-1411; Venkataraman et al., Genes and Dev. (2005) 19:1315-1327; Nourse et al., Biomolecules (2000), 10(915) doi:10.3390/biom10060915; and Vickers et al., Nucleic Acids Research (2001), 29(6) 1293-1299).

### Targeting Polyadenylation to Modulate IRF-5 Levels and or Activity

In embodiments, compounds are provided that include a therapeutic moiety (TM) that modulates polyadenylation of an IRF-5 target transcript. In embodiments, the TM is an antisense compound (AC). The AC may have any properties described elsewhere herein. For example, in embodiments, the AC may be an antisense oligonucleotide (ASO). The term antisense oligonucleotide refers to oligonucleotides that are complementary to a targeted polynucleotide sequence. In embodiments, the ASO is a phosphorodiamidate morpholinos PMO (described elsewhere herein).

In embodiments, the AC binds to a target nucleotide sequence that includes at least a portion of at least one polyadenylation sequence element (PSE) of an IRF-5 target transcript. In embodiments, the target nucleotide sequence includes the entire PSE of an IRF-5 target transcript. In embodiments, the target nucleotide sequence includes the entire PSE and one or more flanking sequences that are upstream and/or downstream of the PSE of an IRF-5 target transcript. In embodiments, the target nucleotide sequence includes a portion, but not the entirety, of the PSE of IRF-5 target transcript. In embodiments, the target nucleotide sequence includes a portion, but not the entirety, of the PSE and one or more flanking sequences that are upstream and/or downstream of the PSE of an IRF-5 target transcript.

In embodiments, the AC binds to a target nucleotide sequence that includes at least a portion of one or more specific PSEs of IRF-5 target transcript. In embodiments, the target nucleotide sequence includes at least a portion of the consensus hexamer sequence of the PAS of an IRF-5 target transcript. In embodiments, the target nucleotide sequence includes at least a portion of the CS of an IRF-5 target transcript. In embodiments, the target nucleotide sequence includes at least a portion of the DSE of an IRF-5 target transcript. In embodiments, the target nucleotide sequence includes at least a portion of a USE of an IRF-5 target transcript. In embodiments, the target nucleotide sequence includes at least a portion of a G-rich sequence (GRS) auxiliary downstream element (AUX DSE) of an IRF-5 target transcript. In embodiments, the target nucleotide sequence includes at least a portion of an element core U-rich element (URE) of an IRF-5 target transcript. In embodiments, the target nucleotide sequence includes at least a portion of an intervening sequence (IS) of an IRF-5 target transcript. In embodiments, the target nucleotide includes at least a portion of more than one PSE of an IRF-5 target transcript. For example, in embodiments, the target nucleotide sequence includes at least a portion of the PAS and at least a portion of the CS of an IRF-5 target transcript. In embodiments, the target nucleotide sequence includes at least a portion of the PAS and at least a portion of the IS between the PAS and the CS of an IRF-5 target transcript.

In embodiments, the AC binds to a target nucleotide sequence that includes one or more PSEs and one or more sequences that flank the one or more PSEs of an IRF-5 target transcript. In embodiments, the flanking sequences is a sequence that is upstream of the PSE. In embodiments, the flanking sequence is a sequence that is downstream of the PSE.

In embodiments, the flanking sequence includes 1 or more, 2 or more, 3 or more, 4 or more, 5 or more, 10 or more, 15 or more, or 20 or more bases on one or both sides of a PSE of an IRF-5 target transcript. In embodiments, the flanking sequence includes 25 or less, 20 or less, 15 or less, 10 or less, 5 or less, 4 or less, 3 or less, or 2 or less bases on one or both sides of a PSE of an IRF-5 target transcript. In embodiments, the flanking sequence includes 1 to 25, 1 to 20, 1 to 15, 1 to 10, 1 to 5, 1 to 4, 1 to 3 or 1 to 2 bases on one or both sides of the PSE of an IRF-5 target transcript. In embodiments, the flanking sequence includes 2 to 25, 2 to 20, 2 to 15, 2 to 10, 2 to 5, 2 to 4, or 2 to 3 bases on one or both sides of a PSE of an IRF-5 target transcript. In embodiments, the flanking sequence includes 3 to 25, 3 to 20, 3 to 15, 3 to 10, 3 to 5, or 3 to 4 bases on one or both sides of a PSE of an IRF-5 target transcript. In embodiments, the flanking sequence includes 4 to 25, 4 to 20, 4 to 15, 4 to 10, or 4 to 5 bases on one or both sides of a PSE of an IRF-5 target transcript. In embodiments, the flanking sequence includes 5 to 25, 5 to 20, 5 to 15, or 5 to 10 bases on one or both sides of a PSE of an IRF-5 target transcript. In embodiments, the flanking sequence includes 10 to 25, 10 to 20, or 10 to 15 bases on one or both sides of a PSE of an IRF-5 target transcript. In embodiments, the flanking sequence includes 15 to 25 or 15 to 20 bases on one or both sides of a PSE an IRF-5 target transcript. In embodiments, the flanking sequence includes 20 to 25 bases on one or both sides of a PSE of an IRF-5 target transcript.

In embodiments, the AC binds to a target nucleotide sequence that does not include a PSE or a portion thereof of an IRF-5 target transcript. In embodiments, the AC binds to a target nucleotide sequence that is in sufficiently close proximity to a PSE to inhibit cleavage and/or addition of a poly(A) tail to the RNA of an IRF-5 target transcript.

In embodiments, the AC binds to a target nucleotide sequence with a 3′ end and/or a 5′ end that is 1 or more, 2 or more, 3 or more, 4 or more, 5 or more, 10 or more, 15 or more, or 20 or more nucleotides from the 5′ end and/or 3′ end of a PSE of an IRF-5 target transcript. In embodiments, the AC binds to a target nucleotide sequence with a 3′ end and/or a 5′ end that is 25 or less, 20 or less, 15 or less, 10 or less, 5 or less, 4 or less, 3 or less, or 2 or less nucleotides from the 5′ end and/or 3′ end of a PSE of an IRF-5 target transcript. In embodiments, the AC binds to a target nucleotide sequence with a 3′ end and/or a 5′ end that is 1 to 25, 1 to 20, 1 to 15, 1 to 10, 1 to 5, 1 to 4, 1 to 3, or 1 to 2 nucleotides form the 5′ end and/or 3′ end of a PSE of an IRF-5 target transcript. In embodiments, the AC binds to a target nucleotide sequence with a 3′ end and/or a 5′ end that is 2 to 25, 2 to 20, 2 to 15, 2 to 10, 2 to 5, 2 to 4, or 2 to 3 nucleotides form the 5′ end and/or 3′ end of a PSE of an IRF-5 target transcript. In embodiments, the AC binds to a target nucleotide sequence with a 3′ end and/or a 5′ end that is 3 to 25, 3 to 20, 3 to 15, 3 to 10, 3 to 5, or 3 to 4 nucleotides form the 5′ end and/or 3′ end of a PSE of an IRF-5 target transcript. In embodiments, the AC binds to a target nucleotide sequence with a 3′ end and/or a 5′ end that is 4 to 25, 4 to 20, 4 to 15, 4 to 10, or 4 to 5 nucleotides form the 5′ end and/or 3′ end of a PSE of an IRF-5 target transcript. In embodiments, the AC binds to a target nucleotide sequence with a 3′ end and/or a 5′ end that is 5 to 25, 5 to 20, 5 to 15, or 5 to 10 nucleotides form the 5′ end and/or 3′ end of a PSE of an IRF-5 target transcript. In embodiments, the AC binds to a target nucleotide sequence with a 3′ end and/or a 5′ end that is 10 to 25 or 10 to 20 nucleotides form the 5′ end and/or 3′ end of a PSE of an IRF-5 target transcript. In embodiments, the AC binds to a target nucleotide sequence with a 3′ end and/or a 5′ end that is 20 to 25 nucleotides form the 5′ end and/or 3′ end of a PSE of an IRF-5 target transcript.

The ACs function via hybridization of an AC with a target nucleotide sequence. Hybridizing of an AC to a target nucleotide sequence that targets polyadenylation of an IRF-5 transcript may elicits a number of different effects. In embodiments, the AC induces the downregulation of IRF-5 transcript levels and/or the expression of gene products, such as IRF-5 proteins. In embodiments, the AC induces the downregulation of the expression of one or more protein isomers encoded by the target IRF-5 transcript/gene. In embodiments, the AC induces upregulation of the expression of the IRF-5 protein encoded by the IRF-5 target transcript/gene. In embodiments, the AC induces increased expression of one or more protein isomers encoded by the IRF-5 target transcript/gene. In embodiments, modulation of cellular concentrations of pre-mRNA or mature mRNA of the IRF-5 target transcript/gene modulates expression of one or more genes other than the target gene, such as downstream genes. In embodiments, the AC induces the downregulation of the expression of one or more proteins that are affected by the expression of the IRF-5 target transcript/gene. In embodiments, the AC induces the upregulation of the expression of one or more proteins that are affected by the expression of the IRF-5 target transcript/gene.

In embodiments, the AC hybridizes to the target nucleotide sequence of the IRF-5 target transcript of thereby increasing stability of the mRNA transcript. In embodiments, the AC hybridizes to the target nucleotide sequence of the IRF-5 target transcript thereby decreasing stability of the mRNA transcript. In embodiments, the AC hybridizes to the target nucleotide sequence of the IRF-5 target transcript thereby resulting in degradation of the mRNA transcript. For example, in embodiments, the AC hybridizes to the target nucleotide sequence of the IRF-5 target transcript thereby resulting in degradation of the polyadenylation site based on a RNase H-mediated mechanism. In embodiments, the AC hybridizes to the target nucleotide sequence of the IRF-5 target transcript does not result in the degradation of the PSE of the mRNA transcript.

In embodiments, the hybridization of an AC to a target transcript regulates transcription, processing, translocation, and/or translation of a target transcript through steric blocking. In embodiments, the AC hybridizes to the target nucleotide sequence of the IRF-5 target transcript thereby sterically blocking the binding of one or more proteins to the mRNA transcript. In embodiments, the AC regulates RNA processing through steric blocking of machinery needed for the polyadenylation of an IRF-5 target transcript (Roberts et al. Nature Reviews Drug Discovery (2020) 19: 673-694). In embodiments, the AC regulates translation and/or protein expression by preventing one or more components of the polyadenylation protein complex from binding to one or more polyadenylation sequence elements of an IRF-5 target transcript. In embodiments, the AC hybridizes to the target nucleotide sequence of an IRF-5 target transcript thereby sterically blocking the binding of CPSF to the mRNA transcript. In embodiments, the AC hybridizes to the target nucleotide sequence of an IRF-5 target transcript thereby sterically blocking the binding of CstF to the mRNA transcript.

IRF-5 target transcripts can include more than one location at which a poly(A) tail may be added (Graham, et al., PNAS (2007), 104(16): 6758-6763). Targeting a PSE that directs addition of the poly(A) tail a particular location may be used to differentially affect the formation and or prevalence of alternative IRF-5 transcripts. In embodiments, binding of the AC to, or in proximity to, a PSE redirects binding of the polyadenylation complex to another polyadenylation site on the IRF5 transcript, resulting in the formation of an alternative transcript. In embodiments, the alternative transcript contains fewer destabilization sequences, such that binding of the AC to, or in proximity to, the PSE results in an increase in IRF-5 target transcript stability. In embodiments, the alternative transcript contains destabilization sequences, such that binding of the AC to the PSE results in decreased IRF-5 target transcript stability (Vickers et al., Nucleic Acids Res. (2001), 29(6):1293-1299). In embodiments, hybridization of the AC to a target nucleotide sequence that includes a PSE that results in steric blockage of the PSE and preferential cleavage at a CS that is not blocked by the AC. In embodiments, the IRF-5 target transcript includes multiple polyadenylation sites and the AC hybridizes to a target nucleotide sequence that includes a PSE of the first (or most 5′) polyadenylation site. In embodiments, the IRF-5 target transcript includes multiple cleavage sites, and the AC hybridizes a target nucleotide sequence that includes the last (or most 3′) cleavage site.

In some embodiments, binding of the AC to the target nucleotide sequence may lead to degradation of the IRF-5 target transcript. In embodiments, where the AC includes DNA bases and/or modified DNA bases, binding of the AC to a target nucleotide sequence creates an DNA/RNA hybrid which can be degraded by RNase H.

In embodiments, an AC binds to a target nucleotide sequence that includes at 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases in the RNA transcript of the following IRF-5 DNA sequence:

(SEQ ID NO: 162)
TCATATCAGATGCTCAAGGCTGGCAGCTACCCCCTTCTTGAGAGT
CCAAGAACCTGGAGCAGAAATAATTTTTATGTATTTTTGGATTAA
TGAATGTTAAAAACA [symbol] CTCAGCTGTTTCTTTCCTTTTACTACTA
CCAGTTGCTCCCATGCTGCTCCACCAGGCCCTGTTTCGGATGCCA
ACTGGCCCACTCCCCAAGCACTTGCCCCCAGCTTGCGACCATTGG
CACTGGGAGGGCCTGGCTTCTGGGCTGATGGGTCAGTTGGGCCTT
CATAAACACTCACCTGGCTGGCTTTGCCTTCCAGGAGGAAGCTGG
CTGAAGCAAGGGTGTGGAATTTTAAATGTGTGCACAGTCTGGAAA
ACTGTCAGAATCAGTTTTCCCATAAAAGGGTGGGCTAGCATTGCA
GCTGCATTTGGGACCATTCAAATCTGTCACTCTCTTGTGTATATT
CCTGTGCTATTAAATATATCAGGGCAGTGCATGTAAATCATCCTG
ATATATTTAATATATTTATTATATTGTCCCCCGAGGTGGGGACAG
TGAGTGAGTTCTCTTAGTCCCCCCAGAGCTGGTTGTTAAAGAGCC
TGGCACCTACCCGCTCTCACTTCATCTGTGTCATCTCTGCACACT
CCAGCCCACTTTCTGCCTTCAGCCATTGAGTGGAAGCTGCCCCAG
GCCCTTACCAGGTGCAGATGCCCAATCTTGATGCCCAGCCATCAG
AACTGTGAGCCAAATAAACCTTTTTCTGTATAAATTA,

where a single underline in bold and italics indicates hexamer polyadenylation signal (PAS) and double underline in bold and italics indicates a cleavage site (CS).

In one embodiment, an AC binds to a target nucleotide sequence that includes at 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases of SEQ ID NO:162 or of variants thereof having 80% or more, 90% or more, 95% or more, 96% or more, 97% or more, 98% or more, 99% or more, 99% or less, 98% or less, 97% or less, 96% or less, 95% or less, 90% or less, 80% to 99;%, 90% to 99%, 95% to 99%, 96% to 99%, 97% to 99%, or 98% to 99% sequence identity to at least a portion of SEQ ID NO:162.

In one embodiment, an AC binds to a target nucleotide sequence that includes at 10 bases or more, 15 bases or more, 20 bases or more, 25 bases more, 40 bases or less, 30 bases or less, 25 bases or less, 20 bases or less, 15 bases or less, 10 to 40 bases, 10 to 30 bases, 10 to 25 bases, 10 to 20 bases, 10 to 15 bases, 15 to 40 bases, 15 to 30 bases, 15 to 25 bases, 15 to 20 bases, 20 to 40 bases, 20 to 30 bases, 20 to 25 bases, 25 to 40 bases, 25 to 30 bases, 30 to 40 bases, 10 bases, 15 bases, 20 bases, 25 bases, 30 bases, or 40 bases wherein said reference to bases denotes consecutive bases of

(nucleotides 712-757 of SEQ ID NO: 162)
5′ . . . GCCATCAGAACTGTGAGCCAAATAAACC
TTTTTCTGTATAAATTA . . . 3′.

In embodiments, the AC is any one of antisense sequences in Table 2, a portion thereof, or a variant thereof that has 80% to 99%, 90 to 99%, 95%-99% sequence identity of a given antisense compound. The ACs in Table 2 target the IRF-5 polyadenylation signal (PS) and/or the CS in which the hexamer PS sequence and CS are in bold. The antisense sequence is the reverse complement of the target nucleotide sequence.

TABLE 2
ASOs of 10, 15, 20, and 30 bases in length for targeting IRF-5

| | SEQ | | SEQ |
|---|---|---|---|
| Antisense Sequence | ID | Target Sequence | ID |
| (5′-3′) | NO: | (5′-3′) | NO: |
| ACACTCGGTT | 163 | TGTGAGCCAA | 266 |
| CACTCGGTTT | 164 | GTGAGCCAAA | 267 |
| ACTCGGTTTA | 165 | TGAGCCAAAT | 268 |
| CTCGGTTTAT | 166 | GAGCCAAATA | 269 |
| TCGGTTTATT | 167 | AGCCAAATAA | 270 |
| CGGTTTATTT | 168 | GCCAAATAAA | 271 |
| GGTTTATTTG | 169 | CCAAATAAAC | 272 |
| GTTTATTTGG | 170 | CAAATAAACC | 273 |
| TTTATTTGGA | 171 | AAATAAACCT | 274 |
| TTATTTGGAA | 172 | AATAAACCTT | 275 |
| TATTTGGAAA | 173 | ATAAACCTTT | 276 |
| ATTTGGAAAA | 174 | TAAACCTTTT | 277 |
| TTTGGAAAAA | 175 | AAACCTTTTT | 278 |
| TTGGAAAAAG | 176 | AACCTTTTTC | 279 |
| TCTTGACACTCGGTT | 177 | AGAACTGTGAGCCAA | 280 |
| CTTGACACTCGGTTT | 178 | GAACTGTGAGCCAAA | 281 |
| TTGACACTCGGTTTA | 179 | AACTGTGAGCCAAAT | 282 |
| TGACACTCGGTTTAT | 180 | ACTGTGAGCCAAATA | 283 |
| GACACTCGGTTTATT | 181 | CTGTGAGCCAAATAA | 284 |
| ACACTCGGTTTATTT | 182 | TGTGAGCCAAATAAA | 285 |
| CACTCGGTTTATTTG | 183 | GTGAGCCAAATAAAC | 286 |
| ACTCGGTTTATTTGG | 184 | TGAGCCAAATAAACC | 287 |
| CTCGGTTTATTTGGA | 185 | GAGCCAAATAAACCT | 288 |
| TCGGTTTATTTGGAA | 186 | AGCCAAATAAACCTT | 289 |
| CGGTTTATTTGGAAA | 187 | GCCAAATAAACCTTT | 290 |
| GGTTTATTTGGAAAA | 188 | CCAAATAAACCTTTT | 291 |
| GTTTATTTGGAAAAA | 189 | CAAATAAACCTTTTT | 292 |
| TTTATTTGGAAAAAG | 190 | AAATAAACCTTTTTC | 293 |
| TTATTTGGAAAAAGA | 191 | AATAAACCTTTTTCT | 294 |
| TATTTGGAAAAAGAC | 192 | ATAAACCTTTTTCTG | 295 |
| ATTTGGAAAAAGACA | 193 | TAAACCTTTTTCTGT | 296 |
| TTTGGAAAAAGACAT | 194 | AAACCTTTTTCTGTA | 297 |
| TTGGAAAAAGACATA | 195 | AACCTTTTTCTGTAT | 298 |
| TGGAAAAAGACATAT | 196 | ACCTTTTTCTGTATA | 299 |
| GGTAGTCTTGACACTCGGTT | 197 | CCATCAGAACTGTGAGCCAA | 300 |
| GTAGTCTTGACACTCGGTTT | 198 | CATCAGAACTGTGAGCCAAA | 301 |
| TAGTCTTGACACTCGGTTTA | 199 | ATCAGAACTGTGAGCCAAAT | 302 |
| AGTCTTGACACTCGGTTTAT | 200 | TCAGAACTGTGAGCCAAATA | 303 |
| GTCTTGACACTCGGTTTATT | 20 | CAGAACTGTGAGCCAAATAA | 304 |
| TCTTGACACTCGGTTTATTT | 202 | AGAACTGTGAGCCAAATAAA | 305 |
| CTTGACACTCGGTTTATTTG | 203 | GAACTGTGAGCCAAATAAAC | 306 |
| TTGACACTCGGTTTATTTGG | 204 | AACTGTGAGCCAAATAAACC | 307 |
| TGACACTCGGTTTATTTGGA | 205 | ACTGTGAGCCAAATAAACCT | 308 |
| GACACTCGGTTTATTTGGAA | 206 | CTGTGAGCCAAATAAACCTT | 309 |
| ACACTCGGTTTATTTGGAAA | 207 | TGTGAGCCAAATAAACCTTT | 310 |
| CACTCGGTTTATTTGGAAAA | 208 | GTGAGCCAAATAAACCTTTT | 311 |
| ACTCGGTTTATTTGGAAAAA | 209 | TGAGCCAAATAAACCTTTTT | 312 |
| CTCGGTTTATTTGGAAAAAG | 210 | GAGCCAAATAAACCTTTTTC | 313 |
| TCGGTTTATTTGGAAAAAGA | 211 | AGCCAAATAAACCTTTTTCT | 314 |
| CGGTTTATTTGGAAAAAGAC | 212 | GCCAAATAAACCTTTTTCTG | 315 |
| GGTTTATTTGGAAAAAGACA | 213 | CCAAATAAACCTTTTTCTGT | 316 |
| GTTTATTTGGAAAAAGACAT | 214 | CAAATAAACCTTTTTCTGTA | 317 |
| TTTATTTGGAAAAAGACATA | 215 | AAATAAACCTTTTTCTGTAT | 318 |
| TTATTTGGAAAAAGACATAT | 216 | AATAAACCTTTTTCTGTATA | 319 |
| TATTTGGAAAAAGACATATT | 217 | ATAAACCTTTTTCTGTATAA | 320 |
| ATTTGGAAAAAGACATATTT | 218 | TAAACCTTTTTCTGTATAAA | 321 |
| TTTGGAAAAAGACATATTTA | 219 | AAACCTTTTTCTGTATAAAT | 322 |
| TTGGAAAAAGACATATTTAA | 220 | AACCTTTTTCTGTATAAATT | 323 |
| CGGTAGTCTTGACACTCGGTTTATT | 221 | GCCATCAGAACTGTGAGCCAAATAA | 324 |
| GGTAGTCTTGACACTCGGTTTATTT | 222 | CCATCAGAACTGTGAGCCAAATAAA | 325 |
| GTAGTCTTGACACTCGGTTTATTTG | 223 | CATCAGAACTGTGAGCCAAATAAAC | 326 |
| TAGTCTTGACACTCGGTTTATTTGG | 224 | ATCAGAACTGTGAGCCAAATAAACC | 327 |
| AGTCTTGACACTCGGTTTATTTGGA | 225 | TCAGAACTGTGAGCCAAATAAACCT | 328 |
| GTCTTGACACTCGGTTTATTTGGAA | 226 | CAGAACTGTGAGCCAAATAAACCTT | 329 |
| TCTTGACACTCGGTTTATTTGGAAA | 227 | AGAACTGTGAGCCAAATAAACCTTT | 330 |
| CTTGACACTCGGTTTATTTGGAAAA | 228 | GAACTGTGAGCCAAATAAACCTTTT | 331 |
| TTGACACTCGGTTTATTTGGAAAAA | 229 | AACTGTGAGCCAAATAAACCTTTTT | 332 |
| TGACACTCGGTTTATTTGGAAAAAG | 230 | ACTGTGAGCCAAATAAACCTTTTTC | 333 |
| GACACTCGGTTTATTTGGAAAAAGA | 231 | CTGTGAGCCAAATAAACCTTTTTCT | 334 |
| ACACTCGGTTTATTTGGAAAAAGAC | 232 | TGTGAGCCAAATAAACCTTTTTCTG | 335 |
| CACTCGGTTTATTTGGAAAAAGACA | 233 | GTGAGCCAAATAAACCTTTTTCTGT | 336 |
| ACTCGGTTTATTTGGAAAAAGACAT | 234 | TGAGCCAAATAAACCTTTTTCTGTA | 337 |
| CTCGGTTTATTTGGAAAAAGACATA | 235 | GAGCCAAATAAACCTTTTTCTGTAT | 338 |
| TCGGTTTATTTGGAAAAAGACATAT | 236 | AGCCAAATAAACCTTTTTCTGTATA | 339 |
| CGGTTTATTTGGAAAAAGACATATT | 237 | GCCAAATAAACCTTTTTCTGTATAA | 340 |
| GGTTTATTTGGAAAAAGACATATTT | 238 | CCAAATAAACCTTTTTCTGTATAAA | 341 |
| GTTTATTTGGAAAAAGACATATTTA | 239 | CAAATAAACCTTTTTCTGTATAAAT | 342 |
| TTTATTTGGAAAAAGACATATTTAA | 240 | AAATAAACCTTTTTCTGTATAAATT | 343 |
| TTATTTGGAAAAAGACATATTTAAT | 241 | AATAAACCTTTTTCTGTATAAATTA | 344 |
| CGGTAGTCTTGACACTCGGTTTATTTGGAA | 242 | GCCATCAGAACTGTGAGCCAAATAAACCTT | 345 |
| GGTAGTCTTGACACTCGGTTTATTTGGAAA | 243 | CCATCAGAACTGTGAGCCAAATAAACCTTT | 346 |
| GTAGTCTTGACACTCGGTTTATTTGGAAAA | 244 | CATCAGAACTGTGAGCCAAATAAACCTTTT | 347 |
| TAGTCTTGACACTCGGTTTATTTGGAAAAA | 245 | ATCAGAACTGTGAGCCAAATAAACCTTTTT | 348 |
| AGTCTTGACACTCGGTTTATTTGGAAAAAG | 246 | TCAGAACTGTGAGCCAAATAAACCTTTTTC | 349 |
| GTCTTGACACTCGGTTTATTTGGAAAAAGA | 247 | CAGAACTGTGAGCCAAATAAACCTTTTTCT | 350 |
| TCTTGACACTCGGTTTATTTGGAAAAAGAC | 248 | AGAACTGTGAGCCAAATAAACCTTTTTCTG | 351 |
| CTTGACACTCGGTTTATTTGGAAAAAGACA | 249 | GAACTGTGAGCCAAATAAACCTTTTTCTGT | 352 |
| TTGACACTCGGTTTATTTGGAAAAAGACAT | 250 | AACTGTGAGCCAAATAAACCTTTTTCTGTA | 353 |
| TGACACTCGGTTTATTTGGAAAAAGACATA | 251 | ACTGTGAGCCAAATAAACCTTTTTCTGTAT | 354 |
| GACACTCGGTTTATTTGGAAAAAGACATAT | 252 | CTGTGAGCCAAATAAACCTTTTTCTGTATA | 355 |
| ACACTCGGTTTATTTGGAAAAAGACATATT | 253 | TGTGAGCCAAATAAACCTTTTTCTGTATAA | 356 |
| CACTCGGTTTATTTGGAAAAAGACATATTT | 254 | GTGAGCCAAATAAACCTTTTTCTGTATAAA | 357 |
| ACTCGGTTTATTTGGAAAAAGACATATTTA | 255 | TGAGCCAAATAAACCTTTTTCTGTATAAAT | 358 |
| CTCGGTTTATTTGGAAAAAGACATATTTAA | 256 | GAGCCAAATAAACCTTTTTCTGTATAAATT | 359 |
| TCGGTTTATTTGGAAAAAGACATATTTAAT | 257 | AGCCAAATAAACCTTTTTCTGTATAAATTA | 360 |
| GCTGAGTCTGTTTTTAACATTCATT | 258 | AATGAATGTTAAAAACAGACTCAGC | 361 |
| TGAGTCTGTTTTTAACATTCATTAA | 259 | TTAATGAATGTTAAAAACAGACTCA | 362 |
| AGTCTGTTTTTAACATTCATTAATC | 260 | GATTAATGAATGTTAAAAACAGACT | 363 |
| TCTGTTTTTAACATTCATTAATCCA | 261 | TGGATTAATGAATGTTAAAAACAGA | 364 |
| AATTTATACAGAAAAAGGTTTATT | 262 | AATAAACCTTTTTCTGTATAAATTA | 365 |
| ATTTATACAGAAAAAGGTTTATTTG | 263 | CAAATAAACCTTTTTCTGTATAAAT | 366 |
| TTATACAGAAAAAGGTTTATTTGGC | 264 | GCCAAATAAACCTTTTTCTGTATAA | 367 |
| TACAGAAAAAGGTTTATTTGGCTC | 265 | GAGCCAAATAAACCTTTTTCTGTA | 368 |
| TAATCATCAGTGGGTTGGCTCTCTG | 369 | CAGAGAGCCAACCCACTGATGATTA | 370 |

The efficacy of the ACs may be assessed by evaluating the antisense activity effected by their administration. As used herein, the term “antisense activity” refers to any detectable and/or measurable activity attributable to the hybridization of an AC to its target nucleotide sequence. Such detection and/or measuring may be direct or indirect. In embodiments, antisense activity is assessed by detecting and or measuring the amount of the protein expressed from the IRF-5 target transcript. In embodiments, antisense activity is assessed by detecting and/or measuring the amount of the transcript of interest. In some embodiments, antisense activity is assessed by detecting and/or measuring the amount of alternative polyadenylation isoforms of the transcript of interest.

### Mechanisms for Targeting the IRF-5 Protein

In embodiments, compounds are provided that include a TM that targets the IRF-5 protein. In embodiments, the TM includes a polypeptide and/or a small molecule. In embodiments, the polypeptide and/or small molecule induces degradation of the IRF-5 protein. In embodiments, the degradation of IRF-5 prevents downstream proinflammatory cytokine production regulated by IRF-5.

In embodiments, the polypeptide and/or small molecule inhibit the activity of IRF-5. In embodiments, the polypeptide and/or small molecule inhibits the activity of IRF-5 by inhibiting the nuclear translocation of an IRF-5 dimer to the nucleus. In embodiments, the polypeptide and/or small molecule inhibits the dimerization of IRF-5. In embodiments, the polypeptide and/or small molecule inhibits the homodimerization of IRF-5 thereby preventing translocation to the nuclease. In embodiments, the polypeptide and/or small molecule inhibits the heterodimerization of IRF-5 thereby preventing translocation to the nucleus. In embodiments, preventing translocation to the nuclease inhibits downstream proinflammatory cytokine production regulated by IRF-5.

In embodiments, the polypeptide is an antigen binding domain. As used herein “antigen binding domain” is a polypeptide that binds to specific target antigen. In embodiments, the target antigen is anyone, or combination of, IRF-5 target protein isoforms. Antigen binding domains and can be designed by screening sequences that bind to an epitope of an antigen, for example, IRF-5. To screen for sequences that bind to an epitope, a cross-blocking assay can be performed such as that described in Antibodies, A Laboratory Manual, Cold Spring Harbor Laboratory, Ed Harlow and David Lane (1988), which is hereby incorporated by reference in its entirety. This assay can be used to determine if, e.g., a CDR for an antibody, or antigen binding fragment thereof binds to the same epitope of an antigen as a different antibody, or antigen binding fragment thereof. Alternately, or additionally, epitope mapping can be performed. For example, the antibody sequence can be mutagenized, such as by alanine scanning, to identify contact residues. In a different method, peptides corresponding to different regions of the target antigen can be used in competition assays with a test antibody. In embodiments, the antigen binding construct is capable of binding to IRF-5. In embodiments, the antigen binding construct includes an antibody (described elsewhere herein) that binds to IRF-5.

In embodiments, the antigen binding domain includes an antibody, antigen binding fragment, or antibody mimetic. In embodiments, the antibody is an anti-IRF5 single chain camelid antibody. In embodiments, the antigen binding fragment includes an a Fab, Fab′, a Fv, scFV, a F(ab)′2, sdAb, minibody, or combinations thereof that binds to the IRF-5 target protein. In embodiments, the single domain antibody (sdAb) is a VHH, VH, or VL capable of binding to target IRF-5 protein. In embodiments, the antibody mimetic includes an affibody, affitin, alphabody, anticalin, avimer, DARPin, fynomer, Kunitz domain peptide, or combinations thereof capable of binding to the target IRF-5 protein.

In embodiments, a compound is provided that includes a TM that induces proteasomal degradation of the target IRF-5 protein. In embodiments, the TM includes a degradation construct. In embodiments, the degradation construct induces polyubiquitylation of the target protein ultimately leading to proteasomal degradation of the target protein.

To maintain homeostasis, cells regulate the degradation of proteins that are misfolded, have aberrant activity, are mutated, or have become obsolete in the cell's current phenotype. Autophagy mediated degradation and proteasomal degradation are the two main mechanism by which cells can degrade proteins.

Proteins may be degraded by the proteasome, a protein complex of various proteases that degrade proteins via proteolysis. Degradation via the proteasome is termed proteasomal degradation. Proteins that have polyubiquitin tags may be targeted for proteasomal degradation. The process of adding a polyubiquitin tag involves several proteins and enzymatic reactions. In embodiments, degradation compounds and degradation constructs are provided that mediate the addition of a polyubiquitin tag on a target protein for proteasomal degradation of the target protein.

Ubiquitin is a 76 amino acid protein that may be conjugated to proteins as a post translational modification to proteins. The process of adding ubiquitin to a target protein is termed ubiquitylation (also known as ubiquitination or ubiquitinylation). Polyubiquitylated proteins may be targeted for proteasomal degradation. A first ubiquitin is conjugated to a target protein through a covalent bond between the C-terminal carboxylate of the ubiquitin and a lysine, cysteine, serine, or threonine side chain or the N-terminus of the target protein. A second ubiquitin can be conjugated to the first ubiquitin through a covalent bond between the C-terminal carboxylate of the second ubiquitin and a lysine or methionine side chain on the first ubiquitin. The nature of the ubiquitin linkages in a polyubiquitin chain specifies the fate of the target protein. For example, chains of 4 or more ubiquitin molecules linked through K48 and chains of ubiquitin linked through K11 often signal for proteasomal degradation of the protein they are conjugated to.

The ubiquitylation process involves three enzymes: a ubiquitin-activating enzyme, a ubiquitin-conjugating enzyme, and a ubiquitin ligase colloquially termed E1, E2, and E3 respectively. E1 activates ubiquitin by catalyzing an ATP dependent reaction resulting in a thioester linkage between the C-terminus of ubiquitin and a cysteine within the active site of E1. E2 catalyzes the transfer of the activated ubiquitin to a cysteine within the active site of E2 via a transthioesterification reaction. E3 catalyzes the transfer of ubiquitin from E2 to the target protein.

There are two known human E1 enzymes, 35 known E2 enzymes, and over 600 known E3 ligases. The large number of E1, E2, and E3 proteins allows for vast diversity and specificity in the ubiquitylation process. The E1 enzymes include UBA1 and UBA6. The E2 enzymes include, but are not limited to, Ube2A, Ube2B, Ube2B, Ube2D1, Ube2D2, Ube2D3, Ube2D4, Ube2E1, Ube2E2, Ube2E3, Ube2G1, Ube2G2, Ube2H, Ube2J1, Ube2J2, Ube2K, Ube2L3, Ube2N, Ube2NL, Ube20, Ube2Q1, Ube2Q2, Ube2QL, Ube2R1, Ube2R2, Ube2S, Ube2T, Ube2V1, Ube2V2, Ube2W, BIRC6, Ube2F, Ube2I, Ube2L6, Ube2M, Ube2Z, ATG10, and ATG3.

The E3 ligases can be classified in several categories including the homologous to E6-associated protein C-terminus (HECT) domain ligases, the Really Interesting New Gene (RING) domain ligases, and the U-box ubiquitin family of ligases (UUL). RING and UUL E3 ligases catalyze the direct transfer of ubiquitin to the target protein. In contrast, HECT E3 ligases require an intermediate step. HECT E3 ligases first catalyze the transfer of the ubiquitin form the E2 to an active cysteine on the HECT E3 ligase before catalyzing the transfer of the ubiquitin from the HECT E3 ligase to the target protein. UULs are a family of modified RING E3 ligases the do not have the full complement of Zn²⁺ binding ligands. While HECT E3 ligases have a direct role in catalysis during ubiquitination, RING and U-box E3 proteins facilitate protein ubiquitination by acting as adaptor molecules that recruit E2 and substrate molecules to promote substrate ubiquitination. Although many RING-type E3 ligases, such as MDM2 (murine double minute clone 2 oncoprotein) and c-Cbl, may act alone, others are found as components of much larger multi-protein complexes, such as the anaphase-promoting complex (“APC”). Table 3A lists some examples of E3 ligases.

Some E3 ligases are E3 ligase complexes that accessory proteins in addition to the protein that is directly involved in catalyzing ubiquitination of the target protein. For example, cullin-RING ligases (CRL) are E3 ligase complexes that catalyze ubiquitinylation of a target protein (Mahon et al., Biomolecules (2014), 13, 4(4):897-930; Jackson et al., Trends Ciochem Sci. (2009), 34(11): 562-570). CRLs include a cullin scaffold protein that recruits a RBX1 or RBX2 (E3 ligases). The cullin scaffold also binds to an adaptor protein. The adaptor protein binds to the target protein. In some cases, the adaptor protein binds to a substrate receptor protein, the substrate receptor protein binds to the target protein. There are seven cullin proteins (Cul1, Cul2, Cul3, Cul4a, Cul4b, Cul5, and Cul7). There are many adaptor proteins including, but not limited to, SKP1, elongin B/C heterodimer, and DDB1. Additionally, there are many substrate receptor proteins including but not limited to, FBP, various SOCS proteins, and various DCAF proteins. Table 2B gives examples of accessory proteins involved in E3 ligase complexes. Table 3B lists some examples of E3 ligase accessory proteins.

TABLE 3A
Example E3 ligases and classes thereof

| E3 ligase | Class | E3 ligase | Class | E3 ligase | Class |
|---|---|---|---|---|---|
| AFF4 | UBOX | PHRF1 | RING | SIAH3 | RING |
| AMFR | RING | PJA1 | RING | SMURF1 | HECTc |
| ANAPC11 | RING | PJA2 | RING | SMURF2 | HECTc |
| ANKIB1 | RING | PLAG1 | RING | STUB1 | UBOX |
| AREL1 | HECTc | PLAGL1 | RING | SYVN1 | RING |
| ARIH1 | RING | PML | RING | TMEM129 | RING |
| ARIH2 | RING | PPIL2 | UBOX | Topors | RING |
| BARD1 | RING | PRPF19 | UBOX | TRAF2 | RING |
| BFAR | RING | RAD18 | RING | TRAF3 | RING |
| BIRC2 | RING | RAG1 | RING | TRAF4 | RING |
| BIRC3 | RING | RAPSN | RING | TRAF5 | RING |
| BIRC7 | RING | RBBP6 | RING | TRAF6 | RING |
| BIRC8 | RING | RBCK1 | RING | TRAF7 | RING |
| BMI1 | RING | RBX1 | RING | TRAIP | RING |
| BRAP | RING | RC3H1 | RING | TRIM10 | RING |
| BRCA1 | RING | RC3H2 | RING | TRIM11 | RING |
| CBL | RING | RCHY1 | RING | TRIM13 | RING |
| CBLB | RING | RFFL | RING | TRIM15 | RING |
| CBLC | RING | RFPL1 | RING | TRIM17 | RING |
| CBLL1 | RING | RFPL2 | RING | TRIM2 | RING |
| CCDC36 | RING | RFPL3 | RING | TRIM21 | RING |
| CCNB1IP1 | RING | RFPL4A | RING | TRIM22 | RING |
| CGRRF1 | RING | RFPL4AL1 | RING | TRIM23 | RING |
| CHER | RING | RFPL4B | RING | TRIM24 | RING |
| CNOT4 | RING | RFWD2 | RING | TRIM25 | RING |
| CUL9 | RING | RFWD3 | RING | TRIM26 | RING |
| CYHR1 | RING | RING1 | RING | TRIM27 | RING |
| DCST1 | RING | RLF | RING | TRIM28 | RING |
| DTX1 | RING | RLIM | RING | TRIM3 | RING |
| DTX2 | RING | RMND5A | RING | TRIM31 | RING |
| DTX3 | RING | RMND5B | RING | TRIM32 | RING |
| DTX3L | RING | RNF10 | RING | TRIM33 | RING |
| DTX4 | RING | RNF103 | RING | TRIM34 | RING |
| DZIP3 | RING | RNF11 | RING | TRIM35 | RING |
| E4F1 | zf- | RNF111 | RING | TRIM36 | RING |
| | C2H2 | | | | |
| FANCL | RING | RNF112 | RING | TRIM37 | RING |
| G2E3 | HECTc | RNF113A | RING | TRIM38 | RING |
| HACE1 | HECTc | RNF113B | RING | TRIM39 | RING |
| HECTD1 | HECTc | RNF114 | RING | TRIM4 | RING |
| HECTD2 | HECTc | RNF115 | RING | TRIM40 | RING |
| HECTD3 | HECT | RNF121 | RING | TRIM41 | RING |
| HECTD4 | HECTc | RNF122 | RING | TRIM42 | RING |
| HECW1 | HECTc | RNF123 | RING | TRIM43 | RING |
| HECW2 | HECTc | RNF125 | RING | TRIM43B | RING |
| HERC1 | HECTc | RNF126 | RING | TRIM45 | RING |
| HERC2 | HECTc | RNF128 | RING | TRIM46 | RING |
| HERC3 | HECTc | RNF13 | RING | TRIM47 | RING |
| HERC4 | HECTc | RNF130 | RING | TRIM48 | RING |
| HERC5 | HECTc | RNF133 | RING | TRIM49 | RING |
| HERC6 | HECTc | RNF135 | RING | TRIM49B | RING |
| HLTF | RING | RNF138 | RING | TRIM49C | RING |
| HUWE1 | HECTc | RNF139 | RING | TRIM49D1 | RING |
| IRF2BP1 | RING | RNF14 | RING | TRIM5 | RING |
| IRF2BP2 | RING | RNF141 | RING | TRIM50 | RING |
| IRF2BPL | RING | RNF144A | RING | TRIM51 | RING |
| Itch | HECTc | RNF144B | RING | TRIM52 | RING |
| KCMF1 | RING | RNF145 | RING | TRIM54 | RING |
| KMT2C | RING | RNF146 | RING | TRIM55 | RING |
| KMT2D | RING | RNF148 | RING | TRIM56 | RING |
| LNX1 | RING | RNF149 | RING | TRIM58 | RING |
| LNX2 | RING | RNF150 | RING | TRIM59 | RING |
| LONRF1 | RING | RNF151 | RING | TRIM6 | RING |
| LONRF2 | RING | RNF152 | RING | TRIM60 | RING |
| LONRF3 | RING | RNF157 | RING | TRIM61 | RING |
| LRSAM1 | RING | RNF165 | RING | TRIM62 | RING |
| LTN1 | RING | RNF166 | RING | TRIM63 | RING |
| MAEA | RING | RNF167 | RING | TRIM64 | RING |
| MAP3K1 | RING | RNF168 | RING | TRIM64B | RING |
| MARCH1 | RING | RNF169 | RING | TRIM64C | RING |
| MARCH10 | RING | RNF17 | RING | TRIM65 | RING |
| MARCH11 | RING | RNF170 | RING | TRIM67 | RING |
| MARCH2 | RING | RNF175 | RING | TRIM68 | RING |
| MARCH3 | RING | RNF180 | RING | TRIM69 | RING |
| MARCH4 | RING | RNF181 | RING | TRIM7 | RING |
| MARCH5 | RING | RNF182 | RING | TRIM71 | RING |
| MARCH6 | RING | RNF183 | RING | TRIM72 | RING |
| MARCH7 | RING | RNF185 | RING | TRIM73 | RING |
| MARCH8 | RING | RNF186 | RING | TRIM74 | RING |
| MARCH9 | RING | RNF187 | RING | TRIM75P | RING |
| Mdm2 | RING | RNF19A | RING | TRIM77 | RING |
| MDM4 | RING | RNF19B | RING | TRIM8 | RING |
| MECOM | RING | RNF2 | RING | TRIM9 | RING |
| MEX3A | RING | RNF20 | RING | TRIML1 | RING |
| MEX3B | RING | RNF207 | RING | TRIML2 | SPRY |
| MEX3C | RING | RNF208 | RING | TRIP12 | HECTc |
| MEX3D | RING | RNF212 | RING | TTC3 | RING |
| MGRN1 | RING | RNF212B | RING | UBE3A | HECTc |
| | | | | (E3A) | |
| MIB1 | RING | RNF213 | RING | UBE3B | HECTc |
| MIB2 | RING | RNF214 | RING | UBE3C | HECTc |
| MID1 | RING | RNF215 | RING | UBE3D | HECT_2 |
| MID2 | RING | RNF216 | RING | UBE4A | RING |
| MKRN1 | RING | RNF217 | RING | UBE4B | RING |
| MKRN2 | RING | RNF219 | RING | UBOX5 | RING |
| MKRN3 | RING | RNF220 | RING | UBR1 | UBR |
| MKRN4P | RING | RNF222 | RING | UBR2 | UBR |
| MNAT1 | RING | RNF223 | RING | UBR3 | UBR |
| MSL2 | RING | RNF224 | RING | UBR4 | UBR |
| MUL1 | RING | RNF225 | RING | UBR5 | HECTc |
| MYCBP2 | RING | RNF24 | RING | UBR7 | UBR |
| MYLIP | RING | RNF25 | RING | UHRF1 | RING |
| NEDD4 | HECTc | RNF26 | RING | UHRF2 | RING |
| NEDD4L | HECTc | RNF31 | RING | UNK | RING |
| NEURL1 | RING | RNF32 | RING | UNKL | RING |
| NEURL1B | RING | RNF34 | RING | VPS11 | RING |
| NEURL3 | RING | RNF38 | RING | VPS18 | RING |
| NFX1 | RING | RNF39 | RING | VPS41 | RING |
| NFXL1 | RING | RNF4 | RING | VPS8 | RING |
| NHLRC1 | RING | RNF40 | RING | WDR59 | RING |
| NOSIP | UBOX | RNF41 | RING | WDSUB1 | RING |
| NSMCE1 | RING | RNF43 | RING | WWP1 | HECTc |
| PARK2 | RING | RNF44 | RING | WWP2 | HECTc |
| PCGF1 | RING | RNF5 | RING | XIAP | RING |
| PCGF2 | RING | RNF6 | RING | ZBTB12 | RING |
| PCGF3 | RING | RNF7 | RING | ZFP91 | zf- |
| | | | | | C2H2 |
| PCGF5 | RING | RNF8 | RING | ZFPL1 | RING |
| PCGF6 | RING | RNFT1 | RING | ZNF280A | HECT |
| PDZRN3 | RING | RNFT2 | RING | ZNF341 | RING |
| PDZRN4 | RING | RSPRY1 | RING | ZNF511 | RING |
| PELI1 | PELI | SCAF11 | RING | ZNF521 | RING |
| PELI2 | PELI | SH3RF1 | RING | ZNF598 | RING |
| PELI3 | PELI | SH3RF2 | RING | ZNF645 | RING |
| PEX10 | RING | SH3RF3 | RING | ZNRF1 | RING |
| PEX12 | RING | SHPRH | RING | ZNRF2 | RING |
| PEX2 | RING | SIAH1 | RING | ZNRF3 | RING |
| PHF7 | RING | SIAH2 | RING | ZNRF4 | RING |
| Zswim2 | RING | ZXDC | RING | LNXp80 | RING |
| CBX4 | — | HUWE2 | HECTc | SNEV | UBOX |
| PIAS1 | — | PIAS2 | — | PIAS3 | |
| PIAS4 | — | RANBP2 | — | ACT1 | UBOX |
| PUB19 | UBOX | | | | |
| RBX2 | RING | | | | |

TABLE 3B
E3 ligase complex accessory proteins

| | | | | | |
|---|---|---|---|---|---|
| ABTB1 | DPF1 | FBXO8 | KCNS3 | KLHL4 | SPSB4 |
| ABTB2 | ECT2L | FBXO9 | KCNV1 | KLHL40 | TCEB3 |
| ANKFY1 | ENC1 | FBXW11 | KCTD1 | KLHL41 | TNFAIP1 |
| ARMC5 | FBXL12 | FBXW2 | KCTD10 | KLHL42 | TNFAIP3 |
| ASB10 | FBXL13 | FBXW4 | KCTD12 | KLHL5 | TULP4 |
| ASB11 | FBXL14 | FBXW5 | KCTD13 | KLHL6 | VHL |
| ASB13 | FBXL15 | FBXW7 | KCTD14 | KLHL7 | WHSC1 |
| ASB14 | FBXL16 | FBXW8 | KCTD15 | KLHL8 | WSB1 |
| ASB15 | FBXL18 | GAN | KCTD16 | KLHL9 | WSB2 |
| ASB16 | FBXL2 | GMCL1 | KCTD17 | LGALS3BP | ZBTB1 |
| ASB17 | FBXL20 | GTF2H2 | KCTD2 | LOC123103 | ZBTB10 |
| ASB18 | FBXL3 | GZF1 | KCTD20 | LRRC29 | ZBTB11 |
| ASB2 | FBXL4 | HIC1 | KCTD21 | LZTR1 | ZBTB14 |
| ASB3 | FBXL5 | HIC2 | KCTD3 | MGC23270 | ZBTB16 |
| ASB4 | FBXL6 | IBTK | KCTD4 | MYNN | ZBTB17 |
| ASB5 | FBXL7 | IPP | KCTD5 | NACC1 | ZBTB18 |
| ASB6 | FBXL8 | IVNS1ABP | KCTD6 | NACC2 | ZBTB2 |
| ASB7 | FBXO10 | KBTBD11 | KCTD7 | NEURL2 | ZBTB20 |
| ASB8 | FBXO11 | KBTBD12 | KCTD7 | OSTM1 | ZBTB21 |
| ASB9 | FBXO15 | KBTBD2 | KCTD8 | OTUD7A | ZBTB22 |
| ATRX | FBXO16 | KBTBD3 | KCTD9 | OTUD7B | ZBTB24 |
| BACH1 | FBXO17 | KBTBD4 | KDM2A | PATZ1 | ZBTB25 |
| BACH2 | FBXO18 | KBTBD6 | KDM2B | RAB40A | ZBTB26 |
| BCL6 | FBXO2 | KBTBD7 | KEAP1 | RAB40AL | ZBTB3 |
| BCL6B | FBXO21 | KBTBD8 | KLHL1 | RAB40B | ZBTB32 |
| BTBD1 | FBXO22 | KBTBD9 | KLHL10 | RAB40C | ZBTB33 |
| BTBD10 | FBXO24 | KCNA1 | KLHL11 | RABGEF1 | ZBTB37 |
| BTBD11 | FBXO25 | KCNA10 | KLHL12 | RCBTB1 | ZBTB38 |
| BTBD17 | FBXO27 | KCNA2 | KLHL13 | RCBTB2 | ZBTB39 |
| BTBD2 | FBXO28 | KCNA3 | KLHL14 | RHOBTB1 | ZBTB4 |
| BTBD3 | FBXO3 | KCNA4 | KLHL15 | RHOBTB2 | ZBTB40 |
| BTBD6 | FBXO30 | KCNA5 | KLHL17 | RHOBTB3 | ZBTB41 |
| BTBD7 | FBXO31 | KCNA6 | KLHL18 | SF3B3 | ZBTB43 |
| BTBD8 | FBXO32 | KCNA7 | KLHL20 | SHKBP1 | ZBTB44 |
| BTBD9 | FBXO33 | KCNB1 | KLHL21 | SKP2 | ZBTB45 |
| BTRC | FBXO34 | KCNB2 | KLHL22 | SLX4 | ZBTB46 |
| CCIN | FBXO36 | KCNC1 | KLHL23 | SOCS1 | ZBTB48 |
| CCNF | FBXO38 | KCNC2 | KLHL24 | SOCS2 | ZBTB49 |
| CISH | FBXO4 | KCNC3 | KLHL25 | SOCS3 | ZBTB5 |
| CPSF1 | FBXO40 | KCNC4 | KLHL26 | SOCS4 | ZBTB6 |
| CUL1 | FBXO41 | KCND1 | KLHL28 | SOCSS | ZBTB7A |
| CUL2 | FBXO42 | KCND2 | KLHL3 | SOCS6 | ZBTB7B |
| CUL3 | FBXO44 | KCND3 | KLHL30 | SOCS7 | ZBTB7C |
| CUL4A | FBXO45 | KCNG1 | KLHL31 | SPOP | ZBTB8A |
| CUL4B | FBXO46 | KCNG3 | KLHL32 | SPOPL | ZBTB9 |
| CUL5 | FBXO5 | KCNRG | KLHL34 | SPSB1 | ZFAND3 |
| CUL7 | FBXO6 | KCNS1 | KLHL36 | SPSB2 | ZFAND4 |
| DDB1 | FBXO7 | KCNS2 | KLHL38 | SPSB3 | ZFAND5 |
| | | | | ZNF131 | ZFAND6 |

In embodiments, the degradation construct includes a targeting moiety and a degradation moiety. The targeting moiety and degradation moiety are operably linked. The targeting moiety binds to the IRF5-target protein. The degradation moiety includes an active E3 ligase, a portion of functional E3 ligase, or an E3 ligase targeting domain. The degradation moiety facilities the polyubiquitylation of the IRF-5 target protein.

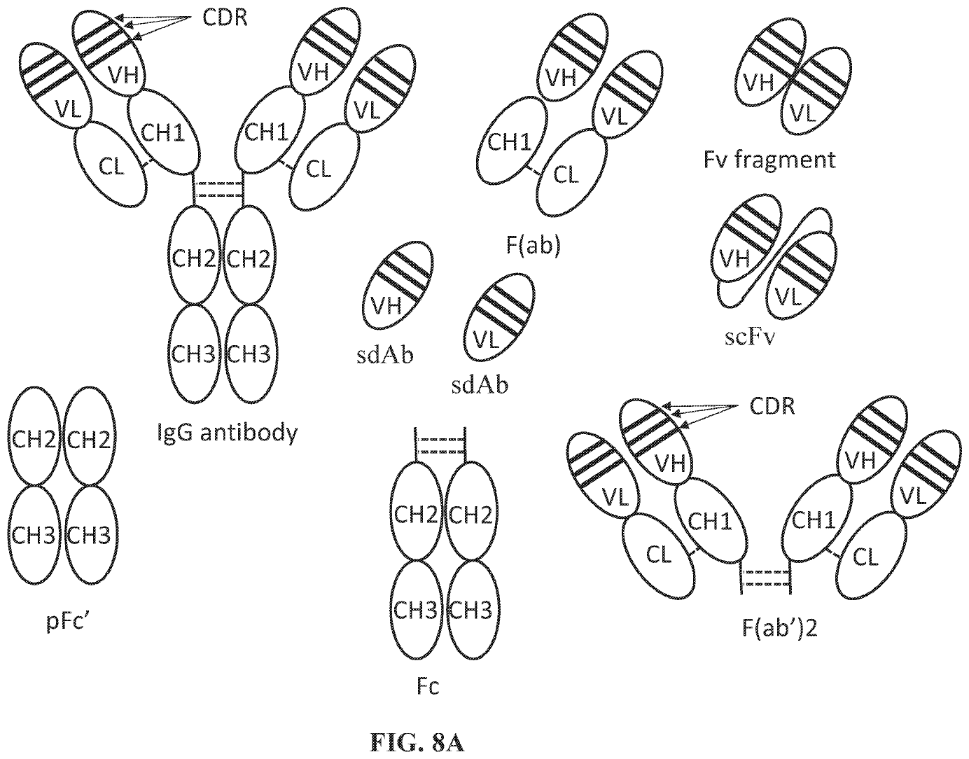

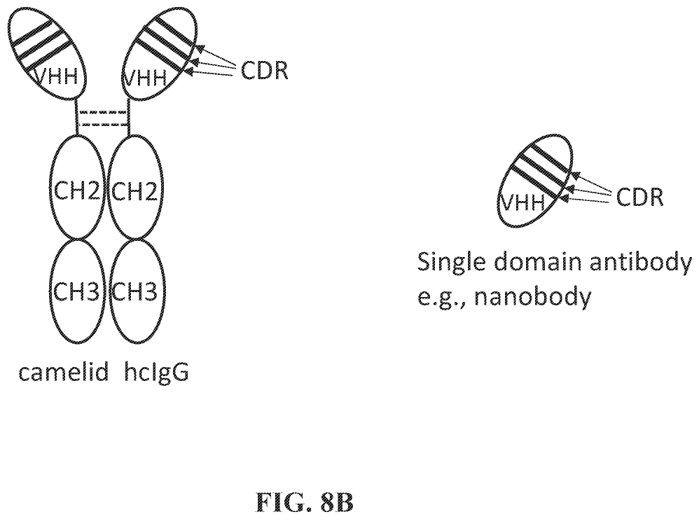

FIG. 8C shows how compounds of the present disclosure that include a degradation construct induce the degradation of the IRF-5 target protein. A compound of the present disclosure is conjugated to a degradation construct 10. The degradation construct includes a targeting moiety 30 and a degradation moiety 20. The targeting moiety 30 is designed to bind to the IRF-5 target protein 40. The degradation moiety is configured to facilitate polyubiquitylation of the target protein which leads to proteasomal degradation of the target protein. In embodiments, where the degradation moiety 20 includes an active E3 ligase or a portion of an active E3 ligase, the degradation moiety catalyzes the polyubiquitylation of the target protein. In embodiments, where the degradation moiety 20 includes an E3 ligase recruiting domain, the E3 ligase recruiting domain interacts with an endogenous E3 ligase or an endogenous E3 ligase complex 50 to catalyze the polyubiquitylation 60 of the target protein 40.

In embodiments, the degradation moiety includes an E3 ligase or a portion thereof. In embodiments, the E3 ligase includes any one of the E3 ligases and/or E3 ligase accessory proteins in Table 3A. In embodiments, the active degradation moiety includes a RING E3 ligase or an active portion of a RING E3 ligase. In embodiments, the active degradation moiety includes a HECT E3 ligase or an active portion of a HECT E3 ligase. In embodiments, the active degradation moiety includes a UBOX E3 ligase or an active portion of a UBOX E3 ligase. In embodiments, the E3 ligase or a portion thereof includes an E3 ligase accessory protein. In embodiments, the E3 ligase includes VHL, MDM2, UBOX5, CRBN, TRIM21, SOCS1, active portions thereof, and combinations thereof.

In embodiments, the degradation moiety includes an E3 ligase recruiter moiety. In embodiments, the E3 ligase recruiter moiety interacts with an endogenous E3 ligase to induce polyubiquitylation and subsequent proteasomal degradation of the IRF-5 target protein. In embodiments, the E3 ligase interacts with any member of an E3 ligase complex to induce polyubiquitylation and subsequent proteasomal degradation of the target protein. In embodiments, the E3 ligase interacts with a CRL. In embodiments, the E3 ligase recruiter moiety interacts with an E3 ligase accessory protein such as, for example, an adaptor protein, substrate receptor protein of an E3 ligase complex.

The E3 ligase recruiter moiety may be a peptide and/or a small molecule. In embodiments, the E3 ligase recruiter moiety can be designed and/or chosen to interact with a specific E3 ligase and/or E3 ligase complex (e.g., CLR) such as those described herein.

In embodiments, the E3 ligase recruiter moiety includes a peptide. In embodiments, the E3 ligase recruiter includes IkBalpha or an active portion thereof. The IkBalpha, or active portion thereof, binds to the beta-TrCP subunit of an E3 ligase, for example, as described in U.S. Pat. No. 7,208,517, which is herein incorporated by reference in its entirety. In embodiments, the E3 ligase recruiter moiety includes IgG1 Fc domain or portion thereof. In embodiments, the IgG1 Fc domain or portion thereof, may include one or more amino acid mutations compared to the wild type IgG1 Fc domain or portion thereof. In embodiments, the IgG1 Fc domain or a portion thereof interacts with E3 ligase TRIM21.

In embodiments, the peptide is a peptidomimetic. In embodiments, the peptidomimetic E3 ligase recruiter motely includes VH032, VH101, VH298, LCL161, methylbestin, derivatives thereof, and combinations thereof. In embodiments, VH032, VH101, VH298, derivatives thereof, and combinations thereof interact with the VHL E3 ligase. In embodiments, LCL161, methylbestin, derivatives thereof, and combinations thereof interact with the cIAP E3 ligase.

In embodiments, the E3 ligase recruiter moiety includes a non-peptidomimetic small molecule. Examples of non-peptidomimetic small molecule that may be used as an E3 ligase recruiting moiety include, but are not limited to, thalidomide, pomalidomide, lenalidomide, bardoxolone methyl, nutlin-3, nimbolide, indisulam, derivatives thereof, and combinations thereof (Ishida et al., SLAS Discov. (2021), 26(4): 484-502; Sun et al., Nature, Signal Transduction and Targeted Therapy (2019), 4(64), doi.org/10.1038/s41392-019-0101-6; Troup et al., Exploration of Target Anti-tumor Therapy (2020), 1:273-312. doi.org/10.37349/etat.2020.00018). In embodiments, thalidomide, lenalidomide, pomalidomide, and derivatives thereof, interact with CRBN. In embodiments, nutlin-3 and derivatives thereof interact with MDM2. In embodiments, nimbolide and derivatives thereof interact with RNF114.

In embodiments, indisulam and derivatives thereof interact with the CUL4 CLR E3 ligase complex. In embodiments, indisulam and derivatives thereof interact with substrate receptor protein DCAF15.

In embodiments, the E3 ligase recruiter moiety is designed to interact with a specific E3 ligase of member of an E3 ligase complex such as those described herein. In embodiments, the E3 ligase recruiter moiety is chosen and/or designed to interact with VHL, CRBN, TRIM21, SOCS1, UBOX5, or combinations thereof.

The degradation construct includes a targeting moiety. The targeting moiety includes an antigen-binding domain that binds to IRF-5. The antigen binding domain may include an antibody, and antigen binding fragment, or an antibody mimetic.

In embodiments, the antigen binding domain includes an antibody. In embodiments, the antibody is an IgG or camelid single chain antibody.

In embodiments, the antigen binding domain includes an antigen binding fragment. In embodiments, the antigen binding fragment includes a Fab, Fab′, F(ab′)₂, pFc′, Fd, scFv, sdFv, sdAb, diabody, bispecific diabody, monobody, or combinations thereof. In embodiments, the single domain antibody (sdAb) includes a VH, VL, or VHH.

In embodiments, the antigen binding domain includes an antibody mimetic. In embodiments, the antibody mimetic includes an affibody, an affilin, an affitin, an alphabody, an anticalin, an avimer, a DARPin, a fynomer, a Kunitz domain peptide, or combinations thereof.

In embodiments, the degradation construct includes additional domains. In embodiments, the degradation construct includes a second targeting moiety. The second targeting moiety may be any targeting moiety as described elsewhere except that it binds specifically to a second antigen other than the target protein. In embodiments, the second targeting domain binds to an extracellular protein.

The targeting moiety, degradation moiety, and any additional domains of the degradation are operably linked. As used herein, the term “operably linked” refers to a direct or indirect covalent linking between the degradation moiety and the targeting moiety as well as any additional domains of the degradation construct. Thus, the degradation moiety and the targeting moiety that are operably linked may be directly covalently coupled to one another. Conversely, the degradation moiety and the targeting moiety may be connected by mutual covalent linking to an intervening component (e.g., a flanking sequence or linker). For example, in embodiments where the degradation construct includes a degradation moiety, a targeting moiety, and a second targeting moiety, the degradation moiety and the second targeting moiety may be separately directly linked to targeting moiety; or the degradation moiety may be directly linked to the targeting moiety and the second targeting moiety may be directly linked to the degradation moiety.

The targeting moiety and the degradation moiety may be operably linked through one or more linkers. The term “linker” as used herein in the context of functional components degradation constructs refers any bond, small molecule, peptide sequence, or other vehicle that physically links the functional components degradation constructs. Linkers can be susceptible to or be substantially resistant to acid-induced cleavage, light-induced cleavage, peptidase-induced cleavage, esterase-induced cleavage, and/or disulfide bond cleavage. Linkers are classified based on the presence of one or more chemical motifs such as, for example, including a disulfide group, a hydrazine group or peptide (cleavable), or a thioester group (non-cleavable). Linkers also include charged linkers, and hydrophilic forms thereof as known in the art.

Suitable linkers for linking the targeting moiety and the degradation moiety of the degradation of the present disclosure include a natural linker, an empirical linker, or a combination of natural and/or empirical linkers. Natural linkers are derived from the amino acid linking sequence of multi-domain proteins, which are naturally present between protein domains. Properties of natural linkers such as, for example, length, hydrophobicity, amino acid residues, and/or secondary structure can be exploited to confer desirable properties to a multi-domain compound that includes natural linkers connecting the components of the degradation constructs of the present disclosure.

The studies of linkers in natural multi-domain proteins have led to the generation of many empirical linkers with various sequences and conformations for the construction of recombinant fusion proteins. Empirical linkers are often classified as three types: flexible linkers, rigid linkers, and cleavable linkers. Flexible linkers can provide a certain degree of movement or interaction at the joined components. Flexible linkers typically include small, non-polar (e.g., Gly) or polar (e.g., Ser or Thr) amino acids, which provide flexibility, and allow for mobility of the connected components. Rigid linkers can successfully keep a fixed distance between the degradation moiety and the targeting moiety of the degradation constructs to maintain their independent functions, which can provide efficient separation of targeting moiety and the degradation moiety and/or sufficiently reduce interference between targeting moiety and the degradation moiety.

In some embodiments, the natural linker or empirical linker is covalently attached to the targeting moiety, degradation moiety, or both, using bioconjugation chemistries. Bioconjugation chemistries are well known in the art and include but are not limited to, NHS-ester ligation, isocyanate ligation, isothiocyanate ligation, benzoyl fluoride ligation, maleimide conjugation, iodoacetamide conjugation, 2-thiopyridine disulfide exchange, 3-arylpropiolonitrile conjugation, diazonium salt conjugation, PTAD conjugation, and Mannich ligation.

In some embodiments, the natural linker or empirical linker, the targeting moiety, the degradation moiety, or both, may include one or more unnatural amino acids that allow for bioorthogonal conjugation reactions. As used herein, “bioorthogonal conjugation” refers to a conjugation reaction that uses one or more unnatural amino acids or modified amino acids as a starting reagent. Examples of bioorthogonal conjugation reactions include but are not limited to, Staudinger ligation, copper-catalyzed azide-alkyne cycloaddition, strain promoted [3+2]cycloadditions, tetrazine ligation, metal-catalyzed coupling reactions, or oxime-hydrazone ligations. Examples of non-natural amino acids include, but are not limited to, azidohomoalanine, 2 homopropargylglycine, 3 homoallylglycine, 4 p-acetyl-Phe, 5 p-azido-Phe, 3-(6-acetylnaphthalen-2-ylamino)-2-aminopropanoic acid, N^{ε}-(cyclooct-2-yn-1-yloxy)carbonyl)L-lysine, N^{ε}-2-azideoethyloxycarbonyl-L-lysine, Nε-p-azidobenzyloxycarbonyl lysine, Propargyl-L-lysine, or trans-cyclooct-2-ene lysine.

In some embodiments, the linker is derived from a small molecule, such as a polymer. Example polymer linkers include but are not limited to, poly-ethylene glycol, poly(N-isopropylacrylamide), and N,N′-dimethylacrylamide)-co-4-phenylazophenyl acrylate. The small molecule linkers generally include one or more reactive handles allowing conjugation to the degradation moiety, targeting moiety, or both. In some embodiments, the reactive handle allows for a bioconjugation or bioorthogonal conjugation. In some embodiments, the reactive handle allows for any organic reaction compatible with conjugating a linker to the targeting moiety, degradation moiety, or both.

The linker may be conjugated at any amino acid location of the targeting moiety. For example, the linker may be conjugated to the N-terminus, C-terminus, or any amino acid between.

In embodiments where the degradation construct includes additional domains, the additional domains may be operably coupled to each other and/or the targeting moiety and/or degradation moiety using one or more of the linkers disclosed elsewhere herein.

In some embodiments where the degradation construct includes an targeting moiety and a degradation moiety comprised of amino acids that are operably coupled by peptide linkers, the degradation construct may be produced by expression in a host cell. In some embodiments where the degradation construct includes a targeting moiety and a degradation moiety comprised of amino acids that are operably coupled by peptide linkers, the degradation construct may be produced by solid phase peptide synthesis.

### Types of Therapeutic Moieties

In embodiments, the TM may be of several class including an antisense compound, a peptide, an antibody, and/or a small molecule. The class and identity of the TM depends on the mechanism being used to modulate the level and/or activity of IRF-5.

### Antisense Compound (AC)

In embodiments, the therapeutic moiety (TM) includes an antisense compound (AC). The term “antisense compound” refers to an oligonucleotide sequence that is complementary, or at least partially complementary to a target nucleotide sequence. An AC is an oligonucleotide that includes traditional DNA bases, modified DNA bases, traditional RNA bases, modified RNA bases, traditional RNA sugars, modified RNA sugars, traditional DNA sugars, modified DNA sugars, traditional internucleoside linkages, modified internucleoside linkages, or combinations thereof. In embodiments, the AC includes a nucleotide sequence that is at least partially complementary to target nucleotide sequence found within an IRF-5 target transcript. ACs include, but are not limited to, RNAi, microRNA, antagomirs, aptamers, ribozymes, immunostimulatory oligonucleotides, decoy oligonucleotides, supermir, miRNA mimics, miRNA inhibitors, U1 adapters, and combinations thereof.

In embodiments, an AC may be used to modulate the activity and/or levels of a the IRF-5 target transcript and/or protein. In embodiments, an AC may be used to modulate the activity and/or levels of the IRF-5 target transcript and/or protein through various mechanism that modulate splicing (described elsewhere). In embodiments, the AC may be used to modulate the activity and/or levels of the IRF-5 target transcript and/or protein through various mechanisms that modulate polyadenylation of the IRF-5 target transcript (described elsewhere).

In embodiments, the ACs hybridize with (e.g., bind to) a target nucleotide sequence of an IRF-5 target transcript. The target nucleotide sequence may be any target nucleotide sequence and/or include any sequence elements as described elsewhere herein. In embodiments, the AC binds to a target nucleotide sequence found within Isoform 1, IRF-5 Isoform 2, IRF-5 Isoform 3, IRF-5 Isoform 4, IRF-5 Isoform 5, IRF-5 Isoform 6, or combinations thereof. In embodiments, the AC binds to a target nucleotide sequence Isoform 1, IRF-5 Isoform 2, IRF-5 Isoform 3, IRF-5 Isoform 4, IRF-5 Isoform 5, IRF-5 Isoform 6 or a sequence that is 80% to 100%, 90% to 100%, 95% to 100%, or 99% to 100% identical to a nucleic acid sequence encoding IRF-5 Isoform 1, IRF-5 Isoform 2, IRF-5 Isoform 3, IRF-5 Isoform 4, IRF-5 Isoform 5, or IRF-5 Isoform 6.

In embodiments, the AC is the same length as the target nucleotide sequence. In embodiments, the AC is a different length than the target nucleotide sequence. In embodiments, the AC is longer than the target nucleic acid sequence.

In embodiments, the AC is 5 or more, 10 or more, 15 or more, 20 or more, 25 or more, 30 or more, 35 or more, 40 or more, or 45 or more nucleic acids in length. In embodiments, the AC is 50 or less, 45 or less, 40 or less, 35 or less, 30 or less, 25 or less, 20 or less, 15 or less, or 10 or less nucleic acids in length. In embodiments, the AC is 5 to 50, 5 to 45, 5 to 40, 5 to 35, 5 to 30, 5 to 25, 5 to 20, 5 to 15, or 5 to 10 nucleic acids in length. In embodiments, the AC is 10 to 50, 10 to 45, 10 to 40, 10 to 35, 10 to 30, 10 to 25, 10 to 20, or 10 to 15 nucleic acids in length. In embodiments, the AC is 15 to 50, 15 to 45, 15 to 40, 15 to 35, 15 to 30, 15 to 25, or 15 to 20 nucleic acids in length. In embodiments, the AC is 20 to 50, 20 to 45, 20 to 40, 20 to 35, 20 to 30, or 20 to 25 nucleic acids in length. In embodiments, the AC is 25 to 50, 25 to 45, 25 to 40, 25 to 35, or 25 to 30 nucleic acids in length. In embodiments, the AC is 30 to 50, 30 to 45, 30 to 40, or 30 to 35 nucleic acids in length. In embodiments, the AC is 35 to 50, 35 to 45, or 35 to 40 nucleic acids in length. In embodiments, the AC is 40 to 50 or 40 to 45 nucleic acids in length. In embodiments, the AC is 45 to 50 nucleic acids in length. In embodiments, the AC is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleic acids in length.

In embodiments, the AC has 100% complementarity to a target nucleotide sequence. In embodiments, the AC does not have 100/complementarity to a target nucleotide sequence. As used herein, the term “percent complementarity” refers to the number of nucleobases of an AC that have nucleobase complementarity with a corresponding nucleobase of an oligomeric compound or nucleic acid (e.g., a target nucleotide sequence) divided by the total length (number of nucleobases) of the AC. One skilled in the art recognizes that the inclusion of mismatches is possible without eliminating the activity of the antisense compound.

In embodiments, the AC includes 20% or less, 15% or less, 10% or less, 5% or less, or zero mismatches to the target nucleotide sequence. In some embodiments, the AC includes 5% or more, 10% or more, or 15% or more mismatched. In embodiments, the AC includes zero to 5%, zero to 10%, zero to 15%, or zero to 20% mismatches to the target nucleotide sequence. In embodiments, the AC includes 5% to 10%, 5% to 15%, or 5% to 20% mismatches to the target nucleotide sequence. In embodiments, the AC includes 10% to 15% or 10% to 20% mismatches to the target nucleotide sequence. In embodiments, the AC includes 10% to 20% mismatches to the target nucleotide sequence.

In embodiments, the AC has 80% or greater, 85% or greater, 90% or greater, 95% or greater, 96% or greater, 97% or greater, 98% or greater, or 99% or greater complementarity to a target nucleotide sequence. In embodiments, the AC has 100% or less, 99% or less, 98% or less, 97% or less 96% or less 95% or less, 90% or less, 85% or less complementarity to a target nucleotide sequence. In embodiments, the AC has 80% to 100%, 80% to 99%, 80% to 98%, 80% to 97% 80% to 96%, 80% to 95%, 80% to 90% or 80⁰/c to 85% complementarity to a target nucleotide sequence. In embodiments, the AC has 85% to 100%, 85% to 99%, 85% to 98%, 85% to 97% 85% to 96%, 85% to 95%, or 85% to 90% complementarity to a target nucleotide sequence. In embodiments, the AC has 90% to 100%, 90% to 99%, 90% to 98%, 90/o to 97%, 90% to 96%, or 90% to 95% complementarity to a target nucleotide sequence. In embodiments, the AC has 95% to 100%, 95% to 99/o, 95% to 98%, 95% to 97%, or 95% to 96% complementarity to a target nucleotide sequence. In embodiments, the AC has 96% to 100%, 96% to 99%, 96% to 98%, or 96% to 97% complementarity to a target nucleotide sequence. In embodiments, the AC has 97% to 100%, 97% to 99%, or 97% to 98% complementarity to a target nucleotide sequence. In embodiments, the AC has 98% to 100% or 98% to 99% complementarity to a target nucleotide sequence. In embodiments, the AC has 99% to 100% complementarity to a target nucleotide sequence. Percent complementarity of an oligonucleotide is calculated by dividing the number of complementarity nucleobases by the total number of nucleobases of the oligonucleotide.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between an AC and a target nucleotide sequence, such as by determining the thermal melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

The ACs described herein may contain one or more asymmetric centers and thus give rise to enantiomers, diastereomers, and other stereoisomeric configurations that may be defined, in terms of absolute stereochemistry, as (R) or (S); α or β; or as (D) or (L). Included in the antisense compounds provided herein are all such possible isomers, as well as their racemic and optically pure forms.

The efficacy of the ACs may be assessed by evaluating the antisense activity effected by their administration. As used herein, the term “antisense activity” refers to any detectable and/or measurable activity attributable to the hybridization of an antisense compound to its target nucleotide sequence. Such detection and/or measuring may be direct or indirect. In embodiments, antisense activity is assessed by detecting and or measuring the amount of the protein expressed from the transcript of interest. In embodiments, antisense activity is assessed by detecting and/or measuring the amount of the transcript of interest. In embodiments, antisense activity is assessed by detecting and/or measuring the amount of alternatively spliced RNA and/or the amount of protein isoforms translated from the target transcript.

### AC Structure

The AC includes an oligonucleotide and/or an oligonucleoside. Oligonucleotides and/or oligonucleotides are nucleotides or nucleosides linked through internucleoside linkages. Nucleosides include a pentose sugar (e.g., ribose or deoxyribose) and a nitrogenous base covalently attached to sugar. The naturally occurring (or traditional basses) bases found in DNA and/or RNA are adenine (A), guanine (G), thymine (T), cytosine (C), and uracil (U). The naturally occurring sugars (or traditional sugars) found in DNA and/or RNA deoxyribose (DNA) and ribose (RNA). The naturally occurring nucleoside linkage (or traditional internucleoside linkage) is a phosphodiester bond. In embodiments, the ACs of the present disclosure may have all natural sugars, bases, and internucleoside linkages.

Chemically modified nucleosides are routinely used for incorporation into antisense compounds to enhance one or more properties, such as nuclease resistance, pharmacokinetics, or affinity for a target RNA. In embodiments, the ACs of the present disclosure may have one or more modified nucleosides. In embodiments, the ACs of the present disclosure may have one or more modified sugars. In embodiments, the ACs of the present disclosure may have one or more modified bases. In embodiments, the ACs of the present disclosure may have one or more modified internucleoside linkages.

In general, a nucleobase is any group that contains one or more atom or groups of atoms capable of hydrogen bonding to a base of another nucleic acid. In addition to “unmodified” or “natural” nucleobases (A, G, T, C, and U) many modified nucleobases or nucleobase mimetics are known to those skilled in the art are amenable with the compounds described herein Generally a modified nucleobase refers to a nucleobase that is fairly similar in structure to the parent nucleobase, such as for example a 7-deaza purine, a 5-methyl cytosine, 2-thio-dT (FIG. 1) or a G-clamp. Generally, a nucleobase mimetic is a nucleobase that includes a structure that is more complicated than a modified nucleobase, such as for example a tricyclic phenoxazine nucleobase mimetic. Methods for preparation of the above noted modified nucleobases are well known to those skilled in the art.

In embodiments, the AC may include one or more nucleosides having a modified sugar moiety. In embodiments, the furanosyl sugar of a natural nucleoside may have a 2′ modification, modifications to make a constrained nucleoside, and others (see FIG. 1). For example, in embodiments, the furanosyl sugar ring of a natural nucleoside can be modified in a number of ways including, but not limited to, addition of a substituent group, bridging of two non-geminal ring atoms to form a bicyclic nucleic acid (BNA) or a locked nucleic acid; exchanging the oxygen of the furanosyl ring with C or N; and/or substitution of an atom or group such (see FIG. 1). Modified sugars are well known and can be used to increase or decrease the affinity of the AC for its target nucleotide sequence. Modified sugars may also be used increase AC resistance to nucleases. Sugars can also be replaced with sugar mimetic groups among others. In embodiments, one or more sugars of the nucleosides of the AC is replaced with a methylenemorpholine ring as shown as 19 in FIG. 1.

In embodiments, the AC includes one or more nucleosides that include a bicyclic modified sugar (BNA; sometimes called bridged nucleic acids). Examples of BNAs include, but are not limited to LNA (4′-(CH₂)—O-2′ bridge), 2′-thio-LNA (4′-(CH₂)—S-2′ bridge), 2′-amino-LNA (4′-(CH₂)—NR-2′ bridge), ENA (4′-(CH₂)₂—O-2′ bridge), 4′-(CH₂)₃-2′ bridged BNA, 4′-(CH₂CH(CH₃))-2′ bridged BNA″ cEt (4′-(CH(CH₃)—O-2′ bridge), and cMOE BNAs (4′-(CH(CH₂OCH₃)—O-2′ bridge). BNA's have been prepared and disclosed in the patent literature as well as in scientific literature (Srivastava, et al. J. Am. Chem. Soc. (2007), ACS Advanced online publication, 10.1021/ja071106y; Albaek et al. J. Org. Chem. (2006), 71, 7731-7740; Fluiter, et al. Chembiochem (2005), 6, 1104-1109; Singh et al., Chem. Commun. (1998), 4, 455-456; Koshkin et al., Tetrahedron (1998), 54, 3607-3630; Wahlestedt et al., Proc. Natl. Acad. Sci. U.S.A. (2000), 97, 5633-5638; Kumar et al., Bioorg. Med. Chem. Lett. (1998), 8, 2219-2222; WO 94/14226; WO 2005/021570; Singh et al., J. Org. Chem. (1998), 63, 10035-10039, WO 2007/090071; U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807).

In embodiments, the AC includes one or more nucleosides that include a locked nucleic acid (LNA). In LNAs the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs (2001), 2, 558-561; Braasch et al., Chem. Biol. (2001), 8 1-7; and Orum et al., Curr. Opinion Mol. Ther. (2001), 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun. (1998), 4,455-456; ENA™; Morita et al., Bioorganic Medicinal Chemistry (2003), 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A. (2000), 97, 5633-5638).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research (2003), 21, 6365-6372).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of LNAanalogs containing oligodeoxyribonucleotide duplexes as substrates for nucleic acid polymerases has also been described (Wengel et al., WO 99/14226). Furthermore, synthesis of 2′-amino-LNA, a conformationally restricted high-affinity oligonucleotide analog has been described (Singh et al., J. Org. Chem. (1998), 63, 10035-10039). In addition, 2′-amino- and 2′-methylamino-LNA's have been prepared and the thermal stability of their duplexes with complementary RNA and DNA strands has been previously reported.

Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and 6,600,032; and WO 2005/121371.

### Internucleoside Linkages

Described herein are internucleoside linking groups that link the nucleosides or otherwise modified nucleoside monomer units together thereby forming an oligonucleotide and/or an oligonucleotide containing AC. The ACs may include naturally occurring internucleoside linkages, unnatural internucleoside linkages, or both.

In naturally occurring DNA and RNA, the internucleoside linking group is a phosphodiester that covalently links adjacent nucleosides to one another to form a linear polymeric compound. In naturally occurring DNA and RNA, phosphodiester is linked to the 2′, 3′ or 5′ hydroxyl moiety of the sugar. Within oligonucleotides, the phosphate groups are commonly referred to as forming the internucleoside backbone of the oligonucleotide. In naturally occurring DNA and RNA, the linkage or backbone of RNA and DNA, is a 3′ to 5′ phosphodiester linkage. In embodiments, the internucleoside linking groups of the ACs are phosphodiesters. In embodiments, the internucleoside linking groups of the ACs are 3′ to 5′ phosphodiester linkages.

The two main classes of unnatural internucleoside linking groups are defined by the presence or absence of a phosphorus atom. Representative phosphorus containing internucleoside linkages include, but are not limited to, phosphotriesters, methylphosphonates, phosphoramidate, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H₂—O—); and N,N′-dimethylhydrazine (—CH₂—N(CH₃)—N(CH₃)—). ACs having phosphorus internucleoside linking groups are referred to as oligonucleotides. Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared as racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, two or more nucleosides having modified sugars and/or modified nucleobases may be joined using a phosphoramidate. In embodiments, two or more nucleosides having a methylenemorpholine ring may be connected through a phosphoramidate internucleoside linkage as shown as 20 in FIG. 1 where B1 and B2 are modified or natural nucleobases.

Antisense compounds that include nucleobases with a methylenemorpholine ring that are linked through phosphoramidate internucleoside linkage may be referred to as phosphoramidate morpholino oligomers (PMOs).

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the AC or the CPP (CPP discussed elsewhere herein).

In embodiments, conjugate groups include lipid moieties such as a cholesterol moiety (Letsinger et al., Proc. Natl. Acad. Sci. USA (1989), 86, 6553); cholic acid (Manoharan et al., Bioorg. Med. Chem. Lett. (1994), 4, 1053); a thioether, e.g., hexyl-S-tritylthiol (Manoharan et al., Ann. N.Y. Acad. Sci. (1992), 660, 306; Manoharan et al., Bioorg. Med. Chem. Let. (1993), 3, 2765); a thiocholesterol (Oberhauser et al., Nucl. Acids Res. (1992), 20, 533); an aliphatic chain, e.g., dodecandiol or undecyl residues (Saison-Behmoaras et al., EMBO J. (1991), 10, 111; Kabanov et al., FEBS Lett. (1990), 259, 327; Svinarchuk et al., Biochimie (1993), 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett. (1995), 36, 3651; Shea et al., Nucl. Acids Res. (1990), 18, 3777); a polyamine or a polyethylene glycol chain (Manoharan et al., Nucleosides & Nucleotides (1995), 14, 969); adamantane acetic acid (Manoharan et al., Tetrahedron Lett. (1995), 36, 3651); a palmityl moiety (Mishra et al., Biochim. Biophys. Acta. (1995), 1264, 229); or an octadecylamine or hexylamino-carbonyl-oxycholesterol moiety (Crooke et al., J. Pharmacol. Exp. Ther. (1996), 277,923).

### Types of Antisense Compounds

Various types of AC may be used for example, including an antisense oligonucleotide, siRNA, microRNA, antagomir, aptamer, ribozyme, supermir, miRNA mimic, miRNA inhibitor, or combinations thereof.

### Antisense Oligonucleotides

In various embodiments, the antisense compound (AC) is an antisense oligonucleotide (ASO) that is complementary to a target nucleotide sequence. The term “antisense oligonucleotide (ASO)” or simply “antisense” is meant to include oligonucleotides that are complementary to a target nucleotide sequence. The term also encompasses ASOs that may not be fully complementary to the desired target nucleotide sequence. ASOs include single strands of DNA and/or RNA that are complementary to a chosen target nucleotide sequence or a target gene. ASOs may include one or more modified DNA and/or RNA bases, modified sugars, and/or unnatural internucleoside linkages. In embodiments, the ASOs may include one or more phosphoramidate internucleoside linkages. In embodiments, the ASO is phosphoramidate morpholino oligomers (PMOs). ASOs may have any characteristic, be any length, bind to any target nucleotide sequence and/or sequence element, and effect any mechanism as described relative to an AC.

Antisense oligonucleotides have been demonstrated to be effective as targeted inhibitors of protein synthesis, and, consequently, can be used to specifically inhibit protein synthesis by a targeted gene. The efficacy of ASO for inhibiting protein synthesis is well established. To date, these compounds have shown promise in several in vitro and in vivo models, including models of inflammatory disease, cancer, and HIV (Agrawal, Trends in Biotech. (1996), 14:376-387). Antisense can also affect cellular activity by hybridizing specifically with chromosomal DNA.

Methods of producing ASOs are known in the art and can be readily adapted to produce an ASO that binds to a target nucleotide sequence of the present disclosure. Selection of ASOs sequences specific for a given target nucleotide sequence is based upon analysis of the chosen target nucleotide sequence and determination of secondary structure, Tm, binding energy, and relative stability. Antisense oligonucleotides may be selected based upon their relative inability to form dimers, hairpins, or other secondary structures that would reduce or prohibit specific binding to the target nucleotide sequence in a host cell. These secondary structure analyses and target site selection considerations can be performed, for example, using v.4 of the OLIGO primer analysis software (Molecular Biology Insights) and/or the BLASTN 2.0.5 algorithm software (Altschul et ai, Nucleic Acids Res. 1997, 25(17):3389-402).

### RNA Interference

In embodiments, the AC includes a molecule that mediates RNA interference (RNAi). As used herein, the phrase “mediates RNAi” refers to the ability to silence, in a sequence specific manner, a target transcript. While not wishing to be bound by theory, it is believed that silencing uses the RNAi machinery or process and a guide RNA, e.g., an siRNA compound of from about 21 to about 23 nucleotides. In embodiments, the AC targets the target transcript for degradation. As such, in embodiments, RNAi molecule may be used to disrupt the expression of a gene or polynucleotide of interest. In embodiments, RNAi molecule is used to induce degradation of the target transcript, such as a pre-mRNA or a mature mRNA.

In embodiments, the AC includes a small interfering RNA (siRNA) that elicits an RNAi response.

Small interfering RNAs (siRNAs) are nucleic acid duplexes normally from about 16 to about 30 nucleotides long that can associate with a cytoplasmic multi-protein complex known as RNAi-induced silencing complex (RISC). RISC loaded with siRNA mediates the degradation of homologous transcripts, therefore siRNA can be designed to knock down protein expression with high specificity. Unlike other antisense technologies, siRNA function through a natural mechanism evolved to control gene expression through non-coding RNA. A variety of RNAi reagents, including siRNAs targeting clinically relevant targets, are currently under pharmaceutical development, as described, e.g., in de Fougerolles, A. et al., Nature Reviews (2007) 6:443-453.

While the first described RNAi molecules were RNA:RNA hybrids that include both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., Molecular Biotechnology (2003), 24:111-119). In embodiments, RNAi molecules are used that include any of these different types of double-stranded molecules. In addition, it is understood that RNAi molecules may be used and introduced to cells in a variety of forms. Accordingly, as used herein, RNAi molecules encompasses any and all molecules capable of mediating an RNAi in cells, including, but not limited to, double-stranded oligonucleotides that include two separate strands, i.e. a sense strand and an antisense strand, e.g., small interfering RNA (siRNA); double-stranded oligonucleotide that includes two separate strands that are linked together by non-nucleotidyl linker; oligonucleotides that include a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is at least about 14, at least about 15, at least about 20, at least about 25, at least about 30, at least about 35, at least about 40, or up to about 50 nucleotides in length. In certain embodiments, the single strand siRNA is less than about 200, about 100, or about 60 nucleotides in length.

Hairpin siRNA compounds may have a duplex region equal to or at least about 17, about 18, about 19, about 20, about 21, about 22, about 23, about 24, or about 25 nucleotide pairs. The duplex region may be equal to or less than about 200, about 100, or about 50 nucleotide pairs in length. In certain embodiments, ranges for the duplex region are from about 15 to about 30, from about 17 to about 23, from about 19 to about 23, and from about 19 to about 21 nucleotides pairs in length. The hairpin may have a single strand overhang or terminal unpaired region. In certain embodiments, the overhangs are from about 2 to about 3 nucleotides in length. In embodiments, the overhang is at the same side of the hairpin and in embodiments on the antisense side of the hairpin.

A “double stranded siRNA compound” as used herein, is an siRNA compound which includes more than one, and in some cases two, strands in which interchain hybridization can form a region of duplex structure.

The antisense strand of a double stranded siRNA compound may be equal to or at least about 14, about 15, about 16 about 17, about 18, about 19, about 20, about 25, about 30, about 40, or about 60 nucleotides in length. It may be equal to or less than about 200, about 100, or about 50 nucleotides in length. Ranges may be from about 17 to about 25, from about 19 to about 23, and from about 19 to about 21 nucleotides in length. As used herein, term “antisense strand” means the strand of an siRNA compound that is sufficiently complementary to a target molecule, e.g., the target nucleotide sequence of a target transcript.

The sense strand of a double stranded siRNA compound may be equal to or at least about 14, about 15, about 16, about 17, about 18, about 19, about 20, about 25, about 30, about 40, or about 60 nucleotides in length. It may be equal to or less than about 200, about 100, or about 50, nucleotides in length. Ranges may be from about 17 to about 25, from about 19 to about 23, and from about 19 to about 21 nucleotides in length.

The double strand portion of a double stranded siRNA compound may be equal to or at least about 14, about 15, about 16, about 17, about 18, about 19, about 20, about 21, about 22, about 23, about 24, about 25, about 30, about 40, or about 60 nucleotide pairs in length. It may be equal to or less than about 200, about 100, or about 50, nucleotides pairs in length. Ranges may be from about 15 to about 30, from about 17 to about 23, from about 19 to about 23, and from about 19 to about 21 nucleotides pairs in length.

In embodiments, the siRNA compound is sufficiently large that it can be cleaved by an endogenous molecule, e.g., by Dicer, to produce smaller siRNA compounds, e.g., siRNAs agents.

The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1 to 3 nucleotides. The overhangs can be the result of one strand being longer than the other, or the result of two strands of the same length being staggered. Some embodiments will have at least one 3′ overhang. In embodiments, both ends of an siRNA molecule will have a 3′ overhang. In embodiments, the overhang is 2 nucleotides.

In embodiments, the length for the duplexed region is from about 15 to about 30, or about 18, about 19, about 20, about 21, about 22, or about 23 nucleotides in length, e.g., in the ssiRNA (siRNA with sticky overhangs) compound range discussed above. ssiRNA compounds can resemble in length and structure the natural Dicer processed products from long dsiRNAs. Embodiments in which the two strands of the ssiRNA compound are linked, e.g., covalently linked are also included. In embodiments, hairpin, or other single strand structures which provide a double stranded region, and a 3′ over hangs are included.

The siRNA compounds described herein, including double-stranded siRNA compounds and single-stranded siRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene.

In embodiments, an siRNA compound is “sufficiently complementary” to a target transcript, such that the siRNA compound silences production of protein encoded by the target mRNA. In embodiments, the siRNA compound is “sufficiently complementary” to at least a portion of a target transcript, such that the siRNA compound silences production of the gene product encoded by the target transcript. In another embodiment, the siRNA compound is “exactly complementary” to a target nucleotide sequence (e.g., a portion of a target transcript) such that the target nucleotide sequence and the siRNA compound anneal, for example to form a hybrid made exclusively of Watson-Crick base pairs in the region of exact complementarity. A “sufficiently complementary” to a target nucleotide sequence can include an internal region (e.g., of at least about 10 nucleotides) that is exactly complementary to a target nucleotide sequence. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

The therapeutic applications of RNAi are extremely broad, since siRNA and miRNA constructs can be synthesized with any nucleotide sequence directed against a target protein. To date, siRNA constructs have shown the ability to specifically down-regulate target proteins in both in vitro and in vivo models, as well as in clinical studies

### MicroRNAs

In embodiments, the AC includes a microRNA molecule. MicroRNAs (miRNAs) are a highly conserved class of small RNA molecules that are transcribed from DNA in the genomes of plants and animals but are not translated into protein. Processed miRNAs are single stranded 17-25 nucleotide RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

### Antagomirs

In embodiments, the AC is an antagomir. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes that include the antagomir and endogenous miRNA, thereby preventing miRNA-induced gene silencing. An example of antagomir-mediated miRNA silencing is the silencing of miR-122, described in Krutzfeldt et al., Nature (2005), 438: 685-689, which is expressly incorporated by reference herein in its entirety. Antagomir RNAs may be synthesized using standard solid phase oligonucleotide synthesis protocols (U.S. patent application Ser. Nos. 11/502,158 and 11/657,341; the disclosure of each of which are incorporated herein by reference).

An antagomir can include ligand-conjugated monomer subunits and monomers for oligonucleotide synthesis. Monomers are described in U.S. application Ser. No. 10/916,185. An antagomir can have a ZXY structure, such as is described in PCT Application No. PCT/US2004/07070. An antagomir can be complexed with an amphipathic moiety. Amphipathic moieties for use with oligonucleotide agents are described in PCT Application No. PCT/US2004/07070.

### Aplamers

In embodiments, the AC includes an aptamer. Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules (Eaton, Curr. Opin. Chem. Biol. (1997), 1: 10-16; Famulok, Curr. Opin. Struct. Biol. (1999), 9:324-9; and Hermann and Patel, Science (2000), 287:820-5). Aptamers may be RNA or DNA based and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, the term “aptamer” also includes “secondary aptamers” containing a consensus sequence derived from comparing two or more known aptamers to a given target. In embodiments, the aptamer is an “intracellular aptamer”, or “intramer”, which specifically recognize intracellular targets (Famulok et al., Chem Biol. (2001), 8(10):931-939; Yoon and Rossi, Adv. Drug Deliv. Rev. (2018), 134:22-35; each incorporated by reference herein).

### Ribozymes

In embodiments, the AC is a ribozyme. Ribozymes are RNA molecules complexes having specific catalytic domains that possess endonuclease activity (Kim and Cech, Proc. Natl. Acad. Sci. USA (1987), 84(24):8788-92; Forster and Symons, Cell (1987) 24, 49(2):211-20). For example, a large number of ribozymes accelerate phosphoester transfer reactions with a high degree of specificity, often cleaving only one of several phosphoesters in an oligonucleotide substrate (Cech et al., Cell (1981), 27 (3 Pt 2):487-96; Michel and Westhof, J. Mol. Biol. (1990), 5, 216(3):585-610; Reinhold-Hurek and Shub, Nature (1992), 14, 357(6374): 173-6). This specificity has been attributed to the requirement that the substrate bind via specific base-pairing interactions to the internal guide sequence (IGS) of the ribozyme prior to chemical reaction.

At least six basic varieties of naturally occurring enzymatic RNAs are known presently. Each can catalyze the hydrolysis of RNA phosphodiester bonds in trans (and thus can cleave other RNA molecules) under physiological conditions, In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. (1992), 20(17):4559-65. Examples of hairpin motifs are described by Hampel et al. (Eur. Pat. Appl. Publ. No. EP 0360257), Hampel and Tritz, Biochemistry (1989), 28(12):4929-33; Hampel et al, Nucleic Acids Res. (1990), 18(2):299-304 and U.S. Pat. No. 5,631,359. An example of the hepatitis virus motif is described by Perrotta and Been, Biochemistry (1992), 31(47): 11843-52; an example of the RNaseP motif is described by Guerrier-Takada et al., Cell (1983), 35 (3 Pt 2):849-57; Neurospora VS RNA ribozyme motif is described by Collins (Saville and Collins, Cell (1990), 61(4). 685-96; Saville and Collins, Proc. Natl. Acad. Sci. USA (1991), 88(19):8826-30; Collins and Olive, Biochemistry (1993), 32 (1 1):2795-9); and an example of the Group I intron is described in U.S. Pat. No. 4,987,071. In embodiments, enzymatic nucleic acid molecules have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein. In embodiments, the ribozyme is targeted to a target nucleotide sequence of a target transcript.

Ribozyme activity can be increased by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem H bases to shorten RNA synthesis times and reduce chemical requirements.

### Supermir

In embodiments, the AC is a supermir. A supermir refers to a single stranded, double stranded, or partially double stranded oligomer or polymer of RNA, polymer of DNA, or both, or modifications thereof, which has a nucleotide sequence that is substantially identical to an miRNA and that is antisense with respect to its target, This term includes oligonucleotides composed of naturally-occurring nucleobases, sugars and covalent internucleoside (backbone) linkages and which contain at least one non-naturally-occurring portion which functions similarly. Such modified or substituted oligonucleotides have desirable properties such as, for example, enhanced cellular uptake, enhanced affinity for nucleic acid target and increased stability in the presence of nucleases. In embodiments, the supermir does not include a sense strand, and in another embodiment, the supermir does not self-hybridize to a significant extent. A supermir can have secondary structure, but it is substantially single-stranded under physiological conditions. A supermir that is substantially single-stranded is single-stranded to the extent that less than about 50% (e.g., less than about 40%, about 30%, about 20%, about 10%, or about 5%) of the supermir is duplexed with itself. The supermir can include a hairpin segment, e.g., sequence, for example, at the 3′ end can self-hybridize and form a duplex region, e.g., a duplex region of at least about 1, about 2, about 3, or about 4 or less than about 8, about 7, about 6, or about 5 nucleotides, or about 5 nucleotides. The duplexed region can be connected by a linker, e.g., a nucleotide linker, e.g., about 3, about 4, about 5, or about 6 dTs, e.g., modified dTs. In another embodiment the supermir is duplexed with a shorter oligo, e.g., of about 5, about 6, about 7, about 8, about 9, or about 10 nucleotides in length, e.g., at one or both of the 3′ and 5′ end or at one end and in the non-terminal or middle of the supermir.

miRNA Mimics

In embodiments, the AC is a mi RNA mimic. miRNA mimics represent a class of molecules that can be used to imitate the gene silencing ability of one or more miRNAs. Thus, the term “microRNA mimic” refers to synthetic non-coding RNAs (i.e., the miRNA is not obtained by purification from a source of the endogenous miRNA) that are capable of entering the RNAi pathway and regulating gene expression. miRNA mimics can be designed as mature molecules (e.g., single stranded) or mimic precursors (e.g., pri- or pre-miRNAs). miRNA mimics can include nucleic acid (modified or modified nucleic acids) including oligonucleotides that include, without limitation, RNA, modified RNA, DNA, modified DNA, locked nucleic acids, or 2′-0,4′-C-ethylene-bridged nucleic acids (ENA), or any combination of the above (including DNA-RNA hybrids). In addition, miRNA mimics can include conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can include 2′ modifications (including 2′-0 methyl modifications and 2′ F modifications) on one or both strands of the molecule and internucleoside modifications (e.g., phosphorothioate modifications) that enhance nucleic acid stability and/or specificity. In addition, miRNA mimics can include overhangs. The overhangs can include from about 1 to about 6 nucleotides on either the 3′ or 5′ end of either strand and can be modified to enhance stability or functionality. In embodiments, a miRNA mimic includes a duplex region of from about 16 to about 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can include 2′ F modification of all of the Cs and Us, phosphorylation of the 5′ end of the oligonucleotide, and stabilized internucleoside linkages associated with a 2 nucleotide 3′ overhang.

miRNA Inhibitor

In embodiments, the AC is a miRNA inhibitor. The terms “antimir” “microRNA inhibitor”, “miR inhibitor”, or “miRNA inhibitor” are synonymous and refer to oligonucleotides or modified oligonucleotides that interfere with the ability of specific miRNAs. In general, the inhibitors are nucleic acid or modified nucleic acids in nature including oligonucleotides that include RNA, modified RNA, DNA, modified DNA, locked nucleic acids (LNAs), or any combination of the above.

Modifications include 2′ modifications (including 2′-0 alkyl modifications and 2′ F modifications) and internucleoside modifications (e.g., phosphorothioate modifications) that can affect delivery, stability, specificity, intracellular compartmentalization, or potency. In addition, miRNA inhibitors can include conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors include contain one or more sequences or portions of sequences that are complementary or partially complementary with the mature strand (or strands) of the miRNA to be targeted. In addition, the miRNA inhibitor may also include additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In embodiments, one or both of the additional sequences are arbitrary sequences capable of forming hairpins. Thus, in embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

Linking groups or bifunctional linking moieties such as those known in the art are amenable to the compounds provided herein. Linking groups are useful for attachment of chemical functional groups, conjugate groups, reporter groups and other groups to selective sites in a parent compound such as for example an AC. In general, a bifunctional linking moiety includes a hydrocarbyl moiety having two functional groups. One of the functional groups is selected to bind to a parent molecule or compound of interest and the other is selected to bind essentially any selected group such as chemical functional group or a conjugate group. Any of the linkers described here may be used. In embodiments, the linker includes a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of substituent groups includes hydroxyl, amino, alkoxy, carboxy, benzyl, phenyl, nitro, thiol, thioalkoxy, halogen, alkyl, aryl, alkenyl and alkynyl.

In embodiments, AC includes nucleotide modification designed to not support RNase H activity. Nucleotide modifications of antisense compounds that do not support RNase H activity are known and include, but are not limited to, 2′-O-methoxy ethyl/phosphorothioate (MOE) modifications. Advantageously, AC with MOE modifications have increased affinity for target RNA and increase nuclease stability.

### Immunostimulatory Oligonucleotides

In embodiments, the therapeutic moiety is an immunostimulatory oligonucleotide. Immunostimulatory oligonucleotides (ISS; single- or double-stranded) are capable of inducing an immune response when administered to a patient, which may be a mammal or other patient. ISS include, e.g., certain palindromes leading to hairpin secondary structures (see Yamamoto S., et al. (1992) J. Immunol. 148: 4072-4076), or CpG motifs, as well as other known ISS features (such as multi-G domains, see WO 96/11266).

The immune response may be an innate or an adaptive immune response. The immune system is divided into a more innate immune system, and acquired adaptive immune system of vertebrates, the latter of which is further divided into humoral cellular components. In particular embodiments, the immune response may be mucosal.

Immunostimulatory nucleic acids are considered to be non-sequence specific when it is not required that they specifically bind to and reduce the expression of a target polynucleotide in order to provoke an immune response. Thus, certain immunostimulatory nucleic acids may include a sequence corresponding to a region of a naturally occurring gene or mRNA, but they may still be considered non-sequence specific immunostimulatory nucleic acids.

In embodiments, the immunostimulatory nucleic acid or oligonucleotide includes at least one CpG dinucleotide. The oligonucleotide or CpG dinucleotide may be unmethylated or methylated. In another embodiment, the immunostimulatory nucleic acid includes at least one CpG dinucleotide having a methylated cytosine. In embodiments, the nucleic acid includes a single CpG dinucleotide, wherein the cytosine in said CpG dinucleotide is methylated. In a specific embodiment, the nucleic acid includes the sequence 5‘ TAACGTTGAGGG’CAT 3′ (SEQ ID NO: 369). In an alternative embodiment, the nucleic acid includes at least two CpG dinucleotides, wherein at least one cytosine in the CpG dinucleotides is methylated. In a further embodiment, each cytosine in the CpG dinucleotides present in the sequence is methylated. In another embodiment, the nucleic acid includes a plurality of CpG dinucleotides, wherein at least one of said CpG dinucleotides includes a methylated cytosine.

Additional specific nucleic acid sequences of oligonucleotides (ODNs) suitable for use in the compositions and methods are described in Raney et al, Journal of Pharmacology and Experimental Therapeutics, 298:1185-1192 (2001). In certain embodiments, ODNs used in the compositions and methods have a phosphodiester (“PO”) backbone or a phosphorothioate (“PS”) backbone, and/or at least one methylated cytosine residue in a CpG motif.

### Decoy Oligonucleotides

In embodiments, the therapeutic moiety is a decoy oligonucleotide. Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest, 2000, 106: 1071-1075, which is expressly incorporated by reference herein, in its entirety.

### U1 Adaptor

In some embodiments, the therapeutic moiety is a U1 adaptor. U1 adaptors inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant Mol Biol 49:77-95). Nucleotides 2-11 of the 5′ end of U1 snRNA base pair bind with the 5′ ss of the pre mRNA. In one embodiment, oligonucleotides are U1 adaptors. In one embodiment, the U1 adaptor can be administered in combination with at least one other iRNA agent.

### (CRISPR) Gene-Editing Machinery

In embodiments, the compounds disclosed herein include one or more CPP (or cCPP) conjugated to CRISPR gene-editing machinery. As used herein, “CRISPR gene-editing machinery” refers to protein, nucleic acids, or combinations thereof, which may be used to edit a genome. Non-limiting examples of gene-editing machinery include gRNAs, nucleases, nuclease inhibitors, and combinations and complexes thereof. The following patent documents describe CRISPR gene-editing machinery: U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, U.S. patent application Ser. No. 14/704,551, and U.S. patent application Ser. No. 13/842,859. Each of the aforementioned patent documents is incorporated by reference herein in its entirety.

In embodiments, a linker conjugates the cCPP to the CRISPR gene-editing machinery. Any linker described in this disclosure or that is known to a person of skill in the art may be utilized.

gRNA

In embodiments, the compounds include the CPP (or cCPP) is conjugated to a gRNA. A gRNA targets a genomic loci in a prokaryotic or eukaryotic cell.

In embodiments, the gRNA is a single-molecule guide RNA (sgRNA). A sgRNA includes a spacer sequence and a scaffold sequence. A spacer sequence is a short nucleic acid sequence used to target a nuclease (e.g., a Cas9 nuclease) to a specific nucleotide region of interest (e.g., a genomic DNA sequence to be cleaved). In embodiments, the spacer may be about 17-24 bases in length, such as about 20 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 bases in length. In embodiments, the spacer may be about 15, about 16, about 17, about 18, about 19, about 20, about 21, about 22, about 23, about 24, about 25, about 26, about 27, about 28, about 29, or about 30 bases in length. In embodiments, the spacer sequence has between about 40% to about 80% GC content.

In embodiments, the spacer targets a site that immediately precedes a 5′ protospacer adjacent motif (PAM). The PAM sequence may be selected based on the desired nuclease. For example, the PAM sequence may be any one of the PAM sequences shown in Table 4 below, wherein N refers to any nucleic acid, R refers to A or G, Y refers to C or T, W refers to A or T, and V refers to A or C or G.

TABLE 4
Exemplary Nucleases and PAM sequences

| PAM sequence (5′ to 3′) | Nuclease | Isolated from |
|---|---|---|
| NGG | SpCas9 | Streptococcus pyogenes |
| NGRRT or NGRRN | SaCas9 | Staphylococcus aureus |
| NNNNGATT | NmeCas9 | Neisseria meningitidis |
| NNNNRYAC | CjCas9 | Campylobacter jejuni |
| NNAGAAW | StCas9 | Streptococcus thermophiles |
| TTTV | LbCpf1 | Lachnospiraceae bacterium |
| TTTV | AsCpf1 | Acidaminococcus sp. |

In embodiments, a spacer may target a sequence of a mammalian gene, such as a human gene. In embodiments, the spacer may target a mutant gene. In embodiments, the spacer may target a coding sequence. In embodiments, the spacer may target an exonic sequence. In embodiments, the spacer may target a polyadenylation site (PS). In embodiments, the spacer may target a sequence element of a PS. In embodiments, the spacer may target a polyadenylation signal (PAS), an intervening sequence (IS), a cleavage site (CS), a downstream element (DES), or a portion or combination thereof. In embodiments, a spacer may target a splicing element (SE) or a cis-splicing regulatory element (SRE).

The scaffold sequence is the sequence within the sgRNA that is responsible for nuclease (e.g., Cas9) binding. The scaffold sequence does not include the spacer/targeting sequence. In embodiments, the scaffold may be about 1 to about 10, about 10 to about 20, about 20 to about 30, about 30 to about 40, about 40 to about 50, about 50 to about 60, about 60 to about 70, about 70 to about 80, about 80 to about 90, about 90 to about 100, about 100 to about 110, about 110 to about 120, or about 120 to about 130 nucleotides in length. In embodiments, the scaffold may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In embodiments, the scaffold may be at least 10, at least 20, at least 30, at least 40, at least 50, at least 60, at least 70, at least 80, at least 90, at least 100, at least 110, at least 120, or at least 125 nucleotides in length.

In embodiments, the gRNA is a dual-molecule guide RNA, e.g, crRNA and tracrRNA. In embodiments, the gRNA may further include a poly(A) tail.

In embodiments, a compound that includes a CPP is conjugated to a nucleic acid that includes a gRNA. In embodiments, the nucleic acid includes about 1, about 2, about 3, about 4, about 5, about 6, about 7, about 8, about 9, about 10, about 11, about 12, about 13, about 14, about 15, about 16, about 17, about 18, about 19, or about 20 gRNAs. In embodiments, the gRNAs recognize the same target. In embodiments, the gRNAs recognize different targets. In embodiments, the nucleic acid that includes a gRNA includes a sequence encoding a promoter, wherein the promoter drives expression of the gRNA.

### Nuclease

In embodiments, the compounds include a cell penetrating peptide conjugated to a nuclease. In embodiments, the nuclease is a Type II, Type V-A, Type V-B, Type VC, Type V-U, Type VI-B nuclease. In embodiments, the nuclease is a transcription, activator-like effector nuclease (TALEN), a meganuclease, or a zinc-finger nuclease. In embodiments, the nuclease is a Cas9, Cas12a (Cpf1), Cas12b, Cas12c, Tnp-B like, Cas13a (C2c2), Cas13b, or Cas14 nuclease. For example, in some embodiments, the nuclease is a Cas9 nuclease or a Cpf1 nuclease.

In embodiments, the nuclease is a modified form or variant of a Cas9, Cas12a (Cpf1), Cas12b, Cas12c, Tnp-B like, Cas13a (C2c2), Cas13b, or Cas14 nuclease. In embodiments, the nuclease is a modified form or variant of a TAL nuclease, a meganuclease, or a zinc-finger nuclease. A “modified” or “variant” nuclease is one that is, for example, truncated, fused to another protein (such as another nuclease), catalytically inactivated, etc. In embodiments, the nuclease may have at least about 80%, at least about 85%, at least about 90%, at least about 95%, at least about 98%, at least about 99%, or about 100% sequence identity to a naturally occurring Cas9, Cas12a (Cpf1), Cas12b, Cas12c, Tnp-B like, Cas13a (C2c2), Cas13b, Cas14 nuclease, or a TALEN, meganuclease, or zinc-finger nuclease. In embodiments, the nuclease is a Cas9 nuclease derived from S. pyogenes (SpCas9). In embodiments, a nuclease has at least about 90%, at least about 95%, at least about 96%, at least about 97%, at least about 98%, or at least about 99% sequence identity to a Cas9 nuclease derived from S. pyogenes (SpCas9). In embodiments, the nuclease is a Cas9 derived from S. aureus (SaCas9). In embodiments, the nuclease has at least about 90%, at least about 95%, at least about 96%, at least about 97%, at least about 98%, or at least about 99% sequence identity to a Cas9 derived from S. aureus (SaCas9). In embodiments, the Cpf1 is a Cpf1 enzyme from Acidaminococcus (species BV3L6, UniProt Accession No. U2UMQ6). In embodiments, the nuclease has at least about 90%, at least about 95%, at least about 96%, at least about 97%, at least about 98%, or at least about 99% sequence identity to a Cpf1 enzyme from Acidaminococcus (species BV3L6, UniProt Accession No. U2UMQ6).

In embodiments, the Cpf1 is a Cpf1 enzyme from Lachnospiraceae (species ND2006, UniProt Accession No. A0A182DWE3). In embodiments, the nuclease has at least about 90%, at least about 95%, at least about 96%, at least about 97%, at least about 98%, or at least about 99% sequence identity to a Cpf1 enzyme from Lachnospiraceae. In embodiments, a sequence encoding the nuclease is codon optimized for expression in mammalian cells. In embodiments, the sequence encoding the nuclease is codon optimized for expression in human cells or mouse cells.

In embodiments, a compound that includes a CPP is conjugated to a nuclease. In embodiments, the nuclease is a soluble protein.

In embodiments, a compound that includes a CPP is conjugated to a nucleic acid encoding a nuclease. In embodiments, the nucleic acid encoding a nuclease includes a sequence encoding a promoter, wherein the promoter drives expression of the nuclease.

gRNA and Nuclease Combinations

In embodiments, the compounds include one or more CPP (or cCPP) conjugated to a gRNA and a nuclease. In embodiments, the one or more CPP (or cCPP) are conjugated to a nucleic acid encoding a gRNA and/or a nuclease. In embodiments, the nucleic acid encoding a nuclease and a gRNA includes a sequence encoding a promoter, wherein the promoter drives expression of the nuclease and the gRNA. In embodiments, the nucleic acid encoding a nuclease and a gRNA includes two promoters, wherein a first promoter controls expression of the nuclease and a second promoter controls expression of the gRNA. In embodiments, the nucleic acid encoding a gRNA and a nuclease encodes from about 1 to about 20 gRNAs, or from about 1, about 2, about 3, about 4, about 5, about 6, about 7, about 8, about 9, about 10, about 11, about 12, about 13, about 14, about 15, about 16, about 17, about 18, or about 19, and up to about 20 gRNAs. In embodiments, the gRNAs recognize different targets. In embodiments, the gRNAs recognize the same target.

In embodiments, the compounds include a cell penetrating peptide (or cCPP) conjugated to a ribonucleoprotein (RNP) that includes a gRNA and a nuclease.

In embodiments, a composition that includes: (a) a CPP conjugated to a gRNA and (b) a nuclease is delivered to a cell. In embodiments, a composition that includes: (a) a CPP conjugated to a nuclease and (b) an gRNA is delivered to a cell.

In embodiments, a composition that includes: (a) a first CPP conjugated to a gRNA and (b) a second CPP conjugated to a nuclease is delivered to a cell. In embodiments, the first CPP and second CPP are the same. In embodiments, the first CPP and second CPP are different.

### Genetic Element of Interest

In embodiments, the compounds disclosed herein include a cell penetrating peptide conjugated to a genetic element of interest. In embodiments, a genetic element of interest replaces a genomic DNA sequence cleaved by a nuclease. Non-limiting examples of genetic elements of interest include genes, a single nucleotide polymorphism, promoter, or terminators.

### Nuclease Inhibitors

In embodiments, the compounds disclosed herein include a cell penetrating peptide conjugated to an inhibitor of a nuclease (e.g., Cas9). A limitation of gene editing is potential off-target editing. The delivery of a nuclease inhibitor will limit off-target editing. In embodiments, the nuclease inhibitor is a polypeptide, polynucleotide, or small molecule. Exemplary nuclease inhibitors are described in U.S. Publication No. 2020/087354, International Publication No. 2018/085288, U.S. Publication No. 2018/0382741, International Publication No. 2019/089761, International Publication No. 2020/068304, International Publication No. 2020/041384, and International Publication No. 2019/076651, each of which is incorporated by reference herein in its entirety.

### Therapeutic Polypeptides

In embodiments, the therapeutic moiety includes a polypeptide. In embodiments, the therapeutic moiety includes a protein or a fragment thereof. In embodiments, the therapeutic moiety includes an RNA binding protein or an RNA binding fragment thereof. In embodiments, the therapeutic moiety includes an enzyme. In embodiments, the therapeutic moiety includes an RNA-cleaving enzyme or an active fragment thereof.

### Antibodies

In embodiments, the therapeutic moiety includes an antibody or an antigen-binding fragment. Antibodies and antigen-binding fragments can be derived from any suitable source, including human, mouse, camelid (e.g., camel, alpaca, llama), rat, ungulates, or non-human primates (e.g., monkey, rhesus macaque).

The term “antibody” includes intact polyclonal or monoclonal antibodies and antigen-binding fragments thereof. For example, a native immunoglobulin molecule includes two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein that includes two heterologous polypeptide chains). The two heterodimeric proteins then associate by virtue of additional interchain disulfide bonds between the heavy chain polypeptides to form an immunoglobulin protein or polypeptide.

In embodiments, the therapeutic moiety is an antigen-binding fragment that binds to IRF-5. In embodiments, the therapeutic moiety is an antigen-binding fragment that binds to an IRF-5 RNA transcript (Ye et al., PNAS (2008), 105(1):82-87; and Jung et al., (RNA (2014), 20(6):805-814). In embodiments, an antigen-binding fragment that binds to IRF-5 includes 1, 2, 3, 4, 5, or all 6 CDRs of a variable heavy chain (VH) and/or a variable light chain (VL) sequence from an antibody that specifically binds to IRF-5. In embodiments, the antigen binding fragment includes 1, 2, or 3 of the CDRs of a camelid single domain antibody such as the VHH region. In embodiments, the antigen-binding fragment that binds to IRF-5 is a portion of a full-length antibody, such as Fab, F(ab′)2, Fab′, Fv fragments, minibodies, diabodies, single domain antibody (dAb), single-chain variable fragments (scFv), multispecific antibodies formed from antibody fragments, or any other modified configuration of the immunoglobulin molecule that includes an antigen-binding site or fragment of the required specificity.

In embodiments, the therapeutic moiety includes a bispecific antibody. Bispecific Antibodies (BsAbs) are antibodies that can simultaneously bind two separate and unique antigens (or different epitopes of the same antigen). In embodiments, the therapeutic moiety includes a bispecific antibody that can simultaneously bind to IRF-5 and TRIM1. In embodiments, BsAbs is redirecting cytotoxic immune effector cells for enhanced killing of tumor cells by antibody-dependent cell-mediated cytotoxicity (ADCC) and other cytotoxic mechanisms mediated by the effector cells.

Recombinant antibody engineering has allowed for the creation of recombinant bispecific antigen-binding antibody fragments that include the variable heavy (VH) and light (VL) domains of the parental monoclonal antibodies (mabs). Non-limiting examples include scFv (single-chain variable fragment), BsDb (bispecific diabody), scBsDb (single-chain bispecific diabody), scBsTaFv (single-chain bispecific tandem variable domain), DNL-(Fab)3 (dock-and-lock trivalent Fab), sdAb (single-domain antibody), and BssdAb (bispecific single-domain antibody).

BsAbs with an Fc region are useful for carrying out Fc mediated effector functions such as ADCC and CDC. They have the half-life of normal IgG. On the other hand, BsAbs without the Fc region (bispecific fragments) rely solely on their antigen-binding capacity for carrying out therapeutic activity. Due to their smaller size, these fragments have better solid-tumor penetration rates. BsAb fragments do not require glycosylation, and they may be produced in bacterial cells. The size, valency, flexibility and half-life of BsAbs to suit the application.

Using recombinant DNA technology, bispecific IgG antibodies can be assembled from two different heavy and light chains expressed in the same cell line. Random assembly of the different chains results in the formation of nonfunctional molecules and undesirable HC homodimers. To address this problem, a second binding moiety (e.g., single chain variable fragment) may be fused to the N or C terminus of the H or L chain resulting in tetravalent BsAbs containing two binding sites for each antigen. Additional methods to address the LC-HC mispairing and HC homodimerization follow.

In embodiments, the therapeutic moiety includes a “diabody”. The term diabody refers to a bispecific antigen-binding antibody fragment in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)). Diabodies may be designed to bind to two distinct antigens and are bi-specific antigen binding constructs.

In embodiments, the therapeutic moiety includes a “nanobody” or a “single domain antibody” (which can also be referred to herein as sdAbs or VHH). Single domain antibody refers to an antigen-binding fragment that includes a single monomeric variable antibody domain comprising one variable domain (VH) of a heavy-chain antibody. In embodiments, the variable chain is the VHH of a camelid single chain antibody. They possess several advantages over traditional monoclonal antibodies (mAbs), including smaller size (15 kD), stability in the reducing intracellular environment, and ease of production in bacterial systems (Schumacher et al., Angew. Chem. Int. Ed. (2018), 57, 2314; Siontorou, International Journal of Nanomedicine, (2013), 8, 4215-27). These features render nanobodies amendable to genetic and chemical modifications (Schumacher et al., (2018) Angew. Chem. Int. Ed. 57, 2314), facilitating their application as research tools and therapeutic agents (Bannas et al., (2017) Frontiers in Immunology, 8, 1603). Over the past decade, sdAbs have been used for protein immobilization (Rothbauer et al., Mol. Cell. Proteomics, (2008) 7, 282-289), imaging (Traenkle et al., Mol. Cell. Proteomics, (2015), 14, 707-723), detection of protein-protein interactions (Herce et al., Nat. Commun, (2014), 4, 2660; Massa et al., Bioconjugate Chem, (2014), 25, 979-988), and as macromolecular inhibitors (Truttmann et al., J. Biol. Chem. (2015), 290, 9087-9100).

In embodiments, the therapeutic moiety includes a minibody.

In embodiments, the therapeutic moiety is an antibody mimetic. Antibody mimetics are compounds that, like antibodies, can specifically bind antigens, but that are not structurally related to antibodies. They are usually artificial peptides or proteins with a molar mass of about 3 to 20 kD (compared to the molar mass of antibodies at ˜150 kDa). Examples of antibody mimetics include affibody molecules affilins, affimers, affitins, alphabodies anticalins, avimers, DARPins, fynomers Kunitz domain peptides and monobodies.

In embodiments, the therapeutic moiety includes “designed ankyrin repeats” or “DARPins”. DARPins are derived from natural ankyrin proteins comprised of at least three repeat motifs proteins, and usually comprise of four or five repeats.

In embodiments, the therapeutic moiety includes “dualvariable-domain-IgG” or “DVD-IgG”. DVD-IgGs are generated from two parental monoclonal antibodies by fusing VL and VH domains of IgG with one specificity to the N-terminal of VL and VH of an IgG of different specificity, respectively, via a linker sequence.

In embodiments, the therapeutic moiety includes a F(ab) fragment. The term “F(ab)” refers to two of the protein fragments resulting from proteolytic cleavage of IgG molecules by the enzyme papain. Each F(ab) includes a covalent heterodimer of the VH chain and VL chain and includes an intact antigen-binding site. Each F(ab) is a monovalent antigen-binding fragment. The term “Fab′” refers to a fragment derived from F(ab′)2 and may contain a small portion of Fc. Each Fab′ fragment is a monovalent antigen-binding fragment.

In embodiments, the therapeutic moiety includes a F(ab′)2 fragment. The term “F(ab′)2” refers to a protein fragment of IgG generated by proteolytic cleavage by the enzyme pepsin. Each F(ab′)2 fragment includes two F(ab′) fragments and is therefore a bivalent antigen-binding fragment.

In embodiments, the therapeutic moiety includes an Fv fragment. An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In embodiments, the antigen-binding fragment is a “single chain variable fragment” or “scFv”. An scFv refers to a fusion protein of the variable regions of the heavy (VH) and light chains (VL) of immunoglobulins, connected with a short linker peptide of ten to about 25 amino acids. Huston et al. (1988) Proc. Nat. Acad. Sci. USA 85(16):5879-5883. The linker can connect the N-terminus of the VH with the C-terminus of the VL, or vice versa. A number of methods have been described to discern chemical structures for converting the naturally aggregated, but chemically separated, light and heavy polypeptide chains from an antibody V region into an scFv molecule which will fold into a three-dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In embodiments, the antigen binding constructs described herein comprise two or more antigen-binding moieties. In such embodiments, the antigen binding constructs are able to bind to two separate and unique antigens or to different epitopes of the same antigen. Knobs-into-holes BsAb IgG. H chain heterodimerization is forced by introducing different mutations into the two CH₃ domains resulting in asymmetric antibodies. Specifically, a “knob” mutation is made into one HC and a “hole” mutation is created in the other HC to promote heterodimerization.

Ig-scFv fusion. The direct addition of a new antigen-binding moiety to full length IgG results in fusion proteins with tetravalency. Examples include IgG C-terminal scFv fusion and IgG N-terminal scFv fusion.

Diabody-Fc fusion. This involves replacing the Fab fragment of an IgG with a bispecific diabody (derivative of the scFv).

Dual-Variable-Domain-IgG (DVD-IgG). VL and VH domains of IgG with one specificity were fused respectively to the N-terminal of VL and VH of an IgG of different specificity via a linker sequence to form a DVD-IgG.

### Peptide Inhibitors

In embodiments, the therapeutic moiety includes a peptide. In embodiments, the peptide acts as an agonist, increasing activity of a target protein. In embodiments, the peptide acts as an antagonist, decreasing activity of a target protein. In embodiments, the peptide is configured to inhibit protein-protein interaction (PPI). Protein-protein interactions (PPIs) are important in many biochemical processes, including transcription of nucleic acid and various post-translational modifications of translated proteins. PPIs can be experimentally determined by biophysical techniques such as X-ray crystallography, NMR spectroscopy, surface plasma resonance (SPR), bio-layer interferometry (BLI), isothermal titration calorimetry (ITC), radio-ligand binding, spectrophotometric assays and fluorescence spectroscopy. Peptides that inhibit protein-protein interaction can be referred to as peptide inhibitors.

In embodiments, the therapeutic moiety includes a peptide inhibitor. In embodiments, the peptide inhibitor includes from about 5 to about 100 amino acids, from about 5 to about 50 amino acids, from about 15 to about 30 amino acids; or from about 20 to about 40 amino acids. In embodiments, the peptide inhibitor includes one or more chemical modifications, for example, to reduce proteolytic degradation and/or to improve in vivo half-life. In embodiments, the peptide inhibitor includes one or more synthetic amino acids and/or a backbone modification. In embodiments, the peptide inhibitor has an α-helical structure.

In embodiments, the peptide inhibitor targets the dimerization domain of IRF-5. In embodiments, the peptide inhibitor targets the IAD of IRF-5.

In embodiments, the peptide inhibitor is configured to disrupt IRF-5 homodimerization. In embodiments, the peptide inhibitor is configured to binding to a surface (referred to herein as an interface surface) of an IRF-5 monomer that interfaces with other IRF-5 monomers to form a homodimer. In embodiments, binding of the peptide inhibitor to the interface surface blocks dimer formation. In embodiments, binding of the peptide inhibitor to the interface surface blocks homodimer formation. In embodiments, binding of the peptide inhibitor to the interface surface blocks heterodimer formation. In embodiments, the peptide inhibitor is configured to disrupt interactions between Helix 2 of a first IRF-5 monomer and Helix 5 of a second IRF-5 monomer, thereby preventing dimerization. In embodiments, the peptide inhibitor includes an IRF-5 Helix 2 sequence. In embodiments, the peptide inhibitor includes an IRF-5 Helix 5 sequence. In embodiments, the peptide inhibitor is configured to disrupt IRF-5 heterodimerization, for example, heterodimerization between IRF-5 and other members of the IRF family. In embodiments, the peptide inhibitor is configured to disrupt heterodimerization between IRF-5 and IRF-3 or IRF-7.

In embodiments, the peptide inhibitor is a decoy peptide that includes at least a portion of a sequence of IRF-5. In embodiments, the peptide inhibitor includes a sequence from the C-terminal dimerization domain of IRF-5. In embodiments, the peptide inhibitor includes the sequence: ELDWDADDIRLQIDNPD (SEQ ID NO:370). In embodiments, the peptide inhibitor associates with IRF-5 and prevents nuclear translocation of IRF-5.

In embodiments, the peptide inhibitor inhibits binding between IRF-5 and an IRF-5 activity regulating protein. In embodiments, the peptide inhibitor inhibits binding between IRF-5 and an IRF-5 activating protein. In embodiments, the IRF-5 activating protein is selected from CBP/p300, histone deacetylase, KAP1, RIP2, TRAF6, MyD88, IRAK1, IRAK4, CSN or LYN. In embodiments, the peptide inhibitor inhibits binding at the IRF-5/RelA-binding interface.

### Small Molecules

In embodiments, the therapeutic moiety includes a small molecule. In embodiments, the therapeutic moiety includes a small molecule that interacts with an endogenous ubiquitin ligase. In embodiments, the therapeutic moiety includes a small molecule conjugated to an antigen-binding moiety. In embodiments, the therapeutic moiety includes a small molecule kinase inhibitor. In embodiments, the therapeutic moiety includes a small molecule that inhibits a kinase that phosphorylates IRF-5. In embodiments, the small molecule inhibits a kinase selected from: TAK1, IKKα, IKKβ, IKKε and TBK1 from the IKK family. In embodiments, inhibition of phosphorylation of IRF-5 blocks nuclear translocation of IRF-5. In embodiments, the therapeutic moiety includes a small molecule inhibitor of MyD88.

### Endosomal Escape Vehicles (EEVs)

An endosomal escape vehicle (EEV) can be used to transport a cargo across a cellular membrane, for example, to deliver the cargo to the cytosol or nucleus of a cell. Cargo can include a therapeutic moiety (TM). The EEV can comprise a cell penetrating peptide (CPP), for example, a cyclic cell penetrating peptide (cCPP). In embodiments, the EEV comprises a cCPP, which is conjugated to an exocyclic peptide (EP). The EP can be referred to interchangeably as a modulatory peptide (MP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 5 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO:1), KHKK (SEQ ID NO:2), KKHK (SEQ ID NO:3), KKKH (SEQ ID NO:4), KHKH (SEQ ID NO:5), HKHK (SEQ ID NO:6), KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), HBHBH (SEQ ID NO:15), HBKBH (SEQ ID NO:16), RRRRR (SEQ ID NO:17), KKKKK (SEQ ID NO:18), KKKRK (SEQ ID NO:19), RKKKK (SEQ ID NO:20), KRKKK (SEQ ID NO:21), KKRKK (SEQ ID NO:22), KKKKR (SEQ ID NO:23), KBKBK (SEQ ID NO:24), RKKKKG (SEQ ID NO:25), KRKKKG (SEQ ID NO:26), KKRKKG (SEQ ID NO:27), KKKKRG (SEQ ID NO:28), RKKKKB (SEQ ID NO:29), KRKKKB (SEQ ID NO:30), KKRKKB (SEQ ID NO:31), KKKKRB (SEQ ID NO:32), KKKRKV (SEQ ID NO:33), RRRRRR (SEQ ID NO:34), HHHHIHH (SEQ ID NO:35), RHRHRH (SEQ ID NO:36), HRHRHR (SEQ ID NO:37), KRKRKR (SEQ ID NO:38), RKRKRK (SEQ ID NO:39), RBRBRB (SEQ ID NO:40), KBKBKB (SEQ ID NO:41), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48), wherein B is beta-alanine. The amino acids in the EP can have D or L stereochemistry.

The EP can comprise KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), KKKKK (SEQ ID NO:18), KKKRK (SEQ ID NO:19), KBKBK (SEQ ID NO:24), KKKRKV (SEQ ID NO:33), PKKKRKV (SEQ ID NO:42), PGKKRKV (SEQ ID NO:43), PKGKRKV (SEQ ID NO:44), PKKGRKV (SEQ ID NO:45), PKKKGKV (SEQ ID NO:46), PKKKRGV (SEQ ID NO:47), or PKKKRKG (SEQ ID NO:48). The EP can comprise PKKKRKV (SEQ ID NO:42), RR, RRR, RHR, RBR, RBRBR (SEQ ID NO:49), RBHBR (SEQ ID NO:50), or HBRBH (SEQ ID NO:51), wherein B is beta-alanine. The amino acids in the EP can have D or L stereochemistry.

The EP can consist of KK, KR, RR, KKK, KGK, KBK, KBR, KRK, KRR, RKK, RRR, KKKK (SEQ ID NO:7), KKRK (SEQ ID NO:8), KRKK (SEQ ID NO:9), KRRK (SEQ ID NO:10), RKKR (SEQ ID NO:11), RRRR (SEQ ID NO:12), KGKK (SEQ ID NO:13), KKGK (SEQ ID NO:14), KKKKK (SEQ ID NO:18), KKKRK (SEQ ID NO:19), KBKBK (SEQ ID NO:24), KKKRKV (SEQ ID NO:33), PKKKRKV (SEQ ID NO:42), PGKKRKV (SEQ ID NO:Z43), PKGKRKV (SEQ ID NO:Z44), PKKGRKV (SEQ ID NO:Z45), PKKKGKV (SEQ ID NO:46), PKKKRGV (SEQ ID NO:47), or PKKKRKG (SEQ ID NO:48). The EP can consist of PKKKRKV (SEQ ID NO:42), RR, RRR, RHR, RBR, RBRBR (SEQ ID NO:49), RBHBR (SEQ ID NO:50), or HBRBH (SEQ ID NO:51), wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO:42). The EP can consist of an NLS comprising the amino acid sequence PKKKRKV (SEQ ID NO:42). The EP can comprise an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NO:Z55), KAKKDEQILKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66). The EP can consist of an NLS comprising an amino acid sequence selected from NLSKRPAAIKKAGQAKKKK (SEQ ID NO:52), PAAKRVKLD (SEQ ID NO:53), RQRRNELKRSF (SEQ ID NO:54), RMRKFKNKGKDTAELRRRRVEVSVELR (SEQ ID NO:55), KAKKDEQILKRRNV (SEQ ID NO:56), VSRKRPRP (SEQ ID NO:57), PPKKARED (SEQ ID NO:58), PQPKKKPL (SEQ ID NO:59), SALIKKKKKMAP (SEQ ID NO:60), DRLRR (SEQ ID NO:61), PKQKKRK (SEQ ID NO:62), RKLKKKIKKL (SEQ ID NO:63), REKKKFLKRR (SEQ ID NO:64), KRKGDEVDGVDEVAKKKSKK (SEQ ID NO:65), and RKCLQAGMNLEARKTKK (SEQ ID NO:66).

All exocyclic sequences can also contain an N-terminal acetyl group. Hence, for example, the EP can have the structure: Ac—PKKKRKV (SEQ ID NO:42).

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

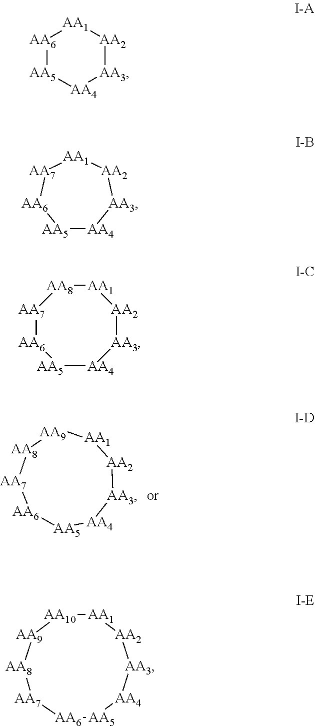

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 5 along with their abbreviations used herein.

TABLE 5
Amino Acid Abbreviations

| | Abbreviations* | Abbreviations* |
|---|---|---|
| Amino Acid | L-amino acid | D-amino acid |
| 2-[2-[2-aminoethoxy]ethoxy]acetic acid | AEEA, | NA |
| | miniPEG | |
| Alanine | Ala (A) | ala (a) |
| Allo-isoleucine | Aile | Aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka pipecolic acid) | Pip (Θ) | pip (θ) |
| isoleucine | Ile (I) | ile (i) |
| leucine | Leu (L) | leu (l) |
| lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| norleucine | Nle (Ω) | nle |
| phenylalanine | Phe (f) | phe (f) |
| phenylglycine | Phg (Ψ) | phg |
| 4-(phosphonodifluoro- | F₂Pmp (Λ) | f₂pmp |
| methyl)phenylalanine | | |
| proline | Pro (P) | pro (p) |
| sarcosine | Sar (Ξ) | sar |
| selenocysteine | Sec (U) | sec (u) |
| serine | Ser (S) | ser (s) |
| threonine | Thr (T) | thr (y) |
| tyrosine | Tyr (Y) | tyr (y) |
| tryptophan | Trp (W) | trp (w) |
| valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: capital letters indicate the L-amino acid form, lower case letter indicate the D-amino acid form.

As used herein, “polyethylene glycol” and “PEG” are used interchangeably. “PEGm,” and “PEGₘ,” are, or are derived from, a molecule of the formula HO(CO)—(CH₂)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and m is 2. In embodiments, n is 1 and m is 4. In embodiments, n is 2 and m is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)—(OCH₂CH₂)ₘ—NH₂ where n is 1 and m is any integer from 1 to 23. For example, “miniPEG2” or “miniPEG₂” is, or is derived from, (2-[2-[2-aminoethoxy]ethoxy]acetic acid), and “miniPEG4” or “miniPEG₄” is, or is derived from, HO(CO)—(CH₂)ₙ—(OCH₂CH₂)ₘ—NH₂ where n is 1 and m is 4.

The cCPP can comprise 4 to 20 amino acids, wherein: (i) at least one amino acid has a side chain comprising a guanidine group, or a protonated form thereof; (ii) at least one amino acid has no side chain or a side chain comprising

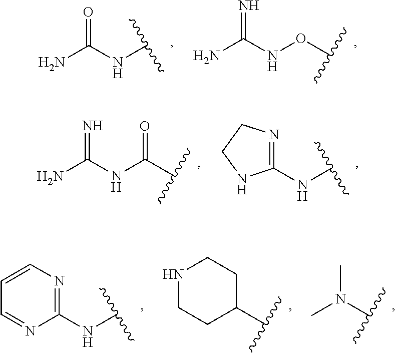

or a protonated form thereof, and (iii) at least two amino acids independently have a side chain comprising an aromatic or heteroaromatic group.

At least two amino acids can have no side chain or a side chain comprising

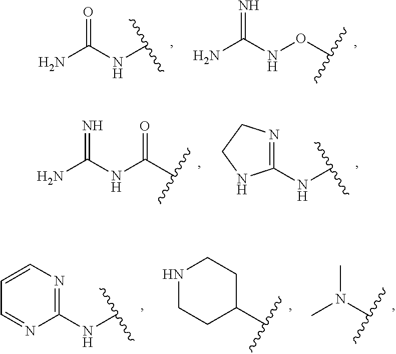

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

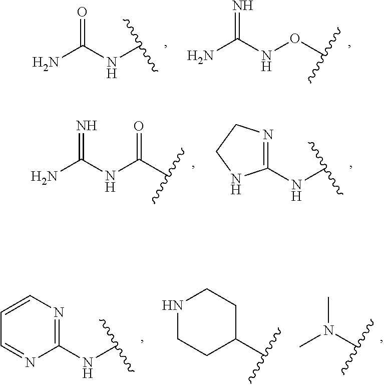

or a protonated form thereof.

The cCPP can comprise from 6 to 20 amino acid residues which form the cCPP, wherein: (i) at least two amino acid can independently be glycine, β-alanine, or 4-aminobutyric acid residues; (ii) at least one amino acid can have a side chain comprising an aryl or heteroaryl group; and (iii) at least one amino acid has a side chain comprising a guanidine group,

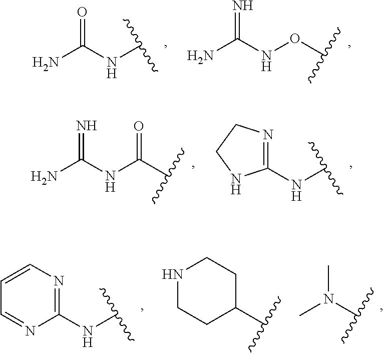

a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

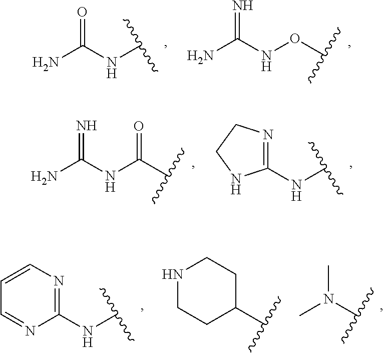

or a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous.

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-41,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

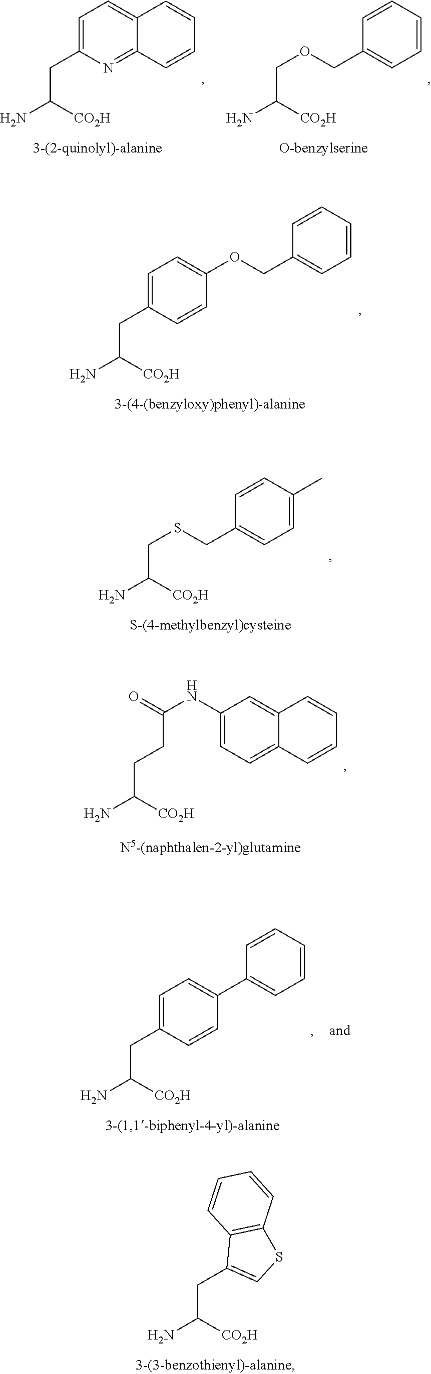

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, p-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 6 lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 6
Amino Acid Hydrophobicity

| Amino | | Eisenberg | Engleman | Kyrie and | Hoop and | |
|---|---|---|---|---|---|---|
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif; a cCPP having a Phe-Nal-Arg motif may exhibit improved cytosolic delivery efficiency compared to an otherwise identical cCPP having a Nal-Phe-Arg motif; and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Ångstroms; Å²) of an amino acid side chain that is accessible to a solvent, SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 7. The SASA values described herein are based on the theoretical values listed in Table 7 below, as reported by Tien, et al. (PLOS ONE 8(11): e80635, available at doi.org/10.1371/journal.pone.0080635), which is herein incorporated by reference in its entirety for all purposes.

TABLE 7
Amino Acid SASA Values

| | | | Miller et | Rose et |
|---|---|---|---|---|
| Residue | Theoretical | Empirical | al. (1987) | al. (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

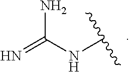 [SMILES: *NC(=N)N]

As used herein, a protonated form of guanidine refers to the structure:

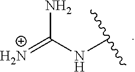 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

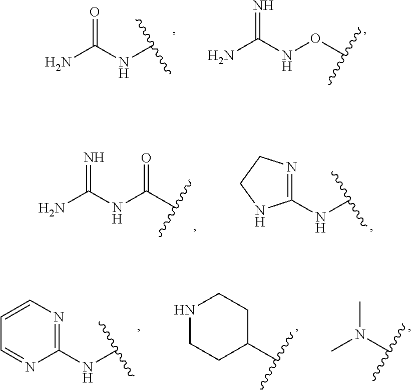

or a protonated form thereof.

The disclosure relates to a cCPP comprising from 4 to 20 amino acids residues, wherein: (i) at least one amino acid has a side chain comprising a guanidine group, or a protonated form thereof, (ii) at least one amino acid residue has no side chain or a side chain comprising

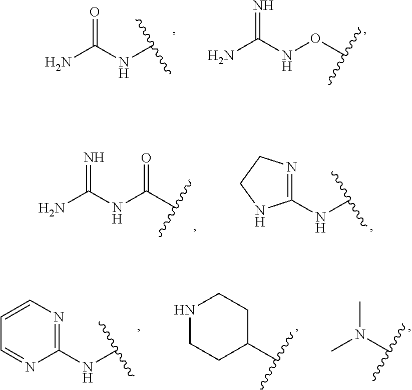

or a protonated form thereof; and (iii) at least two amino acids residues independently have a side chain comprising an aromatic or heteroaromatic group.

At least two amino acids residues can have no side chain or a side chain comprising

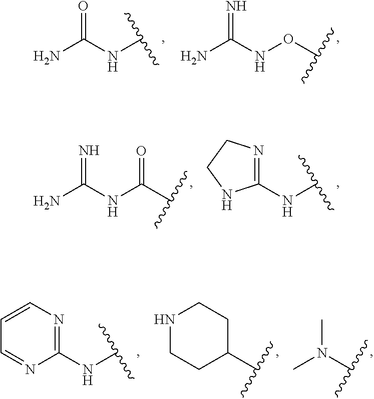

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

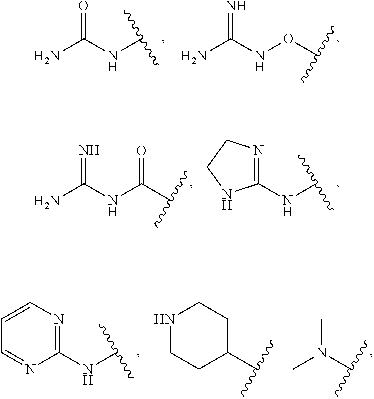

or a protonated form thereof.

The cCPP can comprise at least two amino acids each independently having one of the following moieties

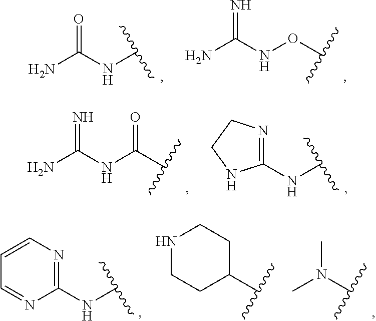

or a protonated form thereof. At least two amino acids can have a side chain comprising the same moiety selected from:

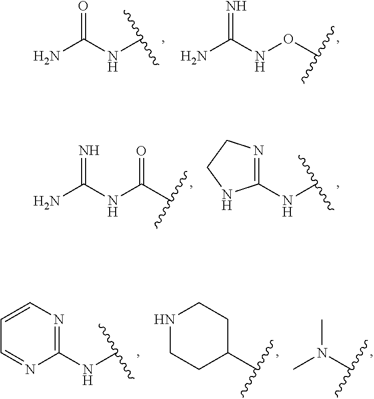

or a protonated form thereof. At least one amino acid can have a side chain comprising

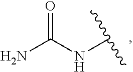 [SMILES: *NC(N)=O]

or a protonated form thereof. At least two amino acids can have a side chain comprising

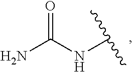 [SMILES: *NC(N)=O]

or a protonated form thereof. One, two, three, or four amino acids can have a side chain comprising

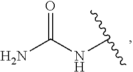 [SMILES: *NC(N)=O]

or a protonated form thereof. One amino acid can have a side chain comprising

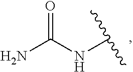 [SMILES: *NC(N)=O]

or a protonated form thereof. Two amino acids can have a side chain comprising

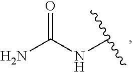 [SMILES: *NC(N)=O]

or a protonated form thereof.

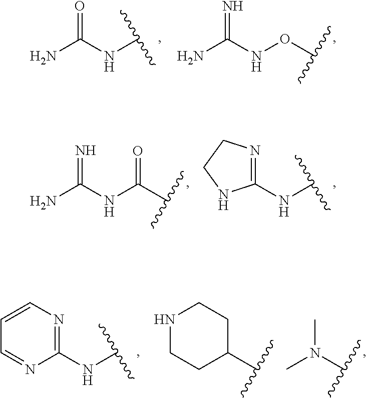

or a protonated form thereof, can be attached to the terminus of the amino acid side chain.

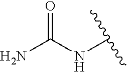 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

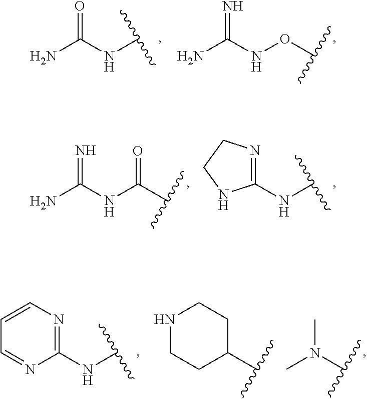

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamic acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

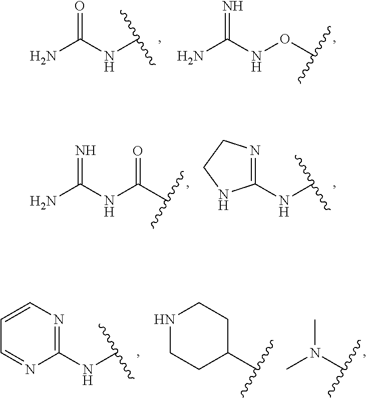

or a protonated form thereof, can be adjacent to an amino acid having a side chain comprising an aromatic or heteroaromatic group. An amino acid having a side chain comprising:

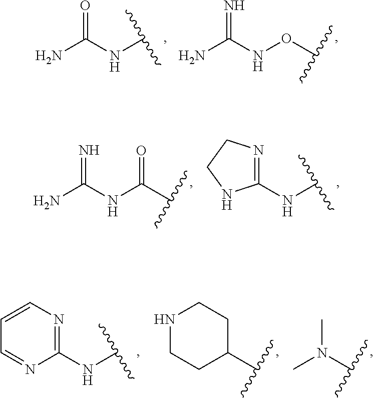

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

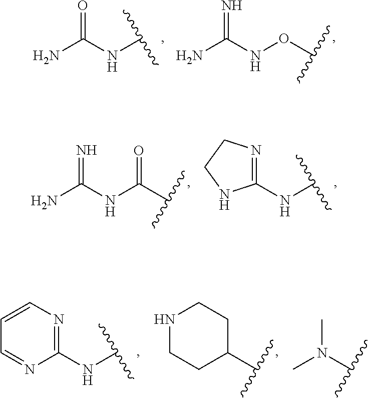

or protonated forms thereof can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

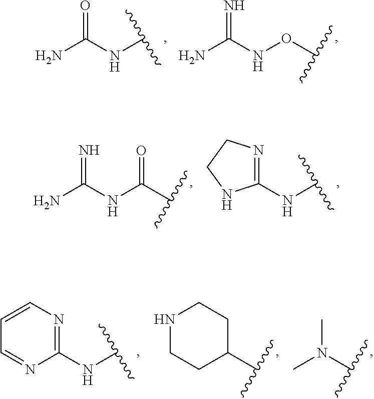

or a protonated form thereof. The cCPPs can comprise at least two contiguous amino acids having a side chain comprising an aromatic or heteroaromatic group and at least two non-adjacent amino acids having a side chain comprising

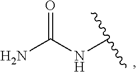 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

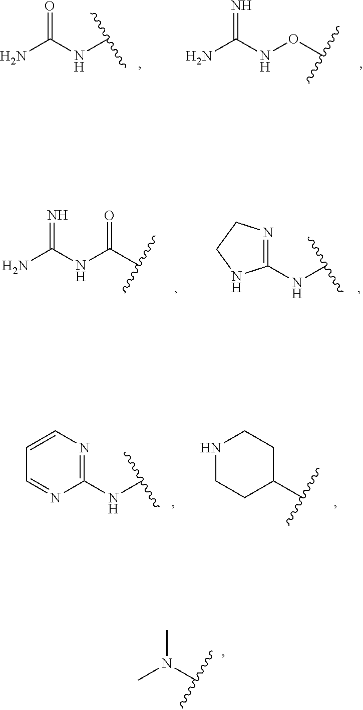

or a protonated form thereof, are alternating with at least two amino acids having a side chain comprising a guanidine group or protonated form thereof.

The cCPP can comprise the structure of Formula (A):

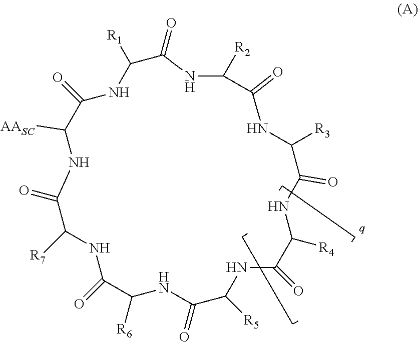

or a protonated form thereof,

wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the cyclic peptide of Formula (A) is not FfΦRrRrQ (SEQ ID NO:67). In embodiments, the cyclic peptide of Formula (A) is FfΦRrRrQ (SEQ ID NO:67).

The cCPP can comprise the structure of Formula (I):

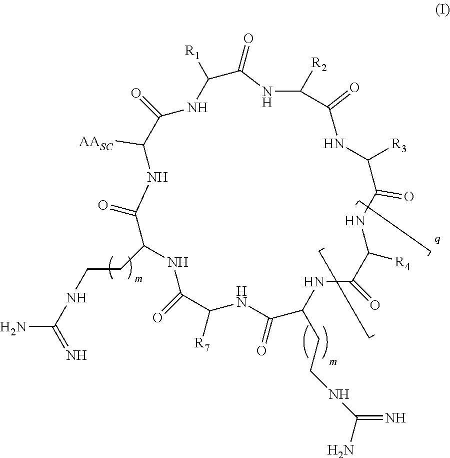

or a protonated form thereof,

wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₇ are independently H or an amino acid side chain;
AA_{SC} is an amino acid side chain;
q is 1, 2, 3 or 4; and
each m is independently an integer of 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of p-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 5 or Table 7. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 5 or Table 7. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃ alkylene-heteroaryl. R₆ can be H or -alkylene-aryl. R₆ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 5 or Table 7. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃ alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 5 or Table 7. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (A) can comprise the structure of Formula (I)

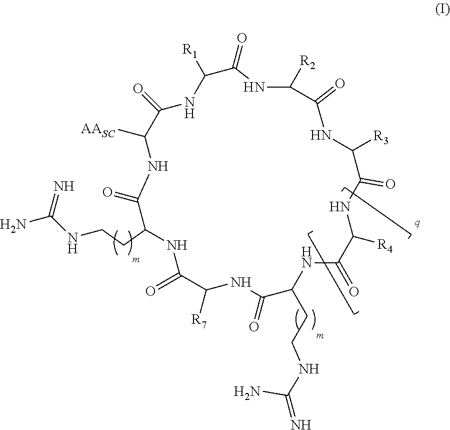

or protonated form thereof, wherein AA_{SC}, R₁, R₂, R₃, R₄, R₅, m, and q are as defined herein.

The cCPP of Formula (A) can comprise the structure of Formula (I-a) or Formula (I-b):

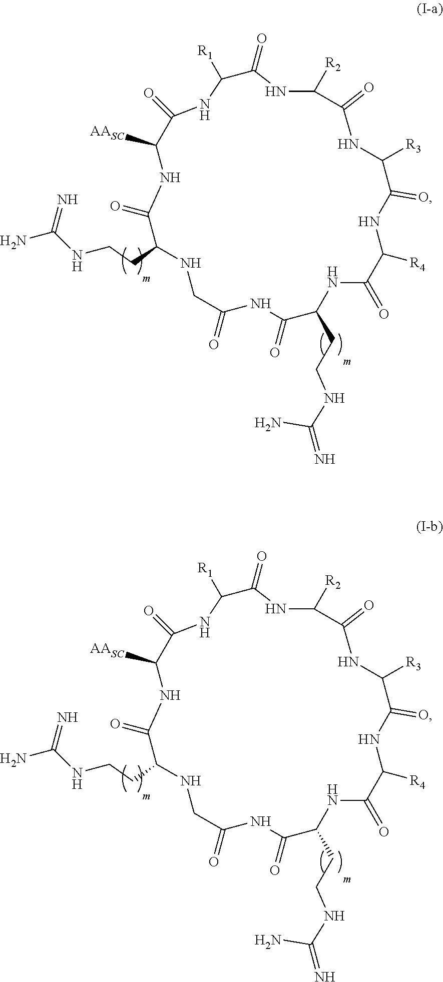

or protonated form thereof, wherein AA_{SC}, R₁, R₂, R₃, R₄, and m are as defined herein.

The cCPP of Formula (A) can comprise the structure of Formula (I-1), (1-2), (I-3), or (I-4):

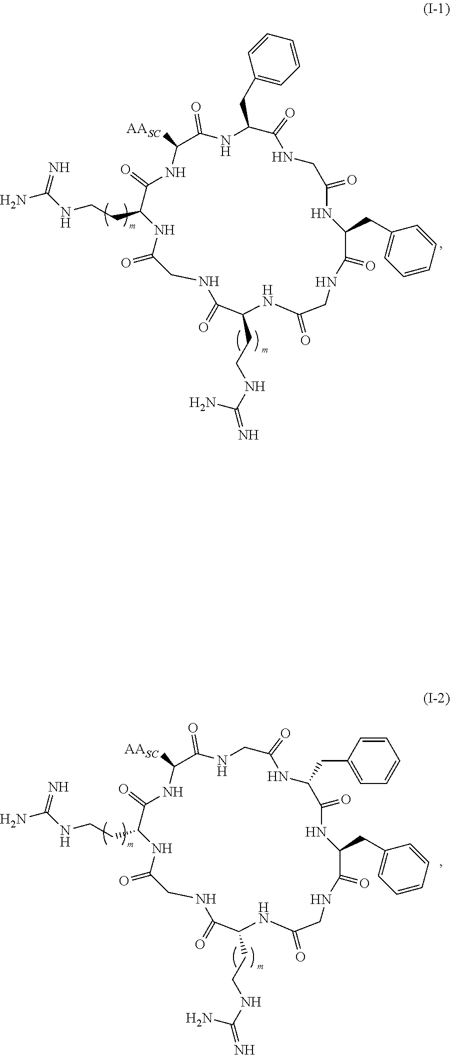

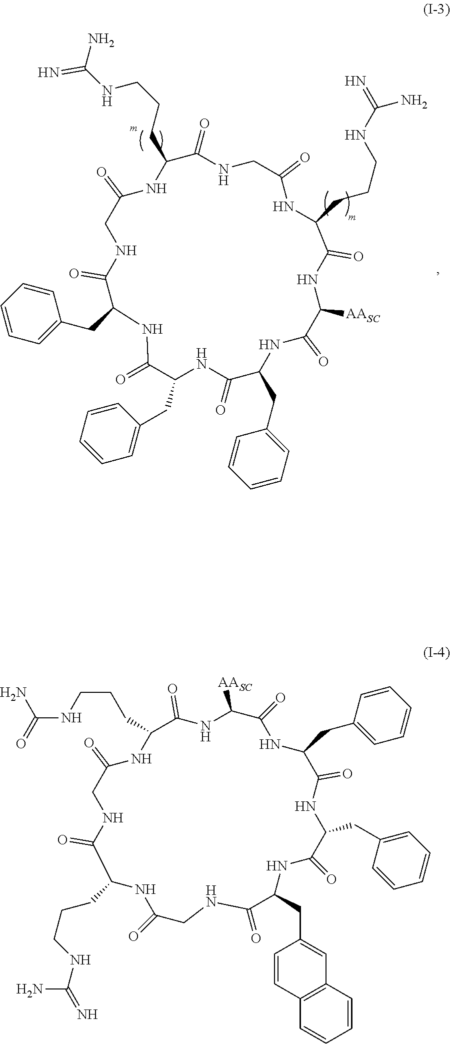

or protonated form thereof, wherein AA_{SC} and m are as defined herein.

The cCPP of Formula (A) can comprise the structure of Formula (I-5) or (I-6):

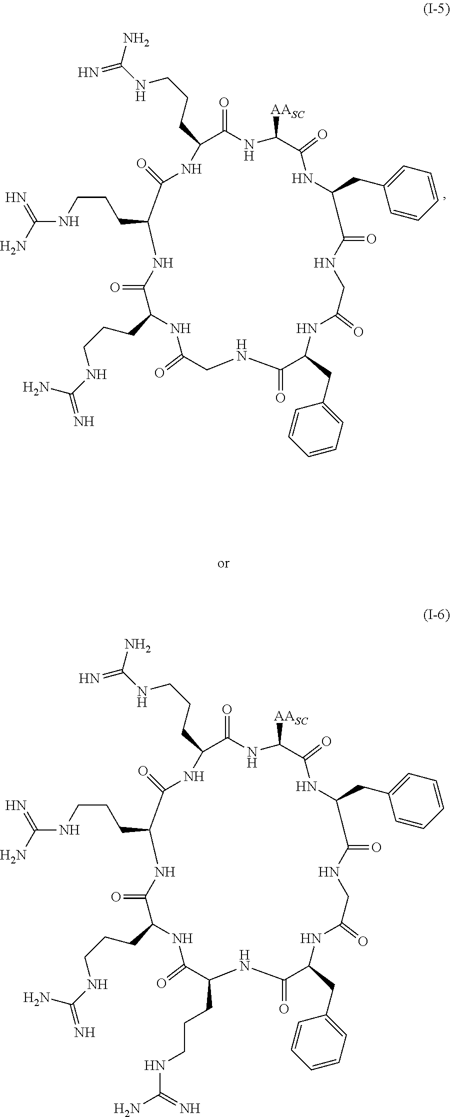

or protonated form thereof, wherein AA_{SC} is as defined herein.

The cCPP of Formula (A) can comprise the structure of Formula (I-1):

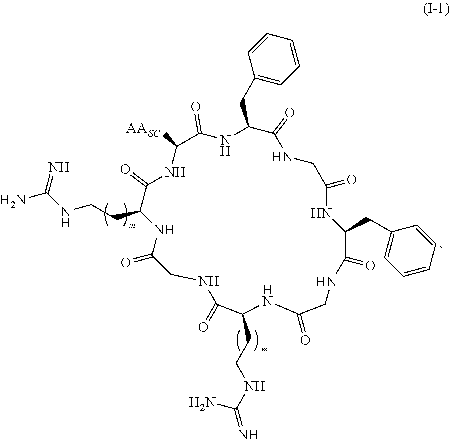

or a protonated form thereof, wherein AA_{SC} and m are as defined herein.

The cCPP of Formula (A) can comprise the structure of Formula (I-2):

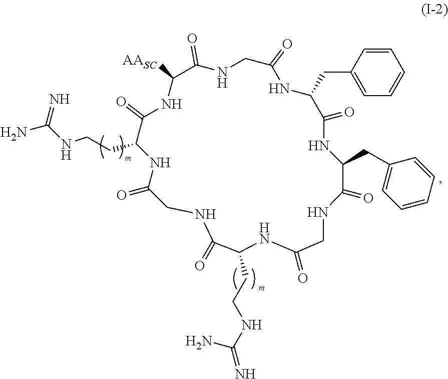

or a protonated form thereof, wherein AA_{SC} and m are as defined herein.

The cCPP of Formula (A) can comprise the structure of Formula (I-3):

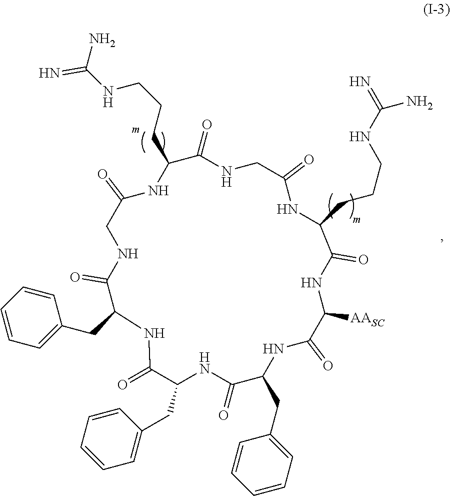

or a protonated form thereof, wherein AA_{SC} and m are as defined herein.

The cCPP of Formula (A) can comprise the structure of Formula (I-4):

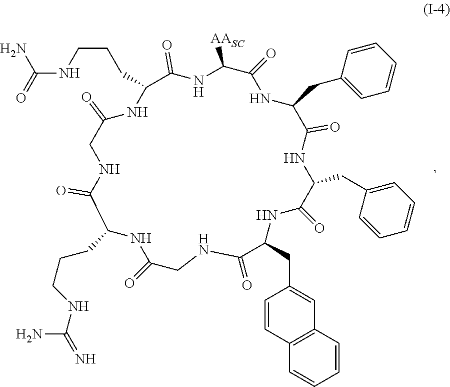

or a protonated form thereof, wherein AA_{SC} and m are as defined herein.

The cCPP of Formula (A) can comprise the structure of Formula (I-5):

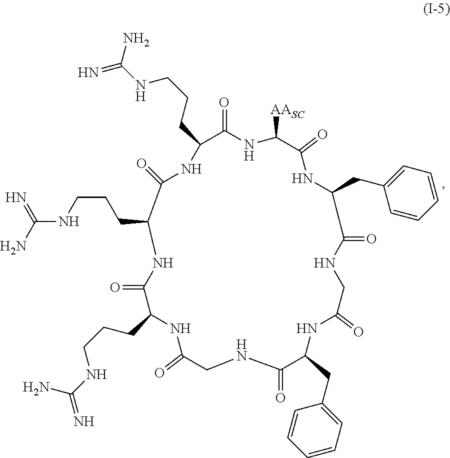

or a protonated form thereof, wherein AA_{SC} and m are as defined herein.

The cCPP of Formula (A) can comprise the structure of Formula (I-6):

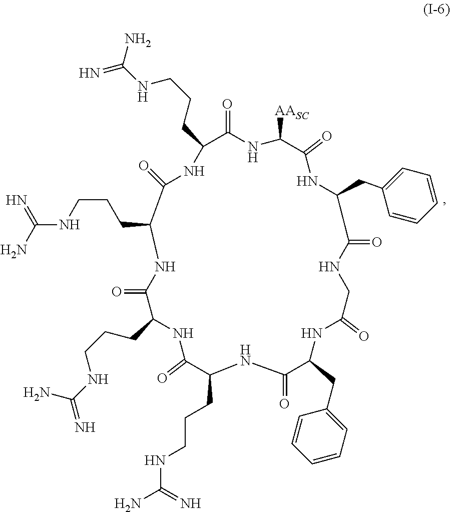

or a protonated form thereof, wherein AA_{SC} and m are as defined herein.

The cCPP can comprise one of the following sequences: FGFGRGR (SEQ ID NO:68); GfFGrGr (SEQ ID NO:69), FfΦGRGR (SEQ ID NO:70); FfFGRGR (SEQ ID NO:71); or FfΦGrGr (SEQ ID NO:72). The cCPP can have one of the following sequences: FGFΦ (SEQ ID NO:73); GfFGrGrQ (SEQ ID NO:74), FfΦGRGRQ (SEQ ID NO:75); FfFGRGRQ (SEQ ID NO:76); or FfΦGrGrQ (SEQ ID NO:77).

The disclosure also relates to a cCPP having the structure of Formula (II):

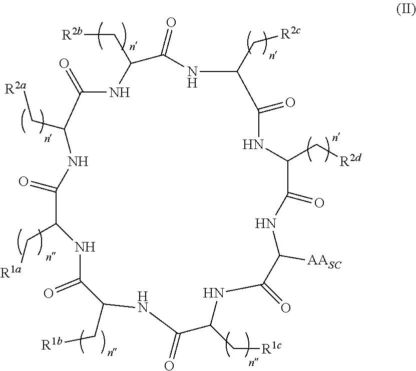

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

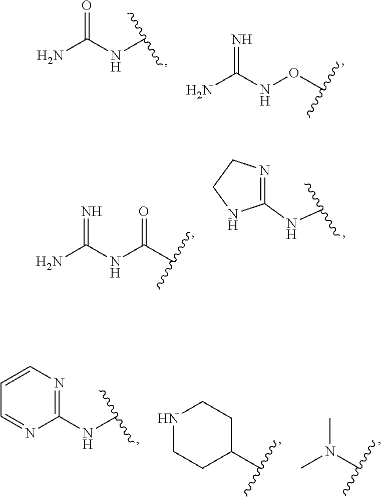

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2b} or R^{2d} is absent.

At least two of R^{2a}, R^{2b}, R^{2c} and R^{2d} can be

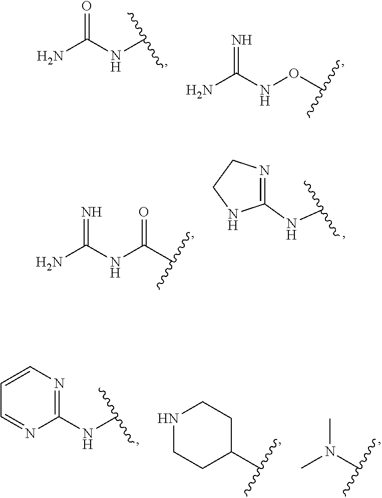

or a protonated form thereof.
Two or three of R^{2a}, R^{2b}, R^{2c} and R^{2d} can be

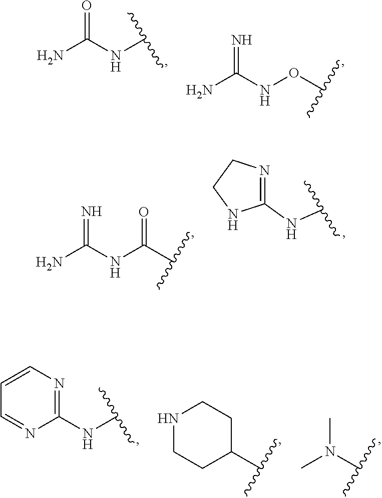

or a protonated form thereof. One of R^{2a}, R^{2b}, R^{2c} and R^{2d} can be

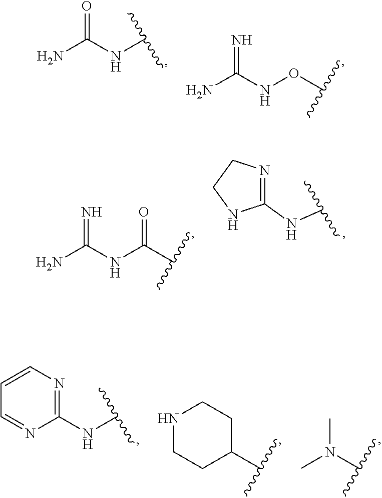

or a protonated form thereof. At least one of R^{2a}, R^{2b}, R^{2c} and R^{2d} can be

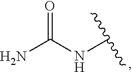 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

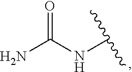 [SMILES: *NC(N)=O]

or a protonated form thereof, and the remaining of R², R^{2b}, R^{2c} and R^{2d} can be guanidine, or a protonated form thereof.

All of R^{2a}, R^{2b}, R^{2c} and R^{2d} can be

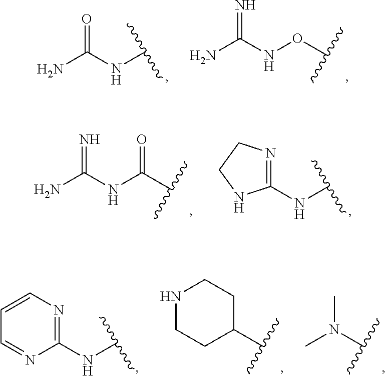

or a protonated form thereof. At least or R^{2a}, R^{2b}, R^{2c} and R^{2d} can be

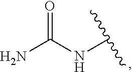 [SMILES: *NC(N)=O]

or a protonated form thereof, and the remaining of R^{2a}, R^{2b}, R^{2c} and R^{2d} can be guanidine or a protonated form thereof. At least two R^{2a}, R^{2b}, R^{2c} and R^{2d} groups can be

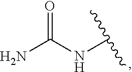 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

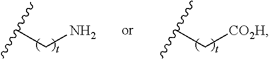

wherein t can be an integer from 0 to 5. AA_{SC} can be

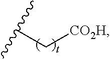

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

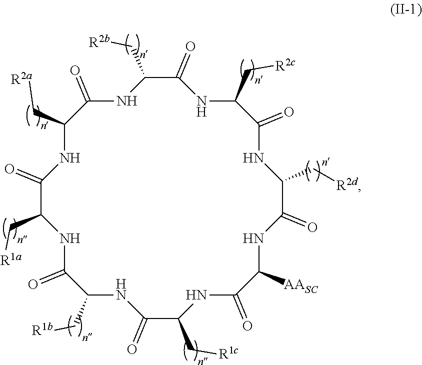

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

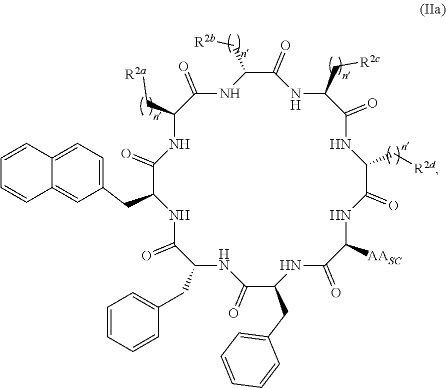

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

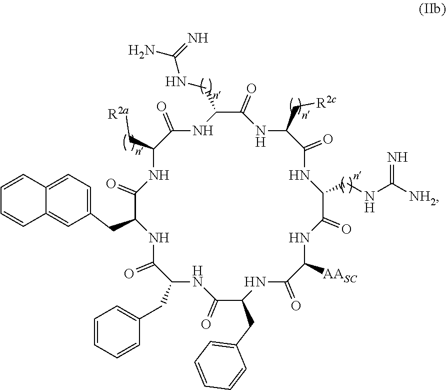

wherein R^{2a}, R^{2b}, AA_{SC}, and n′ are as defined herein.

The cCPP can have the structure of Formula (IIc):

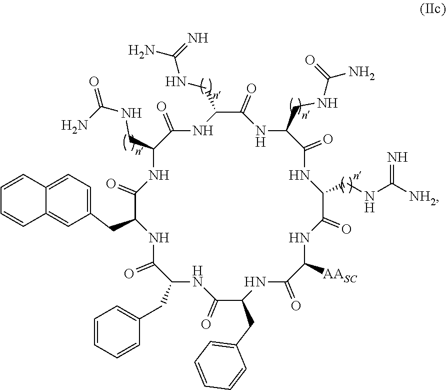

or a protonated form thereof,

wherein:
AA_{SC} and n′ are as defined herein.

The cCPP of Formula (IIa) has one of the following structures:

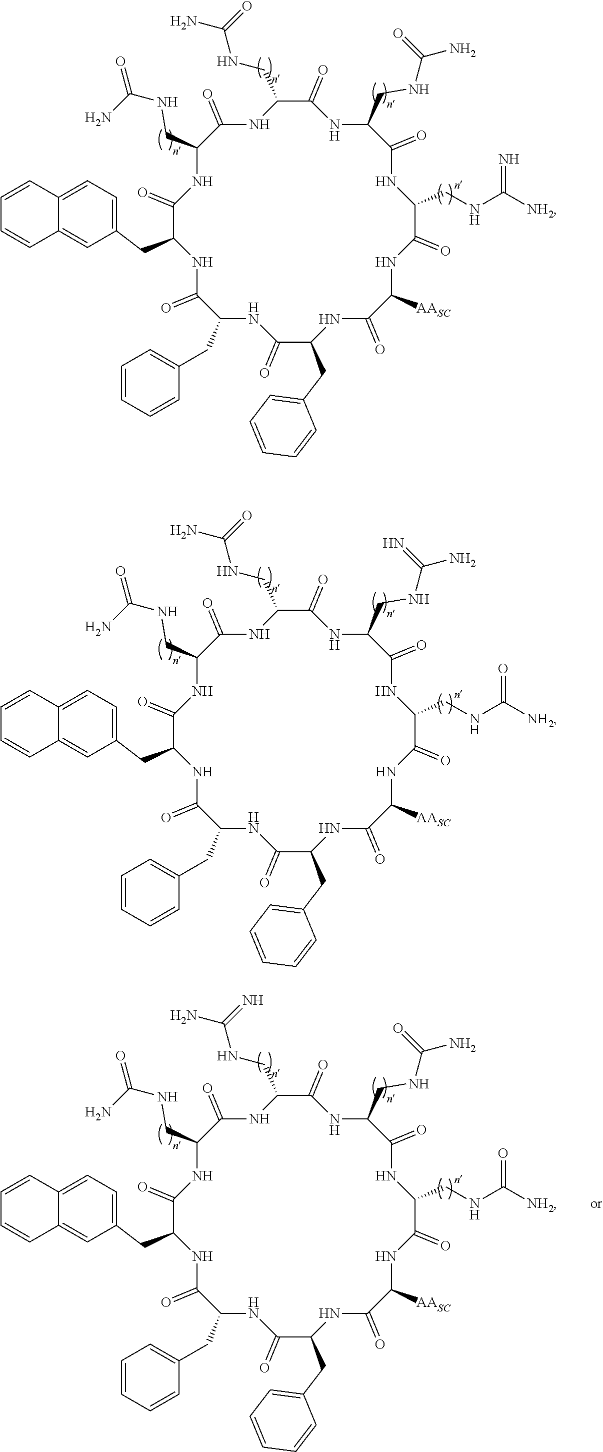

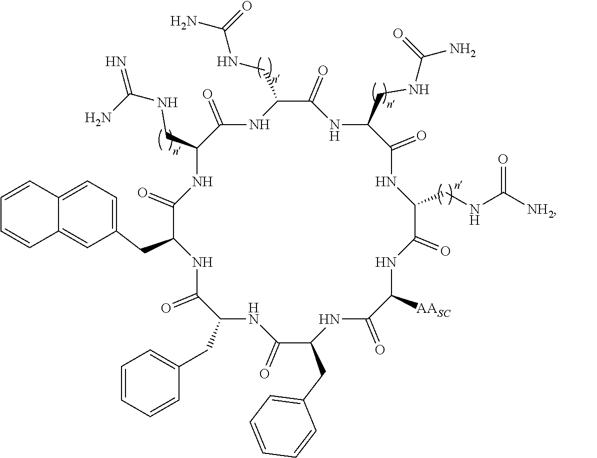

wherein AA_{SC} and n are as defined herein.

The cCPP of Formula (IIa) has one of the following structures:

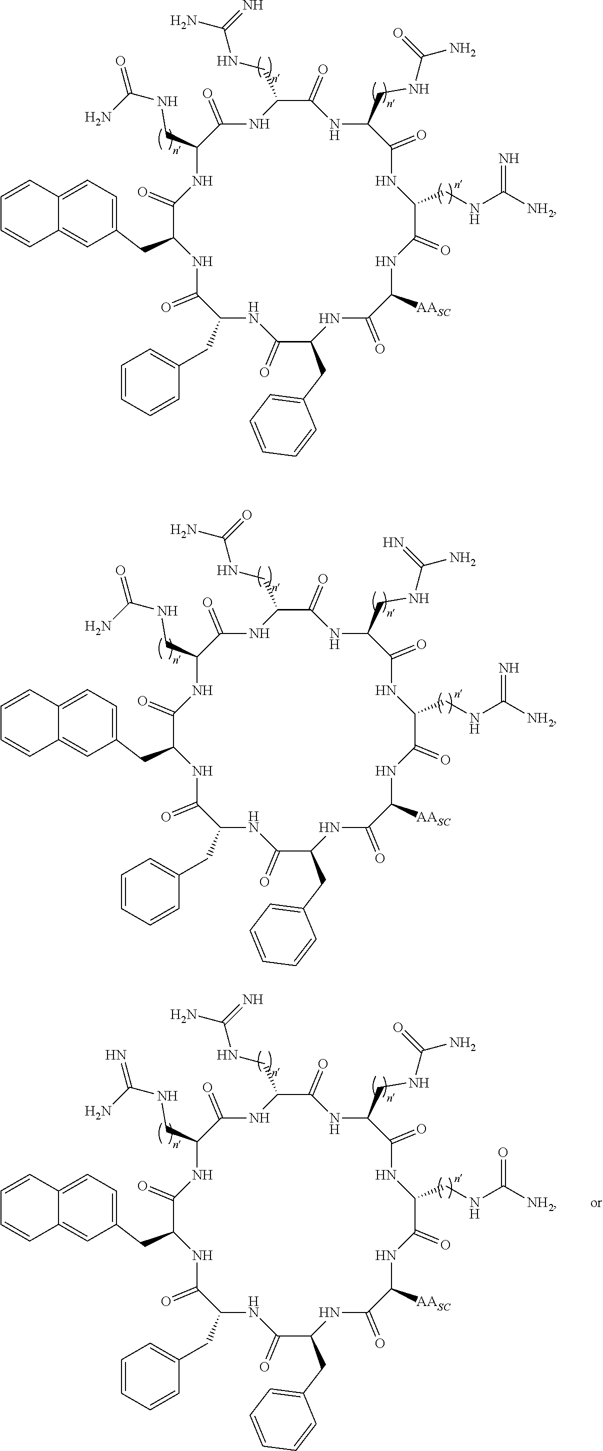

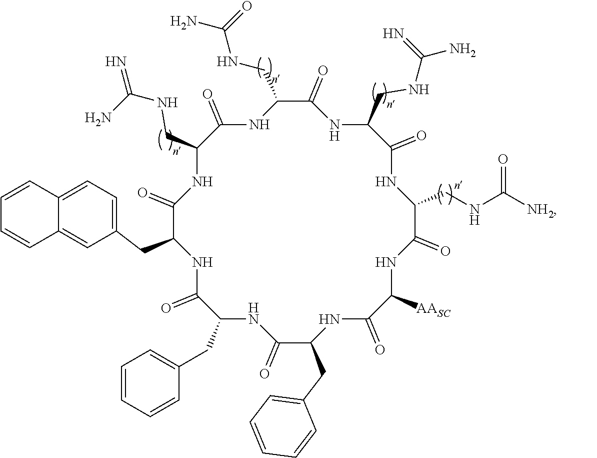

wherein AA_{SC} and n are as defined herein

The cCPP of Formula (IIa) has one of the following structures:

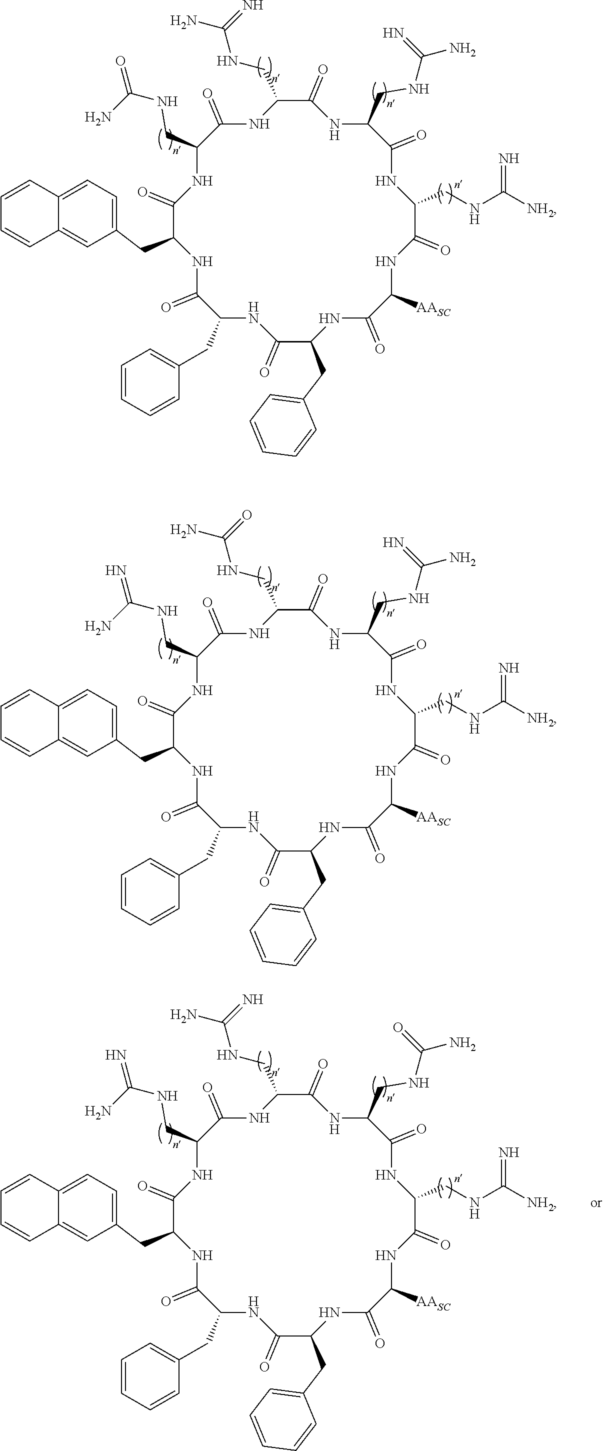

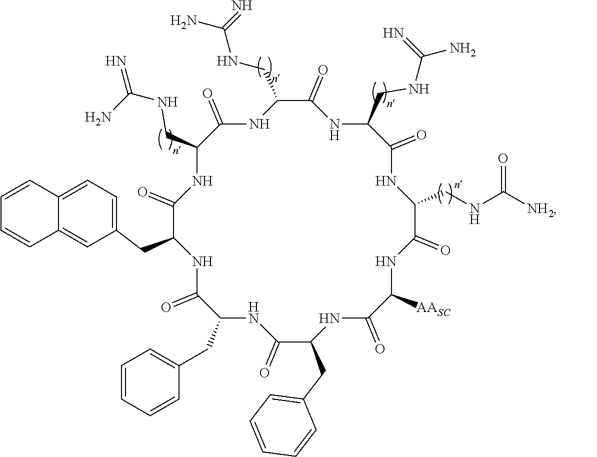

herein AA_{SC} and n are as defined herein.

The cCPP of Formula (II) can have the structure:

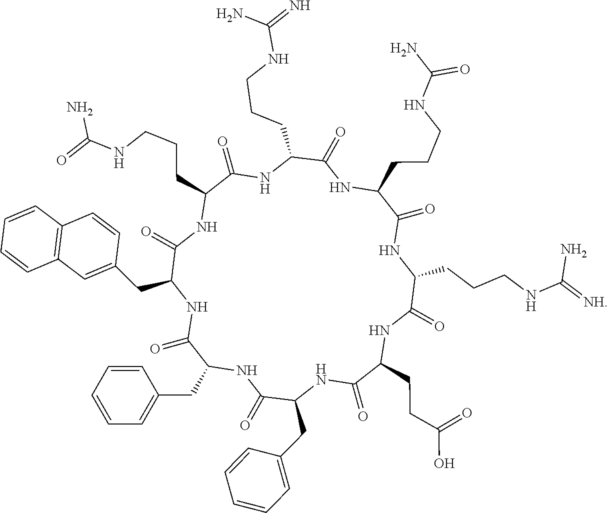 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

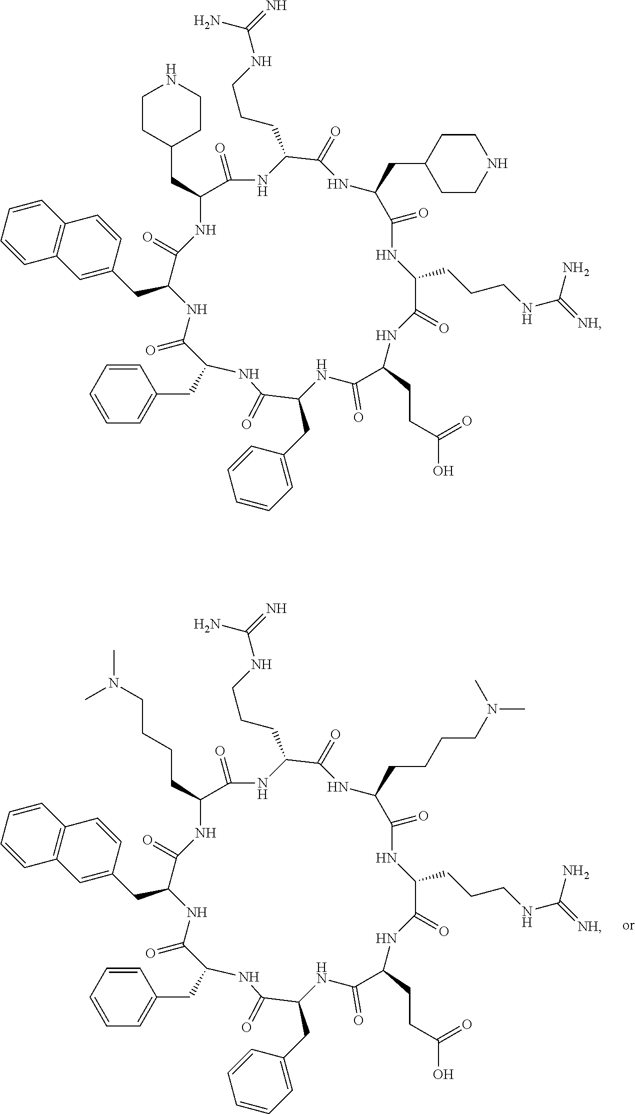 [SMILES: N=C(N)NCCC[C@H]1NC(=O)[C@H](CC2CCNCC2)NC(=O)[C@@H](CCCNC(=N)N)NC(=O)[C@H](CC2CCNCC2)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; CN(C)CCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)[C@H](CCCCN(C)C)NC(=O)[C@@H](CCCNC(=N)N)NC1=O ; "or"]

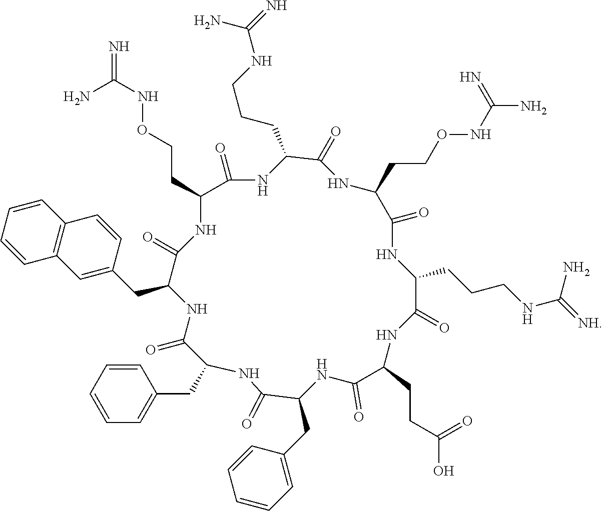 [SMILES: N=C(N)NCCC[C@H]1NC(=O)[C@H](CCONC(=N)N)NC(=O)[C@@H](CCCNC(=N)N)NC(=O)[C@H](CCONC(=N)N)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP can have the structure of Formula (III):

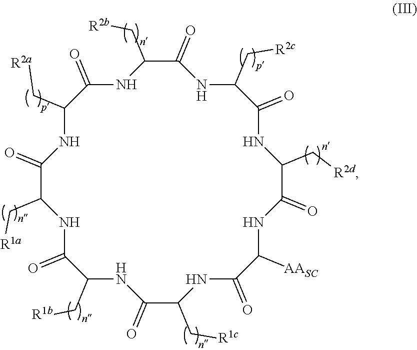

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

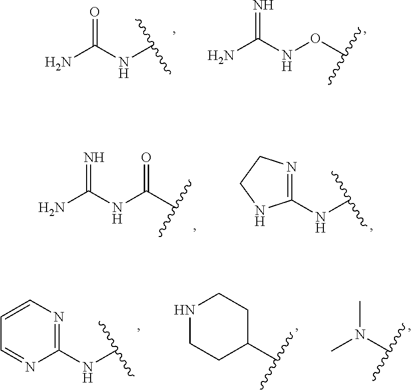

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

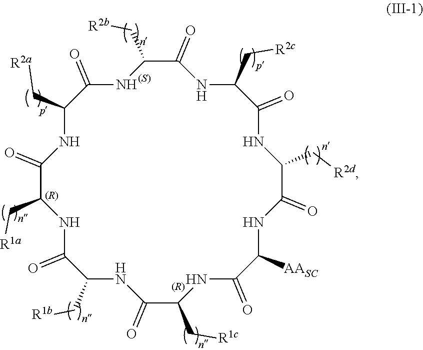

wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

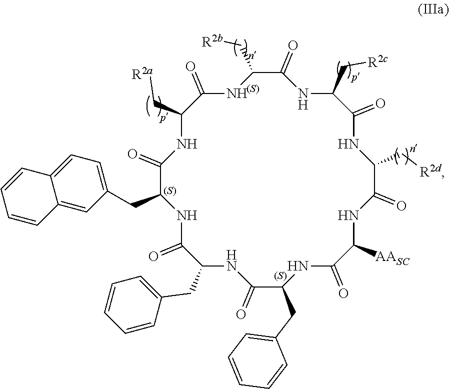

wherein:
AA_{SC}, R^{2a}, R^{2c}, R^{2b}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

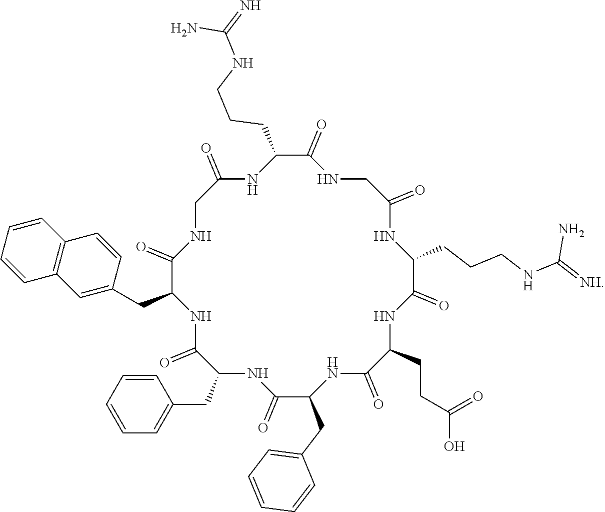 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

| CPP Sequence | SEQ ID NO: |
|---|---|
| (FfΦRrRrQ) | 78 |
| (FfΦCit-r-Cit-rQ) | 79 |
| (FfΦGrGrQ) | 80 |
| (FfFGRGRQ) | 81 |
| (FGFGRGRQ) | 82 |
| (GfFGrGrQ) | 83 |
| (FGFGRRRQ) | 84 |
| (FGFRRRRQ) | 85 |

The cCPP of Formula A can be selected from:

| CPP Sequence | SEQ ID NO: |
|---|---|
| FΦRRRRQ | 86 |
| fΦRrRrQ | 87 |
| FfΦRrRrQ | 78 |
| FfΦCit-r-Cit-rO | 79 |
| FfΦGrGrQ | 80 |
| FfΦRGRGQ | 88 |
| FfFGRGRQ | 81 |
| FGFGRGRQ | 82 |
| GfFGrGrQ | 83 |
| FGFGRRRQ | 84 |
| FGFRRRRQ | 85 |

In embodiments, the cCPP is selected from:

CPP sequences and SEQ ID NOs

| | | | | | |
|---|---|---|---|---|---|
| FΦRRRQ | 89 | RRFRΦRQ | 99 | FΦRRRRQK | 109 |
| FΦRRRC | 90 | FRRRRΦQ | 100 | FΦRRRRQC | 110 |
| FΦRRRU | 91 | TRFRΦRQ | 101 | fΦRrRrRQ | 111 |
| RRRΦFQ | 92 | RRΦFRRQ | 102 | FΦRRRRRQ | 112 |
| RRRRΦF | 93 | CRRRRFWQ | 103 | RRRRΦFDΩC | 113 |
| FΦRRRR | 94 | FfΦRrRrQ | 104 | FΦRRR | 114 |
| FϕrRrRq | 95 | FFΦRRRRQ | 105 | FWRRR | 115 |
| FϕrRrRQ | 96 | RFRFRΦRQ | 106 | RRRΦF | 116 |
| FΦRRRRQ | 97 | URRRRFWQ | 107 | RRRWF | 117 |
| fΦRrRrQ | 98 | CRRRRFWQ | 108 | | |

Where Φ=L-naphthylalanine; ϕ=D-naphthylalanine; Ω=L-norleucine

In embodiments, the cCPP is not selected from:

CPP sequences and SEQ ID NOs

| | | | | | |
|---|---|---|---|---|---|
| FΦRRRQ | 89 | RRFRΦRQ | 99 | FΦRRRRQK | 109 |
| FΦRRRC | 90 | FRRRRΦQ | 100 | FΦRRRRQC | 110 |
| FΦRRRU | 91 | TRFRΦRQ | 101 | fΦRrRrRQ | 111 |
| RRRΦFQ | 92 | RRΦFRRQ | 102 | FΦRRRRRQ | 112 |
| RRRRΦF | 93 | CRRRRFWQ | 103 | RRRRΦFDΩC | 113 |
| FΦRRRR | 94 | FfΦRrRrQ | 104 | FΦRRR | 114 |
| FϕrRrRq | 95 | FFΦRRRRQ | 105 | FWRRR | 115 |
| FϕrRrRQ | 96 | RFRFRΦRQ | 106 | RRRΦF | 116 |
| FΦRRRRQ | 97 | URRRRFWQ | 107 | RRRWF | 117 |
| fΦRrRrQ | 98 | CRRRRFWQ | 108 | | |

Where Φ=L-naphthylalanine; ϕ=D-naphthylalanine; Ω=L-norleucine

AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″— subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″—, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50, or (ix) the linker can comprise one or more of (i) through (x).

The linker can comprise one or more D or L amino acids and/or —(R¹-J-R²)z″—, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z}′— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂)z′ can also be referred to as polyethylene glycol (PEG).

The linker can comprise one or more amino acids. The linker can comprise a peptide. The linker can comprise a —(OCH₂CH₂)_{z}′—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

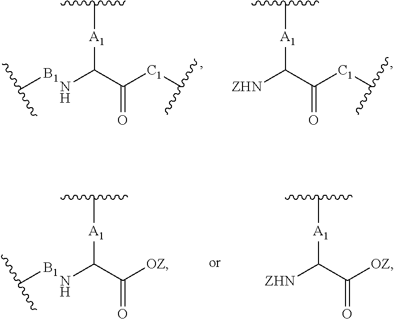

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

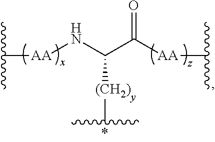

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

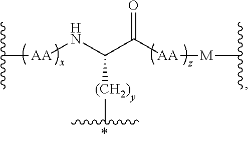

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

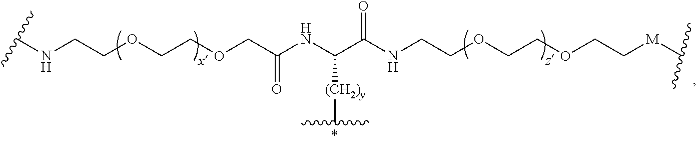

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

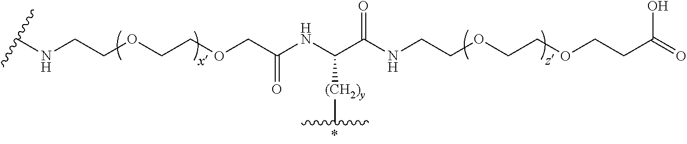

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

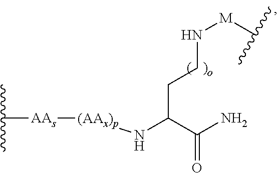

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

The linker can have a structure:

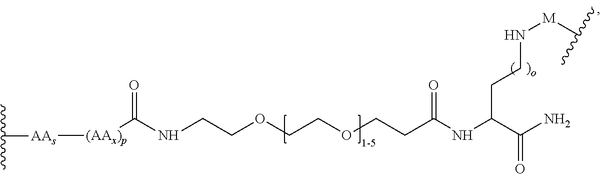

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

M can comprise an alkylene, alkenylene, alkynylene, carbocyclyl, or heterocyclyl, each of which is optionally substituted. M can be selected from:

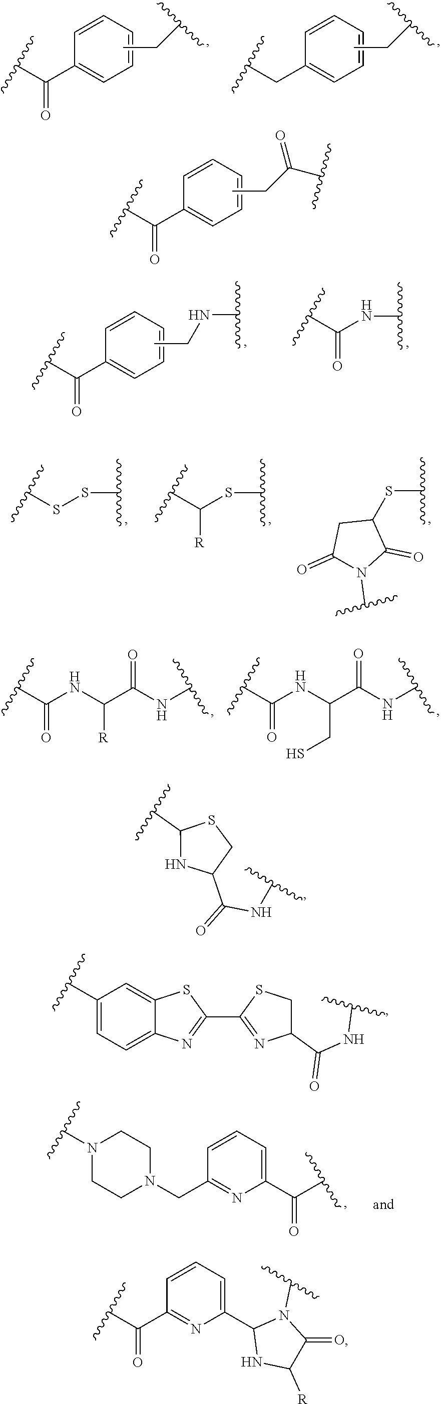

wherein R is alkyl, alkenyl, alkynyl, carbocyclyl, or heterocyclyl.

M can be selected from:

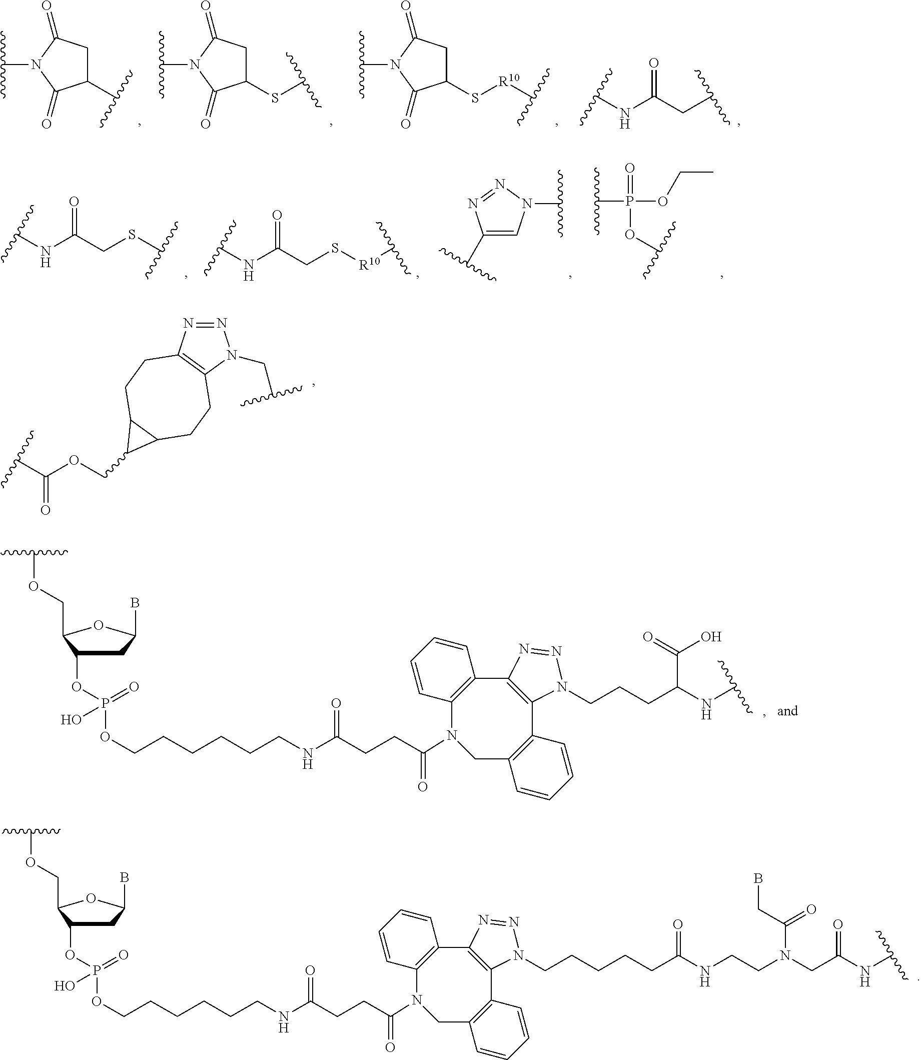

wherein: R¹⁰ is alkylene, cycloalkyl, or

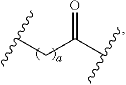

wherein a is 0 to 10.

M can be

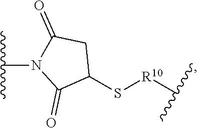

### R¹⁰ can be

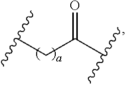

### and a is 0 to 10. M can be

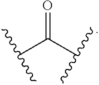 [SMILES: *C(*)=O]

M can be a heterobifunctional crosslinker, e.g.,

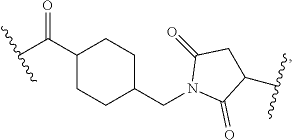 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

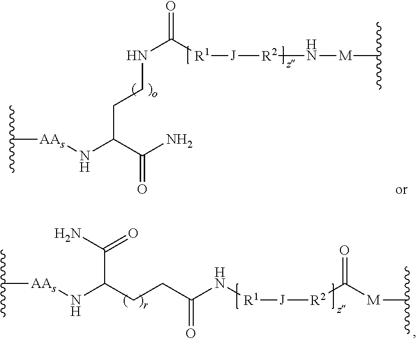

wherein M, AAₛ, each —(R¹-J-R²)z″—, o and z″ are defined herein; r can be 0 or 1.

r can be 0. r can be 1.

The linker can have the structure:

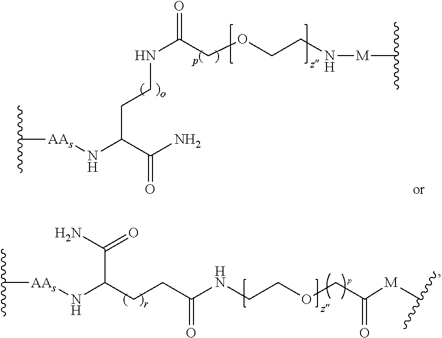

wherein each of M, AAₛ, o, p, q, r and z″ can be as defined herein.

z″ can bean integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

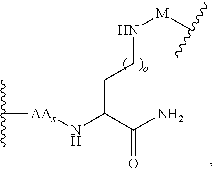

wherein:
M, AAₛ and o are as defined herein.

Other non-limiting examples of suitable linkers include:

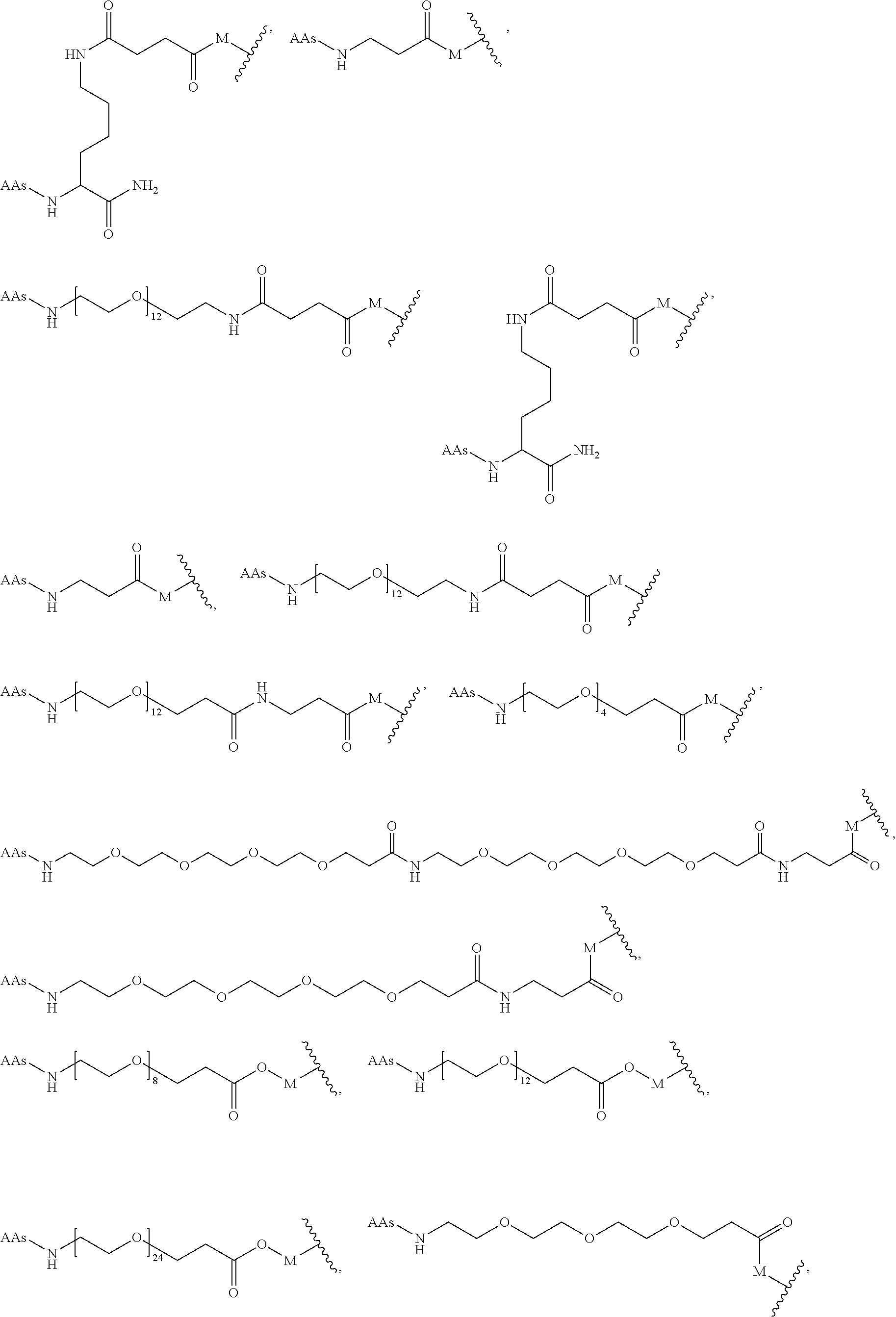

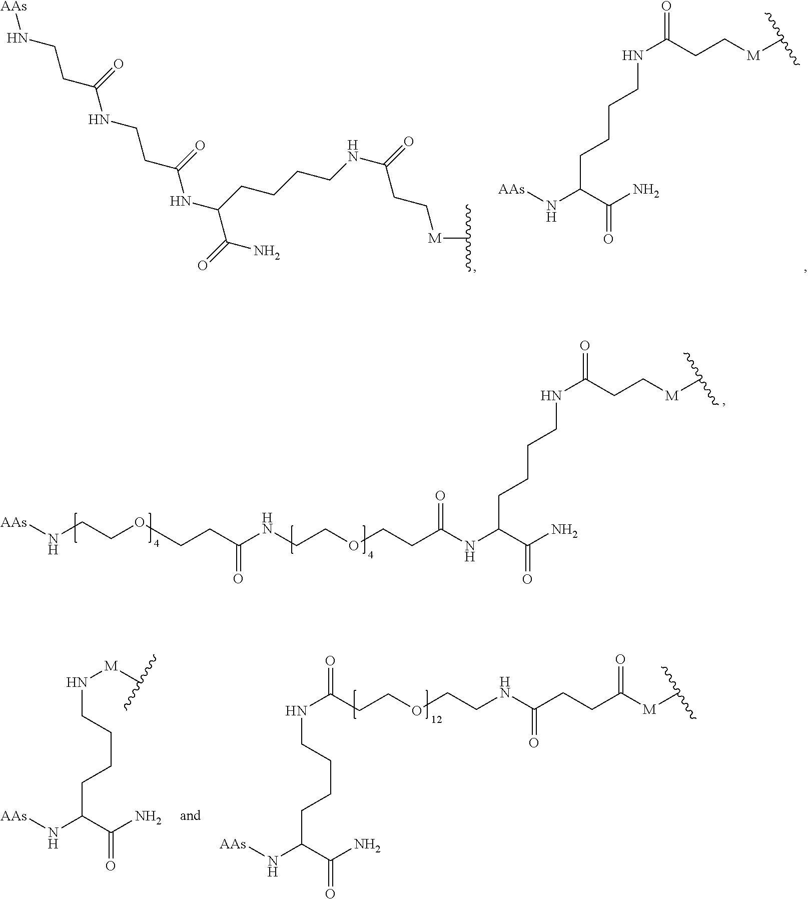

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

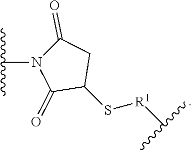

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

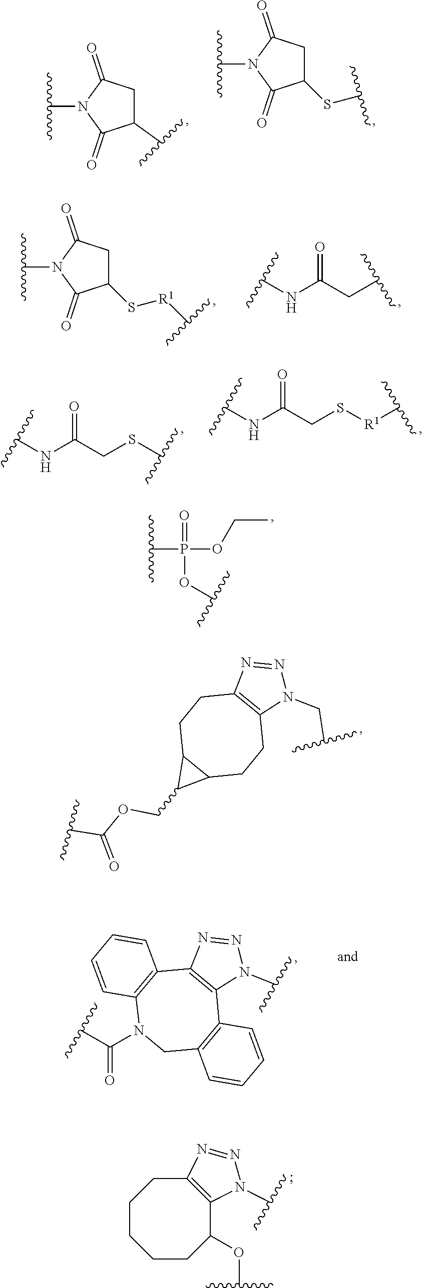

wherein: R¹ is alkylene, cycloalkyl, or

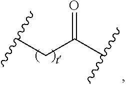

wherein t′ is 0 to 10 wherein each R is independently an alkyl, alkenyl, alkynyl, carbocyclyl, or heterocyclyl, wherein R¹ is

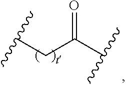

and t′ is 2.

The linker can have the structure:

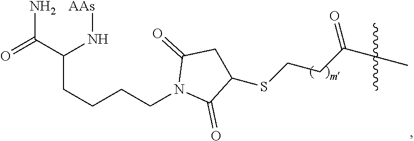

wherein AAₛ is as defined herein, and m′ is 0-10.

The linker can be of the formula:

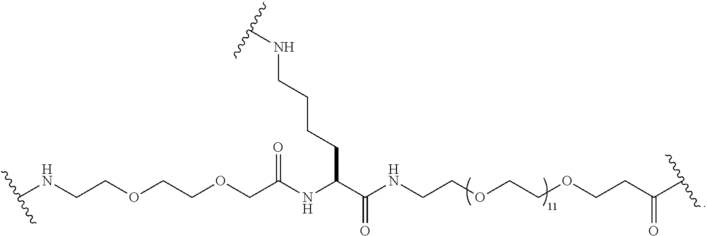

The linker can be of the formula:

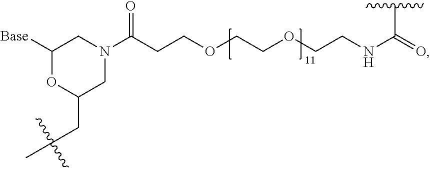

wherein “base” is a nucleobase at the 3′ end of a cargo phosphorodiamidate morpholino oligomer.

The linker can be of the formula:

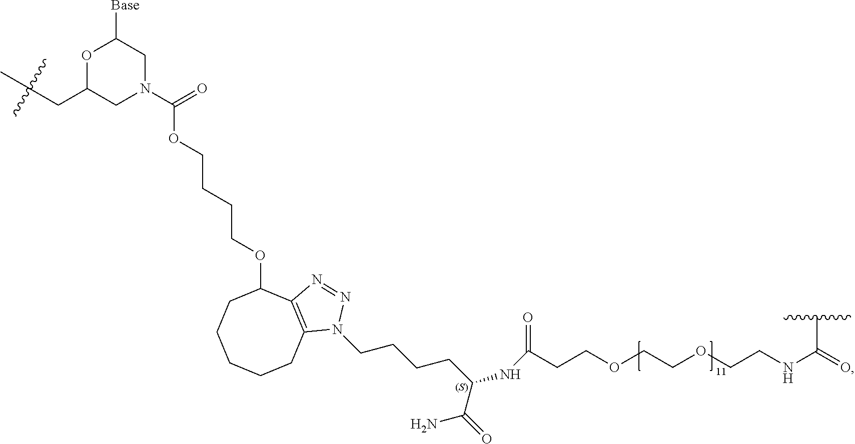

wherein “base” corresponds to a nucleobase at the 3′ end of a cargo phosphorodiamidate morpholino oligomer.

The linker can be of the formula:

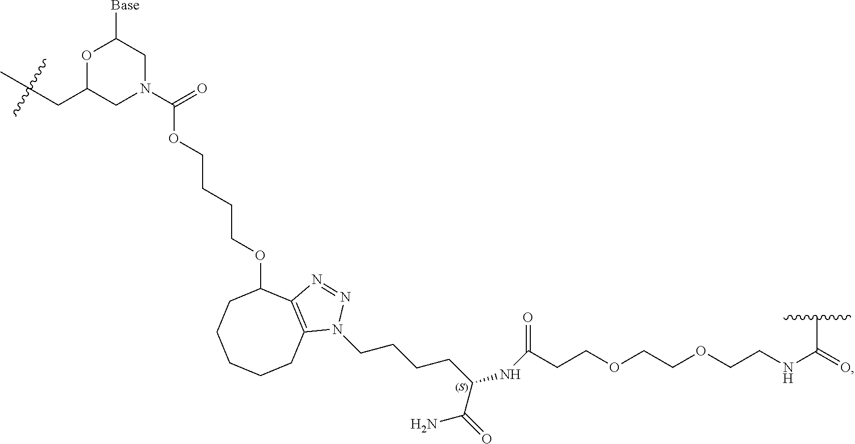

wherein “base” is a nucleobase at the 3′ end of a cargo phosphorodiamidate morpholino oligomer.

The linker can be of the formula:

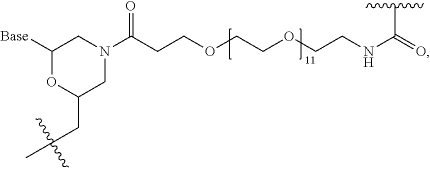

wherein “base” is a nucleobase at the 3′ end of a cargo phosphorodiamidate morpholino oligomer.

The linker can be of the formula:

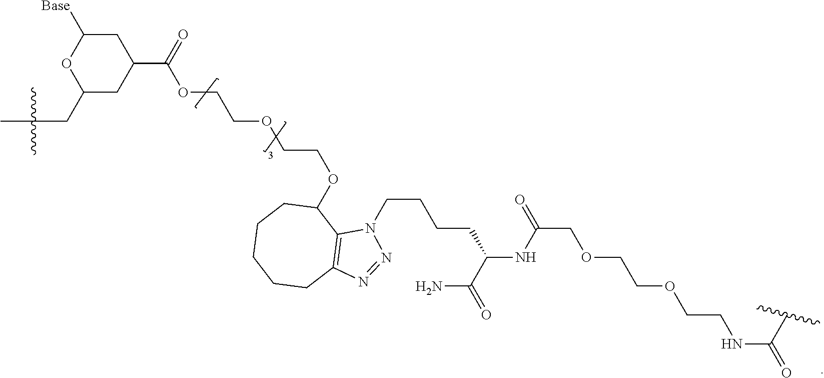

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z}′— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂), is also referred to as PEG. The cCPP-linker conjugate can have a structure selected from Table 8:

TABLE 8
cCPP-linker conjugates and SEQ ID NOs

| | |
|---|---|
| cyclo(FfΦ-4gp-r-4gp-rQ)-PEG₄-K—NH₂ | cyclo(SEQ ID NO: 118)-PEG₄-K—NH₂ |
| cyclo(FfΦ-Cit-r-Cit-rQ)-PEG₄-K—NH₂ | cyclo(SEQ ID NO: 119)-PEG₄-K—NH₂ |
| cyclo(FfΦ-Pia-r-Pia-rQ)-PEG₄-K—NH₂ | cyclo(SEQ ID NO: 120)-PEG₄-K—NH₂ |
| cyclo(FfΦ-Dml-r-Dml -rQ)-PEG₄-K—NH₂ | cyclo(SEQ ID NO: 121)-PEG₄-K—NH₂ |
| cyclo(FfΦ-Cit-r-Cit-rQ)-PEG₁₂-OH | cyclo(SEQ ID NO: 122)-PEG₁₂-OH |
| cyclo(fΦR-Cit-R-Cit-Q)-PEG₁₂-OH | cyclo(SEQ ID NO: 123)-PEG₁₂-OH |
| cyclo(fΦ-Can-r-Can-r-Q)-PEG₁₂-OH | cyclo(SEQ ID NO: 124)-PEG₁₂-OH |
| cyclo(FfΦ-Can-r-Can-r-Q)-PEG₁₂-OH | cyclo(SEQ ID NO: 125)-PEG₁₂-OH |

The linker can comprise a —(OCH₂CH₂)_{z}′— subunit, wherein z′ is an integer from 1 to 23, and a peptide subunit. The peptide subunit can comprise from 2 to 10 amino acids. The cCPP-linker conjugate can have a structure selected from Table 9:

TABLE 9
cCPP-linker conjugate and SEQ ID NOs

| | |
|---|---|
| Ac-PKKK-Can-KV-PEG₂-K(cyclo(FfΦ-Can-r- | Ac-SEQ ID NO: 126-PEG₂-K(cyclo(SEQ ID |
| Can-r-Q)-PEG₁₂-K(N₃)—NH₂ | NO: 125)-PEG₁₂-K(N₃)—NH₂ |
| Ac-PKKKRKV-Lys(cyclo[FfΦ-R-r-Cit-rQ])- | Ac-SEQ ID NO: 42-Lys(cyclo[SEQ ID |
| PEG₁₂-K(N₃)—NH₂ | NO: 127])-PEG₁₂-K(N₃)—NH₂ |
| Ac-PKKKRKV-Lys(cyclo[FfΦ-Cit-r-R-rQ])- | Ac- SEQ ID NO: 42-Lys(cyclo[SEQ ID |
| PEG₁₂-K(N₃)—NH₂ | NO: 1281)-PEG₁₂-K(N₃)—NH₂ |
| Ac-PKKKRKV-K(cyclo(FfΦR-cit-R-cit-Q))- | Ac- SEQ ID NO: 42-K(cyclo(SEQ ID |
| PEG₁₂-K(N₃)—NH₂ | NO:129))-PEG₁₂-K(N₃)—NH₂ |
| Ac-PKKKRKV-PEG2-Lys(cyclo[FfΦ-Cit-r-Cit- | Ac- SEQ ID NO: 42-PEG2-Lys(cyclo[SEQ |
| rQ])-B-k(N₃)—NH₂ | ID NO: 130])-B-k(N₃)—NH₂ |
| Ac-PKKKRKV-PEG2-Lys(cyclo[FfΦ-Cit-r-Cit- | Ac- SEQ ID NO: 42-PEG2-Lys(cyclo[SEQ |
| rQ])-PEG2-k(N₃)—NH₂ | ID NO: 130])-PEG2-k(N₃)—NH₂ |
| Ac-PKKKRKV-PEG2-Lys(cyclo[FfΦ-Cit-r-Cit- | Ac- SEQ ID NO: 42-PEG2-Lys(cyclo[SEQ |
| rQ])-PEG4-k(N₃)—NH₂ | ID NO: 130])-PEG4-k(N₃)—NH₂ |
| Ac-PKKKRKV-Lys(cyclo[FfΦ-Cit-r-Cit-rQ])- | Ac- SEQ ID NO: 42-Lys(cyclo[SEQ ID |
| PEG12-k(N₃)—NH₂ | NO: 130])-PEG12-k(N₃)—NH₂ |
| Ac-pkkkrkv-PEG2-Lys(cyclo[FfΦ-Cit-r-Cit-rQ])- | Ac- SEQ ID NO: 131-PEG2-Lys(cyclo[SEQ |
| PEG12-k(N₃)—NH₂ | ID NO: 130])-PEG12-k(N₃)—NH₂ |
| Ac-rrv-PEG2-Lys(cyclo[FfΦ-Cit-r-Cit-rQ])- | Ac-rrv-PEG2-Lys(cyclo[SEQ ID NO:130])- |
| PEG12-OH | PEG12-OH |
| Ac-PKKKRKV-PEG2-Lys(cyclo[FfΦ-Cit-r-Cit-f- | Ac- SEQ ID NO: 42-PEG2-Lys(cyclo[SEQ |
| Q])-PEG12-k(N₃)—NH₂ | ID NO: 130])-PEG12-k(N₃)—NH₂ |
| Ac-PKKK-Cit-KV-PEG2-Lys(cyclo[FfΦ-Cit-r- | Ac- SEQ ID NO: 126-PEG2-Lys(cyclo[SEQ |
| Cit-r-Q])-PEG12-k(N₃)—NH₂ | ID NO: 130])-PEG12-k(N₃)—NH₂ |
| Ac-PKKKRKV-PEG2-Lys(cyclo[FfΦ-Cit-r-Cit-r- | Ac- SEQ ID NO: 42-PEG2-Lys(cyclo[SEQ |
| Q]-PEG12-K(N₃)—NH₂ | ID NO: 130]-PEG12-K(N₃)—NH₂ |

EEVs comprising a cyclic cell penetrating peptide (cCPP), linker and exocyclic peptide (EP) are provided. An EEV can comprise the structure of Formula (B):

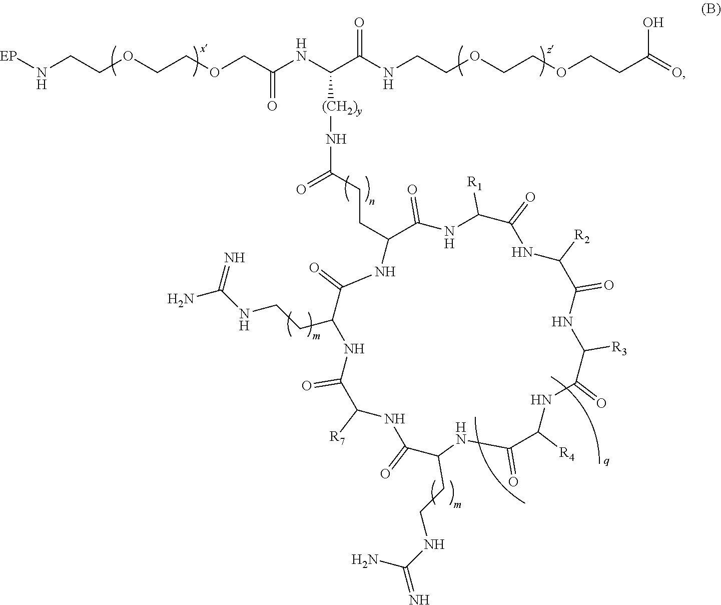

or a protonated form thereof,

wherein:
R₁, R₂, and R₃ are each independently H or an aromatic or heteroaromatic side chain of an amino acid;
R₄ and R₇ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.

R₁, R₂, R₃, R₄, R₇, EP, m, q, y, x′, z′ are as described herein.

n can be 0. n can be 1. n can be 2.

The EEV can comprise the structure of Formula (B-a) or (B-b):

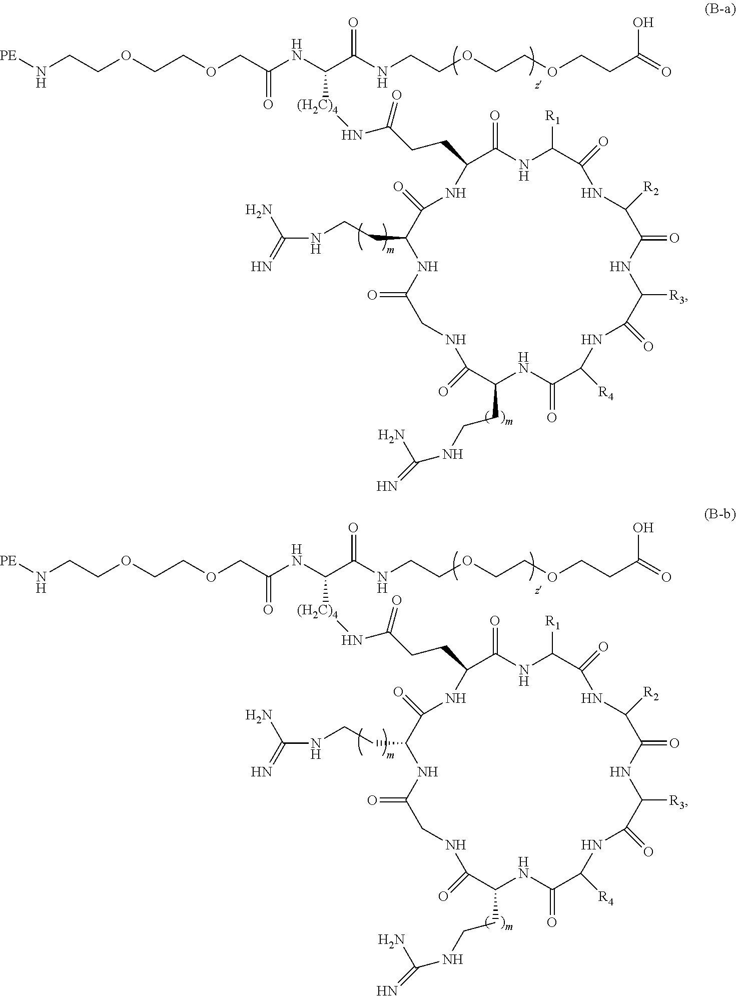

or a protonated form thereof, wherein EP (shown as “PE”), R¹, R², R³, R⁴, m and z′ are as defined above in Formula (B).

The EEV can comprises the structure of Formula (B-c):

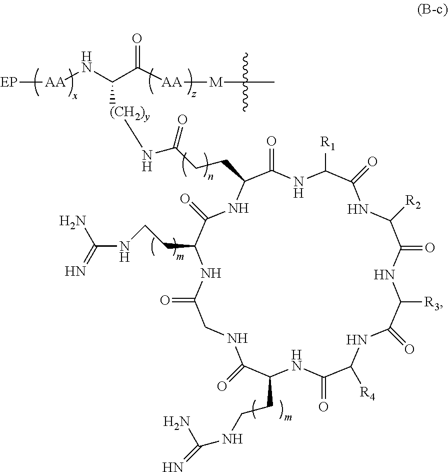

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

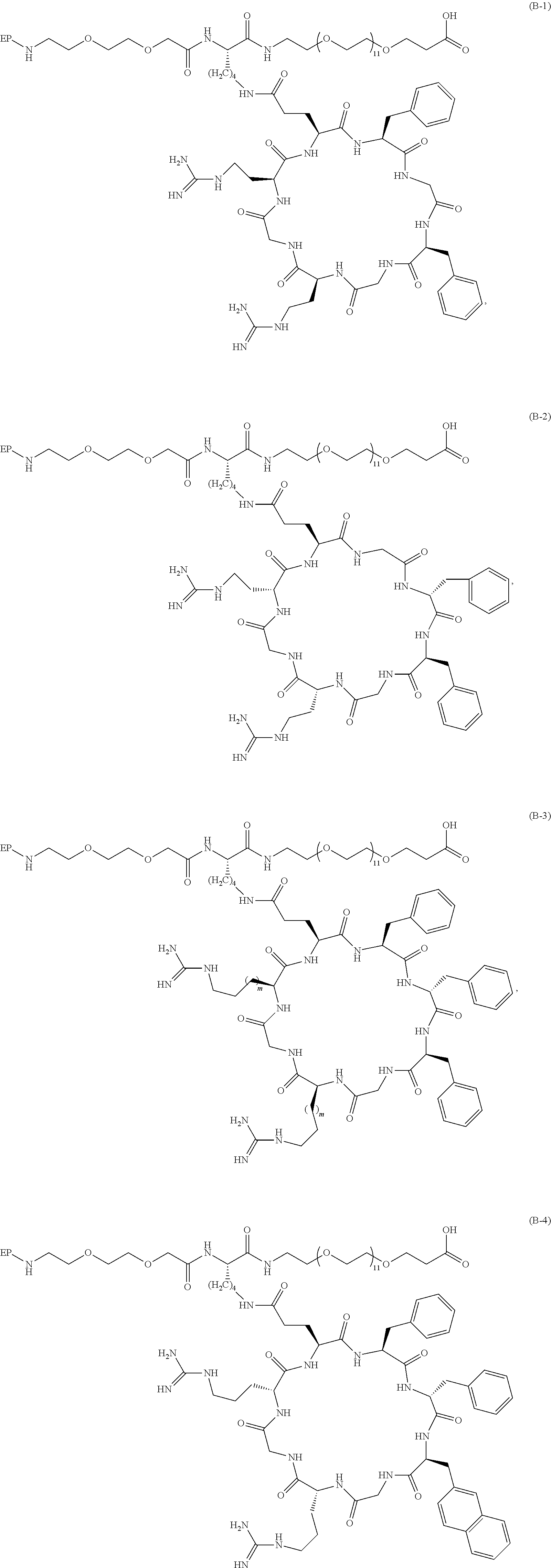

or a protonated form thereof, wherein EP is as defined above in Formula (B).

The EEV can comprise Formula (B) and can have the structure: Ac—PKKKRKVAEEA-K(cyclo[FGFGRGRQ])-PEG₁₂-OH (Ac-SEQ ID NO:132-K(cyclo[SEQ ID NO:82])-PEG₁₂-OH) or Ac—PK—KKR—KV-AEEA-K(cyclo[GfFGrGrQ])-PEG₁₂-OH (Ac-SEQ ID NO:133-K(cyclo[SEQ ID NO:83])PEG₁₂-OH).

The EEV can comprise a cCPP of formula:

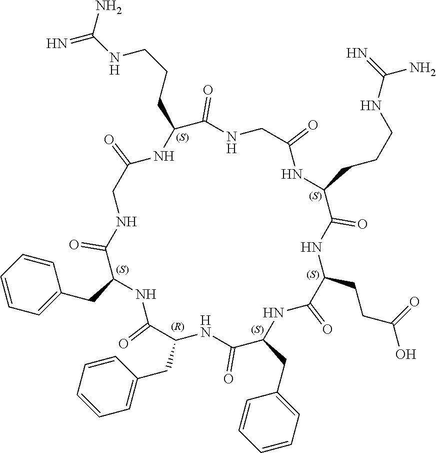

The EEV can comprise formula: Ac—PKKKRKV-miniPEG₂-Lys(cyclo(FfFGRGRQ)-PEG₂-K(N₃) (Ac-SEQ ID NO:42-miniPEG₂-Lys(cyclo(SEQ ID NO:81)-PEG₂-K(N₃)).

The EEV can be:

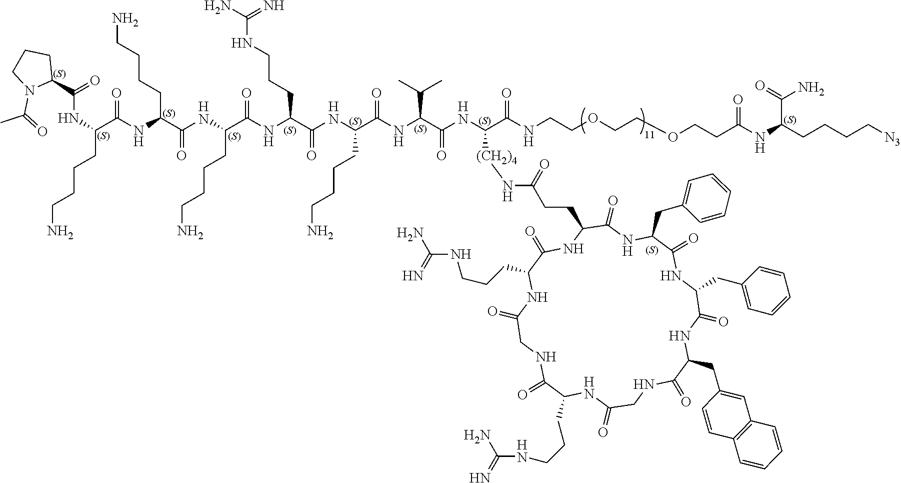

The EEV can be

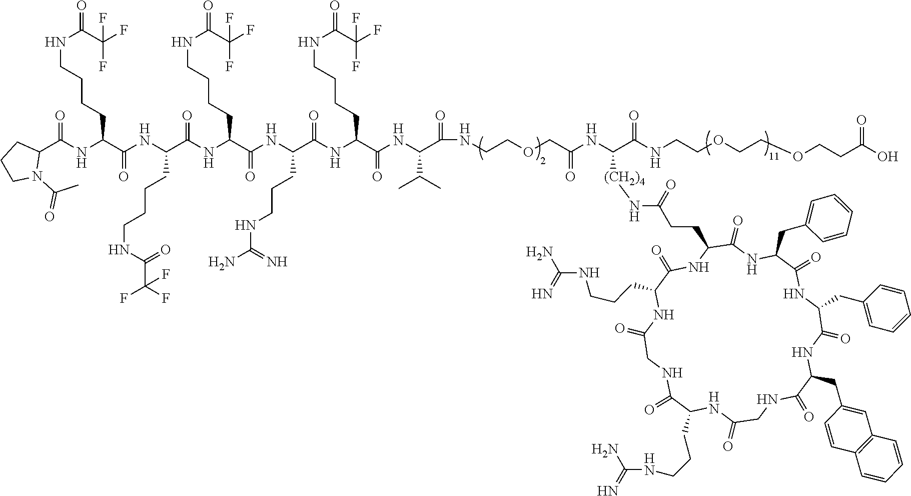

The EEV can be Ac—P—K(Tfa)-K(Tfa)-K(Tfa)-R—K(Tfa)-V-miniPEG₂-K(cyclo(Ff-Nal-GrGrQ)-PEG₁₂-OH (Ac-SEQ ID NO:134-miniPEG₂-K(cyclo(SEQ ID NO:135)-PEG₁₂-OH).

The EEV can be

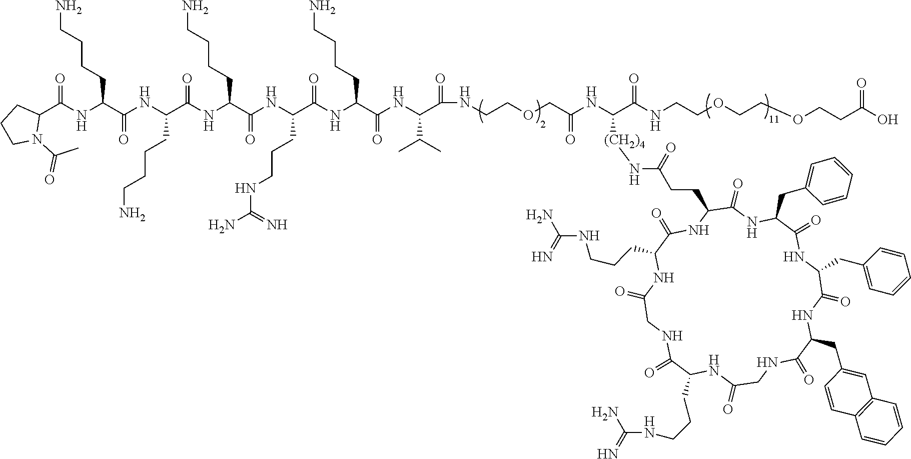

The EEV can be Ac—P—K—K—K—R—K—V-miniPEG₂-K(cyclo(Ff-Nal-GrGrQ)-PEG₁₂-OH (Ac-SEQ ID NO:42-miniPEG₂-K(cyclo(SEQ ID NO:135)-PEG₁₂-OH).

The EEV can be

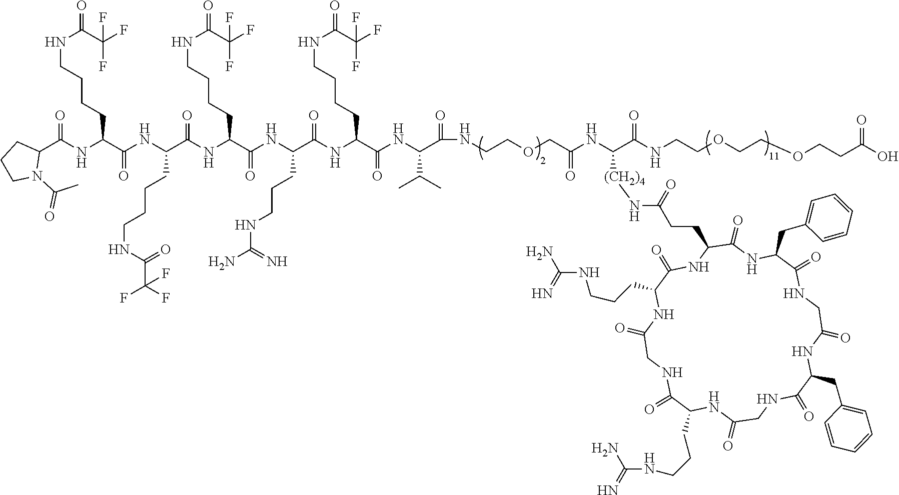

The EEV can be

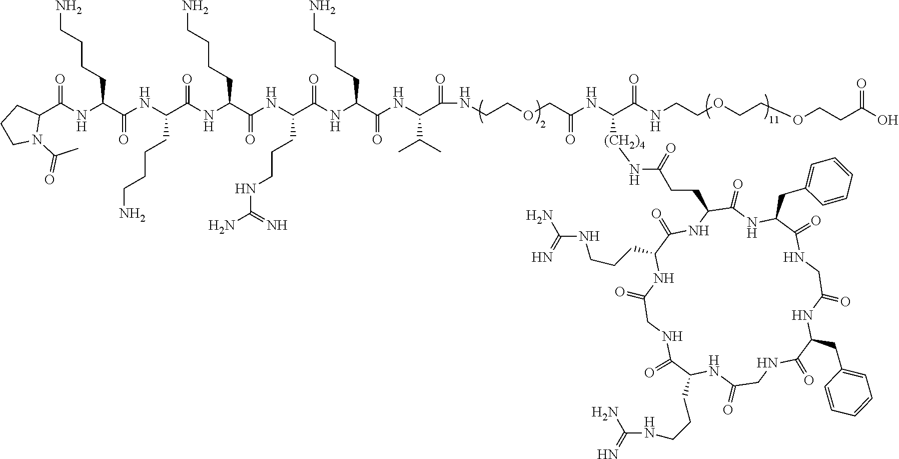

### The EEV can be

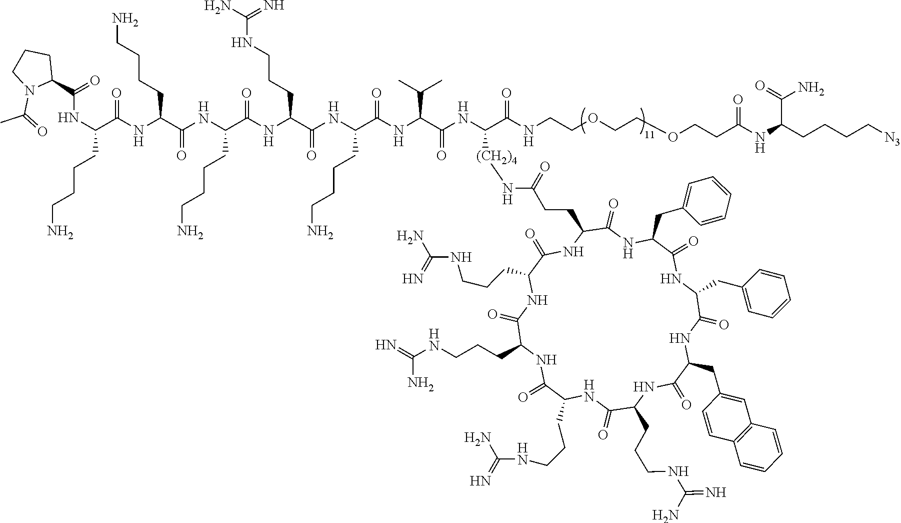

The EEV can be

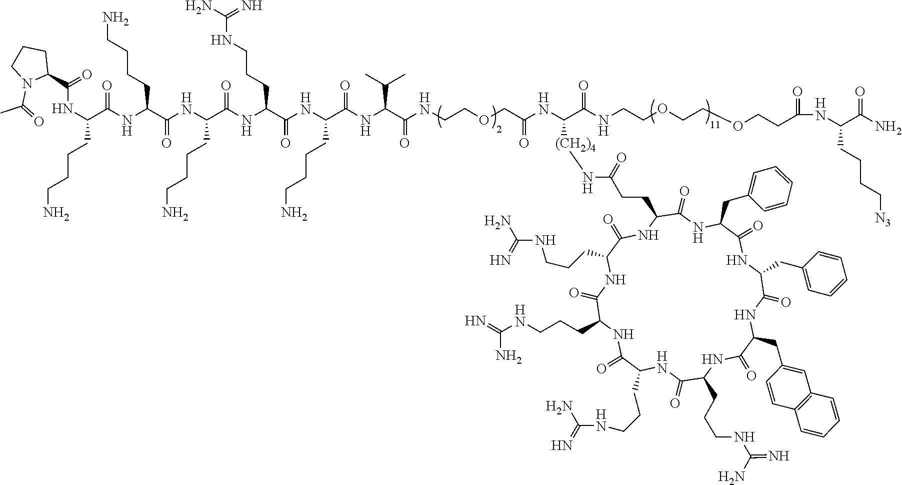

The EEV can be

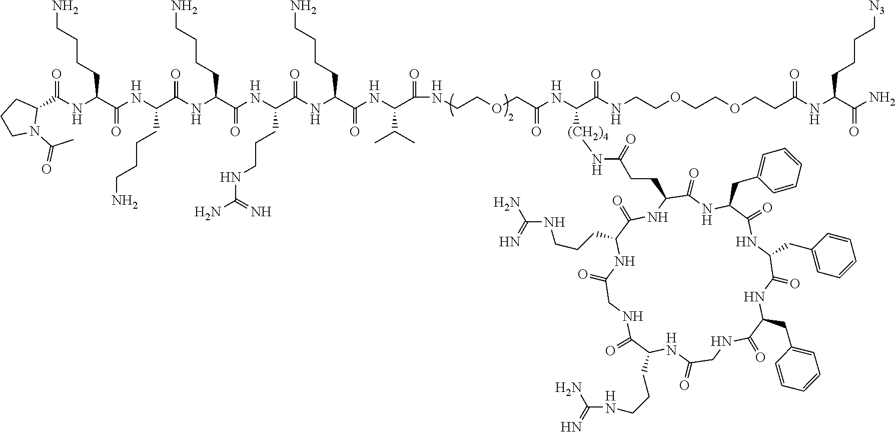

The EEV can be:

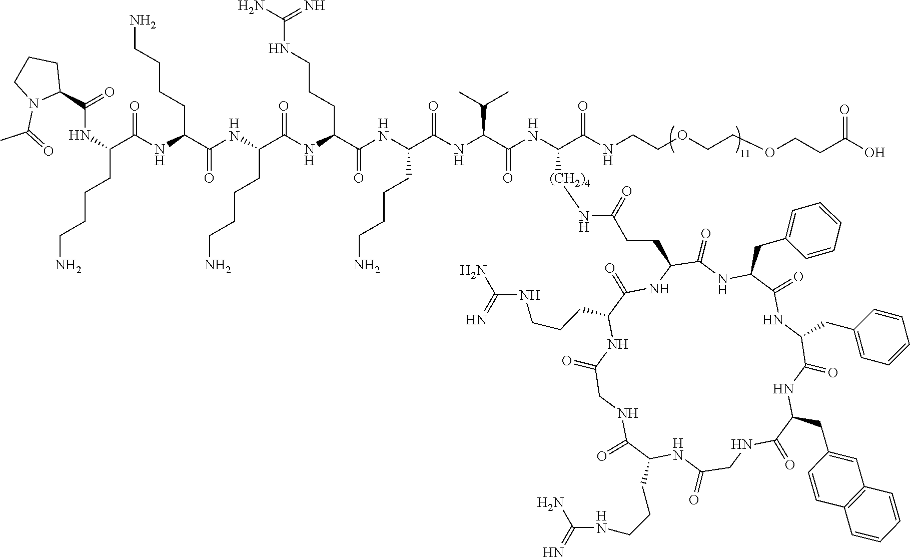

The EEV can be:

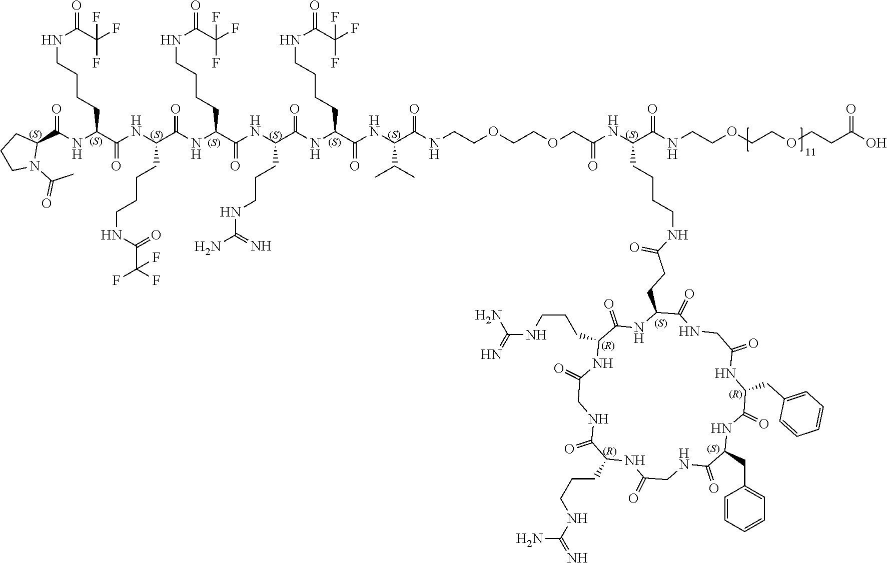

The EEV can be:

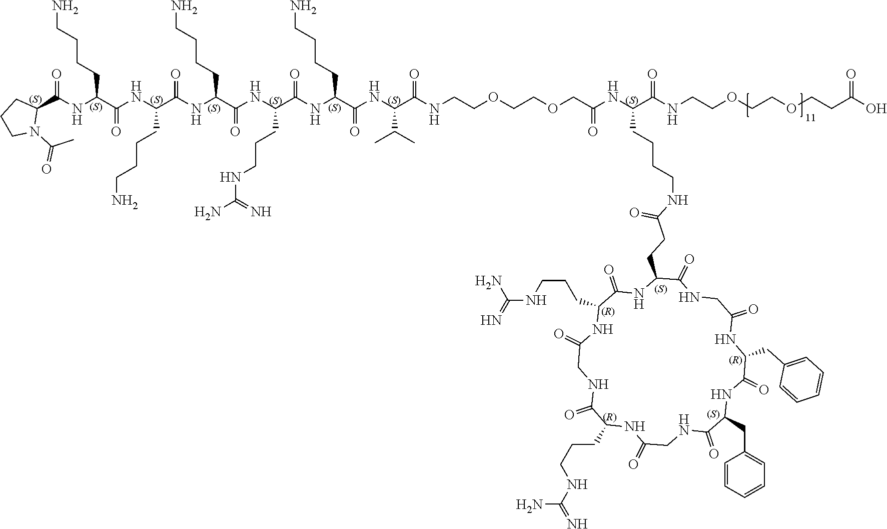

The EEV can be:

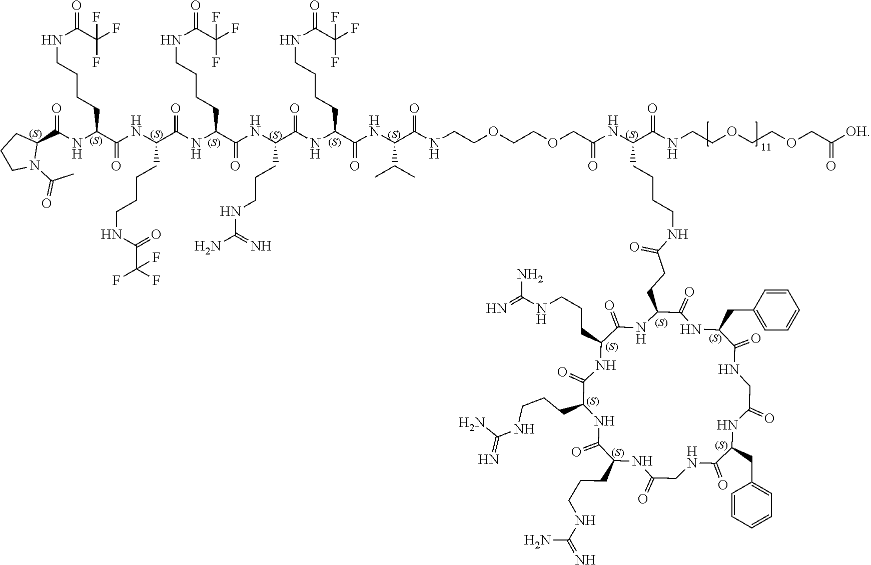

The EEV can be

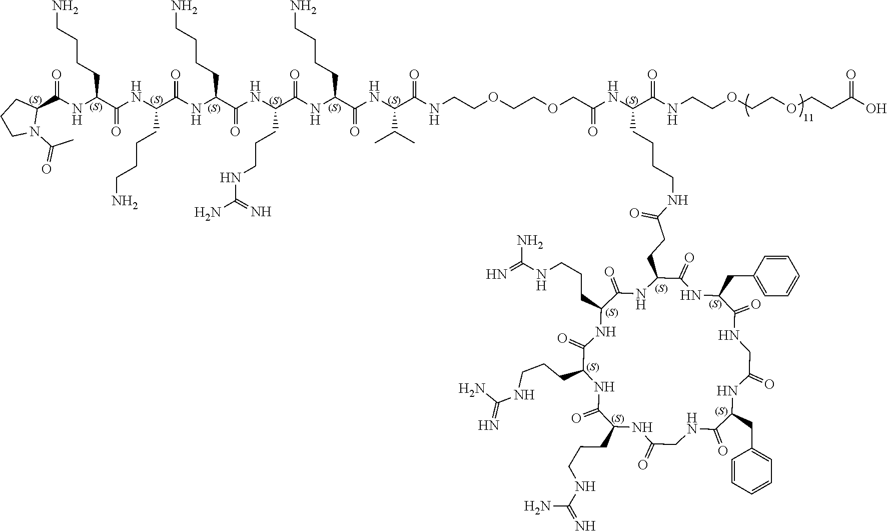

The EEV can be

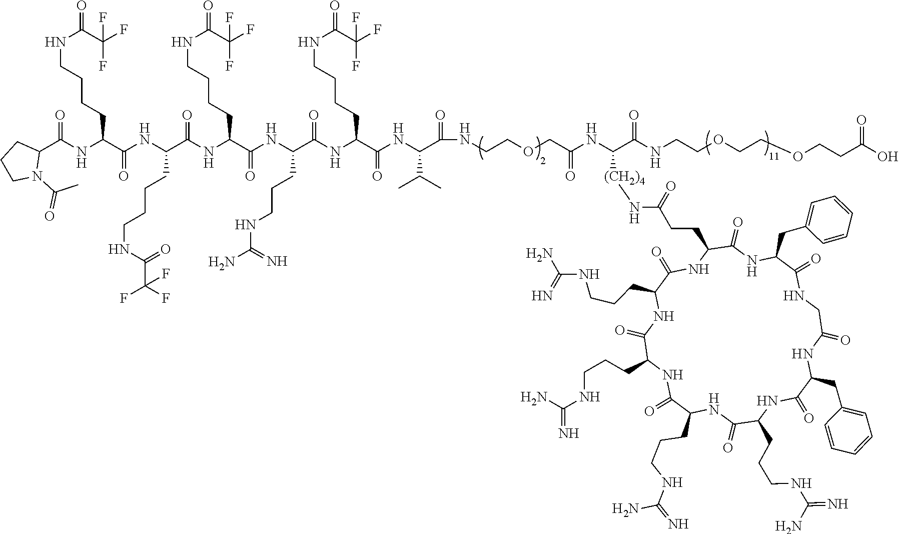

The EEV can be selected from

| | |
|---|---|
| Ac-rr-miniPEG₂-Dap(cyclo[FfΦ-Cit-r-Cit- | Ac-rr-miniPEG₂-Dap(cyclo[SEQ |
| rQ])-PEG₁₂-OH | ID NO: 136])-PEG₁₂-OH |
| Ac-frr-PEG₂-Dap(cyclo[FfΦ-Cit-r-Cit- | Ac-frr-PEG₂-Dap(cyclo[SEQ ID |
| rQ])-PEG₁₂-OH | NO: 136])-PEG₁₂-OH |
| Ac-rfr-PEG₂-Dap(cyclo[FfΦ-Cit-r-Cit- | Ac-rfr-PEG₂-Dap(cyclo[SEQ ID |
| rQ])-PEG₁₂-OH | NO: 136])-PEG₁₂-OH |
| Ac-rbfbr-PEG₂-Dap(cyclo[FfΦ-Cit-r-Cit- | Ac-SEQ ID NO: 137-PEG₂- |
| rQ])-PEG₁₂-OH | Dap(cyclo[SEQ ID NO: 136])- |
| | PEG₁₂-OH |
| Ac-rrr-PEG₂-Dap(cyclo[FfΦ-Cit-r-Cit- | Ac-rrr-PEG₂-Dap(cyclo[SEQ ID |
| rQ])-PEG₁₂-OH | NO: 136])-PEG₁₂-OH |
| Ac-rbr-PEG₂-Dap(cyclo[FfΦ-Cit-r-Cit- | Ac-rbr-PEG₂-Dap(cyclo[SEQ ID |
| rQ])-PEG₁₂-OH | NO: 136])-PEG₁₂-OH |
| Ac-rbrbr-PEG₂-Dap(cyclo[FfΦ-Cit-r-Cit- | Ac-SEQ ID NO:138-PEG₂- |
| rQ)-PEG₂-OH | Dap(cyclo[SEQ ID NO: 136])- |
| | PEG₁₂-OH |
| Ac-hh-PEG₂-Dap(cyclo[FfΦ-Cit-r-Cit- | Ac-hh-PEG₂-Dap(cyclo[SEQ ID |
| rQ])-PEG₁₂-OH | NO: 136])-PEG₁₂-OH |
| Ac-hbh-PEG₂-Dap(cyclo[FfΦ-Cit-r-Cit- | Ac-hbh-PEG₂-Dap(cyclo[SEQ ID |
| rQ])-PEG₁₂-OH | NO: 136])-PEG₁₂-OH |
| Ac-hbhbh-PEG₂-Dap(cyclo[FfΦ-Cit-r-Cit- | Ac-SEQ IN NO: 139-PEG₂- |
| rQ])-PEG₁₂-OH | Dap(cyclo[SEQ ID NO: 136])- |
| | PEG₁₂-OH |
| Ac-rbhbh-PEG₂-Dap(cyclo[FfΦ-Cit-r-Cit- | Ac- SEQ ID NO: 140-PEG₂- |
| rQ])-PEG₁₂-OH | Dap(cyclo[SEQ ID NO: 136])- |
| | PEG₁₂-OH |
| Ac-hbrbh-PEG₂-Dap(cyclo[FfΦ-Cit-r-Cit- | Ac-SEQ ID NO: 141-PEG₂- |
| rQ])-PEG₁₂-OH | Dap(cyclo[SEQ ID NO: 136])- |
| | PEG₁₂-OH |
| Ac-rr-Dap(cyclo[FfΦ-Cit-r-Cit-rQ])-b-OH | Ac-rr-Dap(cyclo[SEQ ID |
| | NO: 136])-b-OH |
| Ac-frr-Dap(cyclo[FfΦ-Cit-r-Cit-rQ])-b-OH | Ac-frr-Dap(cyclo[SEQ ID |
| | NO: 136])-b-OH |
| Ac-rfr-Dap(cyclo[FfΦ-Cit-r-Cit-rQ])-b-OH | Ac-rfr-Dap(cyclo[SEQ ID |
| | NO: 136])-b-OH |
| Ac-rbfbr-Dap(cyclo[FfΦ-Cit-r-Cit-rQ])- | Ac- SEQ ID NO: 137- |
| b-OH | Dap(cyclo[SEQ ID NO: 136])- |
| | b-OH |
| Ac-rrr-Dap(cyclo[FfΦ-Cit-r-Cit-rQ])-b-OH | Ac-rrr-Dap(cyclo[SEQ ID |
| | NO: 136])-b-OH |
| Ac-rbr-Dap(cyclo[FfΦ-Cit-r-Cit-rQ])-b-OH | Ac-rbr-Dap(cyclo[SEQ ID |
| | NO: 136])-b-OH |
| Ac-rbrbr-Dap(cyclo[FfΦ-Cit-r-Cit-rQ])- | Ac- SEQ ID NO: 138- |
| b-OH | Dap(cyclo[SEQ ID NO: 136])-b- |
| | OH |
| Ac-hh-Dap(cyclo[FfΦ-Cit-r-Cit-rQ])-b-OH | Ac-hh-Dap(cyclo[SEQ ID |
| | NO: 136])-b-OH |
| Ac-hbh-Dap(cyclo[FfΦ-Cit-r-Cit-rQ])-b-OH | Ac-hbh-Dap(cyclo[SEQ ID |
| | NO: 136])-b-OH |
| Ac-hbhbh-Dap(cyclo[FfΦ-Cit-r-Cit-rQ])- | Ac- SEQ IN NO: 139- |
| b-OH | Dap(cyclo[SEQ ID NO: 136])-b- |
| | OH |
| Ac-rbhbh-Dap(cyclo[FfΦ-Cit-r-Cit-rQ])- | Ac-SEQ ID NO: 140- |
| b-OH | Dap(cyclo[SEQ ID NO: 136])-b- |
| | OH |
| Ac-hbrbh-Dap(cyclo[FfΦ-Cit-r-Cit-rQ])- | Ac- SEQ ID NO: 141- |
| b-OH | Dap(cyclo[SEQ ID NO: 136])-b- |
| | OH |
| Ac-KKKK-miniPEG2-Lys(cyclo[FfΦGrGrQ])- | Ac- SEQ ID NO:7-miniPEG₂- |
| miniPEG₂-K(N₃)—NH₂ | Lys(cyclo[SEQ ID NO: Z80])- |
| | miniPEG₂-K(N₃)—NH₂ |
| Ac-KGKK-miniPEG2-Lys(cyclo[FfΦGrGrQ])- | Ac- SEQ ID NO: 13-miniPEG₂- |
| miniPEG₂-K(N₃)—NH₂ | Lys(cyclo[SEQ ID NO: Z80])- |
| | miniPEG₂-K(N₃)—NH₂ |
| Ac-KKGK-miniPEG₂-Lys(cyclo[FfΦGrGrQ])- | Ac- SEQ ID NO: 14-miniPEG₂- |
| miniPEG₂-K(N₃)—NH₂ | Lys(cyclo[SEQ ID NO: Z80])- |
| | miniPEG2-K(N₃)—NH₂ |
| Ac-KKK-miniPEG₂-Lys(cyclo[FfΦGrGrQ])- | Ac-KKK-miniPEG₂- |
| miniPEG₂-K(N₃)—NH₂ | Lys(cyclo[SEQ ID NO: 80])- |
| | miniPEG₂-K(N₃)—NH₂ |
| Ac-KK-miniPEG₂-Lys(cyclo[FfΦGrGrQ])- | Ac-KK-miniPEG₂-Lys(cyclo[SEQ |
| miniPEG₂-K(N₃)—NH₂ | ID NO: 80])-miniPEG₂-K(N₃)—NH₂ |
| Ac-KGK-miniPEG₂-Lys(cyclo[FfΦGrGrQ])- | Ac-KGK-miniPEG₂- |
| miniPEG₂-K(N₃)—NH₂ | Lys(cyclo[SEQ ID NO: 80])- |
| | miniPEG₂-K(N₃)—NH₂ |
| Ac-KBK-miniPEG₂-Lys(cyclo[FfΦGrGrQ])- | Ac-KBK-miniPEG₂- |
| miniPEG₂-K(N₃)—NH₂ | Lys(cyclo[SEQ ID NO:80])- |
| | miniPEG₂-K(N₃)—NH₂ |
| Ac-KBKBK-miniPEG₂-Lys(cyclo[FfΦGrGrQ])- | Ac- SEQ ID NO: 24-miniPEG₂- |
| miniPEG₂-K(N₃)—NH₂ | Lys(cyclo[SEQ ID NO: 80])- |
| | miniPEG₂-K(N₃)—NH₂ |
| Ac-KR-miniPEG₂-Lys(cyclo[FfΦGrGrQ])- | Ac-KR-miniPEG₂-Lys(cyclo[SEQ |
| miniPEG₂-K(N₃)—NH₂ | ID NO: 80])-miniPEG₂-K(N₃)—NH₂ |
| Ac-KBR-miniPEG₂-Lys(cyclo[FfΦGrGrQ])- | Ac-KBR-miniPEG₂- |
| miniPEG₂-K(N₃)—NH₂ | Lys(cyclo[SEQ ID NO: 80])- |
| | miniPEG₂-K(N₃)—NH₂ |
| Ac-PKKKRKV-miniPEG₂-Lys(cyclo[FfΦGrGrQ])- | Ac- SEQ ID NO: 42-miniPEG₂- |
| miniPEG₂-K(N₃)-NH₂ | Lys(cyclo[SEQ ID NO: 80])- |
| | miniPEG₂-K(N₃)—NH₂ |
| Ac-PKKKRKV-miniPEG₂-Lys(cyclo[FfΦGrGrQ])- | Ac- SEQ ID NO: 42-miniPEG₂- |
| miniPEG₂-K(N₃)—NH₂ | Lys(cyclo[SEQ ID NO: 80])- |
| | miniPEG₂-K(N₃)—NH₂ |
| Ac-PGKKRKV-miniPEG₂-Lys(cyclo[FfΦGrGrQ])- | Ac- SEQ ID NO: 43-miniPEG₂- |
| miniPEG₂-K(N₃)—NH₂ | Lys(cyclo[SEQ ID NO: 80])- |
| | miniPEG₂-K(N₃)—NH₂ |
| Ac-PKGKRKV-miniPEG₂-Lys(cyclo[FfΦGrGrQ])- | Ac- SEQ ID NO: 44-miniPEG₂- |
| miniPEG₂-K(N₃)—NH₂ | Lys(cyclo[SEQ ID NO: 80])- |
| | miniPEG₂-K(N₃)—NH₂ |
| Ac-PKKGRKV-miniPEG₂-Lys(cyclo[FfΦGrGrQ])- | Ac- SEQ ID NO: 45-miniPEG₂- |
| miniPEG₂-K(N₃)—NH₂ | Lys(cyclo[SEQ ID NO: 80])- |
| | miniPEG₂-K(N₃)—NH₂ |
| Ac-PKKKGKV-miniPEG₂-Lys(cyclo[FfΦGrGrQ])- | Ac- SEQ ID NO: 46-miniPEG₂- |
| miniPEG₂-K(N₃)—NH₂ | Lys(cyclo[SEQ ID NO: 80])- |
| | miniPEG₂-K(N₃)—NH₂ |
| Ac-PKKKRGV-miniPEG₂-Lys(cyclo[FfΦGrGrQ])- | Ac- SEQ ID NO: 47-miniPEG₂- |
| miniPEG₂-K(N₃)—NH₂ | Lys(cyclo[SEQ ID NO: 80])- |
| | miniPEG₂-K(N₃)—NH₂ |
| Ac-PKKKRKG-miniPEG₂-Lys(cyclo[FfΦGrGrQ])- | Ac- SEQ ID NO: 48-miniPEG₂- |
| miniPEG₂-K(N₃)—NH₂ | Lys(cyclo[SEQ ID NO: 80])- |
| | miniPEG₂-K(N₃)—NH₂ |
| Ac-KKKRK-miniPEG₂-Lys(cyclo[FfΦGrGrQ])- | Ac- SEQ ID NO: 19-miniPEG₂- |
| miniPEG₂-K(N₃)—NH₂ | Lys(cyclo[SEQ ID NO: 80])- |
| | miniPEG₂-K(N₃)—NH₂ |
| Ac-KKRK-miniPEG₂-Lys(cyclo[FfΦGrGrQ])- | Ac- SEQ ID NO: 8-miniPEG₂- |
| miniPEG₂-K(N₃)—NH₂ and | Lys(cyclo[SEQ ID NO: 80])- |
| | miniPEG₂-K(N₃)—NH₂ and |
| Ac-KRK-miniPEG₂-Lys(cyclo[FfΦGrGrQ])- | Ac-KRK-miniPEG₂- |
| miniPEG₂-K(N₃)—NH₂. | Lys(cyclo[SEQ ID NO: 80]- |
| | miniPEG₂-K(N₃)—NH₂. |

The EEV can be selected from:
Ac—PKKKRKV-Lys(cyclo[FfΦGrGrQ])-PEG₁₂-K(N₃)—NH₂
(Ac-SEQ ID NO:42-Lys(cyclo[SEQ ID NO:80])-PEG₁₂-K(N₃)—NH₂)
Ac—PKKKRKV-miniPEG₂-Lys(cyclo[FfΦGrGrQ])-miniPEG₂-K(N₃)—NH₂
(Ac-SEQ ID NO:42-miniPEG₂-Lys(cyclo[SEQ ID NO:80])-miniPEG₂-K(N₃)—NH₂)
Ac—PKKKRKV-miniPEG₂-Lys(cyclo[FGFGRGRQ])-miniPEG₂-K(N₃)—NH₂
(Ac-SEQ ID NO:42-miniPEG₂-Lys(cyclo[SEQ ID NO:82])-miniPEG₂-K(N₃)—NH₂)
Ac—KR-PEG₂-K(cyclo[FGFGRGRQ])-PEG₂-K(N₃)—NH₂
(Ac—KR-PEG₂-K(cyclo[SEQ ID NO:82])-PEG₂-K(N₃)—NH₂)
Ac—PKKKGKV-PEG₂-K(cyclo[FGFGRGRQ])-PEG₂-K(N₃)—NH₂
(Ac-SEQ ID NO:46-PEG₂-K(cyclo[SEQ ID NO:82])-PEG₂-K(N₃)—NH₂)
Ac—PKKKRKG-PEG₂-K(cyclo[FGFGRGRQ])-PEG₂-K(N₃)—NH₂
(Ac-SEQ ID NO:48-PEG₂-K(cyclo[SEQ ID NO:82])-PEG₂-K(N₃)—NH₂)
Ac—KKKRK-PEG₂-K(cyclo[FGFGRGRQ])-PEG₂-K(N₃)—NH₂
(Ac-SEQ ID NO:19-PEG₂-K(cyclo[SEQ ID NO:82])-PEG₂-K(N₃)—NH₂)
Ac—PKKKRKV-miniPEG₂-Lys(cyclo[FFΦGRGRQ])-miniPEG₂-K(N₃)—NH₂
(Ac-SEQ ID NO:42-miniPEG₂-Lys(cyclo[SEQ ID NO:80])-miniPEG₂-K(N₃)—NH₂)
Ac—PKKKRKV-miniPEG₂-Lys(cyclo[βhFfΦGrGrQ])-miniPEG₂-K(N₃)—NH₂
(Ac-SEQ ID NO:42-miniPEG₂-Lys(cyclo[SEQ ID NO: 142])-miniPEG₂-K(N₃)—NH₂)
Ac—PKKKRKV-miniPEG₂-Lys(cyclo[FfΦSrSrQ])-miniPEG₂-K(N₃)—NH₂
(Ac-SEQ ID NO:42-miniPEG₂-Lys(cyclo[SEQ ID NO:143])-miniPEG₂-K(N₃)—NH₂).

The EEV can be selected from:
Ac—PKKKRKV-miniPEG₂-Lys(cyclo(GfFGrGrQ])-PEG₁₂-OH
(Ac-SEQ ID NO:42-miniPEG₂-Lys(cyclo(SEQ ID NO:133])-PEG₁₂-OH)
Ac—PKKKRKV-miniPEG₂-Lys(cyclo[FGFKRKRQ])-PEG₁₂-OH
(Ac-SEQ ID NO:42-miniPEG₂-Lys(cyclo[SEQ ID NO:144])-PEG₁₂-OH)
Ac—PKKKRKV-miniPEG₂-Lys(cyclo[FGFRGRGQ])-PEG₁₂-OH
(Ac-SEQ ID NO:42-miniPEG₂-Lys(cyclo[SEQ ID NO:145])-PEG₁₂-OH)
Ac—PKKKRKV-miniPEG₂-Lys(cyclo[FGFGRGRGRQ])-PEG₁₂-OH
(Ac-SEQ ID NO:42-miniPEG₂-Lys(cyclo[SEQ ID NO: 146])-PEG₁₂-OH)
Ac—PKKKRKV-miniPEG₂-Lys(cyclo[FGFGRrRQ])-PEG₁₂-OH
(Ac-SEQ ID NO:42-miniPEG₂-Lys(cyclo[SEQ ID NO:147])-PEG₁₂-OH)
Ac—PKKKRKV-miniPEG₂-Lys(cyclo[FGFGRRRQ])-PEG₁₂-OH
(Ac-SEQ ID NO:42-miniPEG₂-Lys(cyclo[SEQ ID NO:84])-PEG₁₂-OH) and
Ac—PKKKRKV-miniPEG₂-Lys(cyclo[FGFRRRRQ])-PEG₁₂-OH
(Ac-SEQ ID NO:42-miniPEG₂-Lys(cyclo[SEQ ID NO:85])-PEG₁₂-OH).

The EEV can be selected from:
Ac—K—K—K—R—K-G-miniPEG₂-K(cyclo[FGFGRGRQ])-PEG₁₂-OH
(Ac-SEQ ID NO: 148-miniPEG₂-K(cyclo[SEQ ID NO:82])-PEG₁₂-OH)
Ac—K—K—K—R—K-miniPEG₂-K(cyclo[FGFGRGRQ])-PEG₁₂-OH
(Ac-SEQ ID NO:19-miniPEG₂-K(cyclo[SEQ ID NO:82])-PEG₁₂-OH)
Ac—K—K—R—K—K-PEG₄-K(cyclo[FGFGRGRQ])-PEG₁₂-OH
(Ac-SEQ ID NO:22-PEG₄-K(cyclo[SEQ ID NO:82])-PEG₁₂-OH)
Ac—K—R—K—K—K-PEG₄-K(cyclo[FGFGRGRQ])-PEG₁₂-OH
(Ac-SEQ ID NO:21-PEG₄-K(cyclo[SEQ ID NO:82])-PEG₁₂-OH)
Ac—K—K—K—K—R-PEG₄-K(cyclo[FGFGRGRQ])-PEG₁₂-OH
(Ac-SEQ ID NO:23-PEG₄-K(cyclo[SEQ ID NO:82])-PEG₁₂-OH)
Ac—R—K—K—K—K-PEG₄-K(cyclo[FGFGRGRQ])-PEG₁₂-OH
(Ac-SEQ ID NO:20-PEG₄-K(cyclo[SEQ ID NO:82])-PEG₁₂-OH) and
Ac—K—K—K—R—K-PEG₄-K(cyclo[FGFGRGRQ])-PEG₁₂-OH
(Ac-SEQ ID NO: 19-PEG₄-K(cyclo[SEQ ID NO:82])-PEG₁₂-OH).

The EEV can be selected from:
Ac—PKKKRKV-PEG₂-K(cyclo[FGFGRGRQ])-PEG₂-K(N₃)—NH₂
(Ac-SEQ ID NO:42-PEG₂-K(cyclo[SEQ ID NO:82])-PEG₂-K(N₃)—NH₂)
Ac—PKKKRKV-PEG₂-K(cyclo[FGFGRGRQ])-PEG₁₂-OH
(Ac-SEQ ID NO:42-PEG₂-K(cyclo[SEQ ID NO:82])-PEG₁₂-OH)
Ac—PKKKRKV-PEG₂-K(cyclo[GfFGrGrQ])-PEG₂-K(N₃)—NH₂
(Ac-SEQ ID NO:42-PEG₂-K(cyclo[SEQ ID NO:133])-PEG₂-K(N₃)—NH₂) and
Ac—PKKKRKV-PEG₂-K(cyclo[GfFGrGrQ])-PEG₁₂-OH
(Ac-SEQ ID NO:42-PEG₂-K(cyclo[SEQ ID NO: 133])-PEG₁₂-OH).

The cargo can be a protein and the EEV can be selected from:
Ac—PKKKRKV-PEG₂-K(cyclo[FfΦGrGrQ])-PEG₁₂-OH
(Ac-SEQ ID NO:42-PEG₂-K(cyclo[SEQ ID NO:80])-PEG₁₂-OH)
Ac—PKKKRKV-PEG₂-K(cyclo[FfΦCit-r-Cit-rQ])-PEG₁₂-OH
(Ac-SEQ ID NO:42-PEG₂-K(cyclo[SEQ ID NO:79])-PEG₁₂-OH)
Ac—PKKKRKV-PEG₂-K(cyclo[FfFGRGRQ])-PEG₁₂-OH
(Ac-SEQ ID NO:42-PEG₂-K(cyclo[SEQ ID NO:81])-PEG₁₂-OH)
Ac—PKKKRKV-PEG₂-K(cyclo[FGFGRGRQ])-PEG₁₂-OH
(Ac-SEQ ID NO:42-PEG₂-K(cyclo[SEQ ID NO:82])-PEG₁₂-OH)
Ac—PKKKRKV-PEG₂-K(cyclo[GfFGrGrQ])-PEG₁₂-OH
(Ac-SEQ ID NO:42-PEG₂-K(cyclo[SEQ ID NO:133])-PEG₁₂-OH)
Ac—PKKKRKV-PEG₂-K(cyclo[FGFGRRRQ])-PEG₁₂-OH
(Ac-SEQ ID NO:42-PEG₂-K(cyclo[SEQ ID NO:84])-PEG₁₂-OH)
Ac—PKKKRKV-PEG₂-K(cyclo[FGFRRRRQ])-PEG₁₂-OH
(Ac-SEQ ID NO:42-PEG₂-K(cyclo[SEQ ID NO:85])-PEG₁₂-OH)
Ac-rr-PEG₂-K(cyclo[FfΦGrGrQ])-PEG₁₂-OH
(Ac-rr-PEG₂-K(cyclo[SEQ ID NO:80])-PEG₁₂-OH)
Ac-rr-PEG₂-K(cyclo[FfΦCit-r-Cit-rQ])-PEG₁₂-OH
(Ac-rr-PEG₂-K(cyclo[SEQ ID NO:79])-PEG₁₂-OH)
Ac-rr-PEG₂-K(cyclo[FfF-GRGRQ])-PEG₁₂-OH
(Ac-rr-PEG₂-K(cyclo[SEQ ID NO:81])-PEG₁₂-OH)
Ac-rr-PEG₂-K(cyclo[FGFGRGRQ])-PEG₁₂-OH
(Ac-rr-PEG₂-K(cyclo[SEQ ID NO:82])-PEG₁₂-OH)
Ac-rr-PEG₂-K(cyclo[GfFGrGrQ])-PEG₁₂-OH
(Ac-rr-PEG₂-K(cyclo[SEQ ID NO:133])-PEG₁₂-OH)
Ac-rr-PEG₂-K(cyclo[FGFGRRRQ])-PEG₁₂-OH
(Ac-rr-PEG₂-K(cyclo[SEQ ID NO: 84])-PEG₁₂-OH)
Ac-rr-PEG₂-K(cyclo[FGFRRRRQ])-PEG₁₂-OH
(Ac-rr-PEG₂-K(cyclo[SEQ ID NO:85])-PEG₁₂-OH)
Ac-rrr-PEG₂-K(cyclo[FfΦGrGrQ])-PEG₁₂-OH
(Ac-rrr-PEG₂-K(cyclo[SEQ ID NO:80])-PEG₁₂-OH)
Ac-rrr-PEG₂-K(cyclo[FfΦCit-r-Cit-rQ])-PEG₁₂-OH
(Ac-rrr-PEG₂-K(cyclo[SEQ ID NO: 79])-PEG₁₂-OH)
Ac-rrr-PEG₂-K(cyclo[FfFGRGRQ])-PEG₁₂-OH
(Ac-rrr-PEG₂-K(cyclo[SEQ ID NO:81])-PEG₁₂-OH)
Ac-rrr-PEG₂-K(cyclo[FGFGRGRQ])-PEG₁₂-OH
(Ac-rrr-PEG₂-K(cyclo[SEQ ID NO:82])-PEG₁₂-OH)
Ac-rrr-PEG₂-K(cyclo[GfFGrGrQ])-PEG₁₂-OH
(Ac-rrr-PEG₂-K(cyclo[SEQ ID NO:133])-PEG₁₂-OH)
Ac-rrr-PEG₂-K(cyclo[FGFGRRRQ])-PEG₁₂-OH
(Ac-rrr-PEG₂-K(cyclo[SEQ ID NO:84])-PEG₁₂-OH)
Ac-rrr-PEG₂-K(cyclo[FGFRRRRQ])-PEG₁₂-OH
(Ac-rrr-PEG₂-K(cyclo[SEQ ID NO:85])-PEG₁₂-OH)
Ac-rhr-PEG₂-K(cyclo[FfΦGrGrQ])-PEG₁₂-OH
(Ac-rhr-PEG₂-K(cyclo[SEQ ID NO:80])-PEG₁₂-OH)
Ac-rhr-PEG₂-K(cyclo[FfΦCit-r-Cit-rQ])-PEG₁₂-OH
(Ac-rhr-PEG₂-K(cyclo[SEQ ID NO:79])-PEG₁₂-OH)
Ac-rhr-PEG₂-K(cyclo[FfFGRGRQ])-PEG₁₂-OH
(Ac-rhr-PEG₂-K(cyclo[SEQ ID NO:81])-PEG₁₂-OH)
Ac-rhr-PEG₂-K(cyclo[FGFGRGRQ])-PEG₁₂-OH
(Ac-rhr-PEG₂-K(cyclo[SEQ ID NO:82])-PEG₁₂-OH)
Ac-rhr-PEG₂-K(cyclo[GfFGrGrQ])-PEG₁₂-OH
(Ac-rhr-PEG₂-K(cyclo[SEQ ID NO:133])-PEG₁₂-OH)
Ac-rhr-PEG₂-K(cyclo[FGFGRRRQ])-PEG₁₂-OH
(Ac-rhr-PEG₂-K(cyclo[SEQ ID NO:84])-PEG₁₂-OH)
Ac-rhr-PEG₂-K(cyclo[FGFRRRRQ])-PEG₁₂-OH
(Ac-rhr-PEG₂-K(cyclo[SEQ ID NO:85])-PEG₁₂-OH)
Ac-rbr-PEG₂-K(cyclo[FfΦGrGrQ])-PEG₁₂-OH
(Ac-rbr-PEG₂-K(cyclo[SEQ ID NO:80])-PEG₁₂-OH)
Ac-rbr-PEG₂-K(cyclo[FfΦCit-r-Cit-rQ])-PEG₁₂-OH
(Ac-rbr-PEG₂-K(cyclo[SEQ ID NO:79])-PEG₁₂-OH)
Ac-rbr-PEG₂-K(cyclo[FfFGRGRQ])-PEG₁₂-OH
(Ac-rbr-PEG₂-K(cyclo[SEQ ID NO:81])-PEG₁₂-OH)
Ac-rbr-PEG₂-K(cyclo[FGFGRGRQ])-PEG₁₂-OH
(Ac-rbr-PEG₂-K(cyclo[SEQ ID NO:82])-PEG₁₂-OH)
Ac-rbr-PEG₂-K(cyclo[GfFGrGrQ])-PEG₁₂-OH
(Ac-rbr-PEG₂-K(cyclo[SEQ ID NO:133])-PEG₁₂-OH)
Ac-rbr-PEG₂-K(cyclo[FGFGRRRQ])-PEG₁₂-OH
(Ac-rbr-PEG₂-K(cyclo[SEQ ID NO:84])-PEG₁₂-OH)
Ac-rbr-PEG₂-K(cyclo[FGFRRRRQ])-PEG₁₂-OH
(Ac-rbr-PEG₂-K(cyclo[SEQ ID NO:85])-PEG₁₂-OH)
Ac-rbrbr-PEG₂-K(cyclo[FfΦGrGrQ])-PEG₁₂-OH
(Ac-SEQ ID NO:138-PEG₂-K(cyclo[SEQ ID NO:80])-PEG₁₂-OH)
Ac-rbrbr-PEG₂-K(cyclo[FfΦCit-r-Cit-rQ])-PEG₁₂-OH
(Ac-SEQ ID NO: 138-PEG₂-K(cyclo[SEQ ID NO:79])-PEG₁₂-OH)
Ac-rbrbr-PEG₂-K(cyclo[FfFGRGRQ])-PEG₁₂-OH
(Ac-SEQ ID NO:138-PEG₂-K(cyclo[SEQ ID NO:81])-PEG₁₂-OH)
Ac-rbrbr-PEG₂-K(cyclo[FGFGRGRQ])-PEG₁₂-OH
(Ac-SEQ ID NO:138-PEG₂-K(cyclo[SEQ ID NO:82])-PEG₁₂-OH)
Ac-rbrbr-PEG₂-K(cyclo[GfFGrGrQ])-PEG₁₂-OH
(Ac-SEQ ID NO:138-PEG₂-K(cyclo[SEQ ID NO:133])-PEG₁₂-OH)
Ac-rbrbr-PEG₂-K(cyclo[FGFGRRRQ])-PEG₁₂-OH
(Ac-SEQ ID NO:138-PEG₂-K(cyclo[SEQ ID NO:84])-PEG₁₂-OH)
Ac-rbrbr-PEG₂-K(cyclo[FGFRRRRQ])-PEG₁₂-OH
(Ac-SEQ ID NO:138-PEG₂-K(cyclo[SEQ ID NO:85])-PEG₁₂-OH)
Ac-rbhbr-PEG₂-K(cyclo[FfΦGrGrQ])-PEG₁₂-OH
(Ac-SEQ ID NO: 149-PEG₂-K(cyclo[SEQ ID NO:80])-PEG₁₂-OH)
Ac-rbhbr-PEG₂-K(cyclo[FfΦCit-r-Cit-rQ])-PEG₁₂-OH
(Ac-SEQ ID NO: 149-PEG₂-K(cyclo[SEQ ID NO:79])-PEG₁₂-OH)
Ac-rbhbr-PEG₂-K(cyclo[FfFGRGRQ])-PEG₁₂-OH
(Ac-SEQ ID NO: 149-PEG₂-K(cyclo[SEQ ID NO:81])-PEG₁₂-OH)
Ac-rbhbr-PEG₂-K(cyclo[FGFGRGRQ])-PEG₁₂-OH
(Ac-SEQ ID NO: 149-PEG₂-K(cyclo[SEQ ID NO:82])-PEG₁₂-OH)
Ac-rbhbr-PEG₂-K(cyclo[GfFGrGrQ])-PEG₁₂-OH
(Ac-SEQ ID NO: 149-PEG₂-K(cyclo[SEQ ID NO:133])-PEG₁₂-OH)
Ac-rbhbr-PEG₂-K(cyclo[FGFGRRRQ])-PEG₁₂-OH
(Ac-SEQ ID NO: 149-PEG₂-K(cyclo[SEQ ID NO:84])-PEG₁₂-OH)
Ac-rbhbr-PEG₂-K(cyclo[FGFRRRRQ])-PEG₁₂-OH
(Ac-SEQ ID NO:149-PEG₂-K(cyclo[SEQ ID NO:85])-PEG₁₂-OH)
Ac-hbrbh-PEG₂-K(cyclo[FfΦGrGrQ])-PEG₁₂-OH
(Ac-SEQ ID NO: 141-PEG₂-K(cyclo[SEQ ID NO:80])-PEG₁₂-OH)
Ac-hbrbh-PEG₂-K(cyclo[FfΦCit-r-Cit-rQ])-PEG₁₂-OH
(Ac-SEQ ID NO:141-PEG₂-K(cyclo[SEQ ID NO:79])-PEG₁₂-OH)
Ac-hbrbh-PEG₂-K(cyclo[FfFGRGRQ])-PEG₁₂-OH
(Ac-SEQ ID NO: 141-PEG₂-K(cyclo[SEQ ID NO:81])-PEG₁₂-OH)
Ac-hbrbh-PEG₂-K(cyclo[FGFGRGRQ])-PEG₁₂-OH
(Ac-SEQ ID NO:141-PEG₂-K(cyclo[SEQ ID NO:82])-PEG₁₂-OH)
Ac-hbrbh-PEG₂-K(cyclo[GfFGrGrQ])-PEG₁₂-OH
(Ac-SEQ ID NO: 141-PEG₂-K(cyclo[SEQ ID NO:133])-PEG₁₂-OH)
Ac-hbrbh-PEG₂-K(cyclo[FGFGRRRQ])-PEG₁₂-OH
(Ac-SEQ ID NO:141-PEG₂-K(cyclo[SEQ ID NO:84])-PEG₁₂-OH)
Ac-hbrbh-PEG₂-K(cyclo[FGFRRRRQ])-PEG₁₂-OH
(Ac-SEQ ID NO:141-PEG₂-K(cyclo[SEQ ID NO:85])-PEG₁₂-OH),
wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. As used herein, “cargo” is a compound or moiety for which delivery into a cell is desired. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

In embodiments, the amino acid side chain comprises a chemically reactive group to which the linker or cargo is conjugated comprises. The chemically reactive group can comprise an amine group, a carboxylic acid, an amide, a hydroxyl group, a sulfhydryl group, a guanidinyl group, a phenolic group, a thioether group, an imidazolyl group, or an indolyl group. In embodiments, the amino acid of the cCPP to which the cargo is conjugated comprises lysine, arginine, aspartic acid, glutamic acid, asparagine, glutamine, homoglutamine, serine, threonine, tyrosine, cysteine, arginine, tyrosine, methionine, histidine or tryptophan.

The cargo can comprise one or more detectable moieties, one or more therapeutic moieties (TMs), one or more targeting moieties, or any combination thereof. In embodiments, the cargo comprises a TM.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to the linker at the terminal carbonyl group to provide the following structure:

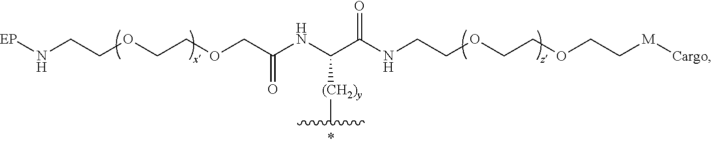

wherein:

EP is an exocyclic peptide and M, AA_{SC}, Cargo, x′, y, and z′ are as defined above, * is the point of attachment to the AA_{SC}, x′ can be 1. y can be 4. z′ can be 11. —(OCH₂CH₂)ₓ′— and/or —(OCH₂CH₂)_{z}′— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

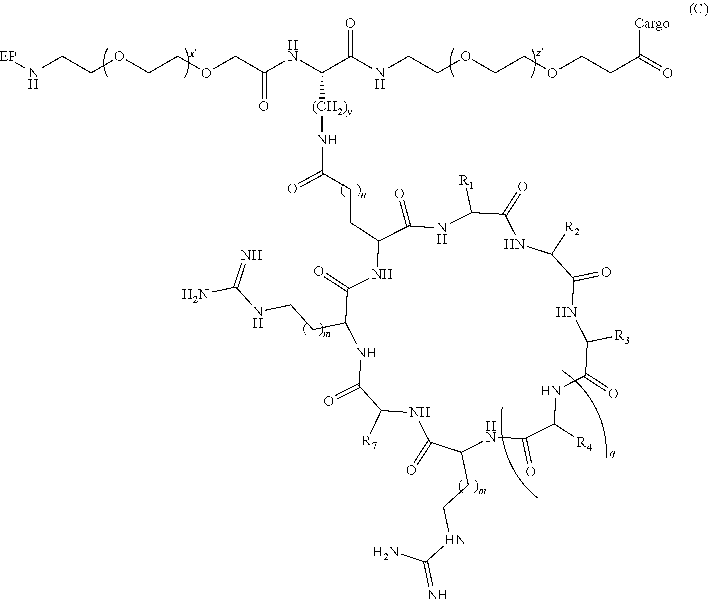

or a protonated form thereof,

wherein:
R₁, R₂, and R₃ can each independently be H or an amino acid residue having a side chain comprising an aromatic group;
R₄ is H or an amino acid side chain;
EP is an exocyclic peptide as defined herein;
Cargo is a moiety as defined herein;
each m is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

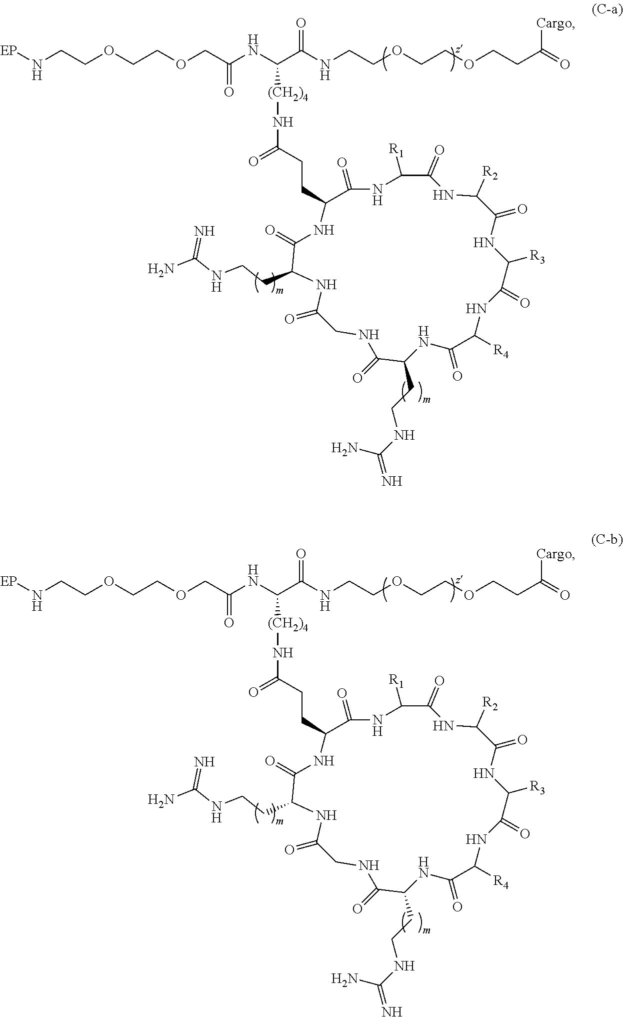

or a protonated form thereof, wherein EP, m and z are as defined above in Formula (C).

The EEV can be conjugated to a cargo and the EEV-conjugate can comprise the structure of Formula (C-c):

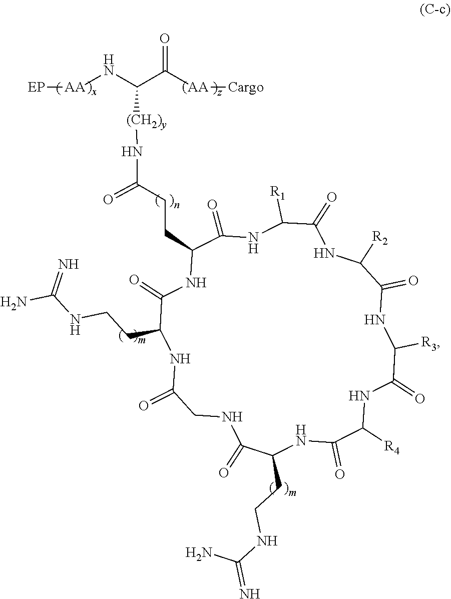

or a protonated form thereof, wherein EP, R¹, R², R³, R⁴, and m are as defined above in Formula (II); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4):

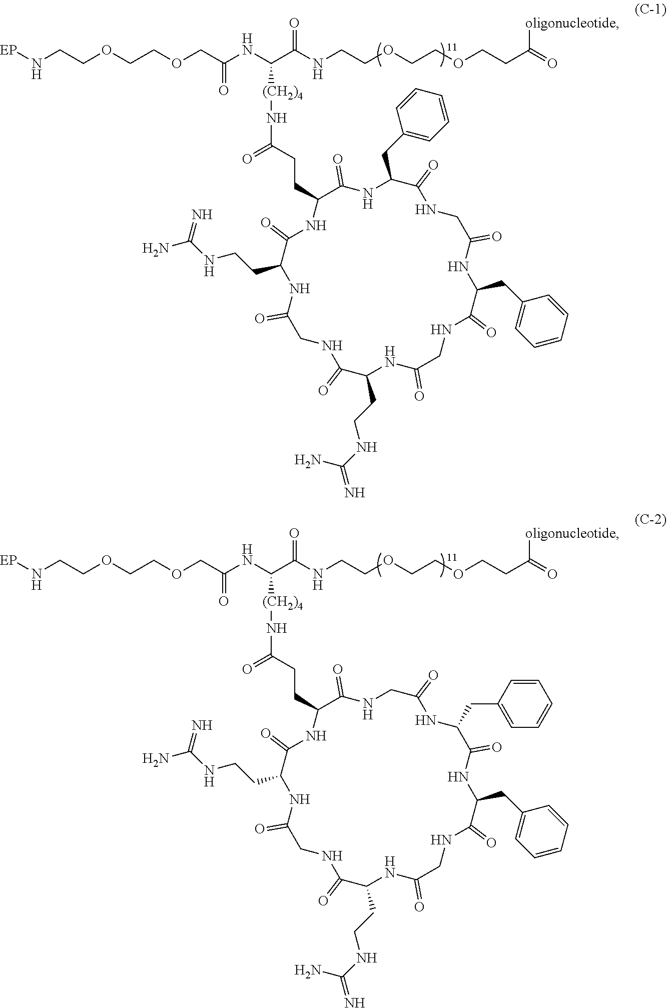

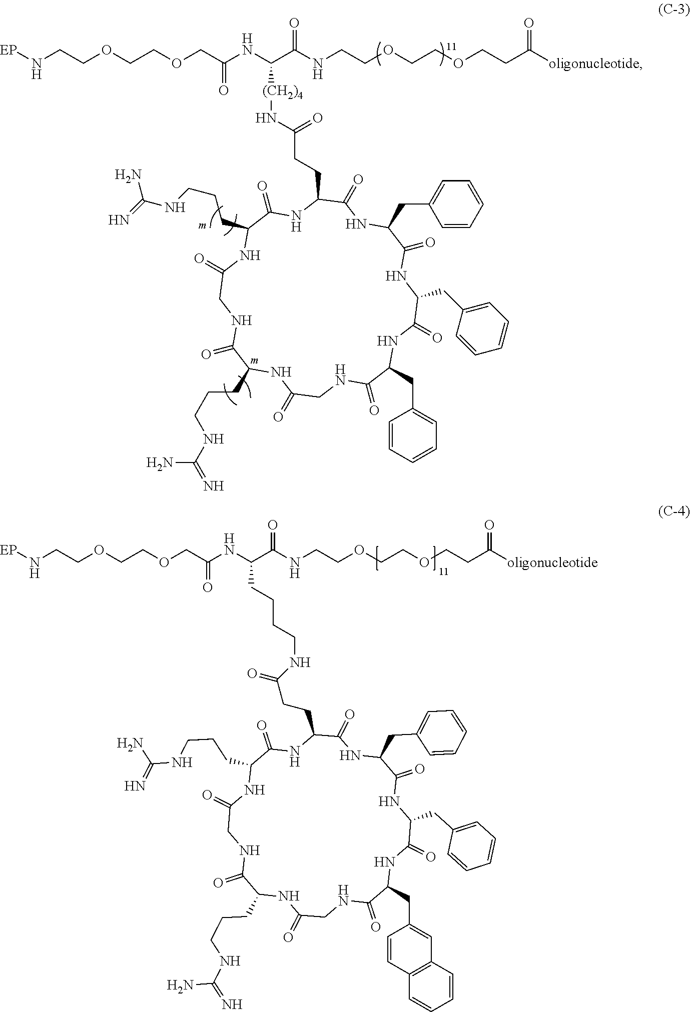

The EEV can be conjugated to an oligonucleotide cargo and the BEV-conjugate can comprise the structure.

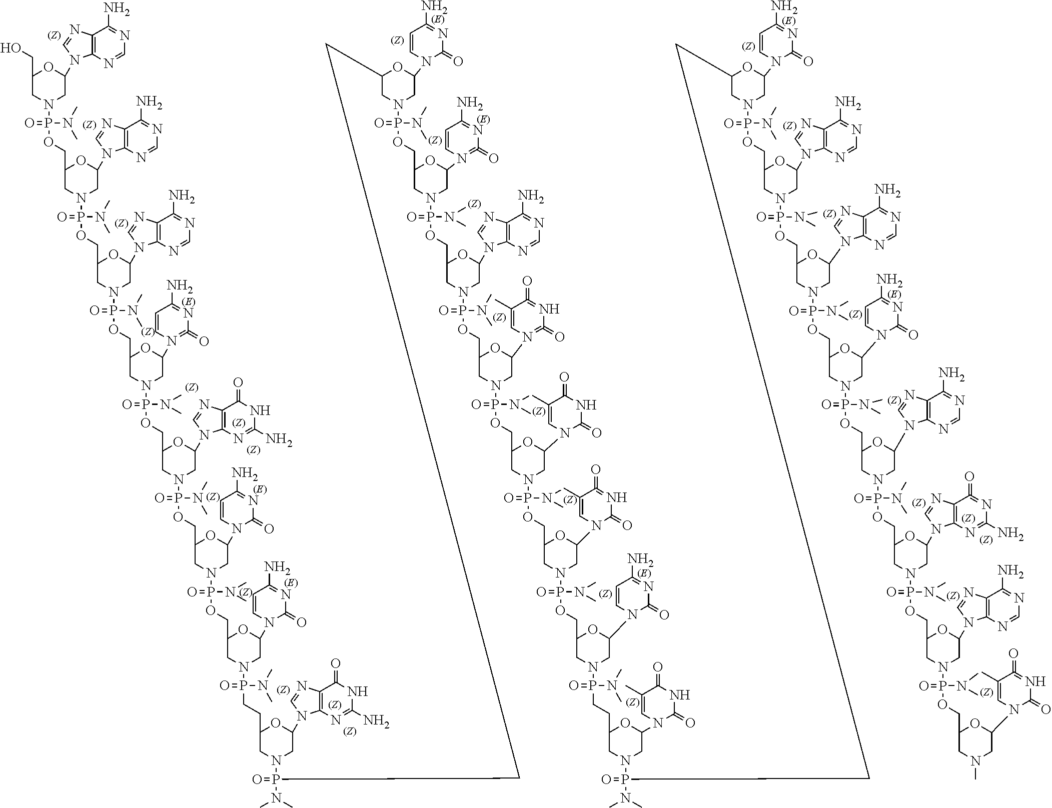

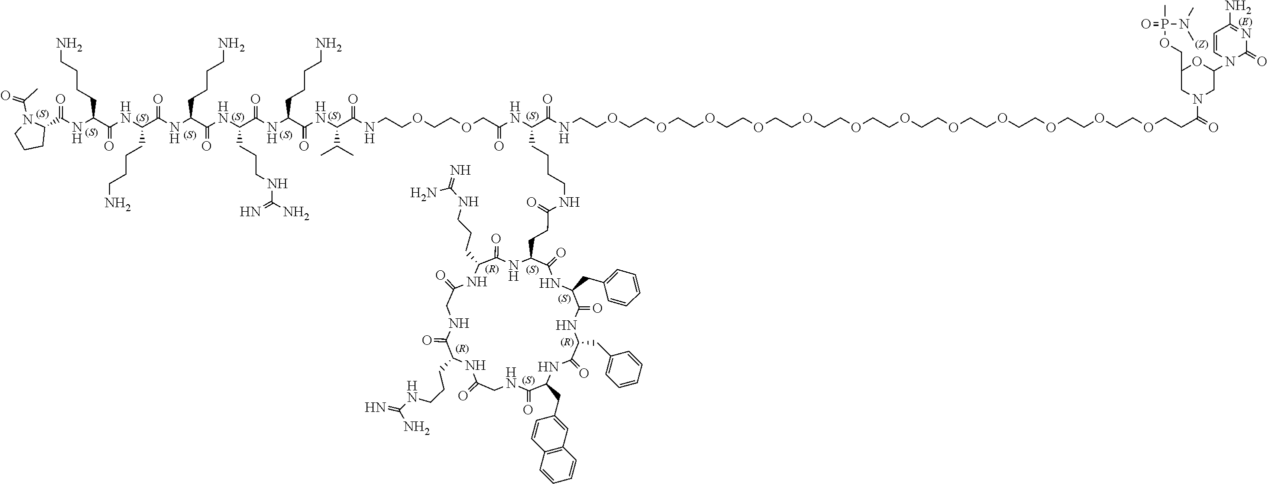

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC so of the cCPP having the modified sequence to a control sequence (as described herein).

The relative cytosolic delivery efficiency of the cCPPs can be in the range of from about 50% to about 450% compared to cyclo(FfΦRrRrQ, SEQ ID NO:150), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween.

The relative cytosolic delivery efficiency of the cCPPs can be improved by greater than about 600% compared to a cyclic peptide comprising cyclo(FfΦRrRrQ, SEQ ID NO:150).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Detectable Moiety

In embodiments, the compound disclosed herein includes a detectable moiety. In embodiments, the detectible moiety is attached to the cell penetrating peptide at the amino group, the carboxylate group, or the side chain of any of the amino acids of the cell penetrating peptide moiety (e.g., at the amino group, the carboxylate group, or the side chain of any amino acid in the CPP). In embodiments, the therapeutic moiety includes a detectable moiety. The detectable moiety can include any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. In embodiments, the label is detectable without the addition of further reagents.

In embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetraphenyl metrylbenzoporphyrin; Pd (II) octaethylporphyrin ketone; Pt (II) octaethylporphyrin ketone; Pd (II) meso-tetra(pentafluorophenyl) porphyrin; Pt (II) meso-tetra(pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine) ruthenium (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); 188enzothiazole) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluor‘sc’in; eosin; 2′,7′-dichlorofluorescein; 5 (6)-carboxy-fluorecsein; carboxynaphthofluorescein; 8-hydroxypyrene-1,3,6-trisulfonic acid; semi-naphthorhodafluor; semi-naphthofluorescein; tris(4,7-diphenyl-1,10-phenanthroline) ruthenium (II) dichloride; (4,7-diphenyl-1,10-phenanthroline) ruthenium (II) tetraphenylboron; platinum (II) octaethylporphyin, dialkylcarbocyanine; dioctadecylcycloxacarbocyanine; fluorenylmethyloxycarbonyl chloride: 7-amino-4-methylcourmarin (Amc), green fluorescent protein (GFP); and derivatives or combinations thereof.

In some examples, the detectable moiety can include Rhodamine B (Rho), fluorescein isothiocyanate (FITC), 7-amino-4-methylcourmarin (Amc), green fluorescent protein (GFP), or derivatives or combinations thereof.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl) phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl) phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl) phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

The above polymers, such as PEG groups, can be attached to an oligonucleotide, such as an AC, under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the AC (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the AC by reductive alkylation, the polymer selected should have a single reactive aldehyde so that the degree of polymerization is controlled. See, for example, Kinstler et al., Adv. Drug. Delivery Rev. (2002), 54:477-485, Roberts et al., Adv. Drug Delivery Rev. (2002), 54. 459-476; and Zalipsky et al., Adv. Drug Delivery Rev. (1995), 16:157-182.

In order to direct covalently link the AC or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, «-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of making AC and conjugating AC to linear CPP are generally described in US Pub. No. 2018/0298383, which is herein incorporated by reference for all purposes. The methods may be applied to the cyclic CPPs disclosed herein.

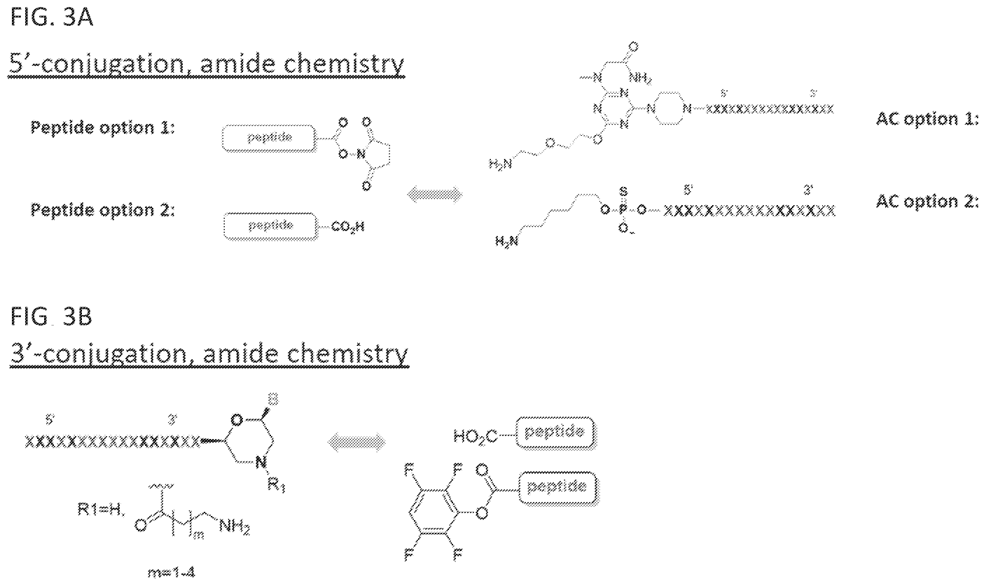

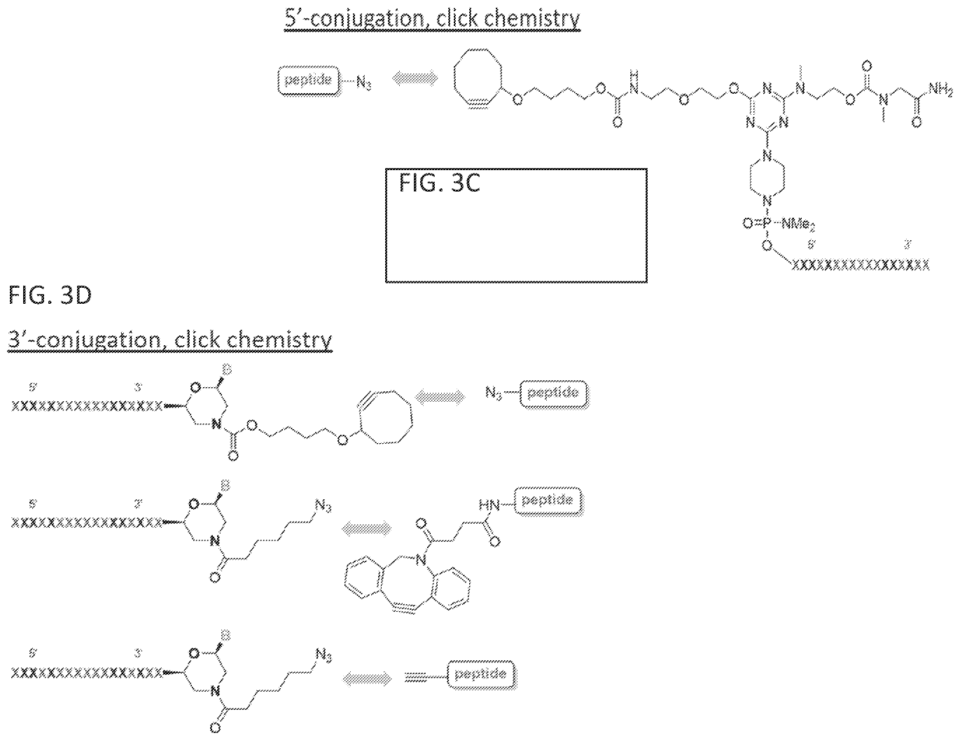

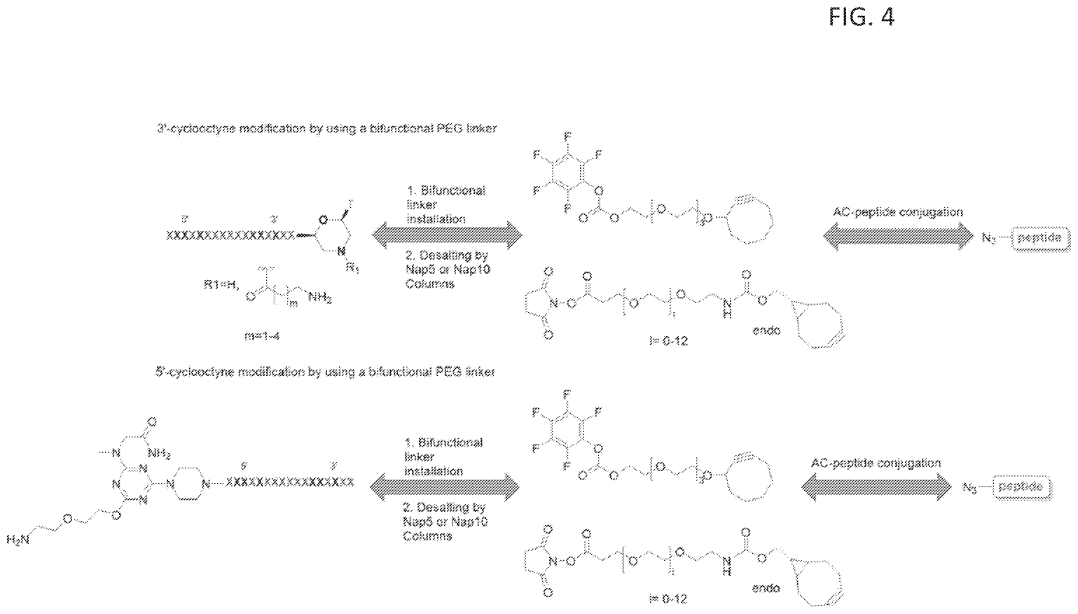

Synthetic schemes are provided in FIG. 3A-3D and FIG. 4.

Non-limiting examples of compounds that include a CPPs and a reactive group useful for conjugation to an AC are shown in Table 10. Example linker groups are also shown. Example reactive groups include tetrafluorophenyl ester (TFP), free carboxylic acid (COOH), and azide (Na). In Table 10, n is an integer from 0 to 20; Pipa6 is AcRXRRBRRXRYQFLIRXRBRXRB wherein B is β-Alanine and X is aminohexanoic acid; Dap is 2,3-diaminopropionic acid; NLS is a nuclear localization sequence; βA is beta alanine; -ss- is a disulfide; PABC is poly(A) binding protein C-terminal domain; Cₓ where x is a number is an alkyl chain of length x; and BCN is bicyclo[6.1.0]nonyne.

TABLE 10
Compounds that include a CPPs and a reactive group

TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N3)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)-PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N₃)
CPP-PEGₙ-Cys-prodisulfide-K(N₃)
CPP-PEGₙ-K(N₃)
CPP-K(CPP)-PEGₙ-K(N₃)
CPP-PEGₙ-K(CPP)-PEGₙ-TFP
CPP-C₆-TFP
CPP-PEGₙ-K(PEGₙ-CPP)PEGₙ-K(N₃)
Ac-T9-PEGₙ-Lys(CPP-PEGₙ)-K(N₃)
Ac-MSP-PEGₙ-K(CPP-PEGₙ)-K(N₃)
CPP-PEGₙ-TFP(ENTRD802)
CPP-C₆-TFP(ENTRD696)
CPP-PEGₙ-K(CPP)-PEGₙ-TFP(ENTRD-344)
CPP-PEGₙ-COOH
CPP-C₁₂-TFP(ENTD-695)
palmitoyl-PEGₙ-K(CPP)-PEGₙ-TFP(ENTD-343)
CPP-PEGₙ-K(N₃)(ENTRD-617)
Ac-T9-PEGₙ-K(CPP)-K(N₃)(ENTRD673)
Ac-MSP-PEGₙ-K(CPP-PEGₙ)-K(N₃)(ENTRD675)
Ac-NLS-K(CPP)-PEGₙ-K(N₃)(ENTRD684)
K(N₃)-PEGₙ-NLS-ss-PEGₙ-CPP(ETRD-681)
K(N₃)-PEGₙ-NLS-K-BA-BA-CPP(ETRD-682)

In embodiments, the CPPs have free carboxylic acid groups that may be utilized for conjugation to an AC In embodiments, the EEVs have free carboxylic acid groups that may be utilized for conjugation to an AC.

The structure below is a 3′ cyclooctyne modified PMO used for a click reaction with a compound that includes an azide:

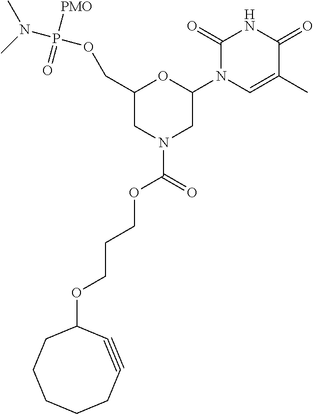

An example scheme of conjugation of a CPP and linker to the 3′ end of an AC via an amide bond is shown below.

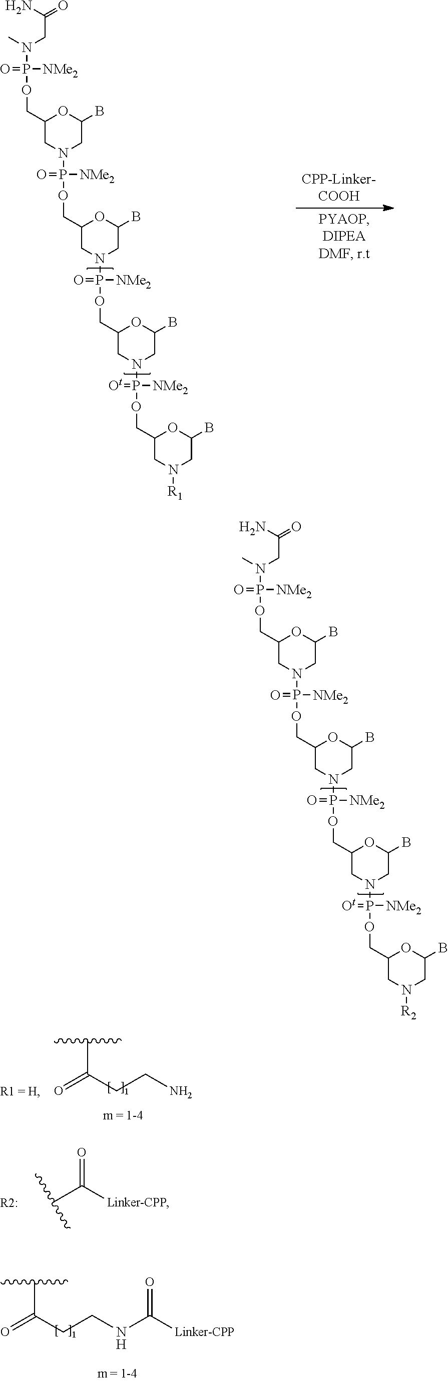

An example scheme of conjugation of a CPP and linker to a 3′-cyclooctyne modified PMO via strain-promoted azide-alkyne cycloaddition is shown below:

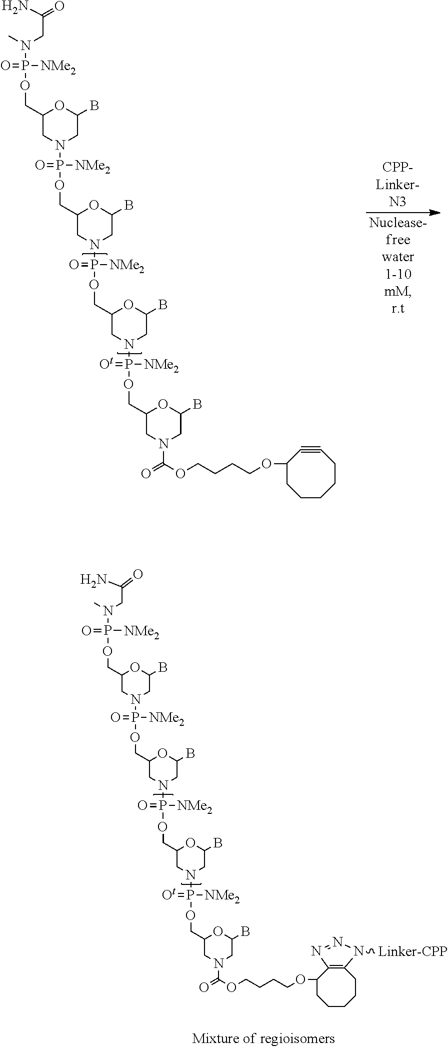

An example of the conjugation chemistry used to connect an AC and CPP with an additional linker containing a polyethylene glycol moiety is shown below:

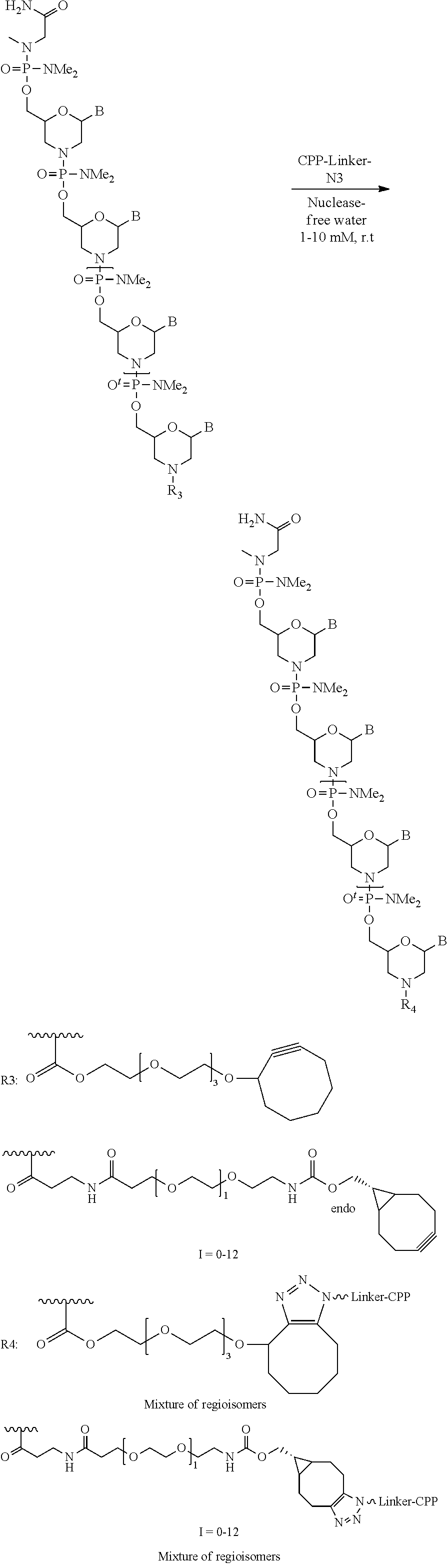

An example of conjugation of a (PP-linker to a 5′-cyclooctyne modified PMO via strain-promoted azide-alkyne cycloaddition (click chemistry) is shown below:

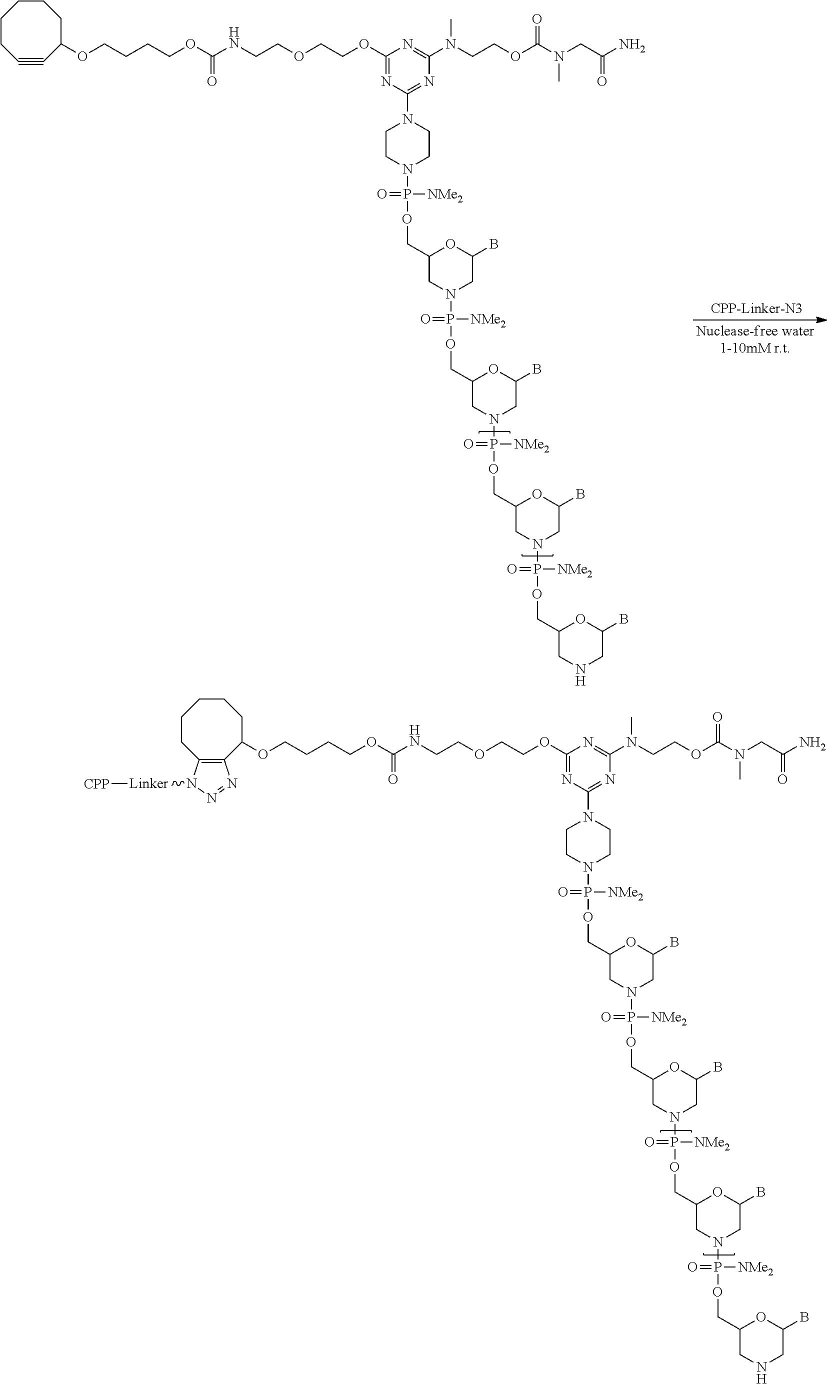

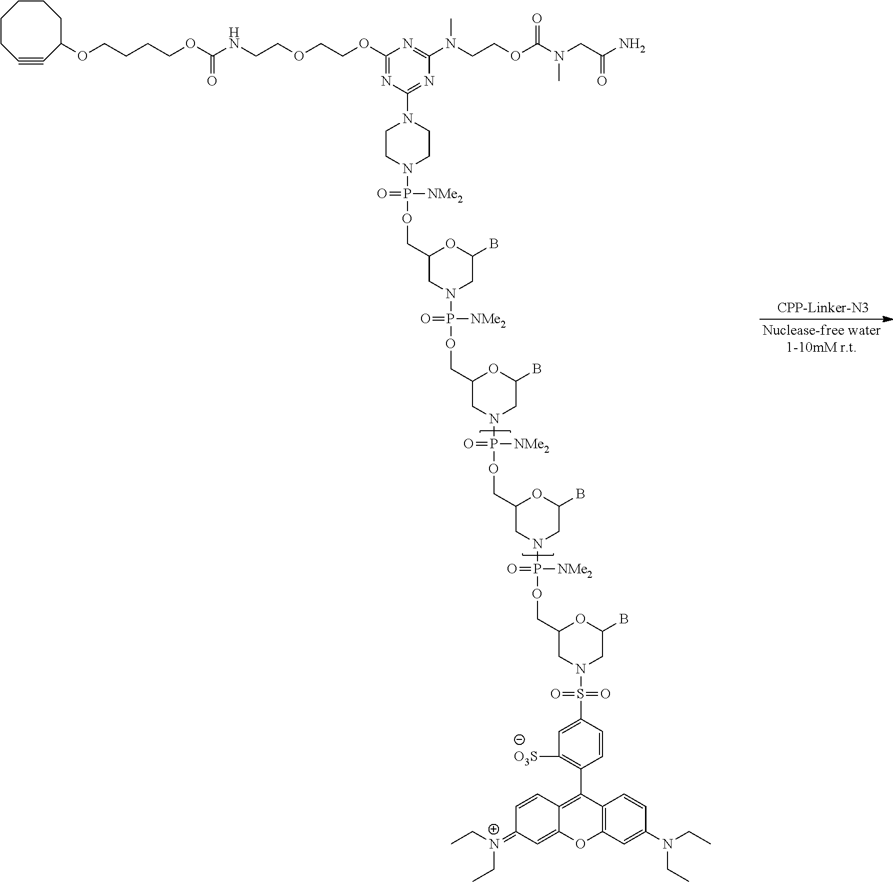

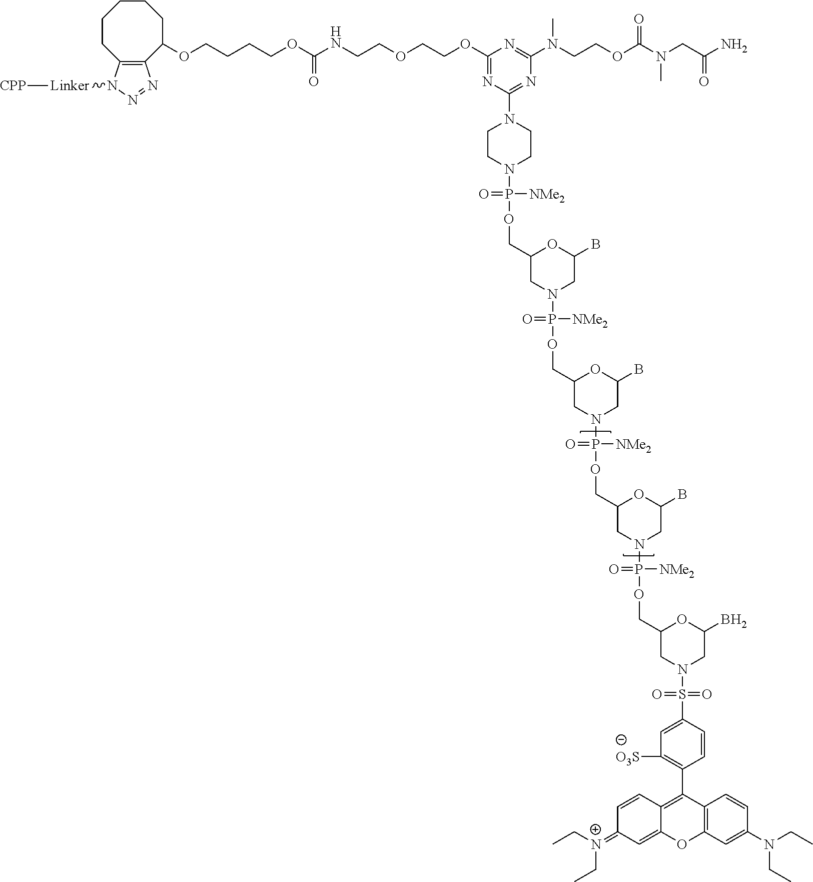

Methods of synthesizing oligomeric antisense compounds are known in the art. The present disclosure is not limited by the method of synthesizing the AC. In embodiments, provided herein are compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds are well known to those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of oligonucleotide purification and analysis are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

### Diseases

In embodiments, a method is provided for treating a disease or disorder associated with Interferon Regulatory Factor-5 (IRF-5). The method includes administering a compound or composition described herein to a subject in need thereof. In embodiments, the method comprises administering a therapeutically effective amount of a compound or composition described herein to a subject in need thereof.

In embodiments, the disease or disorder is associated with IRF-5 genetic variation. In embodiments, the disease or disorder is associated with a genetic mutation in the IRF-5 gene. In embodiments, the genetic mutation in IRF-5 results IRF-5 overexpression. In embodiments, the genetic mutation results in alternate isoform expression. In embodiments, the disease or disorder is associated with IRF-5 overexpression. In embodiments, the disease or disorder is associated with IRF-5 isoform expression.

In embodiments, a method is provided for treating inflammation, autoantibody production, inflammatory cell infiltration, collagen deposits, or inflammatory cytokine production in a patient.

In embodiments, a method of downregulating IRF-5 expression in a patient is provided. In embodiments, IRF-5 expression in a macrophage is reduced. In embodiments, IRF-5 expression in a Kupffer cell is reduced. In embodiments, IRF-5 expression in the gastrointestinal tract is reduced. In embodiments, expression of IRF-5 in the liver is reduced. In embodiments, expression of IRF-5 in the lungs is reduced. In embodiments, expression of IRF-5 in the kidneys is reduced. In embodiments, expression of IRF-5 in the joints is reduced. In embodiments, expression of IRF-5 in the central nervous system is reduced.

In embodiments, the compounds disclosed herein are used for treating a disease associated with IRF-5. Examples of diseases associated with IRF-5 include, but are not limited to, inflammatory bowel disease (IBD), ulcerative colitis, Crohn's disease, systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), Nonalcoholic steatohepatitis (NASH), liver fibrosis, asthma, and severe asthma. In embodiments, the compounds disclosed herein are used to reduce inflammation, cirrhosis, fibrosis, proteinuria, joint inflammation, autoantibody production, inflammatory cell infiltration, collagen deposits, or inflammatory cytokine production in a patient. In embodiments, the compounds disclosed herein are used to reduce inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, or unintended weight loss.

In embodiments, the compounds disclosed herein are used for treating an inflammatory disease. “Inflammatory disease” refers to diseases in which activation of the innate or adaptive immune response is a prominent contributor to the clinical condition. Inflammatory diseases include, but are not limited to, acne vulgaris, asthma, COPD, autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, and ischemic heart disease, type I diabetes, and diabetic nephropathy.

In embodiments, the compounds disclosed herein are used for treating an autoimmune disease. “Autoimmune disease” refers to a disease or disorder in which a patient's immune system attacks the patient's own tissues. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); systemic sclerosis (scleroderma); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, dermatomyositis; granulomatosis and vasculitis; primary biliary cirrhosis; pernicious anemia (Addison's disease); autoimmune gastritis; autoimmune hepatitis; diseases involving leukocyte diapedesis; central nervous system (CNS) inflammatory disorder; vitiligo; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome, allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenia purpura (ITP) or autoimmune thrombocytopenia.

In embodiments, the compounds disclosed herein are used for treating an autoimmune disease such as systemic lupus erythematosus (SLE), systemic sclerosis (scleroderma), polymyositis/dermatomyositis, Crohn's disease, ulcerative colitis, rheumatoid arthritis, Sjogren's syndrome, autoimmune encephalomyelitis, nonalcoholic steatohepatitis (NASH), sarcoidosis, Behcet's disease, myasthenia gravis, lupus nephritis, inflammatory bowel disease (IBD), ankylosing spondylitis, primary biliary cirrhosis, colitis, pulmonary fibrosis, antiphospholipid syndrome, or psoriasis.

In embodiments, the compounds disclosed herein are used for treating cardiovascular disease. In embodiments, the cardiovascular disease is associated with inflammation. In embodiments, the cardiovascular disease includes systemic scleroderma. In embodiments, the cardiovascular disease includes aneurysm; angina; atherosclerosis; cerebrovascular accident (Stroke); cerebrovascular disease; congestive heart failure; coronary artery disease; myocardial infarction (heart attack); or peripheral vascular disease. In embodiments, the cardiovascular disease includes artherosclerosis.

In embodiments, the compounds disclosed herein are used for treating a gastrointestinal disease. In embodiments, the gastrointestinal disease includes Crohn's disease, primary biliary cirrhosis, sclerosing cholangitis, ulcerative colitis, inflammatory bowel disease, or Sjögren's syndrome.

In embodiments, the compounds disclosed herein are used for treating a urinary system disease. In embodiments, the urinary system disease includes systemic lupus erythematosus or systemic scleroderma.

In embodiments, the compounds disclosed herein are used for treating a genetic, familial, or congenital disease. In embodiments, the genetic, familial or congenital disease includes Crohn's disease, primary biliary cirrhosis, systemic scleroderma, systemic lupus erythematosus, ulcerative colitis, psoriasis, or inflammatory bowel disease.

In embodiments, the compounds disclosed herein are used for treating an endocrine system disease. In embodiments, the endocrine system disease includes thyroid gland adenocarcinoma, primary biliary cirrhosis, sclerosing cholangitis, or hypothyroidism.

In embodiments, the compounds disclosed herein are used for treating a cell proliferation disorder. In embodiments, the cell proliferation disorder includes primary biliary cirrhosis, thyroid gland adenocarcinoma, or neoplasm.

In embodiments, the compounds disclosed herein are used for treating an immune system disease. In embodiments, the immune system disease includes Sjögren's syndrome, inflammatory bowel disease, psoriasis, myositis, systemic scleroderma, autoimmune disease, systemic lupus erythematosus, rheumatoid arthritis, Crohn's disease, ulcerative colitis, or ankylosing spondylitis.

In embodiments, the compounds disclosed herein are used for treating a hematologic disease. In embodiments, the hematologic disease includes systemic lupus erythematosus.

In embodiments, the compounds disclosed herein are used for treating a musculoskeletal or connective tissue disease. In embodiments, the musculoskeletal or connective tissue disease includes myositis, systemic scleroderma, systemic lupus erythematosus, rheumatoid arthritis, ankylosing spondylitis or adolescent idiopathic scoliosis.

In embodiments, the compounds disclosed herein are used for treating neuroinflammatory disease. In embodiments, the neuroinflammatory disease or disorder includes inflammation due to traumatic brain injury, acute disseminated encephalomyelitis (ADEM), autoimmune encephalitis, acute optic neuritis (AON), chronic meningitis, anti-myelin oligodendrocyte glycoprotein (MOG) disease, transverse myelitis, neuromyelitis optica (NMO), Alzheimer's disease, Parkinson's disease or multiple sclerosis (MS).

In embodiments, the compounds disclosed herein are used for treating inflammation due to infection by microorganisms such as viruses, bacteria, fungi or parasites.

In embodiments, the compounds disclosed herein are used for treating a disease associated with fibrosis, which is referred to herein as a fibrotic disease. “Fibrosis” refers to a pathological formation of fibrous connective tissue, for example, due to injury, irritation, or chronic inflammation and includes fibroblast accumulation and collagen deposition in excess of normal amounts in a tissue. “Fibrotic disease” refers to a disease associated with pathological fibrosis. Examples of fibrotic disease include, but are not limited to, idiopathic pulmonary fibrosis; scleroderma; scleroderma of the skin; scleroderma of the lungs; a collagen vascular disease (e.g., lupus; rheumatoid arthritis; scleroderma); genetic pulmonary fibrosis (e.g., Hermansky-Pudlak Syndrome); radiation pneumonitis, asthma; asthma with airway remodeling; chemotherapy-induced pulmonary fibrosis (e.g., bleomycin, methotrextate, or cyclophosphamide-induced); radiation fibrosis; Gaucher's disease; interstitial lung disease; retroperitoneal fibrosis; myelofibrosis; interstitial or pulmonary vascular disease; fibrosis or interstitial lung disease associated with drug exposure; interstitial lung disease associated with exposures such as asbestosis, silicosis, and grain exposure; chronic hypersensitivity pneumonitis; an adhesion; an intestinal or abdominal adhesion; cardiac fibrosis; kidney fibrosis; cirrhosis; and nonalcoholic steatohepatitis (NASH)-induced fibrosis. In embodiments, the fibrotic disease includes non-alcoholic steatohepatitis NASH.

In embodiments, the compounds disclosed herein are used for treating a respiratory or thoracic disease such as systemic scleroderma. In embodiments, the compounds disclosed herein are used for treating an integumentary system disease such as psoriasis or systemic scleroderma. In embodiments, the compounds disclosed herein are used for treating a disease of the visual system such as Sjögren's syndrome or systemic scleroderma. In embodiments, the compounds disclosed herein are used for treating a disease associated with eosinophil count, glomular filtration rate, systolic blood pressure, eosinophil percentage of leukocytes. In embodiments, the compounds disclosed herein are used for treating an ulcer disease or an oral ulcer.

### Inflammatory Bowel Disease (IBD))

In embodiments, a method includes administering a compound or composition described herein to a subject in need thereof to treat inflammatory bowel disease (IBD).

IBD refers to two conditions characterized by chronic inflammation of the gastrointestinal (GI) tract: Crohn's disease and ulcerative colitis. Common symptoms of IBD include persistent diarrhea, abdominal pain, rectal bleeding/bloody stool, weight loss and fatigue. In 2015, an estimated 1.3% of US adults (3 million) reported being diagnosed with IBD (either Crohn's disease or ulcerative colitis). IBD is associated with an inflammatory macrophage phenotype in intestinal macrophages that is promoted by IRF-5.

### Rheumatoid Arthritis (RA)

In embodiments, a method includes administering a compound or composition described herein to a subject in need thereof to treat rheumatoid arthritis (RA).

RA is an autoimmune disease that affects 0.5% to 1% of the population worldwide. It causes joint pain and damage throughout a patient's body. Treatment for RA typically includes the use of medications that slow disease and prevent joint deformity, called disease-modifying antirheumatic drugs (DMARDs) and biologics (antibody) that target parts of the immune system that trigger inflammation that causes joint and tissue damage. IRF-5 polymorphisms have been identified as risk factors for RA. Reduced IRF-5 levels is associated with reduced disease phenotype. IRF-5 activation of TLR3 and TLR7 promotes inflammatory cytokine and chemokine production.

### Sjögren's Syndrome (SS)

In embodiments, a method includes administering a compound or composition described herein to a subject in need thereof to treat Sjögren's syndrome (SS).

SS is an immune disorder identified by dry eyes and a dry mouth. The condition often accompanies other immune system disorders, such as rheumatoid arthritis (RA) and systemic lupus erythematosus (SLE). The disease predominantly affects females between the age of 40-60. The prevalence of primary SS in the US was estimated to be between 2 and 10 per 10,000 inhabitants. Existing therapies for SS include treating symptoms of dry eyes and a dry mouth. There is no disease modifying therapy. IRF-5 rs2004640T allele, and CGGGG insertion/deletion have been associated with SS in multiple studies.

### Multiple Sclerosis (MS)

In embodiments, a method includes administering a compound or composition described herein to a subject in need thereof to treat multiple sclerosis (MS).

MS is a debilitating disease of the central nervous system (the brain and spinal cord). In MS, the immune system attacks the protective sheath (myelin) that covers nerve fibers and causes communication problems between the brain and body of a patient. Multiple sclerosis causes a broad spectrum of neurological symptoms, including sensory or motor paresis, visual disturbances, ataxia, impaired coordination, pain, cognitive dysfunction and fatigue. Current estimates suggest that 300,000 to 400,000 individuals are affected in the United States and over 2 million individuals worldwide. Treatment for MS is generally limited to Corticosteroids and plasma replacement therapies.

Two single nucleotide polymorphisms (SNPs) (rs4728142, rs3807306), and a 5 bp insertion-deletion polymorphism located in the promoter and first intron of the IRF-5 gene are strongly associated with MS. Kristjansdottir et al. (2008) “Interferon regulatory factor 5 (IRF-5) gene variants are associated with multiple sclerosis in three distinct populations,” J. Med. Genet. 45 (6): 362-369.

### Scleroderma or Systemic Sclerosis (SSc)

In embodiments, a method includes administering a compound or composition described herein to a subject in need thereof to treat scleroderma.

Scleroderma is a chronic connective tissue disease associated with wide-spread fibrosis of skin and internal organs, small-vessel vasculopathy and immune dysregulation with production of autoantibodies. Sharif et al. (2012) “IRF-5 polymorphism predicts prognosis in patients with systemic sclerosis,” Ann. Rheum. Dis. 71 (7): 1197-1202.

IRF-5 variant rs4728142 is associated with longer survival of SSc patients and lower IRF-5 transcript levels and was predictive of longer survival and milder interstitial lung disease (ILD) in SSc patients. Patients with no copies of IRF-5 rs4728142 had increased IRF-5 expression levels and experienced more severe ILD and shorter survival. Additional single nucleotide polymorphisms (rs10488631 and rs12537284) were identified in a genome-wide association study (GWAS) of systemic sclerosis (SS). Sharif et al. (2012) “IRF-5 polymorphism predicts prognosis in patients with systemic sclerosis,” Ann Rheum Dis. 71 (7): 1197-202.

### Non-Alcoholic Fatty Acid Liver Disease (NFALD))

In embodiments, a method includes administering a compound or composition described herein to a subject in need thereof to treat non-alcoholic fatty liver disease (NAFLD).

NAFLD refers to a range of conditions associated with accumulation of fat in the liver (known as steatosis) and encompasses fatty liver steatosis (NAFL) and nonalcoholic steatohepatitis (NASH). Hepatic fibrosis can develop in NAFLD, which can progress into cirrhosis and hepatocellular carcinoma (HCC). Disease progression in NAFLD can be broken down into 4 stages: 1) Fatty Liver Steatosis (NAFL), associated with a build-up of fat in the liver cells but little or no inflammation or fibrosis: 2) non-alcoholic steatohepatitis (NASH), in which the liver becomes inflamed. Persistent inflammation can result in scar tissue (fibrosis) in the liver and nearby blood vessels; and 4) cirrhosis, in which prolonged inflammation results in extensive damage and fibrosis which increases risk of complications such as liver failure and liver cancer (heptatocellular carcinoma).

### Methods of Treatment

The present disclosure provides a method of treating disease in a patient in need thereof, that includes administering a compound disclosed herein. In embodiments, the disease is any of the diseases provided in the present disclosure. In embodiments, a method of treating disease associated with IRF-5 includes administering to the patient a compound disclosed herein, thereby treating the disease.

In embodiments, the patient is identified as having, or at risk of having, a disease associated with IRF-5.

In embodiments, a method is provided for treating a disease or disorder associated with Interferon Regulatory Factor-5 (IRF-5). In embodiments, the disease or disorder is associated with IRF-5 genetic variation. In embodiments, the disease or disorder is associated with a genetic mutation in the IRF-5 gene. In embodiments, the genetic mutation in IRF-5 results IRF-5 overexpression. In embodiments, the genetic mutation results in alternate isoform expression. In embodiments, the disease or disorder is associated with IRF-5 overexpression.

In embodiments, the disease is an inflammatory disease. In embodiments, the disease is an autoimmune disease. In embodiments, the disease is a fibrotic disease.

In various embodiments, treatment refers to partial or complete alleviation, amelioration, relief, inhibition, delaying onset, reducing severity and/or incidence of one or more symptoms in a patient.

In embodiments, a method is provided for altering the expression of an IRF-5 in a patient in need thereof, that includes administering a compound disclosed herein. In embodiments, treatment results in modulation of IRF-5 activity in a patient. In embodiments, treatment results in modulation of IRF-5 activity in an immune cell of a patient. In embodiments, treatment results in modulation of IRF-5 expression in a patient. In embodiments, treatment results in modulation of IRF-5 expression in an immune cell of a patient. In embodiments, treatment result in a decrease in IRF-5 activity. In embodiments, treatment result in a decrease in IRF-5 expression. In embodiments, treatment inhibits activation of IRF-5. In embodiments, treatment inhibits phosphorylation of IRF-5. In embodiments, treatment inhibits ubiquitination of IRF-5. In embodiments, treatment reduces downstream proinflammatory cytokine activity. In embodiments, treatment reduces downstream proinflammatory cytokine expression. In embodiments, treatment reduces IFNα activity. In embodiments, treatment reduces IFNα expression. In embodiments, treatment reduces IL12p40 activity. In embodiments, treatment reduces IL12p40 expression. In embodiments, treatment inhibits nuclear localization of IRF-5. In embodiments, treatment inhibits DNA-binding by IRF-5. In embodiments, treatment inhibits IRF-5 dimer formation. In one embodiment, treatment results in expression of a re-spliced IRF-5 protein. In embodiments, treatment results in expression of a different IRF-5 isoform. In embodiments, treatment modulates macrophage polarization. In embodiments, treatment modulates macrophage differentiation. In embodiments, treatment modulates macrophage cytokine expression.

In embodiments, treatment modulates activity of IRF-5 in a patient in need thereof. In embodiments, treatment modulates activity of IRF-5 in a cell of a patient. In embodiments, treatment modulates activity of IRF-5 in an immune cell of a patient. In embodiments, immune cell is a monocyte, a lymphocyte or a dendritic cell. In embodiments, the lymphocyte is a B-lymphocyte. In embodiments, the monocyte is a macrophage. In embodiments, the macrophage is a resident tissue macrophage. In embodiments, the macrophage is a monocyte-derived macrophage. In embodiments, the macrophage is a Kupffer cell, an intraglomular mesangial cell, an alveolar macrophage, a sinus histiocyte, a hofbauer cell, microglia or langerhan cell. In embodiments, the immune cell is a Kupffer cell.

In embodiments, the method of treatment includes targeted inhibition of mutation-driven IRF-5 overexpression. In embodiments, the method of treatment includes targeted inhibition of isoform expression.

In embodiments, the treatment results in reduced expression of IRF-5. In some embodiments, the treatment results in the expression of a re-spliced target protein. In embodiments, the treatment results in the increased expression of an IRF-5 isoform.

In embodiments, treatment according to the present disclosure results in decreased IRF-5, activity and/or expression in a patient by 5% or more, 10% or more, 20%, or more, 30% or more, 40% or more, 50% or more, 60% or more, 70% or more, 80% or more, 90% or more as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient by 99% or less, 100% or less, 90% or less, 80% or less, 70% or less, 60% or less, 50% or less, 40% or less, 20% or less, or 10% or less as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient by 5% to 10%, 5% to 20%, 5% to 30%, 5% to 40%, 5% to 50%, 5% to 60%, 5% to 70%, 5% to 80%, 5% to 90%, or 5% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient by 10% to 20%, 10% to 30%, 10% to 40%, 10% to 50%, 10% to 60%, 10% to 70%, 10% to 80%, 10% to 90%, or 10% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient by 20% to 30%, 20% to 40%, 20% to 50%, 20% to 60%, 20% to 70%, 20% to 80%, 20% to 90%, or 20% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5 activity and/or expression in a patient by 30% to 40%, 30% to 50%, 30% to 60%, 30% to 70%, 30% to 80%, 30% to 90%, or 30% to 100% as compared to the average level and/or activity of the target protein in the patient before the treatment, of one or more control individuals with similar disease without treatment, or compared to treatment with a therapeutic moiety not conjugated to a CPP disclosed herein. In embodiments, treatment according to the present disclosure results in decreased IRF-5 activity and/or expression in a patient by 40% to 50%, 40% to 60%, 40% to 70%, 40% to 80%, 40% to 90%, or 40% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient 50% to 60%, 50% to 70%, 50% to 80%, 50% to 90%, or 50% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient 60% to 70%, 60% to 80%, 60% to 90%, or 60% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient 70% to 80%, 70% to 90%, or 70% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5, activity and/or expression in a patient 80% to 90% or 80% to 100% as compared to the average level and/or activity of the target protein in the patient before the treatment, of one or more control individuals with similar disease without treatment, or compared to treatment with an therapeutic moiety not conjugated to a CPP disclosed herein. In embodiments, treatment according to the present disclosure results in decreased IRF-5 activity and/or expression in a patient 90% to 100% as compared to the average level and/or activity of the target protein in the patient before the treatment, of one or more control individuals with similar disease without treatment, or compared to treatment with a therapeutic moiety not conjugated to a CPP disclosed herein.

In embodiments, treatment according to the present disclosure results in decreased IRF-5 expression in an immune cell of a patient by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of IRF-5 expression in the the immune cell of the patient before the treatment, compared to one or more control individuals with similar disease without treatment, or compared to treatment with an therapeutic moiety not conjugated to a CPP disclosed herein.

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have wild type target protein expression or activity levels that are less than about 1-99% of normal protein expression or activity levels in an individual not afflicted with the disease. In embodiments, the range includes, but is not limited to less than about 80-99%, less than about 65-80%, less than about 50-65%, less than about 30-50%, less than about 25-30%, less than about 20-25%, less than about 15-20%, less than about 10-15%, less than about 5-10%, less than about 1-5% of normal thymidine phosphorylase expression or activity levels. In embodiments, the individual may have target protein expression or activity levels that are 1-500% higher than normal wild type target protein expression or activity levels. In embodiments, the range includes, but is not limited to, greater than about 1-10%, about 10-50%, about 50-100%, about 100-200%, about 200-300%, about 300-400%, about 400-500%, or about 500-1000%.

In embodiments, the individual is a patient who has been recently diagnosed with the disease. Typically, early treatment (treatment commencing as soon as possible after diagnosis) reduces the effects of the disease and to increase the benefits of treatment. Methods of Making

The compounds described herein can be prepared in a variety of ways known to one skilled in the art of organic synthesis or variations thereon as appreciated by those skilled in the art. The compounds described herein can be prepared from readily available starting materials. Reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

### Compositions and Methods of Administration

In embodiments, compositions are provided that include one or more of the compounds described herein.

In embodiments, pharmaceutically acceptable salts and/or prodrugs of the disclosed compounds are provided. Pharmaceutically acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically acceptable base addition salts include sodium, potassium, calcium, ammonium, or magnesium salt. Examples of physiologically acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The compounds disclosed herein, and compositions that include them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously include between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions that include them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell includes attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors, poly[bis(p-carboxyphenoxy) propane:sebacic acid] in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders that include the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium that includes, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a patient's skin to reduce the size (and can include complete removal) of malignant or benign growths, or to treat an infection site. Compounds and agents disclosed herein can be applied directly to the growth or infection site. In embodiments, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to improve the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that include a compound disclosed herein in combination with a pharmaceutically acceptable carrier. In embodiments, the pharmaceutical composition is adapted for oral, topical or parenteral administration. The dose administered to a patient, particularly a human, should be sufficient to achieve a therapeutic response in the patient over a reasonable time frame, without lethal toxicity, and without causing more than an acceptable level of side effects or morbidity. One skilled in the art will recognize that dosage will depend upon a variety of factors including the condition (health) of the patient, the body weight of the patient, kind of concurrent treatment, if any, frequency of treatment, therapeutic ratio, as well as the severity and stage of the pathological condition.

Also disclosed are kits that include a compound disclosed herein in one or more containers. The disclosed kits can optionally include pharmaceutically acceptable carriers and/or diluents. In embodiments, a kit includes one or more other components, adjuncts, or adjuvants as described herein. In another embodiment, a kit includes one or more anti-cancer agents, such as those agents described herein. In embodiments, a kit includes instructions or packaging materials that describe how to administer a compound or composition of the kit. Containers of the kit can be of any suitable material, e.g., glass, plastic, metal, etc., and of any suitable size, shape, or configuration.

In embodiments, a compound and/or agent disclosed herein is provided in the kit as a solid, such as a tablet, pill, or powder form. In another embodiment, a compound and/or agent disclosed herein is provided in the kit as a liquid or solution. In embodiments, the kit includes an ampoule or syringe containing a compound and/or agent disclosed herein in liquid or solution form.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “cell penetrating peptide” or “CPP” refers to a peptide that facilitates delivery of a cargo, e.g., a therapeutic moiety (TM) into a cell. In embodiments, the CPP is cyclic, and is represented as “cCPP”. In embodiments, the cCPP is capable of directing a therapeutic moiety to penetrate the membrane of a cell. In embodiments, the cCPP delivers the therapeutic moiety to the cytosol of the cell. In embodiments, the cCPP delivers an antisense compound (AC) to a cellular location where a pre-mRNA is located.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide, or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO:42), the nucleoplasmin bipartite NLS with the sequence NLSKRPAAIKKAGQAKKKK (SEQ ID NO:52), the c-myc nuclear localization sequence having the amino acid sequence PAAKRVKLD (SEQ ID NO:53) or RQRRNELKRSF (SEQ ID NO:54), the sequence RMRKFKNKGKDTAELRRRRVEVSVELRKAKKDEQILKRRNV (SEQ ID NO:50) of the IBB domain from importin-alpha, the sequences VSRKRPRP (SEQ ID NO:57) and PPKKARED (SEQ ID NO:58) of the myoma T protein, the sequence PQPKKKPL (SEQ ID NO:59) of human p53, the sequence SALIKKKKKMAP (SEQ ID NO:60) of mouse c-abl IV, the sequences DRLRR (SEQ ID NO:61) and PKQKKRK (SEQ ID NO:62) of the influenza virus NS1, the sequence RKLKKKIKKL (SEQ ID NO:63) of the Hepatitis virus delta antigen and the sequence REKKKFLKRR (SEQ ID NO:64) of the mouse Mx1 protein, the sequence KRKGDEVDGVDEVAKKKSKK (SEQ ID NO:65) of the human poly(ADP-ribose) polymerase and the sequence RKCLQAGMNLEARKTKK (SEQ ID NO:66) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

As used herein, the term “contiguous” refers to two amino acids, which are connected by a covalent bond. For example, in the context of a representative cyclic cell penetrating peptide (cCPP) such as

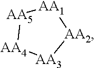

AA₁/AA₂, AA₂/AA₃, AA₃/AA₄ and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

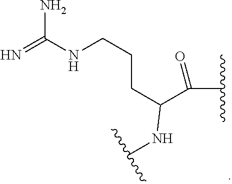 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

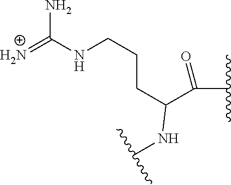 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₈ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes Cu and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, S-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, S-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O) ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O) OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, the symbol “

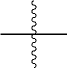

” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “

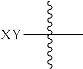

” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “

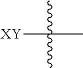

” infers that when R³ is “XY”, the point of attachment bond is the same bond as the bond by which R³ is depicted as being bonded to CH₃.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The terms “inhibit”, “inhibiting” or “inhibition” refer to a decrease in an activity, expression, function or other biological parameter and can include, but does not require complete ablation of the activity, expression, function or other biological parameter. Inhibition can include, for example, at least about a 10% reduction in the activity, response, condition, or disease as compared to a control. In embodiments, expression, activity or function of a gene or protein is decreased by a statistically significant amount. In embodiments, activity or function is decreased by at least about 10%, about 20%, about 30%, about 40%, about 50%, and up to about 60%, about 70%, about 80%, about 90% or about 100%. In embodiments, the expression, activity or function of IRF-5 is inhibited.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to a carrier suitable for administration to a patient. A pharmaceutically acceptable carrier can be a sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In such embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized antisense oligonucleotide. In embodiments, a dosage unit is a vial that includes reconstituted antisense oligonucleotide.

The term “therapeutic moiety” (TM) refers to a compound that can be used for treating, at least one symptom of a disease or disorder and can include, but is not limited to, therapeutic polypeptides, oligonucleotides, small molecules and other agents that can be used to treat at least one symptom of a disease or disorder. In embodiments, the therapeutic moiety modulates Interferon Regulatory Factor-5 (IRF-5) activity. In embodiments, the therapeutic moiety modulates Interferon Regulatory Factor-5 (IRF-5) expression. In embodiments, the therapeutic moiety modulates splicing. In embodiments, the therapeutic moiety induces exon skipping. In embodiments, the therapeutic moiety targets a polyadenylation site or a portion thereof of a target gene or an RNA transcript of a target gene. In embodiments, the therapeutic moiety targets a polyadenylation site or a portion thereof of an mRNA transcript of a target gene. In embodiments, the therapeutic moiety downregulates expression or activity of a target protein. In embodiments, the therapeutic moiety upregulates expression or activity of a target protein.

The terms “modulate”, “modulating” and “modulation” refer to a perturbation of expression, function or activity when compared to the level of expression, function or activity prior to modulation. Modulation can include an increase (stimulation or induction) or a decrease (inhibition or reduction) in expression, function or activity. In embodiments, the compound disclosed herein includes a therapeutic moiety (TM) that decreases IRF-5 expression, function and/or activity. In embodiments, IRF-5 activity is modulated by modulating IRF-5 expression.

“Amino acid” refers to an organic compound that includes an amino group and a carboxylic acid group and has the general formula

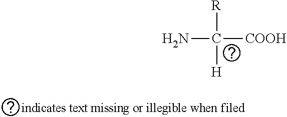

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain” or “side chain” refers to the characterizing substituent (“R”) bound to the α-carbon of a natural or non-natural α-amino acid. An amino acid may be incorporated into a polypeptide via a peptide bond.

As used herein, the term “sequence identity” refers to the percentage of nucleic acids or amino acids between two oligonucleotide or polypeptide sequences, respectively, that are the same and in the same relative position. As such one sequence has a certain percentage of sequence identity compared to another sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between sequences may be determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., Trends Genet. (2000), 16:276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows: (Identical Residues×100)/(Length of Alignment-Total Number of Gaps in Alignment)

In other embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP, gapped BLAST, and PSI-BLAST, in existence as of the date of filing. Such programs are described in Altschul, et al., J. Mol Biol., (1990), 215 (3): 403-410; Altschul, et al., Nucleic Acids Res. (1997), 25:3389-3402; Baxevanis et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, “cell targeting moiety” refers to a molecule or macromolecule that specifically binds to a molecule, such as a receptor, on the surface of a target cell. In embodiments, the cell surface molecule is expressed only on the surface of a target cell. In embodiments, the cell surface molecule is also present on the surface of one or more non-target cells, but the amount of cell surface molecule expression is higher on the surface of the target cells. Examples of a cell targeting moiety include, but are not limited to, an antibody, a peptide, a protein, an aptamer or a small molecule.

As used herein, the terms “antisense compound” and “AC” are used interchangeably to refer to a polymeric nucleic acid structure which is at least partially complementarity to a target nucleic acid molecule to which it (the AC) hybridizes. The AC may be a short (in embodiments, less than 50 bases) polynucleotide or polynucleotide homologue that includes a sequence complimentary to a target sequence. In embodiments, the AC is a polynucleotide or polynucleotide homologue that includes a sequence complimentary to a target sequence in a target pre-mRNA strand. The AC may be formed of natural nucleic acids, synthetic nucleic acids, nucleic acid homologues, or any combination thereof. In embodiments, the AC includes oligonucleosides. In embodiments, AC includes antisense oligonucleotides. In embodiments, the AC includes conjugate groups. Nonlimiting examples of ACs include, but are not limited to, primers, probes, antisense oligonucleotides, external guide sequence (EGS) oligonucleotides, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound. In embodiments, an AC modulates (increases, decreases, or changes) expression of a target nucleic acid.

As used herein, the terms “targeting” or “targeted to” refer to the association of a therapeutic moiety, for example, an antisense compound with a target nucleic acid molecule or a region of a target nucleic acid molecule. In embodiments, the therapeutic moiety includes an antisense compound that is capable of hybridizing to a target nucleic acid under physiological conditions. In embodiments, the antisense compound targets a specific portion or site within the target nucleic acid, for example, a portion of the target nucleic acid having at least one identifiable structure, function, or characteristic such as a particular exon or intron, or selected nucleobases or motifs within an exon or intron.

As used herein, the terms “target nucleic acid sequence” and “target nucleotide sequence” refer to the nucleic acid sequence or the nucleotide sequence to which a therapeutic moiety, such as an antisense compound, binds or hybridizes. Target nucleic acids include, but are not limited, to a portion of a target transcript, target RNA (including, but not limited to pre-mRNA and mRNA or portions thereof), a portion of target cDNA derived from such RNA, as well as a portion of target non-translated RNA, such as miRNA. For example, in embodiments, a target nucleic acid can be a portion of a target cellular gene (or mRNA transcribed from such gene) whose expression is associated with a particular disorder or disease state. The term “portion” refers to a defined number of contiguous (i.e., linked) nucleotides of a nucleic acid.

As used herein, the term “transcript” or “gene transcript” refers an RNA molecule transcribed from DNA and includes, but is not limited to mRNA, pre-mRNA, and partially processed RNA.

The terms “target transcript” and “target RNA” refer to the pre-mRNA or mRNA transcript that is bound by the therapeutic moiety. The target transcript may include a target nucleotide sequence. In embodiments, the target transcript includes a splice site. In embodiments, the target RNA includes a polyadenylation site or a portion thereof.

The term “target gene” and “gene of interest” refer to the gene of which modulation of the expression and/or activity is desired or intended. The target gene may be transcribed into a target transcript that includes a target nucleotide sequence. The target transcript may be translated into a protein of interest.

The term “target protein” refers to the polypeptide or protein encoded by the target transcript (e.g., target mRNA).

As used herein, the term “mRNA” refers to an RNA molecule that encodes a protein and includes pre-mRNA and mature mRNA. “Pre-mRNA” refers to a newly synthesized eukaryotic mRNA molecule directly after DNA transcription. In embodiments, a pre-mRNA is capped with a 5′ cap, modified with a 3′ poly-A tail, and/or spliced to produce a mature mRNA sequence. In embodiments, pre-mRNA includes one or more introns. In one embodiment, the pre-mRNA undergoes a process known as splicing to remove introns and join exons. In embodiments, pre-mRNA includes one or more splicing elements or splice regulatory elements. In embodiments, pre-mRNA includes a polyadenylation site.

As used herein, the term “expression,” “gene expression,” “expression of a gene,” or the like refers to all the functions and steps by which information encoded in a gene is converted into a functional gene product, such as a polypeptide or a non-coding RNA, in a cell. Examples of non-coding RNA include transfer RNA (IRNA) and ribosomal RNA. Gene expression of a polypeptide includes transcription of the gene to form a pre-mRNA, processing of the pre-mRNA to form a mature mRNA, translocating the mature mRNA from the nucleus to the cytoplasm, translation of the mature mRNA into the polypeptide, and assembly of the encoded polypeptide. Expression includes partial expression. For example, expression of a gene may be referred to herein as generation of a gene transcript. Translation of a mature mRNA may be referred to herein as expression of the mature mRNA.

As used herein, “modulation of gene expression” or the like refers to modulation of one or more of the processes associated with gene expression. For example, modification of gene expression may include modification of one or more of gene transcription, RNA processing, RNA translocation from the nucleus to the cytoplasm, and translation of mRNA into a protein.

As used herein, the term “gene” refers to a nucleic acid sequence that encompasses a 5′ promoter region associated with the expression of the gene product, and any intron and exon regions and 3′ untranslated regions (“UTR”) associated with the expression of the gene product.

The term “immune cell” refers to a cell of hematopoietic origin and that plays a role in the immune response. Immune cells include, but are not limited to, lymphocytes (e.g., B cells and T cells), natural killer (NK) cells, and myeloid cells. The term “myeloid cells” includes monocytes, macrophages and granulocytes (e.g., basophils, neutrophils, eosinophils and mast cells). Monocytes are lymphocytes that circulate through the blood for 1-3 days, after which time, they either migrate into tissues and differentiate into macrophages or inflammatory dendritic cells or die. The term “macrophage” as used herein includes fetal-derived macrophages (which also can be referred to as resident tissue macrophages) and macrophages derived from monocytes that have migrated from the bloodstream into a tissue in the body (which can be referred to as monocyte-derived macrophages). Depending on which tissue the macrophage is located, it be referred to as a Kupffer cell (liver), an intraglomular mesangial cell (kidney), an alveolar macrophage (lungs), a sinus histiocyte (lymph nodes), a hofbauer cell (placenta), microglia (brain and spinal cord), or langerhans (skin), among others.

As used herein, “proximate” with respect to an AC and a splice element means that the AC binds to a nucleic acid sequence that is within about 25, about 20, about 15, about 10, about 5, about 4, about 3, about 2 or about 1 nucleotides of a splice regulatory element, including, for example, a 5′ splice site (5′ ss), a branchpoint sequence (BPS), a polypyrimidine (Py) tract, or a 3′ splice site (3′ ss). As used herein, “proximate” with respect to an AC and a polyadenylation sequence element means that the AC binds to a nucleic acid sequence that is within about 25, about 20, about 15, about 10, about 5, about 4, about 3, about 2 or about 1 nucleotides of a polyadenylation site or a sequence element of a polyadenylation site.

As used herein, “splice regulatory element (SRE),” “splicing element (SE),” and “splice element (SE)” are used interchangeably and refer to any nucleotide sequence within the transcript at which splicing occurs or that promotes, inhibits, or alters splicing. Examples of splice elements include terminal stem loop sequence (TLS), branchpoint sequence (BPS), polypyrimidine sequence (Py), 5′ splice site (5′ ss), 3′ splice site (3′ ss), and cis-regulatory elements such as intronic splicing silencer (ISS) sequences, intronic splicing enhancer (ISE) sequences, exon splicing enhancer (ESE) sequences, exonic splicing silencer (ESS) sequences, and sequences that include an exon/intron junction.

As used herein, the terms “splicing” refers to the modification of a pre-mRNA following transcription, in which introns are removed and exons are joined. Splicing occurs in a series of reactions that are catalyzed by a large RNA-protein complex that includes five small nuclear ribonucleoproteins (snRNPs), referred to as a spliceosome. Splice regulatory elements include a 3′ splice site, a 5′ splice site, and a branch site. The 5′ splice site is bound by the U1 snRNP and subsequently by the U6 snRNP. The RNA binding protein SF1 binds the branch point sequence but is later displaced by the U2 snRNP (See, for example, Ward and Cooper (2011) “The pathobiology of splicing.”. J. Pathol. 220 (2): 152-163).

As used herein, “splice site” refers to the junction between an exon and an intron in a pre-mRNA molecule. A “cryptic splice site” is a splice site that is not typically used but may be used when the usual splice site is blocked or unavailable or when a mutation causes a normally dormant site to become an active splice site. An “aberrant splice site” is a splice site that results from a mutation in the native DNA and mRNA. An antisense compound that is “targeted to a splice site” refers to a compound that hybridizes with at least a portion of a target nucleotide sequence that includes a splice site or a compound that hybridizes with an intron or exon in proximity to a splice site, such that splicing of the mRNA is modulated. The targeted splice site may be a usual splice site, a cryptic splice site, or an aberrant splice site.

As used herein “splice donor site” can be used interchangeably with the term “5′ splice site” to refer to the nucleotide sequence immediately surrounding the exon-intron boundary at the 5′ end of the intron. The term “splice acceptor site” can be used interchangeably with the term “3′ splice site” to refer to the nucleic acid sequence immediately surrounding the intron-exon boundary at the 3′ end of the intron. Many splice donor and acceptor sites have been characterized (See, for example, Ohshima et al. (1987). J. Mol. Biol., 195:247-259 (1987)).

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

As used herein, the term “nucleoside” refers to a glycosylamine that includes a nucleobase and a sugar. Nucleosides include, but are not limited to, natural nucleosides, abasic nucleosides, modified nucleosides, and nucleosides having mimetic bases and/or sugar groups A “natural nucleoside” or “unmodified nucleoside” is a nucleoside that includes a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside having a phosphate group covalently linked to the sugar. Nucleotides may be modified with any of a variety of substituents.

As used herein, the term “nucleobase” refers to the base portion of a nucleoside or nucleotide. A nucleobase may include any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase that includes a heterocycle.

As used herein “internucleoside linkage” refers to a covalent linkage between adjacent nucleosides.

As used herein “natural internucleoside linkage” refers to a 3′ to 5′ phosphodiester linkage.

As used herein, the term “modified internucleoside linkage” refers to any linkage between nucleosides or nucleotides other than a naturally occurring internucleoside linkage.

As used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general, a chimeric oligomeric compound will have modified nucleosides that can be in isolated positions or grouped together in regions that will define a particular motif. Any combination of modifications and or mimetic groups can include a chimeric oligomeric compound as described herein.

As used herein, the term “mixed-backbone antisense oligonucleotide” refers to an antisense oligonucleotide wherein at least one internucleoside linkage of the antisense oligonucleotide is different from at least one other internucleoside linkage of the antisense oligonucleotide.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may include up to about 20% nucleotides that are mismatched (i.e., are not nucleobase complementary to the corresponding nucleotides of the target). In embodiments, the antisense compounds contain no more than about 15%, for example, not more than about 10%, for example, not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In embodiments, an antisense oligonucleotide specifically hybridizes to more than one target site. In embodiments, an oligomeric compound specifically hybridizes with its target under stringent hybridization conditions.

“Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

As used herein, the term “2′-modified” or “2′-substituted” means a sugar that includes substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O—C1-C10 alkyl, —OCF3, O—(CH2)2-O—CH3, 2′-O (CH2)2SCH3, O—(CH2)2-O—N(Rm)(Rn), or O—CH2-C(═O)—N(Rm)(Rn), where each Rm and Rn is, independently, H or substituted or unsubstituted C1-C10 alkyl.

As used herein, the term “MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of an antisense compound that includes the modified nucleotide to a target nucleic acid High-affinity modifications include, but are not limited to, BNAs, LNAs and 2′-MOE.

As used herein the term “mimetic” refers to groups that are substituted for a sugar, a nucleobase, and/or internucleoside linkage in an AC. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference) Methods of synthesis of sugar, nucleoside and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH3)-CH2-4′) and Aminooxy BNA (2′-N(CH3)-O—CH2-4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “cap structure” or “terminal cap moiety” refers to chemical modifications, which have been incorporated at either end of an AC.

“Interferon Regulatory Factor-5” or “IRF-5” or “IRF-5” is a member of the IRF family of transcription factors and is involved in innate and adaptive immunity, macrophage polarization, cell growth regulation and differentiation and apoptosis. Aberrant IRF-5 expression is associated with a variety of diseases including, but not limited to, cancer, autoimmune and inflammatory diseases and fibrotic conditions, including, but not limited to, inflammatory bowel disease (IBD), primary biliary cirrhosis, rheumatoid arthritis (RA), systemic lupus erythematosus (SLE), systemic sclerosis and non-alcoholic steatohepatitis (NASH).

The term “therapeutic polypeptide” refers to a naturally occurring or recombinantly produced macromolecule that includes two or more amino acids and has therapeutic, prophylactic or other biological activity.

The term “antigen-binding fragment” or “antigen-binding antibody fragment” are used interchangeable herein and refer to a polypeptide fragment that contains at least one complementarity-determining region (CDR) of an immunoglobulin heavy and/or light chain that binds to at least one epitope of the antigen of interest. The CDR may be derived from a human immunoglobulin or a camelid immunoglobulin. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind to a target molecule. Antigen-binding fragments include proteins that comprise a portion of a full length antibody, generally the antigen binding or variable region thereof, such as Fab, F(ab′)2, Fab′, Fv fragments, minibodies, diabodies, single domain antibodies (dAb), single-chain variable fragments (scFv), multispecific antibodies formed from antibody fragments, and any other modified configuration of the immunoglobulin molecule that comprises an antigen-binding site or fragment of the required specificity.

The term “antigen-binding domain” as used herein refers to a polypeptide that binds to an antigen. The antigen-binding domain me be an antibody, an antigen-binding fragment, or an antibody mimetic.

The term “antibody” as used herein refers to a molecule that contains at least one antigen binding site that immunospecifically binds to a particular antigen target of interest. The term “antibody” thus includes but is not limited to a full-length antibody and/or its variants, a fragment thereof, peptibodies and variants thereof, monoclonal antibodies (including full-length monoclonal antibodies), polyclonal antibodies, multispecific antibodies (for example, bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. Binding of an antibody to a target can cause a variety of effects, such as but not limited to where such binding modulates, decreases, increases, antagonizes, agonizes, mitigates, alleviates, blocks, inhibits, abrogates and/or interferes with at least one target activity or binding, or with receptor activity or binding, in vitro, in situ, and/or in vivo. An antibody of the present disclosure encompasses antibody fragments capable of binding to a particular antigen target of interest, including but not limited to Fab, Fab′, F(ab′)₂, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments. The antibody may be of any type, any class, or any subclass.

When the antibody is a human or mouse antibody, the type may include, for example, IgG, IgE, IgM, IgD, IgA and IgY, and the class may include, for example, IgG1, IgG2, IgG3, IgG4, IgA1 and IgA2. When the antibody is an IgG antibody, the antibody includes a two light chains and two heavy chains. The light chains include two variable regions (VL) and two conserved regions (CL). The heavy chain includes two variable regions (VH) and three conserved regions (CH1, CH2, CH3). Each of the heavy chains associate with a light chain by virtue of interchain disulfide bonds between the heavy and light chain to form two heterodimeric proteins or polypeptides (i.e., a protein comprised of two heterologous polypeptide chains). The two heterodimeric proteins then associate by virtue of additional interchain disulfide bonds between the heavy chains to form an Ig molecule (See FIG. 8A).

When the antibody is a camelid antibody, the type may include, for example, camelid heavy chain IgG (hcIgG), camelid single N-terminal variable domain heavy chain (VHH) region, and single domain antibody comprising the VHH (See FIG. 8B).

The term “antigen-binding fragment” as used herein refers to a polypeptide fragment that contains at least one complementarity-determining region (CDR) of an immunoglobulin heavy and/or light chain that binds to at least one epitope of the antigen of interest. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind to a target molecule. The antigen-binding fragment of the herein described camelid antibodies may comprise 1, 2, or 3 of the CDRs of a camelid VHH region. The antigen-binding fragment of the herein described camelid antibodies may be a single domain antibody (VHH). Antigen-binding fragments include proteins that comprise a portion of a full length antibody, generally the antigen binding or variable region thereof, such as Fab, F(ab′)2, Fab′, Fv fragments, minibodies, single domain antibodies (dAb), single-chain variable fragments (scFv), divalent scFv such as diabodies, multispecific antibodies formed from antibody fragments, and any other modified configuration of the immunoglobulin molecule that comprises an antigen-binding site or fragment of the required specificity (See FIG. 8A/B).

The term “antibody mimetic” refers to a polypeptide that can specifically bind an antigen but is not structurally related to an antibody. Examples of antibody mimetics include monobodies, affibody molecules (constructed on a scaffold of the Z-domain of Protein A, See, Nygren, FEBS J. (2008), 275 (11): 2668-76), affilins (constructed on a scaffold of gamma-B crystalline or ubiquitin, See Ebersbach H et al., J. Mol. Biol. (2007), 372 (1): 172-85), affimers (constructed on a Crystatin scaffold, See Johnson A et al, Anal. Chem. (2012), 84 (15): 6553-60), affitins (constructed on a Sac7d from S. acidocaldarius scaffold, See Krehenbrink M et al., J. Mol. Biol. (2008), 383 (5): 1058-68), alphabodies (constructed on a triple helix coiled coil scaffold, See Desmet, J et al., Nature Communications (2014), 5:5237), anticalins (constructs on scaffold of lipocalins, See Skerra A., FEBS J. (2008), 275 (11): 2677-83), avimers (constructed on scaffolds of various membrane receptors, See Silverman J. et al., Nat. Biotechnol. (2005), 23 (12): 1556-61), DARPins (constructed on scaffolds of ankyrin repeat motifs, See Stumpp et al., Drug Discov. Today (2008), 3 (15-16). 695-701), fynomers (constructed on a scaffold of the SH3 domain of Fyn, See Grabulovski et al., J Biol Chem. (2007), 282 (5): 3196-3204), Kunitz domain peptides (constructed on scaffolds of the Kunitz domains of various protease inhibitors, See Nixon et. al., Curr. Opin. Drug. Discov. Dev. (2006), 9 (2): 261-8), and monobodies (constructed on scaffolds of type III domain of fibronectin, See Koide et al (2007).

The term “monobody” refers to a synthetic binding protein constructed using a fibronectin type III domain (FN3) as a molecular scaffold.

The term “minibody” refers to a CH3 domain fused or linked to an antigen-binding fragment (e.g., a CH3 domain fused or linked to an scFv, a domain antibody, etc.). In embodiments, the term “Mb” signifies a CH3 single domain. In other embodiments, a CH3 domain signifies a minibody. (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment.

The term “F(ab′)2” refers to a protein fragment of IgG generated by proteolytic cleavage by the enzyme pepsin. Each F(ab′)2 fragment comprises two F(ab′) fragments linked by disulfide bonds in the hinge region and is therefore a bivalent antigen-binding fragment. The term “Fab′” refers to a fragment derived from F(ab′)2 and may contain a small portion of the Fc. Each Fab′ fragment is a monovalent antigen-binding fragment.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

“Fc region” or “Fc domain” refers to a polypeptide sequence corresponding to or derived from the portion of an antibody that is capable of binding to Fc receptors on cells and/or the C1q component or complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

A pFc′ fragment refers to an Fc region that is not covalently coupled.

A “single domain antibody” (sdAb) refers to an antibody fragment comprising a single monomeric heavy chain variable domain. In embodiments, where the antibody fragment is from a camelid heavy chain IgG, the variable domain may be the VHH.

The term “antigen” refers to a molecule or a portion of a molecule capable of being bound by an antibody or an antigen-binding fragment thereof and additionally capable of being used in an animal to produce antibodies capable of binding to an epitope of that antigen.

Antigens include, but are not limited to, proteins, polysaccharides, lipids, or glycolipids. In embodiments, an antigen is an antigen of an infectious agent. In embodiments, the antigen is an extracellular antigen. In embodiments, the antigen is a cell surface antigen. In embodiments, the antigen is an intracellular antigen. An antigen may have one or more epitopes.

The term “epitope” refers to a region of an antigen that is bound by an antibody. Epitope determinants can include chemically active surface groupings of molecules such as amino acids, sugar side chains, phosphoryl, or sulfonyl and may have specific three-dimensional structural characteristics, and/or specific charge characteristics.

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs).

The term “immunoglobulin light chain constant region” (also referred to as “light chain constant region” or “CL”) is a constant region from an antibody light chain.

The term “immunoglobulin heavy chain constant region” (also referred to as “heavy chain constant region” or “CH”) refers to the constant region from the antibody heavy chain. The CH is further divisible, depending on the antibody isotype into CH1, CH2, and CH3 (IgA, IgD, IgG), or CH1, CH2, CH3, and CH4 domains (IgE, IgM).

The term “single-chain variable fragment (scFv) refers to fusion between a VH and VL. Generally. The N-terminus of the VH and the C-terminus of the VL or the N-terminus of the VL and the C-terminus of the VH are coupled through a linker peptide.

The term “variable region of heavy chain only” or “variable region of hcIgG” (VHH) refers to the variable region of an hcIgG such as those from camelids. A VHH includes 3 CDRs.

Divalent single chain variable fragment (di-scFv) refers to the association of two or more scFvs either through covalent bonds or non-covalent means such as dimerization. A diabody is a dimer of two scFv where the scFv comprise a VH and VL linked by a peptide linker that is too short allow for intramolecular association.

As used herein, the term “complementarity determining region” or “CDR” refer to an immunoglobulin (antibody) molecule. There are three CDRs per variable domain: CDR1, CDR2 and CDR3 in the variable domain of the light chain and CDR1, CDR2 and CDR3 in the variable domain of the heavy chain. In camelid antibodies and antigen binding fragments thereof, there are three CDRs per VHH.

As used herein “active portion” or “active portion thereof” refers to a fragment of a polypeptide that retains the function of the polypeptide. Functions include but are not limited to, binding and or/enzymatic activity. The binding affinity of a active portion need not be the same as the full polypeptide.

The term “specifically binds” refers to the ability of an antibody or antigen-binding fragment thereof to bind a target antigen with a binding affinity (Ka) of at least 10⁵ M⁻¹ while not significantly binding other components or antigens present in a mixture.

Binding affinity (Ka) refers to an equilibrium association of a particular interaction expressed in the units of 1/M or M⁻¹. Antibodies or antigen-binding antibody fragments thereof can be classified as “high affinity” antibodies or antigen-binding fragments thereof and “low affinity” antibodies or antigen-binding fragments thereof. “High affinity” antibodies or antigen-binding fragments thereof refer to those antibodies or antigen-binding fragments thereof with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” antibodies or antigen-binding fragments thereof refer to those antibodies or antigen-binding fragments thereof with a Ka of up to 10⁷ M⁻¹, up to 10⁶ M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³, or about 500 nM, about 300 nM, about 250 nM, about 200 nM, about 150 nM, about 100 nM, about 50 nM, about 25 nM, about 10 nM, or about 5 nM). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

The term “small molecule” refers to an organic compound with pharmacological activity and a molecular weight of less than about 2000 Daltons, or less than about 1000 Daltons, or less than about 500 Daltons Small molecule therapeutics are typically manufactured by chemical synthesis.

The term “IRF-5 inhibitor” refers to a compound that is capable of reducing or suppressing IRF-5 expression, activity or function. An IRF-5 inhibitor can be a natural or synthetic small molecule compound or a biological molecule such as a polypeptide or oligonucleotide. In embodiments, the IRF-5 inhibitor is an antisense compound (AC), such as an antisense oligonucleotide. In embodiments, the IRF-5 inhibitor is an antisense compound that blocks translation of IRF-5 mRNA, for example, by binding to the mRNA and preventing protein translation. In embodiments, the IRF-5 inhibitor is an antisense compound that binds to IRF-5 mRNA and increases mRNA degradation, thereby decreasing the amount of, and thus, activity of IRF-5 in a cell. In embodiments, the IRF-5 inhibitor reduces transcription from promoters containing IRF-5 binding sites. For example, the IRF-5 inhibitor can block the interaction between IRF-5 and IRF-5 binding sequences. In embodiments, the IRF-5 inhibitor binds to IRF-5 such that IRF-5 cannot bind to the IRF-5 binding sites. In embodiments, the IRF-5 inhibitor blocks the interaction between IRF-5 and other IRF monomers to prevent homo- or heterodimerization. In embodiments, the IRF-5 inhibitor is a selective inhibitor of IRF-5 activity. A “selective inhibitor of IRF-5 activity” is a compound that preferentially inhibits IRF-5 activity over the activity of other members of the IRF family including, but not limited to IRF-1, IRF-2, IRF-3, IRF-4, etc.

The term “immune cell” refers to a cell of hematopoietic origin and that plays a role in the immune response. Immune cells include, but are not limited to, lymphocytes (e.g., B cells and T cells), natural killer (NK) cells, and myeloid cells. The term “myeloid cells” includes monocytes, macrophages and granulocytes (e.g., basophils, neutrophils, eosinophils and mast cells). Monocytes are lymphocytes that circulate through the blood for 1-3 days, after which time, they either migrate into tissues and differentiate into macrophages or inflammatory dendritic cells or die. The term “macrophage” as used herein includes fetal-derived macrophages (which also can be referred to as resident tissue macrophages) and macrophages derived from monocytes that have migrated from the bloodstream into a tissue in the body (which can be referred to as monocyte-derived macrophages). Depending on which tissue the macrophage is located in, it can be referred to as a Kupffer cell (liver), an intraglomular mesangial cell (kidney), an alveolar macrophage (lungs), a sinus histiocyte (lymph nodes), a hofbauer cell (placenta), microglia (brain and spinal cord), or langerhans (skin), among others.

As used herein “polyadenylation” refers to the cellular process in which a chain of adenosine bases (referred to as a poly(A) tail) is added to an RNA transcript, for example, a pre-mRNA sequence. Polyadenylation is a two-step reaction that includes specific endonucleolytic cleavage of the 3′ end of an RNA transcript at and addition of the poly(A) tail. The processing of most human poly(A) sites involves the recognition of a canonical hexamer sequence by a cleavage and polyadenylation specific factor (CPSF), coupled with the binding of cleavage stimulatory factor (CstF) to a GU-rich downstream element (DSE). See, Venkataraman et al. (2005) “Analysis of a noncanonical poly(A) site reveals a tripartite mechanism for vertebrate poly(A) site recognition,” Genes and Dev. 19:1315-1327.

As used herein a “poly(A) tail” or “polyadenosine tail” refers to a chain of adenosine bases on the 3′ end of a mRNA sequence. The length of poly(A) tails is generally specific to a species. For example, poly(A) tails can range from 150 to 250 adenosines in mammals and from 55 to 90 adenosines in yeasts (See, Tian et al. (2005) Nuc. Acid. Res. 33 (1): 201-212 and Neve et al. (2017) RNA Biology, 14 (7): 865-890 and Brown, C. E. and Sachs, A. B. (1998). Mol. Cell. Biol., 18, 6548-6559).

As used herein, the term “polyadenylation site” (PS) refers to a nucleotide sequence found in vertebrate pre-mRNA at which a poly(A) tail is added to form the mature mRNA. The polyadenylation site includes four main sequence elements: a polyadenylation signal (PAS), an intervening sequence (IS), a cleavage site (CS) and a downstream element (DSE). As used herein, “sequence element” refers to a recurring nucleotide sequence motif that is associated with a biological function or activity. The term “cleavage site(s)” refers to a nucleotide pair between which cleavage takes place. The first recognition element is an adenosine-rich hexamer sequence, referred to herein as a “polyadenylation signal” (PAS) that includes a canonical hexamer sequence or a variant thereof. The polyadenylation signal (PAS) is typically found from about 10 to about 35, or about 10 to about 20, or at least about 10, about 11, about 12, about 13, about 14, about 15, about 16, about 17, about 18, about 19, about 20, about 21, about 22, about 23, about 24 or about 25 and up to about 30 or about 35 nucleotides upstream of the cleavage site (CS). The hexamer sequence serves as the binding site for the cleavage and polyadenylation specificity factor (CPSF). The second recognition element is a downstream sequence element (DSE) that includes a U-rich or U/G-rich element. The DSE is typically found from about 10 to about 40, about 10 to about 30, about 20 to about 40, or about 20 to about 30, or at least about 20, about 21, about 22, about 23, about 24, or about 25 and up to about 26, about 27, about 28, about 29, about 30, about 35 or about 40 nucleotides downstream of the cleavage site. The U-rich or U/G-rich element serves as a binding site for the cleavage stimulation factor (CstF). The DSE may be followed by a stretch of 3 or more uracil residues (U) downstream of the cleavage site, often within 40 nucleotides of the cleavage site. In mammals, CA and UA are the most frequent dinucleotides that precede the cleavage site, although the actual cleavage site is known to be heterogeneous. The polyadenylation site can also be determined by the presence of other auxiliary elements, such as upstream U-rich elements (USE). See, Tian et al. (2005) “A large-scale analysis of mRNA polyadenylation of human and mouse genes,” Nuc. Acid. Res. 33 (1): 201-212 and Neve et al. (2017) “Cleavage and polyadenylation: ending the message expands gene regulation,” RNA Biology, 14 (7): 865-890.

As used herein, “cleavage and polyadenylation” or “CPA” refers to a two-step process involving generation of a 3′ end through an initial endonucleolytic cleavage of RNA followed by addition of a chain of adenosine bases (a poly(A) tail) to an RNA sequence.

“Wild type target protein” refers to a native, functional protein isomer produced by a wild type, normal, or unmutated version of the target gene. The wild type target protein also refers to a protein resulting from a target pre-mRNA that has been re-spliced.

A “re-spliced target protein”, as used herein, refers to the protein encoded by the mRNA resulting from the splicing of the target pre-mRNA to which the AC hybridizes. Re-spliced target protein may be identical to a wild type target protein, may be homologous to a wild type target protein, may be a functional variant of a wild type target protein, may be an isoform of a wild type target protein, or may be an active fragment of a wild type target protein.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

### Example 1. Construction of a Cell-Penetrating Peptide-Antisense Compound Conjugate

An antisense compound (AC) of anyone of SEQ ID NOs: 157-161 or SEQ ID NOs: 163-369 is designed to bind to and downregulate IRF-5 mRNA expression of is constructed as a phosphorodiamidate morpholino oligomer (PMO) with a C6-thiol 5′ modification.

An EEV may include a CCP or a CCP and an exocyclic peptide. A cell-penetrating peptide is formulated using Fmoc chemistry and conjugated to the AC, for example, as described in International Application No. PCT/US20/66459, filed by Entrada Therapeutics, Inc., on Dec. 21, 2021, entitled “COMPOSITIONS FOR DELIVERY OF ANTISENSE COMPOUNDS,” the disclosure of which is hereby incorporated in its entirety herein. In embodiments, the cCPP includes the amino acid sequence FfΦRrRrQ (SEQ ID NO:78). In embodiments EEV includes an exocyclic peptide having the sequence KKKRKV (SEQ ID NO:33). In embodiments, the EEV includes KKKRKV-PEG₂-K-(cyclo(FfΦRrRrQ))-PEG₁₂-K(N₃) (SEQ ID NO:33-PEG₂-K-(cyclo(SEQ ID NO:78))-PEG₁₂-K(N₃)). In embodiments, the AC compound is conjugated to the EEV using click chemistry. In embodiments, the compound includes KKKRKV-PEG₂-K-(cyclo(FfΦRrRrQ))-PEG₁₂-K-linker-3′-AC-5′ (SEQ ID NO:33-PEG₂-K-(cyclo(SEQ ID NO:78))-PEG₁-K-linker-3′-AC-5′) where the linker includes the product of a strain promoted click reaction between an azide and a cyclooctyne. The linker may also include other groups such as a carbon chain, PEG chain, carbamate, urea, and the like.

### Example 2. In Vivo Biodistribution of PMOs and EEV-PMO Constructs

The biodistribution of three compounds. 1) PMO, 2) cCCP, and 3) EEV-PMO conjugate was determined using an IVIS whole body imaging system.

The PMO for the compound 1 and compound 3 was GCTATTACCTTAACCCA (SEQ ID NO: 371). Compound 1 had the structure shown below and was conjugated to a fluorescent moiety. Compound 2 was cyclo[FfΦRrRrQ]-PEG₄ (cycloSEQ ID NO: 78-PEG₄) conjugated to a fluorescent moiety. Compound 3 was the product of AcPKKKRKVK-(cyclo[FfΦRrRrQ]-PEG₁₂)-K(N₃) (Ac-SEQ ID NO:42-(cyclo[SEQ ID NO:78]-PEG₁₂)-K(N₃)) with compound 1 via strain promoted click chemistry. Compound 3 also included a fluorescent moiety.

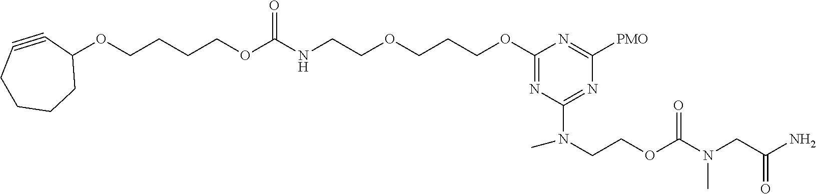

Briefly, BL/10-MDX mice were treated with PMO, PMO-EEV and CPP12-PEG4-dK (LSR) as shown in the table below. 0.9% saline was used as a control. Blood was collected at 1 hour, 6 hour and 24 hours. IVIS body imaging was followed by terminal sac.

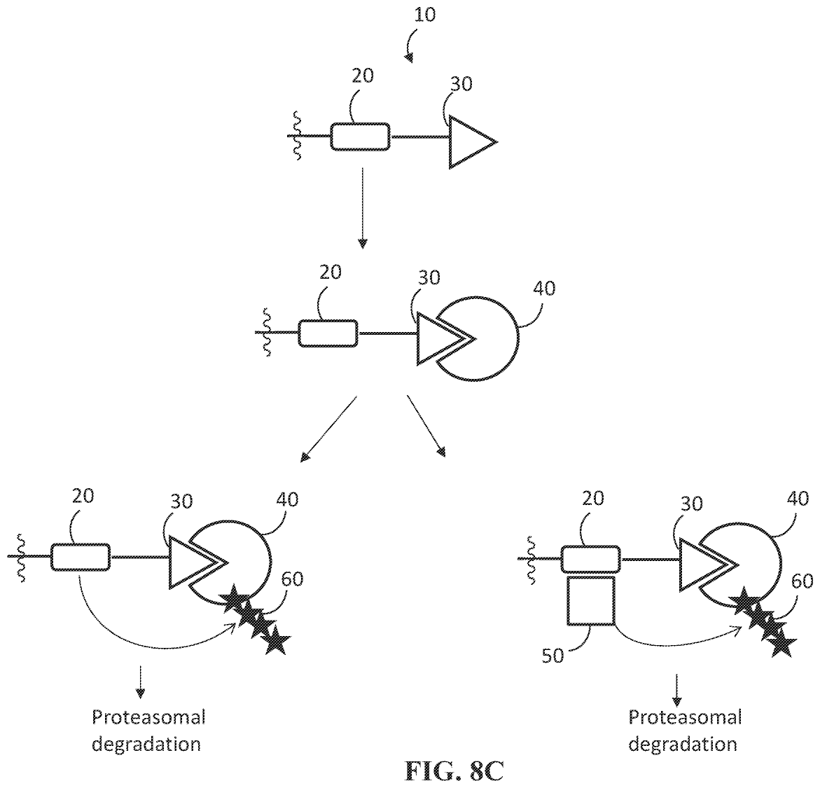

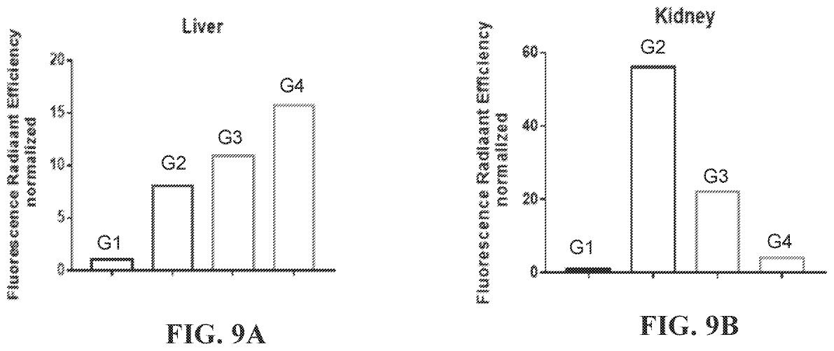

The in vivo mouse biodistribution for liver and kidney (24 hours post dosing) is shown in FIGS. 9A-B where G1 is the vehicle; G2 is compound 1; G3 is compound 3; and G4 is compound 2. Compound 2 (G4) showed the highest uptake signal in the liver compared to compounds 1 and 3 (FIG. 9A). Compound 1 (G2) showed the highest uptake signal in the kidney compared to compounds 2 and 3 (FIG. 9A).

### Example 3. Sequence Evaluation of PMOs Targeting IRF-5 PAS

Various PMOs were evaluated for targeting the IRF-5 polyadenylation signal. The PMOs tested included SEQ ID NO:258 (PAS-1), SEQ ID NO:259 (PAS-2), SEQ ID NO:260 (PAS-3), SEQ ID NO:261 (PAS-4), SEQ ID NO:262 (PAS-1b), SEQ ID NO:263 (PAS-2b), SEQ ID NO: 264 (PAS-3b), and SEQ ID NO:265 (PAS-4b). The PMOs targeted SEQ ID NOS: 361-368 shown in Table 2.

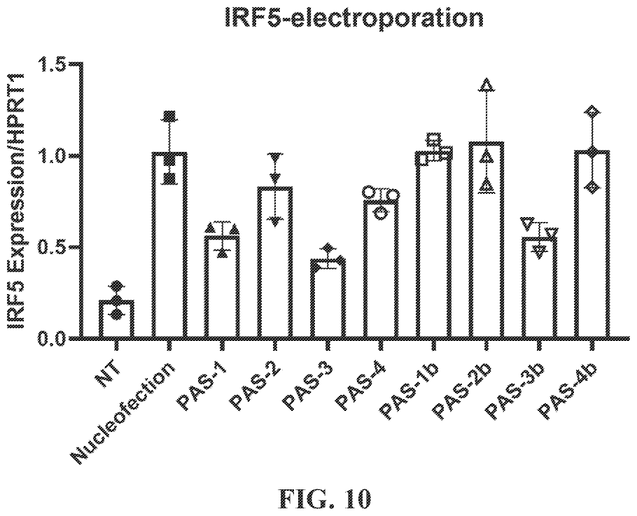

THP1 cells are monocytes isolated from a human acute monocytic leukemia patient and are available from ATCC as TIB-202™ cells. THP1 cells are often used to study monocyte and macrophage functions and mechanisms. THP1 cells were transfected with 5 μM PMO by nucleofection. Various PMOs demonstrated reduced IRF-5 expression (FIG. 10) indicating that targeting IRF-5 polyadenylation site is a viable strategy for downregulating gene expression.

### Example 4: Evaluation of In Vitro Exon Skipping of Various EEV-PMOs

Unstimulated RAW 264.7 monocyte/macrophage cells were used to evaluate IRF-5 expression and exon skipping after treatment with two EEV-PMO compounds 277-1120 and 278-1120. PMO-EEV 277-1120 is PMO sequence ACG TAA TCA TCA GTG GGT TGG CTC T (SEQ ID NO:158) conjugated to EEV 1120 Ac—PKKKRKV-AEEA-Lys-(cyclo[FGFGRGRQ])-PEG₁₂-OH (Ac-SEQ ID NO:42-AEEA-Lys-cyclo(SEQ ID NO:82)-PEG₁₂-OH) through amide conjugation chemistry. PMO-EEV 278-1120 is PMO sequence AGA ACG TAA TCA TCA GTG GGT TGG C (SEQ ID NO:157) conjugated to EEV 1120 Ac—PKKKRKV-AEEA-Lys-(cyclo[FGFGRGRQ])-PEG12-OH (Ac-SEQ ID NO:42-AEEA-Lys-cyclo(SEQ ID NO:82)-PEG₁₂-OH) through amide conjugation chemistry. Briefly, 150K cells/well were seeded in a 24 well plate in 0.5 ml DMEM. After 4 hours, the EEV-PMO compounds were added to the cells giving a total volume of 500 μL. The cells were then incubated for 24 hours. Following incubation, the cell culture media was collected for cytokines, IL6, and TNF-α detection. The RNA was extracted and used for IRF-5 transcript quantification. The protein lysates were used to measure IRF-5 protein level changes. IRF-5 expression levels were determined relative to β-tubulin.

For the exon skipping study, the cells were treated as described above. After incubation with the EEV-PMO compounds, the cells were washed with fresh media then incubated overnight. Following the second incubation, the RNA was harvested and RT-PCR was done using primers that detect exon 5 skipping in the IRF-5 gene.

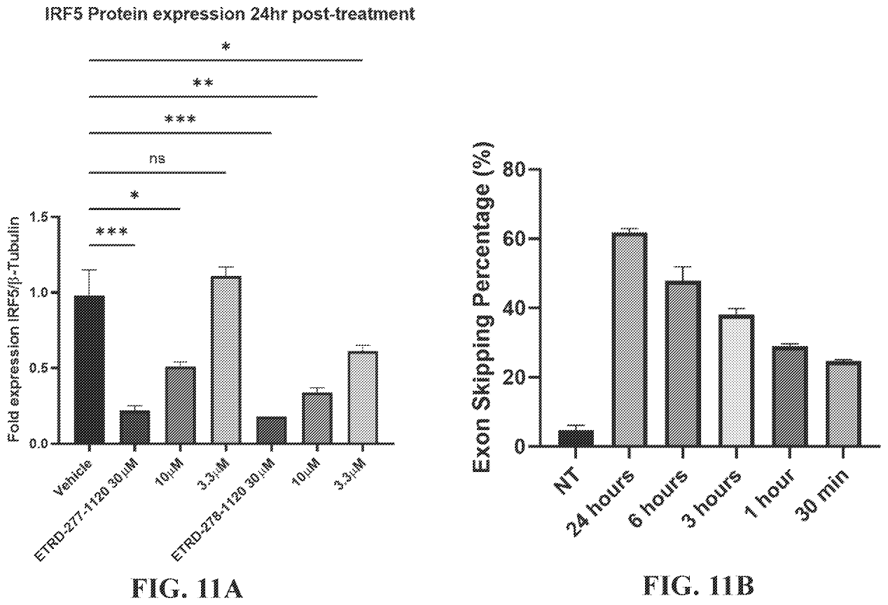

Both 277-1120 and 278-1120 showed target engagement in the RAW 264.7 mouse macrophages/monocytes and significantly reduced IRF-5 protein levels in a dose dependent fashion (FIG. 11A). Compound 277-1120 significantly depleted IRF-5 protein levels by ˜80% at 30 μM, ˜50% at 10 μM, and no substantial changes were observed with lower dosage of 3 3 μM. Compound 278-1120 had stronger effect on IRF-5 depletion than 277-1120. Compound 278-1120 reduced IRF-5 protein levels by ˜80% at 30 μM and ˜65% at 10 μM. Even at lower dosage of 3.3 μM, 278-1120, had an IRF-5 protein depletion of level of ˜40%.

The EEV-PMO compound 0278-1120 induced partial exon skipping as soon as 30 min after exposure with efficacy increasing as exposure time increases (FIG. 11B).

A similar experiment was conducted was additional EEV-PMO compounds where the PMO was PMO 278 (AGA ACG TAA TCA TCA GTG GGT TGG C; SEQ ID NO:157). The PMO 278 was conjugated to EEV various EEVs including Ac—PKKKRKV-PEG₂-K(cyclo[FGFGRGRQ])-PEG₁₂-OH (EEV #1, 1120, Ac-SEQ ID NO:42-PEG₂-K(cyclo[SEQ ID NO: 82])-PEG₁₂-OH); Ac—PKKKRKV-PEG₂-K(cyclo[Ff-Nal-GrGrQ])-PEG₁₂-OH (EEV #2, 1113, Ac-SEQ ID NO:42-PEG₂-K(cyclo[SEQ ID NO: 135])-PEG₁₂-OH); Ac—PKKKRKV-PEG₂-K(cyclo[FGFGRRRQ])-PEG₁₂-OH (EEV #3; 1184, Ac-SEQ ID NO:42-PEG₂-K(cyclo[SEQ ID NO: 84])-PEG₁₂-OH); and Ac—PKKKRKV-PEG₂-K(cyclo[FGFRRRRQ])-PEG₁₂-OH (EEV #4, 1185, Ac-SEQ ID NO:42-PEG₂-K(cyclo[SEQ ID NO:85])-PEG₁₂-OH) using amide conjugation chemistry.

Similar methods were used as described above except that the cells were pre-treated with the EEV-PMO compound followed by stimulation with R848 overnight. R484 is a Toll-like receptor agonist and leads to the induction of IRF-5 expression. The total treatment time was 24 hours.

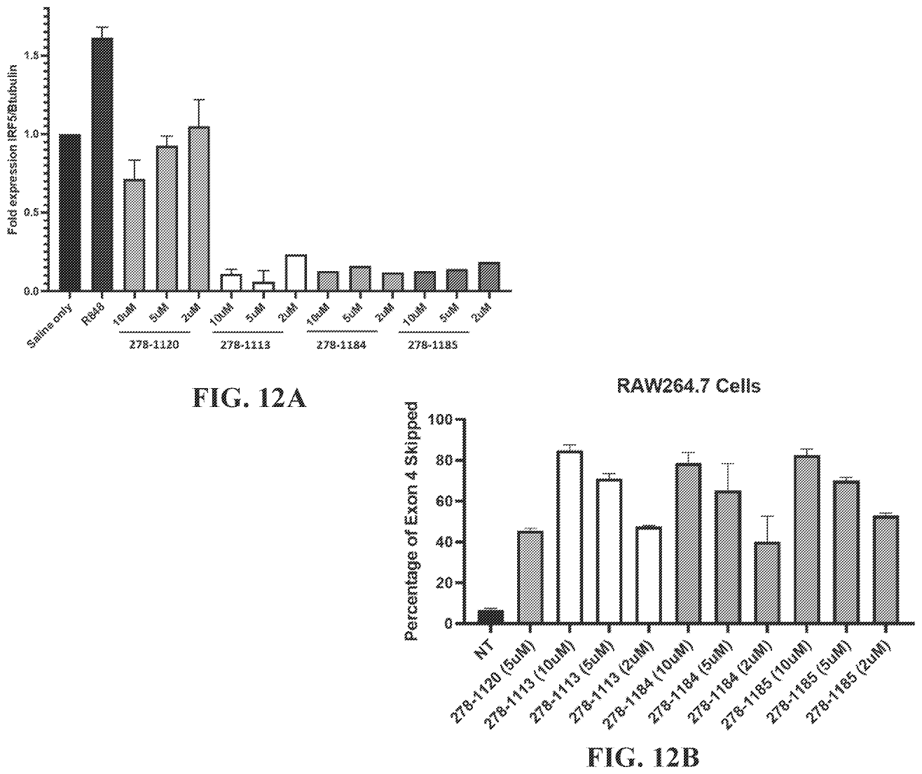

R848 significantly increases IRF-5 Protein expression in RAW264.7 cells. All EEV-PMO treated samples at all the tested concentrations showed a significant reduction in IRF-5 protein expression when compared to cells stimulated with R848 (FIG. 12A). EEV-PMO compounds 278-1113, 278-1184, and 278-1185 were on average 5-fold more efficacious than 278-1120 with about 80% IRF-5 protein reduction at concentrations as low as 2 μM when compared to IRF-5 levels in cells stimulation with R848

EEV-PMO compounds 278-1113, 278-1184, and 278-1185 exhibited higher exon skipping at 5 μM than 278-1120 (FIG. 12B). No substantial difference in exon skipping was observed between 278-1113, 278-1184, and 278-1185.

### Example 5: Evaluation of Various EEV-PMO Compounds in Human THP1 Cells

Human THP1 cells were used to evaluate IRF-5 expression and exon skipping after treatment with various PMO compounds and various EEV-PMO compounds. The PMO compounds tested include 344 (TTGGCAACATCCTCTGCAGCTGAAG; SEQ ID NO:159, Hs-IRF-5-E4N6); 345 (GCAACATCCTCTGCAGCTG; SEQ ID NO:160, Hs-IRF-5-E4N₃); 346 (TCAGGCTTGGCAACATCCTCTGCAG; SEQ ID NO:161, Hs-IRF-5-E5P0; IRF5-E4N₃ (TAATCATCAGTGGGTTGGCTCTCTG, SEQ ID NO: 369); 278 (AGA ACG TAA TCA TCA GTG GGT TGG C; SEQ ID NO: 157, Hs-IRF-5-E4P3), and 277 (ACG TAA TCA TCA GTG GGT TGG CTC T; SEQ ID NO:158, Hs-IRF-5-E4PO). The EEV-PMO compounds included PMOs 344, 345, and 346 were individually conjugated via amide conjugation chemistry to EEV 1120 (Ac—PKKKRKV-AEEA-Lys-(cyclo[FGFGRGRQ])-PEG₁₂-OH) (Ac-SEQ ID NO:42-AEEA-Lys-cyclo(SEQ ID NO:82)-PEG₁₂-OH).

Briefly, for the PMO only study, the nucleofection method was used to transfect PMO compounds into the THP1 cells. Cells were plated after nucleofection in PMA containing media and incubated for 24 hours before harvest. The RNA was harvested, and RT-PCR was done using primers that detect both exon 4 and exon 5 skipping in the IRF-5 gene.

Briefly, for the EEV-PMO study, the THP1 cells were differentiated by PMA overnight. The cells were then treated with various EEV-PMO conjugates and incubated for 24 hours before harvest. The RNA was harvested, and RT-PCR was done using primers that detect exon 5 skipping in the IRF-5 gene.

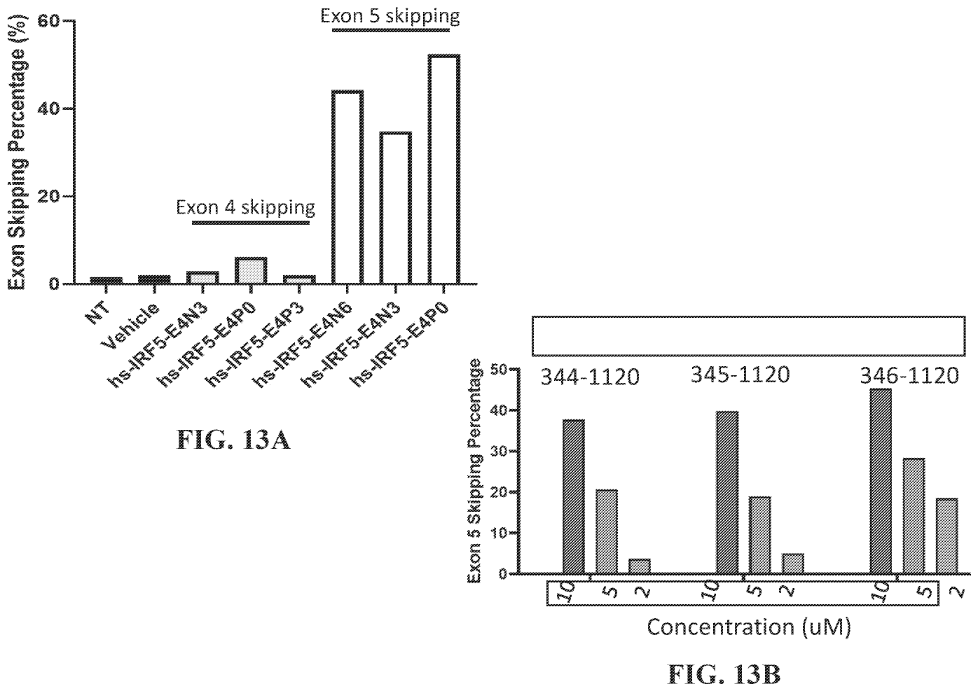

FIG. 13A shows the exon 4 and exon 5 skipping levels after treatment with various PMO compounds. The PMO compounds that worked well in mouse cells do not necessarily translate to human cells. For example, low levels of exon skipping were observed for Hs-IRF-5-E4P3 (PMO 278) and Hs-IRF-5-E4PO (PMO 277). Exon skipping was observed for the Hs-IRF-5-E5N₆ (PMO 344), Hs-IRF-5-ESN3 (PMO 345), and Hs-IRF-5-ESP0 (PMO 346).

FIG. 13B shows the exon 5 skipping levels after treatment with various PMO-EEV compounds. The results indicate that EEV-PMO conjugates can induce exon skipping and downregulation of target gene in THP1 cells.

### Example 6: IFR-5 Ablation Using Two Doses of EEV-PMO Mouse Study

A two-dose mouse study was used to study the effectiveness of PM-EEV 278-1120. Mice were dosed with either 40 milligrams per kilogram (mpk) or 20 mpk of PMO 278 (AGA ACG TAA TCA TCA GTG GGT TGG C; SEQ ID NO: 157) or EEV-PMO compound 278-1120 at day zero and again at day three. PMO-EEV 278-1120 includes PMO 278 conjugated to EEV 1120 (Ac—PKKKRKV-PEG₂-K(cyclo[FGFGRGRQ])-PEG₁₂-OH) (Ac-SEQ ID NO:42-PEG₂-K(cyclo[SEQ ID NO: 82])-PEG₁₂-OH). Five days following the second dose, the mice were sacrificed, and the blood and tissue collected.

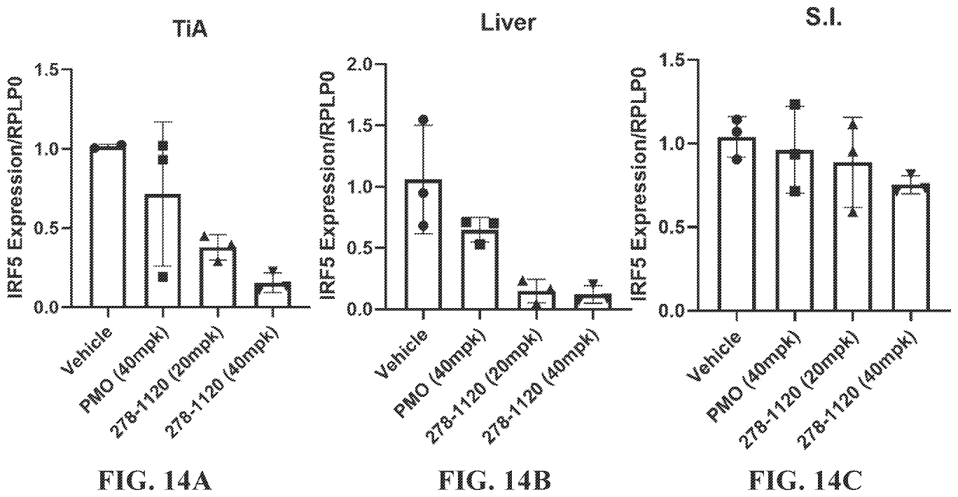

FIG. 14A-C shows the IRF-5 expression levels in various tissues after treatment. IRF-5 expression knockdown was observed in mouse TiA and liver tissues, but not in the small intestine tissue.

### Example 7: IFR-5 Ablation Using a Single Dose of EEV-PMO Mouse Study

A single dose mouse study was used to study the effectiveness of PM-EEV 278-1120. PMO-EEV 278-1120 is PMO 278 (AGA ACG TAA TCA TCA GTG GGT TGG C; SEQ ID NO: 157) conjugated to EEV 1120 (Ac—PKKKRKV-PEG₂-K(cyclo[FGFGRGRQ])-PEG₁₂-OH) (Ac-SEQ ID NO:42-PEG₂-K(cyclo[SEQ ID NO:82])-PEG₁₂-OH). At day zero, mice were dosed with 80 milligrams per kilogram (mpk) PMO 278; 40 mpk or 80 mpk of PMO-EEV 278-1120 via IV; or 120 mpk PMO-EEV 278-1120 subcutaneously (SC). Seven days following the second dose, the mice were sacrificed, and the blood and tissue collected.

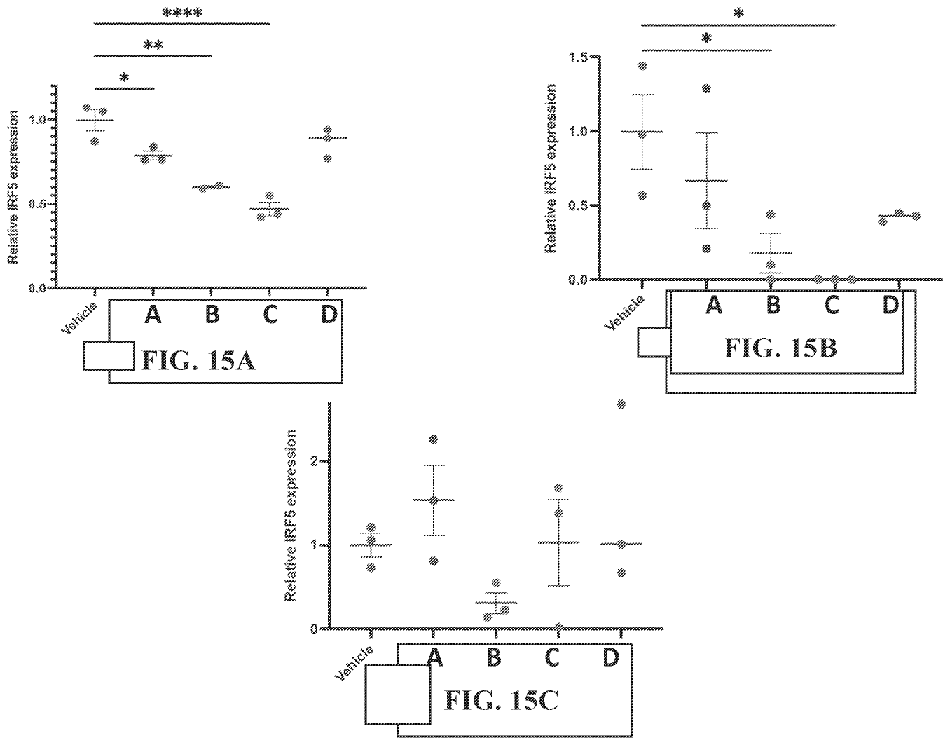

FIG. 15 shows IRF-5 expression levels in the liver (A), kidney (B), and tibialis anterior (C) tissues where 80 mpk PMO 278; A is 80 mpk PMO, B is 40 mpk PMO-EEV 278-1120 delivered via IV; C is 80 mpk PMO-EEV 278-1120 delivered via IV; and D is 120 mpk PMO-EEV 278-1120 delivered subcutaneously. There was a significant decrease in IRF-5 protein expression in the liver tissue of mice with a single dose administration of 278-1120 at both 40 and 80 mpk, corresponding to a 40% and 53% reduction, respectively (A).

The IRF-5 levels in the kidney tissue were low compared to other tissues examined. The data shows variability, likely due to difficulty quantifying band intensity vs background. Additionally, variability in the tibialis anterior tissue data was observed due to samples that did not run well on the gel (data not shown). Overall, the data shows a similar trend in the kidney as in the liver; there is a significant reduction in IRF-5 protein levels with a single dose administration of 278-1120 at both 40 and 80 mpk in mice.

### Example 8. Knockdown of IRF5 Expression Via Exon Skipping In Vitro and In Vivo Study

Four EEV-PMO conjugates were used to induce exon skipping of exon 4 to introduce a premature termination codon resulting in nonsense mediated decay of the IRF-5 target transcript. The PMO sequence for each of the four conjugates was 5′-AGA ACG TAA TCA TCA GTG GGT TGG C-3′ (SEQ ID NO:157). The EEVs used were Ac—PKKKRKV-PEG₂-K(cyclo[FGFGRGRQ])-PEG₁₂-OH (EEV #1, 1120, Ac-SEQ ID NO:42-PEG₂-K(cyclo[SEQ ID NO: 82])-PEG₁₂-OH); Ac—PKKKRKV-PEG₂-K(cyclo[Ff-Nal-GrGrQ])-PEG₁₂-OH (EEV #2, 1113, Ac-SEQ ID NO:42-PEG₂-K(cyclo[SEQ ID NO: 135])-PEG₁₂-OH); Ac—PKKKRKV-PEG₂-K(cyclo[FGFGRRRQ])-PEG₁₂-OH (EEV #4; 1184, Ac-SEQ ID NO:42-PEG₂-K(cyclo[SEQ ID NO: 84])-PEG₁₂-OH); and 1185: Ac—PKKKRKV-PEG₂-K(cyclo[FGFRRRRQ])-PEG₁₂-OH (EEV #4, 1185, Ac-SEQ ID NO:42-PEG₂-K(cyclo[SEQ ID NO:85])-PEG₁₂-OH). EEVs were conjugated to the PMOs using amide conjugation chemistry.

Wild type mice were treated with two doses of EEV #1-PMO on days 0 and 3. Samples were collected on day 7 for qPCR to measure mRNA levels. For the in vitro studies, mouse macrophage cells treated with the EEV #1-PMO or were pre-treated with 2 μM of EEV-PMOs #1-4 for 4 hours, followed by stimulation with R848, an imidazoquinolinone compound that is a specific activator of toll-like receptor (TLR) 7/8, overnight. At 24 hours post treatment, cells were harvested and evaluated by Western Blot.

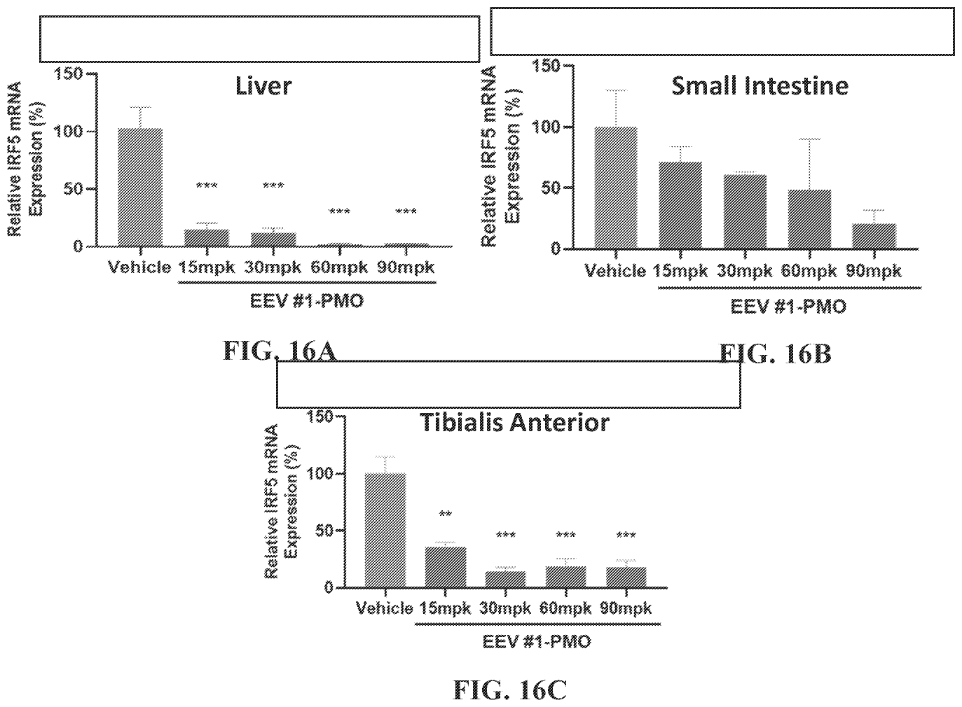

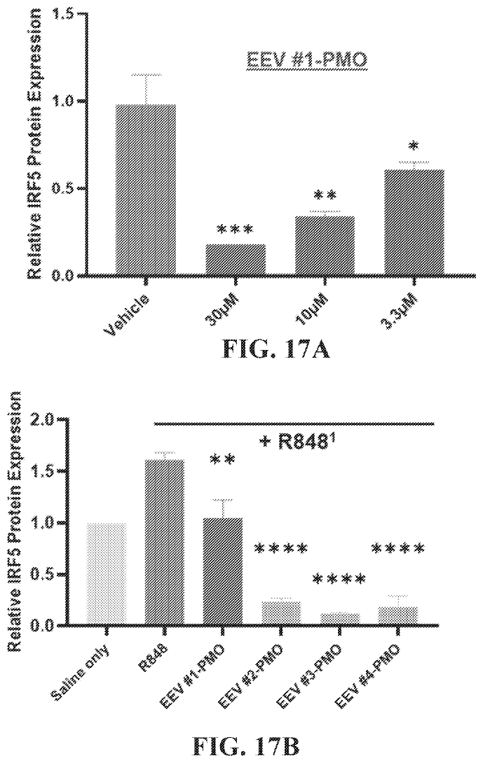

A significant knockdown of IRF5 levels was observed in the liver (FIG. 16A), small intestine (FIG. 16B), and tibias anterior (FIG. 16C) after treatment with EEV #1-PMO. In all tissues the knockdown was dose dependent. Additionally, mouse macrophage cells treated with the EEV #1-PMO had a statistically significant reduction of IRF5 protein levels at doses of 30 μM, 10 μM and 3 μM (FIG. 17A). However, mouse macrophage cells pretreated with EEV #2-PMO, EEV #3-PMO, and EEV #4-PMO followed by stimulation with R848, had significant improvement in relative potency when compared to EEV #1-PMO, as measured by IRF-5 protein expression (FIG. 17B). mRNA levels are relative to the vehicle control which was set to 100%.

A number of embodiments have been described herein. Nevertheless, it will be understood that various modifications may be made without departing from the spirit and scope of the invention. Accordingly, other embodiments are within the scope of the following claims.

1-129. (canceled)

130. A compound comprising:
(a) a cyclic cell penetrating peptide of the formula: 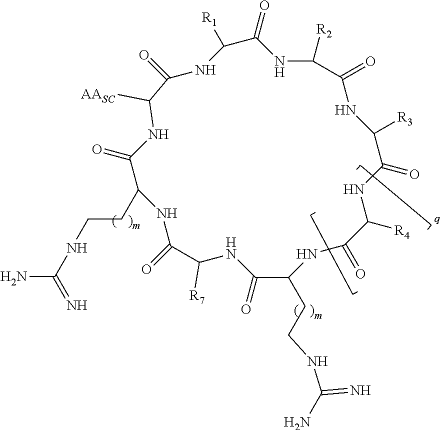
or a protonated form or salt thereof, wherein:
R₁, R₂, and R₃ are each independently H or an aromatic or heteroaromatic side chain of an amino acid;
at least two of R₁, R₂, and R₃ are the side chain of phenylalanine;
R₄ and R₇ are independently H or an amino acid side chain;
each m is independently an integer from 0 to 3;
AA_{SC} is an amino acid side chain; and
q is 1, 2, 3, or 4;
(b) an exocyclic peptide comprising from 2 to 10 amino acid residues, wherein 2, 3, or 4 of the residues are lysine residues;
(c) a linker of formula: 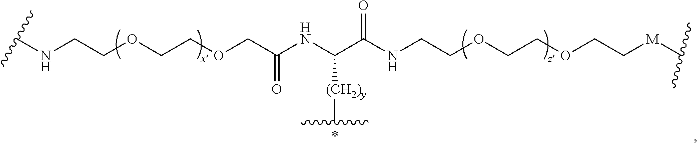
wherein:
x′ is an integer from 1-23;
y is an integer from 1-5;
z′ is an integer from 1-23;
* is the point of attachment to the AA_{SC} of the cyclic peptide;
and M is a bonding group;
(d) an oligonucleotide that modulates Interferon Regulatory Factor-5 (IRF-5) activity, the oligonucleotide comprising an antisense compound complementary to a target nucleotide sequence encoding IRF-5.

131. The compound of claim 130, wherein the exocyclic peptide comprises at least 2 amino acid residues with a hydrophobic side chain.

132. The compound of claim 130, wherein the exocyclic peptide comprises PKKKRKV.

133. The compound of claim 130, wherein z′ is 11.

134. The compound of claim 130, wherein x′ is 1.

135. The compound of claim 130, wherein M comprises

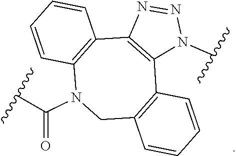 [SMILES: *C(=O)N1Cc2ccccc2-c2c(nnn2*)-c2ccccc21]

136. The compound of claim 130, wherein M comprises

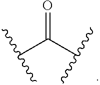 [SMILES: *C(*)=O]

137. The compound of claim 130, wherein the cyclic cell penetrating peptide is selected from: 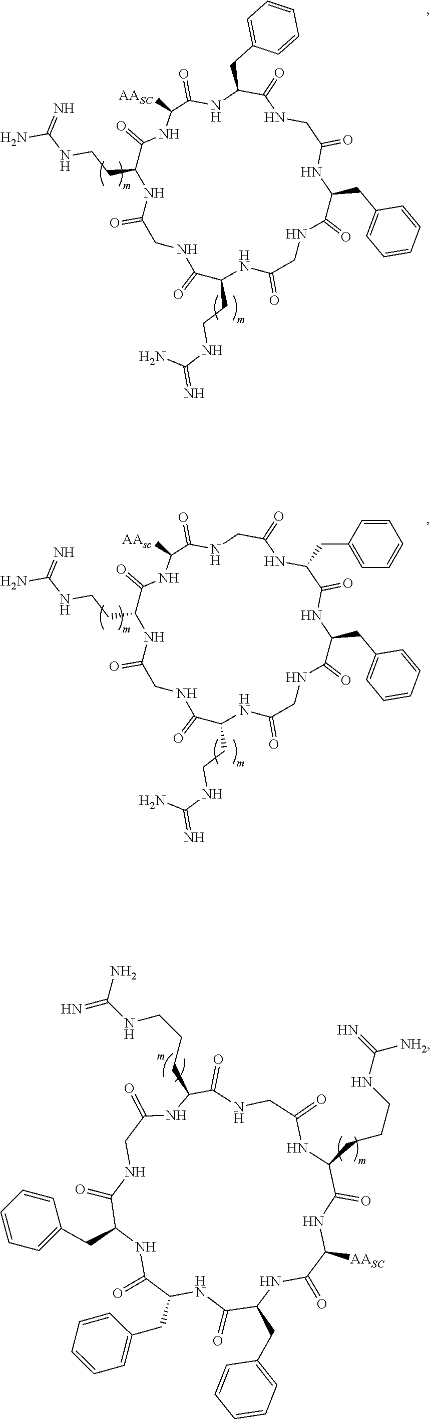 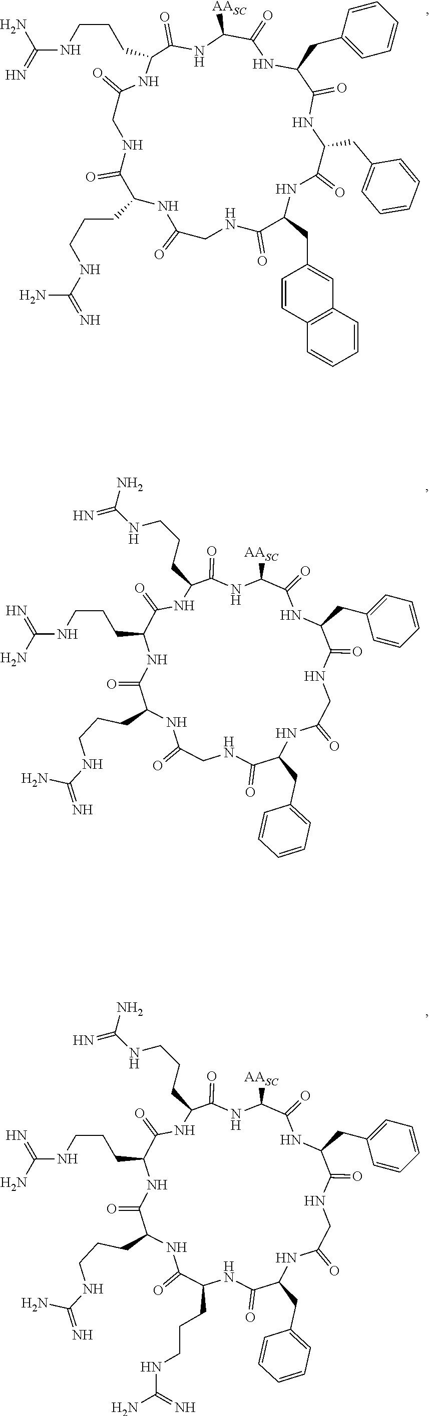 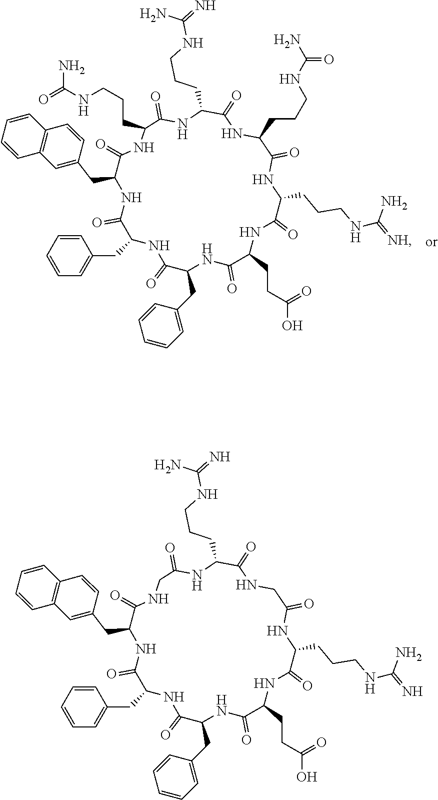 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]
or a protonated form or salt thereof, wherein if the structure does not include AA_{sc} then at least one atom of an amino acid side chain is replaced by the linker or at least one lone pair forms a bond to the linker.

138. The compound of claim 130, comprising an oligonucleotide conjugated to an endosomal escape vehicle with a sequence selected from:
Ac—PKKKRKV-PEG₂-K(cyclo[Ff-Nal-Cit-r-Cit-r-Q])-PEG₁₂-lys(N₃)—NH₂;
Ac—PKKKRKV—K(cyclo[Ff-Nal-GrGrQ])-PEG₁₂-Lys(N₃)—NH₂;
Ac—PKKKRKV-miniPEG₂-Lys(cyclo(FfFGRGRQ)-PEG₂-K(N₃);
Ac—PKKKRKV-PEG₂-K(cyclo[FGFGRGRQ])-PEG₂-Lys(N₃)—NH₂;
Ac—PKKKRKV-PEG₂-K(cyclo[GfFGrGrQ])-PEG₂-Lys(N₃)—NH₂;
Ac—PKKKRKV-PEG₂-Lys(cyclo[FfFGRGRQ)-miniPEG₂-K(N₃)—NH₂;
Ac—PKKKRKV-PEG₂-K(cyclo(Ff-Nal-GrGrQ)-PEG₁₂-OH; Ac—PKKKRKV-PEG₂-K(cyclo[FGFGRGRQ])-PEG₁₂-OH;
Ac—PKKKRKV-PEG₂-K(cyclo[GfFGrGrQ])-PEG₁₂-OH;
Ac—PKKKRKV-PEG₂-K(cyclo[FGFGRRRQ])-PEG₁₂-OH; and
Ac—PKKKRKV-PEG₂-K(cyclo[FGFRRRRQ])-PEG₁₂-OH.

139. The compound of claim 130, wherein the oligonucleotide comprises a phosphorodiamidate morpholino nucleotide.

140. The compound of claim 130, wherein the oligonucleotide comprises from 10 to 50 nucleotides in length.

141. The compound of claim 130, wherein the oligonucleotide decreases IRF-5 expression.

142. The compound of claim 130, wherein the oligonucleotide induces exon skipping to produce a premature termination codon in a target IRF-5 transcript.

143. The compound of claim 130, wherein the oligonucleotide modulates polyadenylation of a target IRF-5 transcript.

144. The compound of claim 130, wherein the oligonucleotide comprises SEQ ID: 157; SEQ ID NO:158, SEQ ID NO:159, SEQ ID NO: 160, or SEQ ID NO:161.

145. The compound of claim 130, wherein the oligonucleotide comprises any one of SEQ ID NO: 163 through SEQ ID NO:265 or SEQ ID NO:369.

146. The compound of claim 130, wherein modulates Interferon Regulatory Factor-5 (IRF-5) activity in an immune cell comprising a macrophage, a resident tissue macrophage, or a monocyte-derived macrophage; the macrophage, the resident tissue macrophage, or the monocyte-derived macrophage comprising a Kupffer cell, an intraglomular mesangial cell, an alveolar macrophage, a sinus histiocyte, a hofbauer cell, microglia or langerhan cell.

147. A pharmaceutical composition comprising the compound of claim 130 and a pharmaceutically acceptable carrier.

148. A method of treating a disease or disorder associated with Interferon Regulatory Factor-5 (IRF-5) in a patient, comprising administering to the patient a therapeutically effective amount of the compound of claim 130 to the patient.

149. The method of claim 148, wherein the disease or disorder comprises an autoimmune disorder, an inflammatory disease, a neuroinflammatory disease, cardiovascular disease, or a fibrotic condition.

150. The method of claim 149, where the disease comprises systemic lupus erythematosus (SLE), systemic sclerosis (scleroderma), polymyositis/dermatomyositis, Crohn's disease, ulcerative colitis, rheumatoid arthritis, Sjogren's syndrome, autoimmune encephalomyelitis, nonalcoholic steatohepatitis (NASH), sarcoidosis, Behcet's disease, myasthenia gravis, lupus nephritis, inflammatory bowel disease (IBD), ankylosing spondylitis, primary biliary cirrhosis, colitis, pulmonary fibrosis, antiphospholipid syndrome, psoriasis, liver fibrosis, atherosclerosis, Alzheimer's disease, or non-alcoholic steatohepatitis.


SEQUENCE LISTING (379 sequences)

SEQ ID NO: 1 - protein, 4 aa, Artificial Sequence
  Description of Artificial Sequence: Synthetic peptide
HHHH

SEQ ID NO: 2 - protein, 4 aa, Artificial Sequence
  Description of Artificial Sequence: Synthetic peptide
KHKK

SEQ ID NO: 3 - protein, 4 aa, Artificial Sequence
  Description of Artificial Sequence: Synthetic peptide
KKHK

SEQ ID NO: 4 - protein, 4 aa, Artificial Sequence
  Description of Artificial Sequence: Synthetic peptide
KKKH

SEQ ID NO: 5 - protein, 4 aa, Artificial Sequence
  Description of Artificial Sequence: Synthetic peptide
KHKH

SEQ ID NO: 6 - protein, 4 aa, Artificial Sequence
  Description of Artificial Sequence: Synthetic peptide
HKHK

SEQ ID NO: 7 - protein, 4 aa, Artificial Sequence
  Description of Artificial Sequence: Synthetic peptide
KKKK

SEQ ID NO: 8 - protein, 4 aa, Artificial Sequence
  Description of Artificial Sequence: Synthetic peptide
KKRK

SEQ ID NO: 9 - protein, 4 aa, Artificial Sequence
  Description of Artificial Sequence: Synthetic peptide
KRKK

SEQ ID NO: 10 - protein, 4 aa, Artificial Sequence
  Description of Artificial Sequence: Synthetic peptide
KRRK

SEQ ID NO: 11 - protein, 4 aa, Artificial Sequence
  Description of Artificial Sequence: Synthetic peptide
RKKR

SEQ ID NO: 12 - protein, 4 aa, Artificial Sequence
  Description of Artificial Sequence: Synthetic peptide
RRRR

SEQ ID NO: 13 - protein, 4 aa, Artificial Sequence
  Description of Artificial Sequence: Synthetic peptide
KGKK

SEQ ID NO: 14 - protein, 4 aa, Artificial Sequence
  Description of Artificial Sequence: Synthetic peptide
KKGK

SEQ ID NO: 15 - protein, 5 aa, Artificial Sequence
  Description of Artificial Sequence: Synthetic peptide
  MOD_RES (2)..(2): Beta-alanine
  MOD_RES (4)..(4): Beta-alanine
HAHAH

SEQ ID NO: 16 - protein, 5 aa, Artificial Sequence
  Description of Artificial Sequence: Synthetic peptide
  MOD_RES (2)..(2): Beta-alanine
  MOD_RES (4)..(4): Beta-alanine
HAKAH

SEQ ID NO: 17 - protein, 5 aa, Artificial Sequence
  Description of Artificial Sequence: Synthetic peptide
RRRRR

SEQ ID NO: 18 - protein, 5 aa, Artificial Sequence
  Description of Artificial Sequence: Synthetic peptide
KKKKK

SEQ ID NO: 19 - protein, 5 aa, Artificial Sequence
  Description of Artificial Sequence: Synthetic peptide
KKKRK

SEQ ID NO: 20 - protein, 5 aa, Artificial Sequence
  Description of Artificial Sequence: Synthetic peptide
RKKKK

SEQ ID NO: 21 - protein, 5 aa, Artificial Sequence
  Description of Artificial Sequence: Synthetic peptide
KRKKK

SEQ ID NO: 22 - protein, 5 aa, Artificial Sequence
  Description of Artificial Sequence: Synthetic peptide
KKRKK

SEQ ID NO: 23 - protein, 5 aa, Artificial Sequence
  Description of Artificial Sequence: Synthetic peptide
KKKKR

SEQ ID NO: 24 - protein, 5 aa, Artificial Sequence
  Description of Artificial Sequence: Synthetic peptide
  MOD_RES (2)..(2): Beta-alanine
  MOD_RES (4)..(4): Beta-alanine
KAKAK

SEQ ID NO: 25 - protein, 6 aa, Artificial Sequence
  Description of Artificial Sequence: Synthetic peptide
RKKKKG

SEQ ID NO: 26 - protein, 6 aa, Artificial Sequence
  Description of Artificial Sequence: Synthetic peptide
KRKKKG

SEQ ID NO: 27 - protein, 6 aa, Artificial Sequence
  Description of Artificial Sequence: Synthetic peptide
KKRKKG

SEQ ID NO: 28 - protein, 6 aa, Artificial Sequence
  Description of Artificial Sequence: Synthetic peptide
KKKKRG

SEQ ID NO: 29 - protein, 6 aa, Artificial Sequence
  Description of Artificial Sequence: Synthetic peptide
  MOD_RES (6)..(6): Beta-alanine
RKKKKA

SEQ ID NO: 30 - protein, 6 aa, Artificial Sequence
  Description of Artificial Sequence: Synthetic peptide
  MOD_RES (6)..(6): Beta-alanine
KRKKKA

SEQ ID NO: 31 - protein, 6 aa, Artificial Sequence
  Description of Artificial Sequence: Synthetic peptide
  MOD_RES (6)..(6): Beta-alanine
KKRKKA

SEQ ID NO: 32 - protein, 6 aa, Artificial Sequence
  Description of Artificial Sequence: Synthetic peptide
  MOD_RES (6)..(6): Beta-alanine
KKKKRA

SEQ ID NO: 33 - protein, 6 aa, Artificial Sequence
  Description of Artificial Sequence: Synthetic peptide
KKKRKV

SEQ ID NO: 34 - protein, 6 aa, Artificial Sequence
  Description of Artificial Sequence: Synthetic peptide
RRRRRR

SEQ ID NO: 35 - protein, 6 aa, Artificial Sequence
  Description of Artificial Sequence: Synthetic peptide
HHHHHH

SEQ ID NO: 36 - protein, 6 aa, Artificial Sequence
  Description of Artificial Sequence: Synthetic peptide
RHRHRH

SEQ ID NO: 37 - protein, 6 aa, Artificial Sequence
  Description of Artificial Sequence: Synthetic peptide
HRHRHR

SEQ ID NO: 38 - protein, 6 aa, Artificial Sequence
  Description of Artificial Sequence: Synthetic peptide
KRKRKR

SEQ ID NO: 39 - protein, 6 aa, Artificial Sequence
  Description of Artificial Sequence: Synthetic peptide
RKRKRK

SEQ ID NO: 40 - protein, 6 aa, Artificial Sequence
  Description of Artificial Sequence: Synthetic peptide
  MOD_RES (2)..(2): Beta-alanine
  MOD_RES (4)..(4): Beta-alanine
  MOD_RES (6)..(6): Beta-alanine
RARARA

SEQ ID NO: 41 - protein, 6 aa, Artificial Sequence
  Description of Artificial Sequence: Synthetic peptide
  MOD_RES (2)..(2): Beta-alanine
  MOD_RES (4)..(4): Beta-alanine
  MOD_RES (6)..(6): Beta-alanine
KAKAKA

SEQ ID NO: 42 - protein, 7 aa, Artificial Sequence
  Description of Artificial Sequence: Synthetic peptide
PKKKRKV

SEQ ID NO: 43 - protein, 7 aa, Artificial Sequence
  Description of Artificial Sequence: Synthetic peptide
PGKKRKV

SEQ ID NO: 44 - protein, 7 aa, Artificial Sequence
  Description of Artificial Sequence: Synthetic peptide
PKGKRKV

SEQ ID NO: 45 - protein, 7 aa, Artificial Sequence
  Description of Artificial Sequence: Synthetic peptide
PKKGRKV

SEQ ID NO: 46 - protein, 7 aa, Artificial Sequence
  Description of Artificial Sequence: Synthetic peptide
PKKKGKV

SEQ ID NO: 47 - protein, 7 aa, Artificial Sequence
  Description of Artificial Sequence: Synthetic peptide
PKKKRGV

SEQ ID NO: 48 - protein, 7 aa, Artificial Sequence
  Description of Artificial Sequence: Synthetic peptide
PKKKRKG

SEQ ID NO: 49 - protein, 5 aa, Artificial Sequence
  Description of Artificial Sequence: Synthetic peptide
  MOD_RES (2)..(2): Beta-alanine
  MOD_RES (4)..(4): Beta-alanine
RARAR

SEQ ID NO: 50 - protein, 5 aa, Artificial Sequence
  Description of Artificial Sequence: Synthetic peptide
  MOD_RES (2)..(2): Beta-alanine
  MOD_RES (4)..(4): Beta-alanine
RAHAR

SEQ ID NO: 51 - protein, 5 aa, Artificial Sequence
  Description of Artificial Sequence: Synthetic peptide
  MOD_RES (2)..(2): Beta-alanine
  MOD_RES (4)..(4): Beta-alanine
HARAH

SEQ ID NO: 52 - protein, 19 aa, Artificial Sequence
  Description of Artificial Sequence: Synthetic peptide
NLSKRPAAIKKAGQAKKKK

SEQ ID NO: 53 - protein, 9 aa, Artificial Sequence
  Description of Artificial Sequence: Synthetic peptide
PAAKRVKLD

SEQ ID NO: 54 - protein, 11 aa, Artificial Sequence
  Description of Artificial Sequence: Synthetic peptide
RQRRNELKRSF

SEQ ID NO: 55 - protein, 27 aa, Artificial Sequence
  Description of Artificial Sequence: Synthetic peptide
RMRKFKNKGKDTAELRRRRVEVSVELR

SEQ ID NO: 56 - protein, 14 aa, Artificial Sequence
  Description of Artificial Sequence: Synthetic peptide
KAKKDEQILKRRNV

SEQ ID NO: 57 - protein, 8 aa, Artificial Sequence
  Description of Artificial Sequence: Synthetic peptide
VSRKRPRP

SEQ ID NO: 58 - protein, 8 aa, Artificial Sequence
  Description of Artificial Sequence: Synthetic peptide
PPKKARED

SEQ ID NO: 59 - protein, 8 aa, Artificial Sequence
  Description of Artificial Sequence: Synthetic peptide
PQPKKKPL

SEQ ID NO: 60 - protein, 12 aa, Artificial Sequence
  Description of Artificial Sequence: Synthetic peptide
SALIKKKKKMAP

SEQ ID NO: 61 - protein, 5 aa, Artificial Sequence
  Description of Artificial Sequence: Synthetic peptide
DRLRR

SEQ ID NO: 62 - protein, 7 aa, Artificial Sequence
  Description of Artificial Sequence: Synthetic peptide
PKQKKRK

SEQ ID NO: 63 - protein, 10 aa, Artificial Sequence
  Description of Artificial Sequence: Synthetic peptide
RKLKKKIKKL

SEQ ID NO: 64 - protein, 10 aa, Artificial Sequence
  Description of Artificial Sequence: Synthetic peptide
REKKKFLKRR

SEQ ID NO: 65 - protein, 20 aa, Artificial Sequence
  Description of Artificial Sequence: Synthetic peptide
KRKGDEVDGVDEVAKKKSKK

SEQ ID NO: 66 - protein, 17 aa, Artificial Sequence
  Description of Artificial Sequence: Synthetic peptide
RKCLQAGMNLEARKTKK

SEQ ID NO: 67 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 68 - protein, 7 aa, Artificial Sequence
  Description of Artificial Sequence: Synthetic peptide
FGFGRGR

SEQ ID NO: 69 - protein, 7 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGR

SEQ ID NO: 70 - protein, 7 aa, Artificial Sequence
  Description of Artificial Sequence: Synthetic peptide
  MOD_RES (2)..(2): D-phenylalanine
  MOD_RES (3)..(3): L-naphthylalanine
FFAGRGR

SEQ ID NO: 71 - protein, 7 aa, Artificial Sequence
  Description of Artificial Sequence: Synthetic peptide
  MOD_RES (2)..(2): D-phenylalanine
FFFGRGR

SEQ ID NO: 72 - protein, 7 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGR

SEQ ID NO: 73 - protein, 4 aa, Artificial Sequence
  Description of Artificial Sequence: Synthetic peptide
  MOD_RES (4)..(4): L-naphthylalanine
FGFA

SEQ ID NO: 74 - protein, 8 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGRQ

SEQ ID NO: 75 - protein, 8 aa, Artificial Sequence
  Description of Artificial Sequence: Synthetic peptide
  MOD_RES (2)..(2): D-phenylalanine
  MOD_RES (3)..(3): L-naphthylalanine
FFAGRGRQ

SEQ ID NO: 76 - protein, 8 aa, Artificial Sequence
  Description of Artificial Sequence: Synthetic peptide
  MOD_RES (2)..(2): D-phenylalanine
FFFGRGRQ

SEQ ID NO: 77 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGRQ

SEQ ID NO: 78 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 79 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (4)..(4): Citrulline
  MOD_RES (5)..(5): D-arginine
  MOD_RES (6)..(6): Citrulline
  MOD_RES (7)..(7): D-arginine
FFAXRXRQ

SEQ ID NO: 80 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFAGRGRQ

SEQ ID NO: 81 - protein, 8 aa, Artificial Sequence
  Description of Artificial Sequence: Synthetic peptide
  MOD_RES (2)..(2): D-phenylalanine
FFFGRGRQ

SEQ ID NO: 82 - protein, 8 aa, Artificial Sequence
  Description of Artificial Sequence: Synthetic peptide
FGFGRGRQ

SEQ ID NO: 83 - protein, 8 aa, Artificial Sequence
  Description of Artificial Sequence: Synthetic peptide
  MOD_RES (2)..(2): D-phenylalanine
  MOD_RES (5)..(5): D-arginine
  MOD_RES (7)..(7): D-arginine
GFFGRGRQ

SEQ ID NO: 84 - protein, 8 aa, Artificial Sequence
  Description of Artificial Sequence: Synthetic peptide
FGFGRRRQ

SEQ ID NO: 85 - protein, 8 aa, Artificial Sequence
  Description of Artificial Sequence: Synthetic peptide
FGFRRRRQ

SEQ ID NO: 86 - protein, 7 aa, Artificial Sequence
  Description of Artificial Sequence: Synthetic peptide
  MOD_RES (2)..(2): L-naphthylalanine
FARRRRQ

SEQ ID NO: 87 - protein, 7 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRQ

SEQ ID NO: 88 - protein, 8 aa, Artificial Sequence
  Description of Artificial Sequence: Synthetic peptide
  MOD_RES (2)..(2): D-phenylalanine
  MOD_RES (3)..(3): L-naphthylalanine
FFARGRGQ

SEQ ID NO: 89 - protein, 6 aa, Artificial Sequence
  Description of Artificial Sequence: Synthetic peptide
  MOD_RES (2)..(2): L-naphthylalanine
FARRRQ

SEQ ID NO: 90 - protein, 6 aa, Artificial Sequence
  Description of Artificial Sequence: Synthetic peptide
  MOD_RES (2)..(2): L-naphthylalanine
FARRRC

SEQ ID NO: 91 - protein, 6 aa, Artificial Sequence
  Description of Artificial Sequence: Synthetic peptide
  MOD_RES (2)..(2): L-naphthylalanine
  MOD_RES (6)..(6): Selenocysteine
FARRRX

SEQ ID NO: 92 - protein, 6 aa, Artificial Sequence
  Description of Artificial Sequence: Synthetic peptide
  MOD_RES (4)..(4): L-naphthylalanine
RRRAFQ

SEQ ID NO: 93 - protein, 6 aa, Artificial Sequence
  Description of Artificial Sequence: Synthetic peptide
  MOD_RES (5)..(5): L-naphthylalanine
RRRRAF

SEQ ID NO: 94 - protein, 6 aa, Artificial Sequence
  Description of Artificial Sequence: Synthetic peptide
  MOD_RES (2)..(2): L-naphthylalanine
FARRRR

SEQ ID NO: 95 - protein, 7 aa, Artificial Sequence
  Description of Artificial Sequence: Synthetic peptide
  MOD_RES (2)..(2): D-naphthylalanine
  MOD_RES (3)..(3): D-arginine
  MOD_RES (5)..(5): D-arginine
  MOD_RES (7)..(7): D-glutamine
FARRRRQ

SEQ ID NO: 96 - protein, 7 aa, Artificial Sequence
  Description of Artificial Sequence: Synthetic peptide
  MOD_RES (2)..(2): D-naphthylalanine
  MOD_RES (3)..(3): D-arginine
  MOD_RES (5)..(5): D-arginine
FARRRRQ

SEQ ID NO: 97 - protein, 7 aa, Artificial Sequence
  Description of Artificial Sequence: Synthetic peptide
  MOD_RES (2)..(2): L-naphthylalanine
FARRRRQ

SEQ ID NO: 98 - protein, 7 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRQ

SEQ ID NO: 99 - protein, 7 aa, Artificial Sequence
  Description of Artificial Sequence: Synthetic peptide
  MOD_RES (5)..(5): L-naphthylalanine
RRFRARQ

SEQ ID NO: 100 - protein, 7 aa, Artificial Sequence
  Description of Artificial Sequence: Synthetic peptide
  MOD_RES (6)..(6): L-naphthylalanine
FRRRRAQ

SEQ ID NO: 101 - protein, 7 aa, Artificial Sequence
  Description of Artificial Sequence: Synthetic peptide
  MOD_RES (1)..(1): D-arginine
  MOD_RES (5)..(5): L-naphthylalanine
RRFRARQ

SEQ ID NO: 102 - protein, 7 aa, Artificial Sequence
  Description of Artificial Sequence: Synthetic peptide
  MOD_RES (3)..(3): L-naphthylalanine
RRAFRRQ

SEQ ID NO: 103 - protein, 8 aa, Artificial Sequence
  Description of Artificial Sequence: Synthetic peptide
CRRRRFWQ

SEQ ID NO: 104 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 105 - protein, 8 aa, Artificial Sequence
  Description of Artificial Sequence: Synthetic peptide
  MOD_RES (3)..(3): L-naphthylalanine
FFARRRRQ

SEQ ID NO: 106 - protein, 8 aa, Artificial Sequence
  Description of Artificial Sequence: Synthetic peptide
  MOD_RES (6)..(6): L-naphthylalanine
RFRFRARQ

SEQ ID NO: 107 - protein, 8 aa, Artificial Sequence
  Description of Artificial Sequence: Synthetic peptide
  MOD_RES (1)..(1): Selenocysteine
XRRRRFWQ

SEQ ID NO: 108 - protein, 8 aa, Artificial Sequence
  Description of Artificial Sequence: Synthetic peptide
CRRRRFWQ

SEQ ID NO: 109 - protein, 8 aa, Artificial Sequence
  Description of Artificial Sequence: Synthetic peptide
  MOD_RES (2)..(2): L-naphthylalanine
FARRRRQK

SEQ ID NO: 110 - protein, 8 aa, Artificial Sequence
  Description of Artificial Sequence: Synthetic peptide
  MOD_RES (2)..(2): L-naphthylalanine
FARRRRQC

SEQ ID NO: 111 - protein, 8 aa, Artificial Sequence
  Description of Artificial Sequence: Synthetic peptide
  MOD_RES (1)..(1): D-phenylalanine
  MOD_RES (2)..(2): L-naphthylalanine
  MOD_RES (4)..(4): D-arginine
  MOD_RES (6)..(6): D-arginine
FARRRRRQ

SEQ ID NO: 112 - protein, 8 aa, Artificial Sequence
  Description of Artificial Sequence: Synthetic peptide
  MOD_RES (2)..(2): L-naphthylalanine
FARRRRRQ

SEQ ID NO: 113 - protein, 9 aa, Artificial Sequence
  Description of Artificial Sequence: Synthetic peptide
  MOD_RES (5)..(5): L-naphthylalanine
  MOD_RES (8)..(8): Norleucine
RRRRAFDXC

SEQ ID NO: 114 - protein, 5 aa, Artificial Sequence
  Description of Artificial Sequence: Synthetic peptide
  MOD_RES (2)..(2): L-naphthylalanine
FARRR

SEQ ID NO: 115 - protein, 5 aa, Artificial Sequence
  Description of Artificial Sequence: Synthetic peptide
FWRRR

SEQ ID NO: 116 - protein, 5 aa, Artificial Sequence
  Description of Artificial Sequence: Synthetic peptide
  MOD_RES (4)..(4): L-naphthylalanine
RRRAF

SEQ ID NO: 117 - protein, 5 aa, Artificial Sequence
  Description of Artificial Sequence: Synthetic peptide
RRRWF

SEQ ID NO: 118-149 - skipped (32 entries)

SEQ ID NO: 150 - protein, 8 aa, Artificial Sequence
  Description of Artificial Sequence: Synthetic peptide
  MOD_RES (2)..(2): D-phenylalanine
  MOD_RES (3)..(3): L-naphthylalanine
  MOD_RES (5)..(5): D-arginine
  MOD_RES (7)..(7): D-arginine
FFARRRRQ

SEQ ID NO: 151 - protein, 497 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGM

SEQ ID NO: 152 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 153 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 154 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 155 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 156 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 157 - DNA, 25 bp, Artificial Sequence
  Description of Artificial Sequence: Synthetic oligonucleotide
agaacgtaatcatcagtgggttggc

SEQ ID NO: 158 - DNA, 25 bp, Artificial Sequence
  Description of Artificial Sequence: Synthetic oligonucleotide
acgtaatcatcagtgggttggctct

SEQ ID NO: 159 - DNA, 25 bp, Artificial Sequence
  Description of Artificial Sequence: Synthetic oligonucleotide
ttggcaacatcctctgcagctgaag

SEQ ID NO: 160 - DNA, 19 bp, Artificial Sequence
  Description of Artificial Sequence: Synthetic oligonucleotide
gcaacatcctctgcagctg

SEQ ID NO: 161 - DNA, 25 bp, Artificial Sequence
  Description of Artificial Sequence: Synthetic oligonucleotide
tcaggcttggcaacatcctctgcag

SEQ ID NO: 162 - DNA, 757 bp, Homo sapiens
tcatatcagatgctcaaggctggcagctacccccttcttgagagtccaagaacctggagcagaaataatttttatgtatttttggattaatgaatgttaaaaacagactcagctgtttctttccttttactactaccagttgctcccatgctgctccaccaggccctgtttcggatgccaactggcccactccccaagcacttgcccccagcttgcgaccattggcactgggagggcctggcttctgggctgatgggtcagttgggccttcataaacactcacctggctggctttgccttccaggaggaagctggctgaagcaagggtgtggaattttaaatgtgtgcacagtctggaaaactgtcagaatcagttttcccataaaagggtgggctagcattgcagctgcatttgggaccattcaaatctgtcactctcttgtgtatattcctgtgctattaaatatatcagggcagtgcatgtaaatcatcctgatatatttaatatatttattatattgtcccccgaggtggggacagtgagtgagttctcttagtccccccagagctggttgttaaagagcctggcacctacccgctctcacttcatctgtgtcatctctgcacactccagcccactttctgccttcagccattgagtggaagctgccccaggcccttaccaggtgcagatgcccaatcttgatgcccagccatcagaactgtgagccaaataaacctttttctgtataaatta

SEQ ID NO: 163 - DNA, 10 bp, Artificial Sequence
  Description of Artificial Sequence: Synthetic oligonucleotide
acactcggtt

SEQ ID NO: 164 - DNA, 10 bp, Artificial Sequence
  Description of Artificial Sequence: Synthetic oligonucleotide
cactcggttt

SEQ ID NO: 165 - DNA, 10 bp, Artificial Sequence
  Description of Artificial Sequence: Synthetic oligonucleotide
actcggttta

SEQ ID NO: 166 - DNA, 10 bp, Artificial Sequence
  Description of Artificial Sequence: Synthetic oligonucleotide
ctcggtttat

SEQ ID NO: 167 - DNA, 10 bp, Artificial Sequence
  Description of Artificial Sequence: Synthetic oligonucleotide
tcggtttatt

SEQ ID NO: 168 - DNA, 10 bp, Artificial Sequence
  Description of Artificial Sequence: Synthetic oligonucleotide
cggtttattt

SEQ ID NO: 169 - DNA, 10 bp, Artificial Sequence
  Description of Artificial Sequence: Synthetic oligonucleotide
ggtttatttg

SEQ ID NO: 170 - DNA, 10 bp, Artificial Sequence
  Description of Artificial Sequence: Synthetic oligonucleotide
gtttatttgg

SEQ ID NO: 171 - DNA, 10 bp, Artificial Sequence
  Description of Artificial Sequence: Synthetic oligonucleotide
tttatttgga

SEQ ID NO: 172 - DNA, 10 bp, Artificial Sequence
  Description of Artificial Sequence: Synthetic oligonucleotide
ttatttggaa

SEQ ID NO: 173 - DNA, 10 bp, Artificial Sequence
  Description of Artificial Sequence: Synthetic oligonucleotide
tatttggaaa

SEQ ID NO: 174 - DNA, 10 bp, Artificial Sequence
  Description of Artificial Sequence: Synthetic oligonucleotide
atttggaaaa

SEQ ID NO: 175 - DNA, 10 bp, Artificial Sequence
  Description of Artificial Sequence: Synthetic oligonucleotide
tttggaaaaa

SEQ ID NO: 176 - DNA, 10 bp, Artificial Sequence
  Description of Artificial Sequence: Synthetic oligonucleotide
ttggaaaaag

SEQ ID NO: 177 - DNA, 15 bp, Artificial Sequence
  Description of Artificial Sequence: Synthetic oligonucleotide
tcttgacactcggtt

SEQ ID NO: 178 - DNA, 15 bp, Artificial Sequence
  Description of Artificial Sequence: Synthetic oligonucleotide
cttgacactcggttt

SEQ ID NO: 179 - DNA, 15 bp, Artificial Sequence
  Description of Artificial Sequence: Synthetic oligonucleotide
ttgacactcggttta

SEQ ID NO: 180 - DNA, 15 bp, Artificial Sequence
  Description of Artificial Sequence: Synthetic oligonucleotide
tgacactcggtttat

SEQ ID NO: 181 - DNA, 15 bp, Artificial Sequence
  Description of Artificial Sequence: Synthetic oligonucleotide
gacactcggtttatt

SEQ ID NO: 182 - DNA, 15 bp, Artificial Sequence
  Description of Artificial Sequence: Synthetic oligonucleotide
acactcggtttattt

SEQ ID NO: 183 - DNA, 15 bp, Artificial Sequence
  Description of Artificial Sequence: Synthetic oligonucleotide
cactcggtttatttg

SEQ ID NO: 184 - DNA, 15 bp, Artificial Sequence
  Description of Artificial Sequence: Synthetic oligonucleotide
actcggtttatttgg

SEQ ID NO: 185 - DNA, 15 bp, Artificial Sequence
  Description of Artificial Sequence: Synthetic oligonucleotide
ctcggtttatttgga

SEQ ID NO: 186 - DNA, 15 bp, Artificial Sequence
  Description of Artificial Sequence: Synthetic oligonucleotide
tcggtttatttggaa

SEQ ID NO: 187 - DNA, 15 bp, Artificial Sequence
  Description of Artificial Sequence: Synthetic oligonucleotide
cggtttatttggaaa

SEQ ID NO: 188 - DNA, 15 bp, Artificial Sequence
  Description of Artificial Sequence: Synthetic oligonucleotide
ggtttatttggaaaa

SEQ ID NO: 189 - DNA, 15 bp, Artificial Sequence
  Description of Artificial Sequence: Synthetic oligonucleotide
gtttatttggaaaaa

SEQ ID NO: 190 - DNA, 15 bp, Artificial Sequence
  Description of Artificial Sequence: Synthetic oligonucleotide
tttatttggaaaaag

SEQ ID NO: 191 - DNA, 15 bp, Artificial Sequence
  Description of Artificial Sequence: Synthetic oligonucleotide
ttatttggaaaaaga

SEQ ID NO: 192 - DNA, 15 bp, Artificial Sequence
  Description of Artificial Sequence: Synthetic oligonucleotide
tatttggaaaaagac

SEQ ID NO: 193 - DNA, 15 bp, Artificial Sequence
  Description of Artificial Sequence: Synthetic oligonucleotide
atttggaaaaagaca

SEQ ID NO: 194 - DNA, 15 bp, Artificial Sequence
  Description of Artificial Sequence: Synthetic oligonucleotide
tttggaaaaagacat

SEQ ID NO: 195 - DNA, 15 bp, Artificial Sequence
  Description of Artificial Sequence: Synthetic oligonucleotide
ttggaaaaagacata

SEQ ID NO: 196 - DNA, 15 bp, Artificial Sequence
  Description of Artificial Sequence: Synthetic oligonucleotide
tggaaaaagacatat

SEQ ID NO: 197 - DNA, 20 bp, Artificial Sequence
  Description of Artificial Sequence: Synthetic oligonucleotide
ggtagtcttgacactcggtt

SEQ ID NO: 198 - DNA, 20 bp, Artificial Sequence
  Description of Artificial Sequence: Synthetic oligonucleotide
gtagtcttgacactcggttt

SEQ ID NO: 199 - DNA, 20 bp, Artificial Sequence
  Description of Artificial Sequence: Synthetic oligonucleotide
tagtcttgacactcggttta

SEQ ID NO: 200 - DNA, 20 bp, Artificial Sequence
  Description of Artificial Sequence: Synthetic oligonucleotide
agtcttgacactcggtttat

SEQ ID NO: 201 - DNA, 20 bp, Artificial Sequence
  Description of Artificial Sequence: Synthetic oligonucleotide
gtcttgacactcggtttatt

SEQ ID NO: 202 - DNA, 20 bp, Artificial Sequence
  Description of Artificial Sequence: Synthetic oligonucleotide
tcttgacactcggtttattt

SEQ ID NO: 203 - DNA, 20 bp, Artificial Sequence
  Description of Artificial Sequence: Synthetic oligonucleotide
cttgacactcggtttatttg

SEQ ID NO: 204 - DNA, 20 bp, Artificial Sequence
  Description of Artificial Sequence: Synthetic oligonucleotide
ttgacactcggtttatttgg

SEQ ID NO: 205 - DNA, 20 bp, Artificial Sequence
  Description of Artificial Sequence: Synthetic oligonucleotide
tgacactcggtttatttgga

SEQ ID NO: 206 - DNA, 20 bp, Artificial Sequence
  Description of Artificial Sequence: Synthetic oligonucleotide
gacactcggtttatttggaa

SEQ ID NO: 207 - DNA, 20 bp, Artificial Sequence
  Description of Artificial Sequence: Synthetic oligonucleotide
acactcggtttatttggaaa

SEQ ID NO: 208 - DNA, 20 bp, Artificial Sequence
  Description of Artificial Sequence: Synthetic oligonucleotide
cactcggtttatttggaaaa

SEQ ID NO: 209 - DNA, 20 bp, Artificial Sequence
  Description of Artificial Sequence: Synthetic oligonucleotide
actcggtttatttggaaaaa

SEQ ID NO: 210 - DNA, 20 bp, Artificial Sequence
  Description of Artificial Sequence: Synthetic oligonucleotide
ctcggtttatttggaaaaag

SEQ ID NO: 211 - DNA, 20 bp, Artificial Sequence
  Description of Artificial Sequence: Synthetic oligonucleotide
tcggtttatttggaaaaaga

SEQ ID NO: 212 - DNA, 20 bp, Artificial Sequence
  Description of Artificial Sequence: Synthetic oligonucleotide
cggtttatttggaaaaagac

SEQ ID NO: 213 - DNA, 20 bp, Artificial Sequence
  Description of Artificial Sequence: Synthetic oligonucleotide
ggtttatttggaaaaagaca

SEQ ID NO: 214 - DNA, 20 bp, Artificial Sequence
  Description of Artificial Sequence: Synthetic oligonucleotide
gtttatttggaaaaagacat

SEQ ID NO: 215 - DNA, 20 bp, Artificial Sequence
  Description of Artificial Sequence: Synthetic oligonucleotide
tttatttggaaaaagacata

SEQ ID NO: 216 - DNA, 20 bp, Artificial Sequence
  Description of Artificial Sequence: Synthetic oligonucleotide
ttatttggaaaaagacatat

SEQ ID NO: 217 - DNA, 20 bp, Artificial Sequence
  Description of Artificial Sequence: Synthetic oligonucleotide
tatttggaaaaagacatatt

SEQ ID NO: 218 - DNA, 20 bp, Artificial Sequence
  Description of Artificial Sequence: Synthetic oligonucleotide
atttggaaaaagacatattt

SEQ ID NO: 219 - DNA, 20 bp, Artificial Sequence
  Description of Artificial Sequence: Synthetic oligonucleotide
tttggaaaaagacatattta

SEQ ID NO: 220 - DNA, 20 bp, Artificial Sequence
  Description of Artificial Sequence: Synthetic oligonucleotide
ttggaaaaagacatatttaa

SEQ ID NO: 221 - DNA, 25 bp, Artificial Sequence
  Description of Artificial Sequence: Synthetic oligonucleotide
cggtagtcttgacactcggtttatt

SEQ ID NO: 222 - DNA, 25 bp, Artificial Sequence
  Description of Artificial Sequence: Synthetic oligonucleotide
ggtagtcttgacactcggtttattt

SEQ ID NO: 223 - DNA, 25 bp, Artificial Sequence
  Description of Artificial Sequence: Synthetic oligonucleotide
gtagtcttgacactcggtttatttg

SEQ ID NO: 224 - DNA, 25 bp, Artificial Sequence
  Description of Artificial Sequence: Synthetic oligonucleotide
tagtcttgacactcggtttatttgg

SEQ ID NO: 225 - DNA, 25 bp, Artificial Sequence
  Description of Artificial Sequence: Synthetic oligonucleotide
agtcttgacactcggtttatttgga

SEQ ID NO: 226 - DNA, 25 bp, Artificial Sequence
  Description of Artificial Sequence: Synthetic oligonucleotide
gtcttgacactcggtttatttggaa

SEQ ID NO: 227 - DNA, 25 bp, Artificial Sequence
  Description of Artificial Sequence: Synthetic oligonucleotide
tcttgacactcggtttatttggaaa

SEQ ID NO: 228 - DNA, 25 bp, Artificial Sequence
  Description of Artificial Sequence: Synthetic oligonucleotide
cttgacactcggtttatttggaaaa

SEQ ID NO: 229 - DNA, 25 bp, Artificial Sequence
  Description of Artificial Sequence: Synthetic oligonucleotide
ttgacactcggtttatttggaaaaa

SEQ ID NO: 230 - DNA, 25 bp, Artificial Sequence
  Description of Artificial Sequence: Synthetic oligonucleotide
tgacactcggtttatttggaaaaag

SEQ ID NO: 231 - DNA, 25 bp, Artificial Sequence
  Description of Artificial Sequence: Synthetic oligonucleotide
gacactcggtttatttggaaaaaga

SEQ ID NO: 232 - DNA, 25 bp, Artificial Sequence
  Description of Artificial Sequence: Synthetic oligonucleotide
acactcggtttatttggaaaaagac

SEQ ID NO: 233 - DNA, 25 bp, Artificial Sequence
  Description of Artificial Sequence: Synthetic oligonucleotide
cactcggtttatttggaaaaagaca

SEQ ID NO: 234 - DNA, 25 bp, Artificial Sequence
  Description of Artificial Sequence: Synthetic oligonucleotide
actcggtttatttggaaaaagacat

SEQ ID NO: 235 - DNA, 25 bp, Artificial Sequence
  Description of Artificial Sequence: Synthetic oligonucleotide
ctcggtttatttggaaaaagacata

SEQ ID NO: 236 - DNA, 25 bp, Artificial Sequence
  Description of Artificial Sequence: Synthetic oligonucleotide
tcggtttatttggaaaaagacatat

SEQ ID NO: 237 - DNA, 25 bp, Artificial Sequence
  Description of Artificial Sequence: Synthetic oligonucleotide
cggtttatttggaaaaagacatatt

SEQ ID NO: 238 - DNA, 25 bp, Artificial Sequence
  Description of Artificial Sequence: Synthetic oligonucleotide
ggtttatttggaaaaagacatattt

SEQ ID NO: 239 - DNA, 25 bp, Artificial Sequence
  Description of Artificial Sequence: Synthetic oligonucleotide
gtttatttggaaaaagacatattta

SEQ ID NO: 240 - DNA, 25 bp, Artificial Sequence
  Description of Artificial Sequence: Synthetic oligonucleotide
tttatttggaaaaagacatatttaa

SEQ ID NO: 241 - DNA, 25 bp, Artificial Sequence
  Description of Artificial Sequence: Synthetic oligonucleotide
ttatttggaaaaagacatatttaat

SEQ ID NO: 242 - DNA, 30 bp, Artificial Sequence
  Description of Artificial Sequence: Synthetic oligonucleotide
cggtagtcttgacactcggtttatttggaa

SEQ ID NO: 243 - DNA, 30 bp, Artificial Sequence
  Description of Artificial Sequence: Synthetic oligonucleotide
ggtagtcttgacactcggtttatttggaaa

SEQ ID NO: 244 - DNA, 30 bp, Artificial Sequence
  Description of Artificial Sequence: Synthetic oligonucleotide
gtagtcttgacactcggtttatttggaaaa

SEQ ID NO: 245 - DNA, 30 bp, Artificial Sequence
  Description of Artificial Sequence: Synthetic oligonucleotide
tagtcttgacactcggtttatttggaaaaa

SEQ ID NO: 246 - DNA, 30 bp, Artificial Sequence
  Description of Artificial Sequence: Synthetic oligonucleotide
agtcttgacactcggtttatttggaaaaag

SEQ ID NO: 247 - DNA, 30 bp, Artificial Sequence
  Description of Artificial Sequence: Synthetic oligonucleotide
gtcttgacactcggtttatttggaaaaaga

SEQ ID NO: 248 - DNA, 30 bp, Artificial Sequence
  Description of Artificial Sequence: Synthetic oligonucleotide
tcttgacactcggtttatttggaaaaagac

SEQ ID NO: 249 - DNA, 30 bp, Artificial Sequence
  Description of Artificial Sequence: Synthetic oligonucleotide
cttgacactcggtttatttggaaaaagaca

SEQ ID NO: 250 - DNA, 30 bp, Artificial Sequence
  Description of Artificial Sequence: Synthetic oligonucleotide
ttgacactcggtttatttggaaaaagacat

SEQ ID NO: 251 - DNA, 30 bp, Artificial Sequence
  Description of Artificial Sequence: Synthetic oligonucleotide
tgacactcggtttatttggaaaaagacata

SEQ ID NO: 252 - DNA, 30 bp, Artificial Sequence
  Description of Artificial Sequence: Synthetic oligonucleotide
gacactcggtttatttggaaaaagacatat

SEQ ID NO: 253 - DNA, 30 bp, Artificial Sequence
  Description of Artificial Sequence: Synthetic oligonucleotide
acactcggtttatttggaaaaagacatatt

SEQ ID NO: 254 - DNA, 30 bp, Artificial Sequence
  Description of Artificial Sequence: Synthetic oligonucleotide
cactcggtttatttggaaaaagacatattt

SEQ ID NO: 255 - DNA, 30 bp, Artificial Sequence
  Description of Artificial Sequence: Synthetic oligonucleotide
actcggtttatttggaaaaagacatattta

SEQ ID NO: 256 - DNA, 30 bp, Artificial Sequence
  Description of Artificial Sequence: Synthetic oligonucleotide
ctcggtttatttggaaaaagacatatttaa

SEQ ID NO: 257 - DNA, 30 bp, Artificial Sequence
  Description of Artificial Sequence: Synthetic oligonucleotide
tcggtttatttggaaaaagacatatttaat

SEQ ID NO: 258 - DNA, 25 bp, Artificial Sequence
  Description of Artificial Sequence: Synthetic oligonucleotide
gctgagtctgtttttaacattcatt

SEQ ID NO: 259 - DNA, 25 bp, Artificial Sequence
  Description of Artificial Sequence: Synthetic oligonucleotide
tgagtctgtttttaacattcattaa

SEQ ID NO: 260 - DNA, 25 bp, Artificial Sequence
  Description of Artificial Sequence: Synthetic oligonucleotide
agtctgtttttaacattcattaatc

SEQ ID NO: 261 - DNA, 25 bp, Artificial Sequence
  Description of Artificial Sequence: Synthetic oligonucleotide
tctgtttttaacattcattaatcca

SEQ ID NO: 262 - DNA, 24 bp, Artificial Sequence
  Description of Artificial Sequence: Synthetic oligonucleotide
aatttatacagaaaaaggtttatt

SEQ ID NO: 263 - DNA, 25 bp, Artificial Sequence
  Description of Artificial Sequence: Synthetic oligonucleotide
atttatacagaaaaaggtttatttg

SEQ ID NO: 264 - DNA, 25 bp, Artificial Sequence
  Description of Artificial Sequence: Synthetic oligonucleotide
ttatacagaaaaaggtttatttggc

SEQ ID NO: 265 - DNA, 24 bp, Artificial Sequence
  Description of Artificial Sequence: Synthetic oligonucleotide
tacagaaaaaggtttatttggctc

SEQ ID NO: 266 - DNA, 10 bp, Homo sapiens
tgtgagccaa

SEQ ID NO: 267 - DNA, 10 bp, Homo sapiens
gtgagccaaa

SEQ ID NO: 268 - DNA, 10 bp, Homo sapiens
tgagccaaat

SEQ ID NO: 269 - DNA, 10 bp, Homo sapiens
gagccaaata

SEQ ID NO: 270 - DNA, 10 bp, Homo sapiens
agccaaataa

SEQ ID NO: 271 - DNA, 10 bp, Homo sapiens
gccaaataaa

SEQ ID NO: 272 - DNA, 10 bp, Homo sapiens
ccaaataaac

SEQ ID NO: 273 - DNA, 10 bp, Homo sapiens
caaataaacc

SEQ ID NO: 274 - DNA, 10 bp, Homo sapiens
aaataaacct

SEQ ID NO: 275 - DNA, 10 bp, Homo sapiens
aataaacctt

SEQ ID NO: 276 - DNA, 10 bp, Homo sapiens
ataaaccttt

SEQ ID NO: 277 - DNA, 10 bp, Homo sapiens
taaacctttt

SEQ ID NO: 278 - DNA, 10 bp, Homo sapiens
aaaccttttt

SEQ ID NO: 279 - DNA, 10 bp, Homo sapiens
aacctttttc

SEQ ID NO: 280 - DNA, 15 bp, Homo sapiens
agaactgtgagccaa

SEQ ID NO: 281 - DNA, 15 bp, Homo sapiens
gaactgtgagccaaa

SEQ ID NO: 282 - DNA, 15 bp, Homo sapiens
aactgtgagccaaat

SEQ ID NO: 283 - DNA, 15 bp, Homo sapiens
actgtgagccaaata

SEQ ID NO: 284 - DNA, 15 bp, Homo sapiens
ctgtgagccaaataa

SEQ ID NO: 285 - DNA, 15 bp, Homo sapiens
tgtgagccaaataaa

SEQ ID NO: 286 - DNA, 15 bp, Homo sapiens
gtgagccaaataaac

SEQ ID NO: 287 - DNA, 15 bp, Homo sapiens
tgagccaaataaacc

SEQ ID NO: 288 - DNA, 15 bp, Homo sapiens
gagccaaataaacct

SEQ ID NO: 289 - DNA, 15 bp, Homo sapiens
agccaaataaacctt

SEQ ID NO: 290 - DNA, 15 bp, Homo sapiens
gccaaataaaccttt

SEQ ID NO: 291 - DNA, 15 bp, Homo sapiens
ccaaataaacctttt

SEQ ID NO: 292 - DNA, 15 bp, Homo sapiens
caaataaaccttttt

SEQ ID NO: 293 - DNA, 15 bp, Homo sapiens
aaataaacctttttc

SEQ ID NO: 294 - DNA, 15 bp, Homo sapiens
aataaacctttttct

SEQ ID NO: 295 - DNA, 15 bp, Homo sapiens
ataaacctttttctg

SEQ ID NO: 296 - DNA, 15 bp, Homo sapiens
taaacctttttctgt

SEQ ID NO: 297 - DNA, 15 bp, Homo sapiens
aaacctttttctgta

SEQ ID NO: 298 - DNA, 15 bp, Homo sapiens
aacctttttctgtat

SEQ ID NO: 299 - DNA, 15 bp, Homo sapiens
acctttttctgtata

SEQ ID NO: 300 - DNA, 20 bp, Homo sapiens
ccatcagaactgtgagccaa

SEQ ID NO: 301 - DNA, 20 bp, Homo sapiens
catcagaactgtgagccaaa

SEQ ID NO: 302 - DNA, 20 bp, Homo sapiens
atcagaactgtgagccaaat

SEQ ID NO: 303 - DNA, 20 bp, Homo sapiens
tcagaactgtgagccaaata

SEQ ID NO: 304 - DNA, 20 bp, Homo sapiens
cagaactgtgagccaaataa

SEQ ID NO: 305 - DNA, 20 bp, Homo sapiens
agaactgtgagccaaataaa

SEQ ID NO: 306 - DNA, 20 bp, Homo sapiens
gaactgtgagccaaataaac

SEQ ID NO: 307 - DNA, 20 bp, Homo sapiens
aactgtgagccaaataaacc

SEQ ID NO: 308 - DNA, 20 bp, Homo sapiens
actgtgagccaaataaacct

SEQ ID NO: 309 - DNA, 20 bp, Homo sapiens
ctgtgagccaaataaacctt

SEQ ID NO: 310 - DNA, 20 bp, Homo sapiens
tgtgagccaaataaaccttt

SEQ ID NO: 311 - DNA, 20 bp, Homo sapiens
gtgagccaaataaacctttt

SEQ ID NO: 312 - DNA, 20 bp, Homo sapiens
tgagccaaataaaccttttt

SEQ ID NO: 313 - DNA, 20 bp, Homo sapiens
gagccaaataaacctttttc

SEQ ID NO: 314 - DNA, 20 bp, Homo sapiens
agccaaataaacctttttct

SEQ ID NO: 315 - DNA, 20 bp, Homo sapiens
gccaaataaacctttttctg

SEQ ID NO: 316 - DNA, 20 bp, Homo sapiens
ccaaataaacctttttctgt

SEQ ID NO: 317 - DNA, 20 bp, Homo sapiens
caaataaacctttttctgta

SEQ ID NO: 318 - DNA, 20 bp, Homo sapiens
aaataaacctttttctgtat

SEQ ID NO: 319 - DNA, 20 bp, Homo sapiens
aataaacctttttctgtata

SEQ ID NO: 320 - DNA, 20 bp, Homo sapiens
ataaacctttttctgtataa

SEQ ID NO: 321 - DNA, 20 bp, Homo sapiens
taaacctttttctgtataaa

SEQ ID NO: 322 - DNA, 20 bp, Homo sapiens
aaacctttttctgtataaat

SEQ ID NO: 323 - DNA, 20 bp, Homo sapiens
aacctttttctgtataaatt

SEQ ID NO: 324 - DNA, 25 bp, Homo sapiens
gccatcagaactgtgagccaaataa

SEQ ID NO: 325 - DNA, 25 bp, Homo sapiens
ccatcagaactgtgagccaaataaa

SEQ ID NO: 326 - DNA, 25 bp, Homo sapiens
catcagaactgtgagccaaataaac

SEQ ID NO: 327 - DNA, 25 bp, Homo sapiens
atcagaactgtgagccaaataaacc

SEQ ID NO: 328 - DNA, 25 bp, Homo sapiens
tcagaactgtgagccaaataaacct

SEQ ID NO: 329 - DNA, 25 bp, Homo sapiens
cagaactgtgagccaaataaacctt

SEQ ID NO: 330 - DNA, 25 bp, Homo sapiens
agaactgtgagccaaataaaccttt

SEQ ID NO: 331 - DNA, 25 bp, Homo sapiens
gaactgtgagccaaataaacctttt

SEQ ID NO: 332 - DNA, 25 bp, Homo sapiens
aactgtgagccaaataaaccttttt

SEQ ID NO: 333 - DNA, 25 bp, Homo sapiens
actgtgagccaaataaacctttttc

SEQ ID NO: 334 - DNA, 25 bp, Homo sapiens
ctgtgagccaaataaacctttttct

SEQ ID NO: 335 - DNA, 25 bp, Homo sapiens
tgtgagccaaataaacctttttctg

SEQ ID NO: 336 - DNA, 25 bp, Homo sapiens
gtgagccaaataaacctttttctgt

SEQ ID NO: 337 - DNA, 25 bp, Homo sapiens
tgagccaaataaacctttttctgta

SEQ ID NO: 338 - DNA, 25 bp, Homo sapiens
gagccaaataaacctttttctgtat

SEQ ID NO: 339 - DNA, 25 bp, Homo sapiens
agccaaataaacctttttctgtata

SEQ ID NO: 340 - DNA, 25 bp, Homo sapiens
gccaaataaacctttttctgtataa

SEQ ID NO: 341 - DNA, 25 bp, Homo sapiens
ccaaataaacctttttctgtataaa

SEQ ID NO: 342 - DNA, 25 bp, Homo sapiens
caaataaacctttttctgtataaat

SEQ ID NO: 343 - DNA, 25 bp, Homo sapiens
aaataaacctttttctgtataaatt

SEQ ID NO: 344 - DNA, 25 bp, Homo sapiens
aataaacctttttctgtataaatta

SEQ ID NO: 345 - DNA, 30 bp, Homo sapiens
gccatcagaactgtgagccaaataaacctt

SEQ ID NO: 346 - DNA, 30 bp, Homo sapiens
ccatcagaactgtgagccaaataaaccttt

SEQ ID NO: 347 - DNA, 30 bp, Homo sapiens
catcagaactgtgagccaaataaacctttt

SEQ ID NO: 348 - DNA, 30 bp, Homo sapiens
atcagaactgtgagccaaataaaccttttt

SEQ ID NO: 349 - DNA, 30 bp, Homo sapiens
tcagaactgtgagccaaataaacctttttc

SEQ ID NO: 350 - DNA, 30 bp, Homo sapiens
cagaactgtgagccaaataaacctttttct

SEQ ID NO: 351 - DNA, 30 bp, Homo sapiens
agaactgtgagccaaataaacctttttctg

SEQ ID NO: 352 - DNA, 30 bp, Homo sapiens
gaactgtgagccaaataaacctttttctgt

SEQ ID NO: 353 - DNA, 30 bp, Homo sapiens
aactgtgagccaaataaacctttttctgta

SEQ ID NO: 354 - DNA, 30 bp, Homo sapiens
actgtgagccaaataaacctttttctgtat

SEQ ID NO: 355 - DNA, 30 bp, Homo sapiens
ctgtgagccaaataaacctttttctgtata

SEQ ID NO: 356 - DNA, 30 bp, Homo sapiens
tgtgagccaaataaacctttttctgtataa

SEQ ID NO: 357 - DNA, 30 bp, Homo sapiens
gtgagccaaataaacctttttctgtataaa

SEQ ID NO: 358 - DNA, 30 bp, Homo sapiens
tgagccaaataaacctttttctgtataaat

SEQ ID NO: 359 - DNA, 30 bp, Homo sapiens
gagccaaataaacctttttctgtataaatt

SEQ ID NO: 360 - DNA, 30 bp, Homo sapiens
agccaaataaacctttttctgtataaatta

SEQ ID NO: 361 - DNA, 25 bp, Homo sapiens
aatgaatgttaaaaacagactcagc

SEQ ID NO: 362 - DNA, 25 bp, Homo sapiens
ttaatgaatgttaaaaacagactca

SEQ ID NO: 363 - DNA, 25 bp, Homo sapiens
gattaatgaatgttaaaaacagact

SEQ ID NO: 364 - DNA, 25 bp, Homo sapiens
tggattaatgaatgttaaaaacaga

SEQ ID NO: 365 - DNA, 25 bp, Homo sapiens
aataaacctttttctgtataaatta

SEQ ID NO: 366 - DNA, 25 bp, Homo sapiens
caaataaacctttttctgtataaat

SEQ ID NO: 367 - DNA, 25 bp, Homo sapiens
gccaaataaacctttttctgtataa

SEQ ID NO: 368 - DNA, 24 bp, Homo sapiens
gagccaaataaacctttttctgta

SEQ ID NO: 369 - DNA, 15 bp, Artificial Sequence
  Description of Artificial Sequence: Synthetic oligonucleotide
taacgttgagggcat

SEQ ID NO: 370 - DNA, 25 bp, Homo sapiens
cagagagccaacccactgatgatta

SEQ ID NO: 371 - DNA, 17 bp, Artificial Sequence
  Description of Artificial Sequence: Synthetic oligonucleotide
gctattaccttaaccca

SEQ ID NO: 372 - DNA, 45 bp, Homo sapiens
gccatcagaactgtgagccaaataaacctttttctgtataaatta

SEQ ID NO: 373 - RNA, 17 nt, Artificial Sequence
  Description of Artificial Sequence: Synthetic oligonucleotide
caaaaaaaaaaaaaaaa

SEQ ID NO: 374 - protein, 41 aa, Artificial Sequence
  Description of Artificial Sequence: Synthetic polypeptide
RMRKFKNKGKDTAELRRRRVEVSVELRKAKKDEQILKRRNV

SEQ ID NO: 375 - DNA, 25 bp, Artificial Sequence
  Description of Artificial Sequence: Synthetic oligonucleotide
taatcatcagtgggttggctctctg

SEQ ID NO: 376 - protein, 17 aa, Artificial Sequence
  Description of Artificial Sequence: Synthetic peptide
ELDWDADDIRLQIDNPD

SEQ ID NO: 377 - protein, 7 aa, Artificial Sequence
  Description of Artificial Sequence: Synthetic peptide
PKKKRKV

SEQ ID NO: 378 - RNA, 250 nt, Artificial Sequence
  Description of Artificial Sequence: Synthetic polynucleotide
  misc_feature (1)..(250): This sequence may encompass 150-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 379 - RNA, 90 nt, Artificial Sequence
  Description of Artificial Sequence: Synthetic oligonucleotide
  misc_feature (1)..(90): This sequence may encompass 55-90 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa